(19) United States
(12) Patent Application Publication
(10) Pub. No.: US 2016/0319367 A1
(43) Pub. Date: Nov. 3, 2016

(54) MEANS AND METHODS FOR TYPING A BREAST CANCER PATIENT AND ASSIGNING THERAPY BASED ON THE TYPING

(71) Applicant: STICHTING HET NEDERLANDS KANKER INSTITUUT-ANTONI VAN LEEUWENHOEK ZIEKENHUIS, Amsterdam (NL)

(72) Inventors: Rene Bernards, Amsterdam (NL); Sander Valentijn Maria Canisius, Amsterdam (NL); Willem Teunis Zwart, Amsterdam (NL); Eleonore Marielle Hijmans, Amsterdam (NL); Hendrika Maria Oosterkamp, Amsterdam (NL)

(21) Appl. No.: 15/104,863

(22) PCT Filed: Dec. 17, 2014
(86) PCT No.: PCT/NL2014/050870

§ 371 Date: Jun. 15, 2016

(30) Foreign Application Priority Data
Dec. 17, 2013 (EP) 13197871.0

(51) Int. Cl. C12Q 1/68

(52) U.S. Cl. CPC C12Q 1/6886; C12Q 2600/158; C12Q 2600/106

(57) ABSTRACT

The invention relates to a method of predicting whether a breast cancer patient is or becomes resistant to anti-estrogen directed therapy. More specifically, the invention relates to methods and means for typing of breast cancer cells as having a good or a poor outcome to anti-estrogen directed therapy. The invention further relates to methods of assigning therapy to a breast cancer patient.

### FIELD OF THE INVENTION

The invention relates to the field of oncology. More specifically, the invention relates to a method for typing breast cancer cells. The invention provides means and methods for classification of breast cancer cells and provides a treatment protocol based on the typing of the cells.

### BACKGROUND OF THE INVENTION

About 70% of human breast cancers are ERα positive and depend on this hormone receptor for their proliferation (Harvey et al., 1999. J Clin Oncol 17: 1474-81), rendering ERα an ideal target for endocrine treatment. Tamoxifen is one of the most commonly used drugs in the management of ERα positive breast cancer. In early breast cancer, 5 years of adjuvant treatment with tamoxifen almost halves the rate of disease recurrence and reduces the annual breast cancer death rate by one-third (EBCTCG, 2005. Lancet 365: 1687-717). Despite this adjuvant treatment with tamoxifen, one-third of women still develop recurrent disease in the next 15 years (EBCTCG, 2005. Lancet 365: 1687-717), illustrating that endocrine resistance is a major problem in the management of breast cancer.

Several mechanisms may contribute to tamoxifen resistance. At presentation, not all ERα positive tumours are sensitive to tamoxifen. This intrinsic endocrine resistance can be the result of ERα phosphorylation (Musgrove and Sutherland, 2009. Nat Rev Cancer 9: 631-43; Michalides et al., 2004. Cancer Cell 5: 597-605; Campbell et al., 2001. J Biol Chem 276: 9817-24). In addition, intrinsic tamoxifen resistance is found to correlate with increased levels or activity of ERα co-activators (AIB1), growth factor receptors (EGFR, HER2, IGF1R), kinases (AKT and ERK1/2) or adaptor proteins (BCAR1, c-SRC and PAK1) (Musgrove and Sutherland, 2009. Nat Rev Cancer 9: 631-43; Beelen et al., 2012. Nature reviews Clinical oncology 9: 529-41). Loss of CDK10 expression (Iorns et al., 2008. Cancer Cell 13: 91-104) and loss of insulin-like growth factor binding protein 5 (IGFBP5) expression (Ahn et al., 2010. Cancer Res 70: 3013-3019) can also lead to tamoxifen resistance. Furthermore, high levels of lemur tyrosine kinase-3 (LMTK3) or CUEDC2 protein are associated with tamoxifen resistance (Giamas et al., 2011. Nat Med 17: 715-719; Pan et al., Nat Med 17: 708-149). Acquired endocrine resistance develops in a certain proportion of metastasized ERα-positive breast cancer that was initially sensitive to tamoxifen palliative treatment. One possible mechanism of this resistance is upregulation of the PI3K-mTOR pathway, leading to ligand independent phosphorylation of ERα at serine 167 by S6K1 (Yamnik et al., 2009. J Biol Chem 284: 6361-9; Yue et al., 2007. J Steroid Biochem Mol Biol 106: 102-10; Miller et al., 2010. J Clin Invest 120: 2406-13). It is nevertheless likely that additional mechanisms of unresponsiveness to endocrine treatment play a role, that remain to be identified.

### SUMMARY OF THE INVENTION

To elucidate novel mechanisms of tamoxifen resistance in breast cancer, a shRNA screen was performed in the hormone-dependent human luminal breast cancer cell line ZR-75-1 to identify genes whose suppression can induce tamoxifen resistance. The present inventors surprisingly found that loss of USP9X enhances ERα/chromatin interactions in the presence of tamoxifen, leading to tamoxifen-stimulated gene expression of ERα target genes and cell proliferation.

The present inventors have developed a gene expression profile that is indicative of the activity of USP9X in a breast cancer cell in the presence of tamoxifen. Methods of typing a sample from a breast cancer patient to determine the presence or absence of activity of USP9X, comprise determining the level of expression of genes from the gene profile.

The invention provides a method of typing a sample from a breast cancer patient that is treated with tamoxifen, the method comprising determining a level of expression for USP9X and/or for at least two genes that are selected from Table 1 in a relevant sample from the breast cancer patient, whereby the sample comprises expression products from a cancer cell of the patient; comparing said determined level of expression of USP9X or of the at least two genes to the level of expression of USP9X or the at least two genes in a reference; and typing said sample as being responsive to treatment with tamoxifen or not, based on the comparison of the determined levels of expression.

In a preferred method according to the invention, the sample is typed by determining a level of RNA expression for at least two genes that are selected from Table 1 and comparing said determined RNA level of expression to the level of RNA expression of the at least two genes in a reference. Said reference is preferably a measure of the average level of said at least two genes in at least 10 independent individuals.

A further preferred method according to the invention comprises determining a level of expression of at least five genes from Table 1, more preferred 10 genes from Table 1, more preferred 20 genes from Table 1, more preferred 50 genes from Table 1, more preferred 100 genes from Table 1, more preferred all genes from Table 1.

The invention further provides a method of assigning anti-estrogen receptor-directed therapy (antiER) comprising tamoxifen to a breast cancer patient, comprising typing a sample from the breast cancer patient with a method according to the invention; and assigning anti-estrogen receptor-directed therapy comprising tamoxifen to a patient of which the sample is typed as being responsive to treatment with tamoxifen.

The invention further provides a method of assigning further antiER directed therapy or chemotherapy to a breast cancer patient, comprising typing a sample from the breast cancer patient with a method according to the invention; and assigning chemotherapy to a patient of which the sample is typed as being non-responsive to treatment with tamoxifen.

Said further antiER directed therapy comprises the administration of a selective estrogen receptor modulator not being tamoxifen, an aromatase inhibitor, preferably anastrozole, and/or GnRH or a GnRH-analogue.

Said chemotherapy preferably comprises administration of a platinum agent, preferably cisplatin, and/or a PARP inhibitor, preferably ABT-888.

### BRIEF DESCRIPTION OF THE FIGURES

FIG. 1. shRNA screen identifies USP9X involvement in tamoxifen resistance

(A) Set up of the screen. ZR-75-1 cells stably expressing the murine ecotropic receptor were infected with retroviral supernatants containing a selection of the NM pRS-shRNA library divided in 44 pools—each pool contains 285 distinct short hairpin RNA's against 95 genes—or pRS as control. After puromycin selection 2×105 cells of each pool and control were plated in 15 cm dishes and cultured in DMEM with 1 μM 4OH-tamoxifen for 4-6 weeks. Tamoxifen resistant individual colonies were isolated and one of the rescuing shRNAs was identified as USP9X.

(B) Knockdown of USP9X rescues tamoxifen induced growth arrest. ZR-75-1 cells were infected with the single shRNA against USP9X recovered from the initial screen or pRS-GFP as control. Cells were cultured for 4-6 weeks in the presence of 1 μM 4OH-tamoxifen. When colonies appeared, cells were fixed and stained.

(C) USP9X hit validation. Five independent shRNAs targeting different regions of the USP9X gene were designed and colony formation assays with ZR-75-1 cells infected with each shRNA were performed. Rescue from tamoxifen induced growth arrest by USP9X knockdown was validated by three independent shRNAs.

(D) Knockdown of USP9X decreases USP9X mRNA levels.

(E) Knockdown of USP9X decreases USP9X protein levels.

(F) Knockdown of USP9X rescues tamoxifen-induced growth arrest in T47D cells. T47D cells were infected with the shRNA against USP9X recovered from the initial screen, pRS-USP9X II or pRS-GFP as control. Cells were cultured for 4-6 weeks in the presence of 1 μM 4OHtamoxifen. When colonies appeared cells were fixed and subsequently stained.

FIG. 2. Knockdown of USP9X increases ERα activity

(A) Knockdown of USP9X increases activity on an ERE luciferase reporter in serum supplemented DMEM, in the absence and presence of tamoxifen. Data are represented as mean and standard deviation (SD) of three independent experiments.

(B) Knockdown of USP9X increases ERE luciferase in hormone-deprived, estradiol and 4OH20 tamoxifen treated cells. Data are represented as mean and SD of three independent experiments.

(C) USP9X knockdown in the presence of estradiol increases mRNA levels of the ERα target genes PGR, TFF1 and ERα. Data are represented as mean and SD of three independent experiments.

(D) Knockdown of USP9X increases ERα and PR protein levels in hormone-deprived, estradiol or 4OH-tamoxifen treated cells.

FIG. 3. Physical interactions between USP9X and ERα

(A) Exogenous expressed ERα binds to endogenous USP9X in Phoenix cells. 48 hours after transfection with ERα, immunoprecipitations were performed for either anti-ERα (third lane) or anti-USP9X (fourth lane) and Westerns were stained for ERα and USP9X. The first lane shows 10% input of the whole cell lysate (wcl), the second lane shows immunoprecipitation with normal mouse serum (nms) as control.

(B) Endogenous ERα binds to endogenous USP9X in ZR-75-1 breast cancer cells. (Experimental conditions were identical to A).

FIG. 4. USP9X loss selectively enhances ERα/chromatin interactions upon tamoxifen treatment. Hormone-deprived monoclonal ZR-75-1 cells stably expressing pRS-USP9X or pRS-GFP as control were treated with vehicle (veh), estradiol (E2), or 4OH-tamoxifen (4-OHT) after which ChIP-seq analysis was performed on ERα.

(A) ERα ChIP-seq signal in control cells (top part) and shUSP9X cells (lower part) in the presence of indicated ligand. Tag counts (Y-axis) and genomic locations (X-axis) are indicated.

(B) Heatmap visualization, depicting a vertical alignment of all identified peaks of control (shGFP, left) and USP9XKD (shUSP9X, right) raw read counts of veh, E2, or 4-OHT treated cells. Arrowhead indicates top of the peak and scale bar is indicated.

(C) Read count quantification of data presented in Fig. B showing enrichment of ERα/DNA interactions in the presence of 4-OHT in the shUSP9X cells compared to the control (shGFP) cells. Y-axis: average tag count (arbitrary units). X-axis shows distance from centre of the peak (−2.5 kb, +2.5 kb).

(D) Venn diagrams showing a significant increase in the number of ERα/chromatin binding events in the shUSP9X (right) cells compared to control (shGFP) cells (left) in the presence of 4-OHT, representing a subset of the E2-induced binding patterns. Numbers indicate binding events in each subgroup (veh; dark grey, E2; black, 4-OHT; light gray).

(E) Venn diagrams showing shared and unique peaks for control cells (left hand circles) and shUSP9X cells (right hand circles) under vehicle (left), E2 (middle) and 4-OHT (right) conditions. Numbers indicate binding events in each subgroup.

(F) Genomic distributions of peaks under all tested conditions. Locations are indicated relative to the most proximal genes. 4-OHT shUSP9X unique: unique binding sites in tamoxifen treated shUSP9X cells as compared to tamoxifen-treated control cells.

(G) De novo motif enrichment analysis identified ESR motifs enriched for 4-OHT shUSP9X unique peaks and peaks shared by 4-OHT-treated shGFP control cells and shUSP9X cells.

FIG. 5. USP9X and global gene expression analyses Hormone-deprived monoclonal ZR-75-1 cells stably expressing pRS-USP9X or pRS-GFP as control were treated with vehicle (veh), estradiol (E2), or 4OH-tamoxifen (4-OHT) after which RNA-seq analysis was performed.

(A) Left panel: Venn diagram showing differentially expressed genes in control cells (shGFP) after treatment with E2 (black) or 4-OHT, (grey), as compared to vehicle control (p<0.05). The 1906 differentially expressed genes after 4-OHT treatment represent a subset of the 8794 E2 induced genes. Right panel: Venn diagram showing differentially expressed genes after E2 treatment in control cells (left hand circle) and differentially expressed genes in 4-OHT-treated shUSP9X cells compared to 4-OHT-treated control (right hand circle). Differentially expressed genes in 4-OHT-treated shUSP9X cells represent a subset of E2-responsive genes in the control cells.

(B) Proximal ERα binding events for differentially expressed genes in 4-OHT-treated shUSP9X cells. ERα binding events found only in 4-OHT-treated control cells (left), 4-OHTtreated shUSP9X cells (middle) or shared between both conditions (right) were analysed for proximal binding (<20 kb) to transcription start sites of differential expressed genes in 4-OHTtreated shUSP9X cells and 4-OHT-treated control cells. Y-axis shows absolute number of differentially expressed genes.

(C) Average ERα read count intensity of ERα chromatin binding sites in 4-OHT-treated shUSP9X cells, proximal to (<20 kb) TSS regions of genes, differential expressed between 4-OHT-treated shUSP9X cells and 4-OHT-treated control cells. Y-axis shows average read count (a.u.). X-axis distance from centre of the peak (−2.5 kb, +2.5 kb).

(D) USP9X-differentially expressed genes in the presence of 4-OHT, with proximal ERα binding sites, were analysed for containing genes from the Perou-signature basal and luminal genes. Y-as shows percentage.

(E) Heatmap showing differentially expressed genes between 250 patients with primary ERα-positive breast cancer who received adjuvant tamoxifen. X-axis: patients. Y-axis: genes.

(F) Left panel: A USP9X knockdown tamoxifen gene signature identifies breast cancer patients with poor outcome after adjuvant tamoxifen treatment. Kaplan-Meier survival curves for distant metastasis free survival (DMFS) in a publically available cohort of primary ERα positive breast cancer patients treated with adjuvant tamoxifen (n=250). Middle panel: The USP9X knockdown tamoxifen gene signature is validated in a second cohort of primary ERα positive breast cancer patients treated with adjuvant tamoxifen. Kaplan-Meier survival curves for DMFS in a cohort of primary ERα positive breast cancer patients treated with adjuvant tamoxifen (n=134). Right panel: The USP9X knockdown tamoxifen gene signature does not correlate with outcome in breast cancer patients who did not receive any adjuvant endocrine treatment. Kaplan-Meier survival curves for DMFS in a cohort of primary ERα positive breast cancer patients that did not receive adjuvant endocrine treatment (n=209).

FIG. 6 Validation of the USP9X classifier in independent patient cohorts Validation of the 155 genes USP9X classifier in 5 independent cohorts. Cohort 1, cross-validated predictions (GSE6532; Loi et al., 2007. J Clin Oncol 25: 1239-46); cohort 2 (GSE12093; Zhang et al., 2009. Breast Cancer Res Treat 116: 303-9), cohort 3 (GSE26971; Filipits et al., 2011. Clin Cancer Res 17:6012-20), cohort 4 (GSE9195; Loi et al., 2008. BMC Genomics 9:239), and cohort 5 (GSE17705; Symmans et al., 2010. J Clin Oncol 28:4111-9).

FIG. 7 Validation of a minimal USP9X classifier in independent patient cohorts Validation of a 5 genes USP9X classifier in 5 independent cohorts. Cohort 1, cross-validated predictions (GSE6532; Loi et al., 2007. J Clin Oncol 25: 1239-46); cohort 2 (GSE12093; Zhang et al., 2009. Breast Cancer Res Treat 116: 303-9), cohort 3 (GSE26971; Filipits et al., 2011. Clin Cancer Res 17:6012-20), cohort 4 (GSE9195; Loi et al., 2008. BMC Genomics 9:239), and cohort 5 (GSE17705; Symmans et al., 2010. J Clin Oncol 28:4111-9).

FIG. 8 Performance of 200 random subsets of between 2 and 50 genes from the USP9X, in comparison to the performance of the USP9X signature, and in comparison to the separation of poor survival from good survival.

### DETAILED DESCRIPTION OF THE INVENTION

The term USP9X, as used herein, refers to a ubiquitin specific peptidase 9 which is X-linked. Alternative names for this gene are ubiquitin specific protease 9, X-linked; FAF-X; Drosophila Fat Facets related, X-Linked (DFFRX); Fat Facets Protein-Related, X-Linked; and Ubiquitin Thioesterase.

The term tamoxifen, as used herein, refers to a compounds that bind to the estrogen receptor and that blocks the effects of the hormone estrogen on cancer cells, thereby lowering the chance that breast cancer cells will grow. The term tamoxifen includes the compound tamoxifen ((Z)2-[4-(1,2-diphenyl-1-butenyl) phenoxy]-N, N-dimethylethanamine 2-hydroxy-1,2,3-propanetricarboxylate (1:1)) and variants thereof such as toremifene (2-{4-[(1Z)-4-chloro-1,2-diphenyl-but-1-en-1-yl]phenoxy}-N,N-dimethylethanamine).

The term further antiER directed therapy, as used herein, refers to compounds that modulate the levels of estrogen, the binding of estrogen to the receptor, and/or gene activation by the estrogen receptor. The term further antiER directed therapy excludes tamoxifen. Examples of further antiER directed therapy are provided by selective estrogen receptor modulators apart from tamoxifen, GnRH or a GnRH-analogue and/or of an aromatase inhibitor.

The term typing refers to the classification of a sample from a cancer patient, preferably a breast cancer patient. Said typing is preferably used to predict whether the individual has a high risk of being or becoming resistant to treatment with anti-estrogen receptor-directed therapy selected from tamoxifen, or a low risk of being or becoming resistant to treatment with said anti-estrogen receptor-directed therapy. For this, the level of expression of USP9X or of at least two genes of the set of genes selected from Table 1 is determined in a relevant sample from the individual. Modulation of the level of expression of USP9X, when compared to the level of expression of USP9X in a reference, or modulation of the level of expression of the at least two genes of the set of genes selected from Table 1, compared to the level of expression of the at least two genes of the set of genes selected from Table 1 in a reference, is indicative of a high risk of being or becoming resistant to treatment with tamoxifen.

The term sample, as used herein, refers to a relevant sample comprising expression products from a cancer cell of the patient, preferably a breast cancer cell. Said sample is preferably derived from a primary or metastasized breast cancer. A sample comprising expression products from a cancer cell of an individual suffering from breast cancer is provided after the removal of all or part of a cancerous growth from the individual, for example after biopsy. For example, a sample comprising expression products may be obtained from a needle biopsy sample or from a tissue sample comprising breast cancer cells that was previously removed by surgery. The surgical step of removing a relevant tissue sample, preferably a part of the cancer, from an individual is not part of a method according to the invention. It is preferred that at least 10% of the cells or tissue from which a relevant sample comprising expression products is derived, are breast cancer cells, more preferred at least 20%, more preferred at least 30%, more preferred at least 50%. The sample may have been fixed, for example a formalin-fixed paraffin-embedded (FFPE) sample.

The term expression products, as is used herein, refers to protein expression products or, preferably, RNA expression products. A sample from an individual suffering from breast cancer comprising protein expression products from a cancer of the patient can be obtained in numerous ways, as is known to a skilled person. For example, proteins can be isolated from a sample using, for example, cell disruption and extraction of cellular contents. Suitable methods and means are known in the art, such as dounce pestles and sonication methods. In addition, preferred methods include reagent-based lysis methods using detergents. These methods not only lyse cells but also solubilize proteins. Cell disruption may be followed by methods for enrichment of specific proteins, including subcellular fractionation and depletion of high abundant proteins. Differences in protein expression between a sample from an individual suffering from cancer and a reference sample is studied, for example, by two-dimensional (2D) gel electrophoresis and/or mass spectrometry techniques such as, for example, electrospray ionization and matrix-assisted laser desorption ionization.

The term reference, as used herein, refers to a sample comprising expression products from a related or an unrelated source. A preferred reference comprises expression products from a cancer cell, preferably a breast cancer cell, that is known to be resistant to tamoxifen, from a cancer cell, preferably a breast cancer cell, that is known not to be resistant to tamoxifen, or from a mixture of resistant and non-resistant cancer cells.

The term functionally inactivated, as used herein, refers to an alteration that diminishes or abolishes the expression and/or activity of USP9X. Said alteration can be a genetic alteration, for example an insertion, a point mutation, or, preferably, two or more point mutations in the gene encoding USPX, or an alteration in one of more genes of which the expression product is involved, preferably required, in a USP9X-mediated activity or pathway.

The term target protein, as is used herein, refers to the USP9X protein and/or to a protein product of a gene that is depicted in Table 1.

Methods of Typing a Sample from a Breast Cancer Patient

The present inventors surprisingly found that downregulation of USP9X induces tamoxifen-stimulatory effects on ERα action, leading to resistance to ER-targeting therapy such as tamoxifen. Furthermore, it is shown that a tamoxifen-induced gene expression signature in USP9X knockdown cells can be used to identify cancer patients, especially breast cancer patients, with a poor outcome after tamoxifen treatment and that are likely not to benefit from further tamoxifen treatment.

As is indicated hereinabove, USP9X is an X-linked ubiquitin-specific peptidase. Ubiquitination serves a role in both protein degradation and regulation of protein function. The level of protein ubiquitination is highly regulated by two families of enzymes with opposing activities: the ubiquitin ligases, which add ubiquitin moieties to proteins and deubiquitinating enzymes (DUBs) that remove them. The X-linked deubiquitinase USP9X is a member of the family of DUB enzymes and regulates multiple cellular functions by deubiquitinating and stabilizing its substrates. USP9X has been shown to regulate, amongst others, cell adhesion molecules like 6-catenin and E-cadherin, cell polarity, chromosome segregation, NOTCH, mTOR and TGF-beta signalling as well as apoptosis (Taya et al., (1998) J Cell Biol 142, 1053-1062; Taya et al., (1999) Genes Cells 4, 757-767; Murray et al., (2004) Mol Biol Cell 15, 1591-1599; Théard et al., (2010) EMBO J 29, 1499-1509; Dupont et al., (2009) Cell 136, 123-35).

A shRNA screen in the hormone-dependent human luminal breast cancer cell line ZR-75-1 was employed to identify genes whose suppression can induce tamoxifen resistance. An unexpected role for USP9X in the response to tamoxifen was identified. Loss of expression products of USP9X enhance ERα/chromatin interactions in the presence of tamoxifen, leading to tamoxifen stimulated gene expression of ERα target genes and cell proliferation.

Furthermore, a Tamoxifen-Induced Gene Expression Signature (TIGES) was identified in USP9X knockdown cells that can be used to identify cancer patients, especially breast cancer patients, with a poor outcome after tamoxifen treatment. These genes, as indicated in Tables 1A and 1B, were identified as their relative level of expression was found to be modulated by the presence or absence of USP9X. The term relative is used to indicate that the level of expression was compared to the level of expression in a reference, for example pooled breast cancer samples. The expression of each of the genes depicted in Table 1 correlates with one of two phenotypes. This correlation is represented as a UP or DOWN, indicating upregulation (UP) in the absence of USP9X, and downregulation (DOWN) in the absence of USP9X. For example, upregulation of A1BG or AKT2, and downregulation of ABAT, is indicative of the presence of functionally inactived USP9X.

Methods of classifying a sample from a breast cancer patient that is treated with anti-estrogen receptor-directed therapy selected from tamoxifen according to the presence or absence of a TIGES profile in a breast cancer cell comprise determining the level of expression of at least 2 genes from the gene profile, as indicated in Table 1. The methods of the invention allow classifying a breast cancer sample as likely to become resistant to treatment with anti-estrogen receptor-directed therapy, or not. Therefore, the TIGES profile allows the functional classification of functional inactivation of USP9X in a breast cancer sample. In addition, the TIGES profile can also be used to classify a sample from a breast cancer patient in which a process or signaling pathway involving USP9X is functionally inactivated by functional inactivation of one or more genes encoding other necessary components of the process or pathway.

In a preferred method according to the invention, a level of expression of at least five genes from Table 1 is determined, more preferred a level of expression of at least ten genes from Table 1, more preferred a level of expression of at least twenty genes from Table 1, more preferred a level of expression of at least thirty genes from Table 1, more preferred a level of expression of at least forty genes from Table 1, more preferred a level of expression of at least fifty genes from Table 1, more preferred a level of RNA expression of all two hundred thirty four genes from Table 1.

Said tamoxifen-induced gene expression signature preferably comprises at least two genes from Table 1. Said at least two genes preferably comprise genes with the highest Z-scores. Said at least two genes preferably comprise zinc finger protein 608 ((Z-score −1.008943904) and BUB1 mitotic checkpoint serine/threonine kinase B (Z-score 1.065024239). Said at least two genes preferably comprise zinc finger protein 608 ((Z-score −1.008943904), calpain 2, (m/II) large subunit (Z-score −0.936786567), BUB1 mitotic checkpoint serine/threonine kinase B (Z-score 1.065024239) and centromere protein A (Z-score 1.01511874). Said at least two genes preferably comprise zinc finger protein 608 ((Z-score −1.008943904), calpain 2, (m/II) large subunit (Z-score −0.936786567), FBJ murine osteosarcoma viral oncogene homolog (Z-score −0.920787895), ets homologous factor (Z-score −0.912814779), chondroitin sulfate synthase 1 (Z-score −0.897709367), BUB1 mitotic checkpoint serine/threonine kinase B (Z-score 1.065024239), centromere protein A (Z-score 1.01511874), cell division cycle 45 (Z-score 0.983080062), cell division cycle associated 3 (Z-score 0.97567222), and solute carrier family 25 (mitochondrial thiamine pyrophosphate carrier), member 19 (Z-score 0.974852744).

It is further preferred that said tamoxifen-induced gene expression signature comprises v-myb avian myeloblastosis viral oncogene homolog-like 2 and chondroitin sulfate synthase 1 (P value 1.25E-06 (Loi); 2.32E-05 (Buffa)), v-myb avian myeloblastosis viral oncogene homolog-like 2 and calpain 2, (m/II) large subunit (P value 1.56E-05 (Loi); 4.59E-05 (Buffa)), BUB1 mitotic checkpoint serine/threonine kinase B and calpain 2, (m/II) large subunit (P value 2.67E-06 (Loi); 1.37E-05 (Buffa)), v-myb avian myeloblastosis viral oncogene homolog-like 2, isocitrate dehydrogenase 3 (NAD+) alpha, and calpain 2, (m/II) large subunit (P value 4.77E-08 (Loi); 2.19E-05 (Buffa)), v-myb avian myeloblastosis viral oncogene homolog-like 2, isocitrate dehydrogenase 3 (NAD+) alpha, and calpain 2, (m/II) large subunit (P value 1.56E-06 (Loi); 4.59E-05 (Buffa)), v-myb avian myeloblastosis viral oncogene homolog-like 2, BUB1 mitotic checkpoint serine/threonine kinase B and calpain 2, (m/II) large subunit (P value 4.90E-05 (Loi); 5.42E-06 (Buffa)), chondroitin sulfate synthase 1, BUB1 mitotic checkpoint serine/threonine kinase B and calpain 2, (m/II) large subunit (P value 4.77E-08 (Loi); 2.19E-05 (Buffa)), and/or v-myb avian myeloblastosis viral oncogene homolog-like 2, chondroitin sulfate synthase 1, isocitrate dehydrogenase 3 (NAD+) alpha, and calpain 2, (m/II) large subunit (P value 6.99E-09 (Loi); 1.70E-05 (Buffa)).

More preferably, said signature comprises v-myb avian myeloblastosis viral oncogene homolog-like 2, chondroitin sulfate synthase 1 and isocitrate dehydrogenase 3 (NAD+) alpha (P value 7.75E-06 (Loi); 3.34E-08 (Buffa)), v-myb avian myeloblastosis viral oncogene homolog-like 2, chondroitin sulfate synthase 1, isocitrate dehydrogenase 3 (NAD+) alpha and BUB1 mitotic checkpoint serine/threonine kinase B (P value 8.95E-07 (Loi); 5.78E-08 (Buffa)), v-myb avian myeloblastosis viral oncogene homolog-like 2, chondroitin sulfate synthase 1, isocitrate dehydrogenase 3 (NAD+) alpha, BUB1 mitotic checkpoint serine/threonine kinase B and calpain 2, (m/II) large subunit (P value 6.36E-07 (Loi); 2.28E-07 (Buffa)), and/or chondroitin sulfate synthase 1, isocitrate dehydrogenase 3 (NAD+) alpha, BUB1 mitotic checkpoint serine/threonine kinase B and calpain 2, (m/II) large subunit (P value 3.70E-07 (Loi); 4.46E-07 (Buffa)).

The term P value (Loi) refers to the P-value obtained from a set of 250 ER+ patients that were treated with tamoxifen, as described in Loi et al., 2007. J Clin Oncol 25: 1239-46. The term P value (Buffa) refers to the P-value obtained from a set of 134 ER+ patients that were treated with tamoxifen, as described in Buffa et al., 2011. Cancer Res 71: 5635-45.

A preferred subset comprises calpain 2 (CAPN2). A further preferred subset comprises CAPN2 and BUB1B. A further preferred subset comprises MYBL2, IDH3A, CHSY1, BUB1B, CAPN2. A selection of MYBL2, IDH3A, CHSY1, BUB1B, CAPN2 gave rise to the largest survival differences among 5 independent cohorts that were tested: Cohort 1 (GSE6532; Loi et al., 2007. J Clin Oncol 25: 1239-46); cohort 2 (GSE12093; Zhang et al., 2009. Breast Cancer Res Treat 116: 303-9), cohort 3 (GSE26971; Filipits et al., 2011. Clin Cancer Res 17:6012-20), cohort 4 (GSE9195; Loi et al., 2008. BMC Genomics 9:239), and cohort 5 (GSE17705; Symmans et al., 2010. J Clin Oncol 28:4111-9).

Downregulation of USP9X and/or modulation of the expression of at least two of the genes identified in Table 1, can be monitored at the RNA and protein level. Quantitation of the expression of a gene at the protein level can be either in absolute amount (e.g., μg/ml) or a relative amount (e.g., relative intensity of signals). Usually such procedures are performed by dedicated biochemical assays, such as chromatographic, mass spectrometric or hybridization assays.

Preferred chromatographic assays include Western-blotting assays, following one- or two-dimensional gel electrophoresis.

Hybridization techniques, such as ELISA techniques, immunohistochemistry (IHC), and in situ hybridization, and are very suitable to determine the concentration of a protein in a biological sample. Such techniques preferably involve the production of a calibration curve of label intensity, for example fluorescence intensity, vs. protein concentration, or the use of a competitive ELISA format, wherein known amounts of unlabeled protein are provided in the test. Alternatively, multiple sandwich ELISA can be developed using as second antibody, for instance an antibody raised by peptide immunisation against a second epitope of the target protein (a second synthetic peptide), or against a determinant that is formed by a complex that is formed between the target protein and the antibody.

In this regard, it is preferred to generate a non-natural intermediate, for example an antibody-gene product complex, by reaction of the sample with a first antibody that is directed against the target protein, followed by the application of a detection agent that detects the antibody-target protein complex. It is noted that the antibody-target protein complex does not exist in nature.

Preferred mass spectrometric assays include liquid chromatography-mass spectrometry (LC-MS, or alternatively HPLC-MS), tandem mass spectrometry (MS-MS), matrix assisted laser desorption (MALDI); matrix assisted laser desorption/ionisation time-of-flight (MALDI-TOF), MALDI-Fourier transform ion cyclotron resonance (MALDI-FTICR).

Methods to quantify expression levels of USP9X and/or of at least two of the genes identified in Table 1 at the RNA level are known to a skilled person and include, but are not limited to, Northern blotting, quantitative Polymerase chain reaction (qPCR), also termed real time PCR (rtPCR), microarray analysis and RNA sequencing, preferably next generation sequencing such as whole transcriptome shotgun sequencing. The term qPCR refers to a method that allows amplification of relatively short (usually 100 to 1000 basepairs) of DNA sequences. In order to measure messenger RNA (mRNA), the method involves a reverse transcriptase to convert mRNA into complementary DNA (cDNA) which is then amplified by PCR. The amount of product that is amplified can be quantified using, for example, TaqMan® (Applied Biosystems, Foster City, Calif., USA), Molecular Beacons, Scorpions® and SYBR® Green (Molecular Probes). Methods such as self sustained sequence replication (3SR), loop mediated isothermal amplification (LAMP), strand displacement amplification (SDA), rolling circle amplification (RCA) and quantitative nucleic acid sequence based amplification (qNASBA) can be used as an alternative for qPCR, as is known to the skilled person.

RNA may be isolated from a sample by any technique known in the art, including but not limited to Trizol (Invitrogen; Carlsbad, Calif.), RNAqueous® (Applied Biosystems/Ambion, Austin, Tx), Qiazol® (Qiagen, Hilden, Germany), RNeasy Isolation Kit (Qiagen, Hilden, Germany) Agilent Total RNA Isolation Kits (Agilent; Santa Clara, Calif.), RNA-Bee® (Tel-Test. Friendswood, Tex.), and Maxwell™ Total RNA Purification Kit (Promega; Madison, Wis.). A preferred RNA isolation procedure involves the use of Qiazol® (Qiagen, Hilden, Germany). A further preferred RNA isolation procedure involves the use of the Qiagen RNeasy FFPE RNA isolation Kits (Qiagen, Hilden, Germany). RNA can be extracted from a whole sample or from a portion of a sample generated from the cell sample by, for example, section or laser dissection.

A preferred method for determining a level of RNA expression is microarray analysis. For microarray analysis, a hybridization mixture is prepared by extracting and labelling of RNA. The extracted RNA is preferably converted into a labelled sample comprising either complementary DNA (cDNA) or cRNA using a reverse-transcriptase enzyme and labelled nucleotides. A preferred labelling introduces fluorescently-labelled nucleotides such as, but not limited to, cyanine-3-CTP or cyanine-5-CTP. Examples of labelling methods are known in the art and include Low RNA Input Fluorescent Labelling Kit (Agilent Technologies), MessageAmp Kit (Ambion) and Microarray Labelling Kit (Stratagene).

A labelled sample may comprise two dyes that are used in a so-called two-colour array. For this, the sample is split in two or more parts, and one of the parts is labelled with a first fluorescent dye, while a second part is labelled with a second fluorescent dye. The labelled first part and the labelled second part are independently hybridized to a microarray. The duplicate hybridizations with the same samples allow compensating for dye bias.

More preferably, a sample is labelled with a first fluorescent dye, while a reference, for example a sample from a breast cancer pool or a sample from a relevant cell line or mixture of cell lines, is labelled with a second fluorescent dye (known as dual channel). The labelled sample and the labelled reference are co-hybridized to a microarray.

Even more preferred, a sample is labelled with a fluorescent dye and hybridized to a microarray without a reference (known as single channel).

The labelled sample can be hybridized against the probe molecules that are spotted on the array. A molecule in the labelled sample will bind to its appropriate complementary target sequence on the array. Before hybridization, the arrays are preferably incubated at high temperature with solutions of saline-sodium buffer (SSC), Sodium Dodecyl Sulfate (SDS) and bovine serum albumin (BSA) to reduce background due to nonspecific binding, as is known to a skilled person.

The arrays are preferably washed after hybridization to remove labelled sample that did not hybridize on the array, and to increase stringency of the experiment by reducing cross hybridization of the labelled sample to a partial complementary probe sequence on the array. An increased stringency will substantially reduce non-specific hybridization of the sample, while specific hybridization of the sample is not substantially reduced. Stringent conditions include, for example, washing steps for five minutes at room temperature 0.1× Sodium chloride-Sodium Citrate buffer (SSC)/0.005% Triton X-102. More stringent conditions include washing steps at elevated temperatures, such as 37 degrees Celsius, 45 degrees Celsius, or 65 degrees Celsius, either or not combined with a reduction in ionic strength of the buffer to 0.05×SSC or 0.01×SSC as is known to a skilled person.

Image acquisition and data analysis can subsequently be performed to produce an image of the surface of the hybridised array. For this, the slide can be dried and placed into a laser scanner to determine the amount of labelled sample that is bound to a target spot. Laser excitation yields an emission with characteristic spectra that is indicative of the labelled sample that is hybridized to a probe molecule. In addition, the amount of labelled sample can be quantified.

The level of expression, preferably mRNA expression levels of genes depicted in Table 1, is preferably compared to levels of expression of the same genes in a template. A template is preferably an RNA sample isolated from a tissue of a healthy individual, preferably comprising breast cells. A preferred template comprises a RNA sample from a relevant cell line or mixture of cell lines. The RNA from a cell line or cell line mixture can be produced in-house or obtained from a commercial source such as, for example, Stratagene Human Reference RNA. A further preferred template comprises RNA isolated and pooled from normal breast tissue that is adjacent to the cancer tissue.

A more preferred template comprises an RNA sample from an individual suffering from breast cancer, more preferred from multiple individuals suffering from breast cancer. It is preferred that said multiple samples are pooled from more than 10 individuals, more preferred more than 20 individuals, more preferred more than 30 individuals, more preferred more than 40 individuals, most preferred more than 50 individuals. A most preferred template comprises a pooled RNA sample that is isolated from tissue comprising breast cancer cells from multiple individuals suffering from breast cancer.

As an alternative, a static template can be generated which enables performing single channel hybridizations. A preferred static template is calculated by measuring the median/mean background-subtracted level of expression (for example green-median/MeanSignal or red-median/MeanSignal) of a gene across 1-5 hybridization replicates of a probe sequence. The level of expression may be normalized as is known by a skilled person. Subsequently, a log transformation of each gene/probe gene signal is generated. With this transformation, the variance is stabilized (as with linear values as the signal gets higher the variance also increases; it compresses the range of data) and it makes the data more normally distributed, which allows statistics to be applied to the data. The signal intensity measurements obtain a distribution that is closer to a normal distribution with the variation being independent of the magnitude, allowing statistics to be applied to the data.

Typing of a sample can be performed in various ways. In one method, a coefficient is determined that is a measure of a similarity or dissimilarity of a sample with said template. A number of different coefficients can be used for determining a correlation between the RNA expression level in an RNA sample from an individual and a template. Preferred methods are parametric methods which assume a normal distribution of the data.

The result of a comparison of the determined expression levels with the expression levels of the same genes in at least one template is preferably displayed or outputted to a user interface device, a computer readable storage medium, or a local or remote computer system. The storage medium may include, but is not limited to, a floppy disk, an optical disk, a compact disk read-only memory (CD-ROM), a compact disk rewritable (CD-RW), a memory stick, and a magneto-optical disk.

The expression data are preferably normalized. Normalization refers to a method for adjusting or correcting a systematic error in the measurements of detected label.

Systemic bias results in variation by inter-array differences in overall performance, which can be due to for example inconsistencies in array fabrication, staining and scanning, and variation between labelled RNA samples, which can be due for example to variations in purity. Systemic bias can be introduced during the handling of the sample in a microarray experiment.

To reduce systemic bias, the determined RNA levels are preferably corrected for background non-specific hybridization and normalized using, for example, Feature Extraction software (Agilent Technologies). Other methods that are or will be known to a person of ordinary skill in the art, such as a dye swap experiment (Martin-Magniette et al., Bioinformatics 21:1995-2000 (2005)) can also be applied to normalize differences introduced by dye bias. Normalization of the expression levels results in normalized expression values.

Conventional methods for normalization of array data include global analysis, which is based on the assumption that the majority of genetic markers on an array are not differentially expressed between samples [Yang et al., Nucl Acids Res 30: 15 (2002)]. Alternatively, the array may comprise specific probes that are used for normalization. These probes preferably detect RNA products from housekeeping genes such as glyceraldehyde-3-phosphate dehydrogenase and 18S rRNA levels, of which the RNA level is thought to be constant in a given cell and independent from the developmental stage or prognosis of said cell.

Therefore, a preferred method according to the invention further comprises normalizing the determined RNA levels of said set of at least ten of the genes listed in Table 1 in said sample.

Said normalization preferably comprises previously mentioned global analysis “median centering”, in which the “centers” of the array data are brought to the same level under the assumption that the majority of genes are not changed between conditions (with median being more robust to outliers than the mean). Said normalization preferably comprises Lowess (LOcally WEighted Scatterplot Smoothing) local regression normalization to correct for both print-tip and intensity-dependent bias (for dual channel arrays) or “quantile normalization” (which transforms all the arrays to have a common distribution of intensities) for single channel arrays

In a preferred embodiment, genes are selected of which the RNA expression levels are largely constant between individual tissue samples comprising cancer cells from one individual, and between tissue samples comprising cancer cells from different individuals. It will be clear to a skilled artisan that the RNA levels of said set of normalization genes preferably allow normalization over the whole range of RNA levels. An example of a set of normalization genes is provided in WO 2008/039071, which is hereby incorporated by reference.

The levels of expression of genes from the TIGES signature in a sample of a patient are compared to the levels of expression of the same genes in a reference. Said comparison may result in an index score indicating a similarity of the determined expression levels in a sample of a patient with the expression levels in the reference. For example, an index can be generated by determining a fold change/ratio between the median value of gene expression across samples that have been typed as being responsive to treatment with tamoxifen and the median value of gene expression across samples that are typed as being non-responsive to treatment with tamoxifen. The significance of this fold change/ratio as being significant between the two respective groups can be tested primarily in an ANOVA (Analysis of variance) model. Univariate p-values can be calculated in the model and after multiple correction testing (Benjamini & Hochberg, 1995, JRSS, B, 57, 289-300) can be used as a threshold for determining significance that the gene expression shows a clear difference between the groups. Multivariate analysis may also be performed in adding covariates such as hormone expression, tumor stage/grade/size into the ANOVA model. Significant genes can be imputed into a prediction model such as Diagonal Linear Discriminant analysis (DLDA) to determine the minimal and most reliable group of gene signals that can predict the factor (response to therapy).

As an alternative, an index can be determined by Pearson correlation between the expression levels of the genes in a sample of a patient and the expression levels in one or more breast cancer samples that are known to respond to tamoxifen, and the average expression levels in one or more breast cancer samples that are known not to respond to tamoxifen. The resultant Pearson scores can be used to provide an index score. Said score may vary between +1, indicating a prefect similarity, and −1, indicating a reverse similarity. Preferably, an arbitrary threshold is used to type samples as being responsive or as not being responsive. More preferably, samples are classified as responsive or as not responsive based on the respective highest similarity measurement. A similarity score is preferably displayed or outputted to a user interface device, a computer readable storage medium, or a local or remote computer system.

### Methods of Assigning Treatment to a Breast Cancer Patient

The present invention further provides a method of assigning treatment to a breast cancer patient, the method comprising typing a sample from the breast cancer patient with a method according to the invention, and assigning treatment comprising tamoxifen to a patient of which the sample is typed as being responsive to treatment with tamoxifen.

Tamoxifen and tamoxifen derivatives such as toremifene, are known antagonistic compounds of the estrogen receptor. Methods for providing tamoxifen and/or toremifene to an individual in need thereof suffering from breast are known in the art. For example, tamoxifen may be administered at 20 to 200 mg/kg per day, for example as Tamoxifen Citrate Tablets USP for oral administration. Toremifene similarly can be administered as toremifene citrate at 10 to 800 mg/d orally.

The present invention further provides a method of not assigning tamoxifen-comprising therapy to a breast cancer patient, comprising typing a sample from the breast cancer patient with a method according to the invention; and not assigning tamoxifen to a patient of which the sample is typed as being non-responsive to treatment with tamoxifen. Said method preferably comprises the assignment of further antiER directed therapy and/or chemotherapy to a breast cancer patient of which the sample is typed as being non-responsive to treatment with tamoxifen.

Said further antiER directed therapy comprises selective estrogen receptor modulators (SERM), not including tamoxifen, GnRH or a GnRH-analogue and/or of an aromatase inhibitor.

A preferred non-tamoxifen SERM is provided by fulvestrant (7α,17β)-7-{9-[(4,4,5,5,5-pentafluoropentyl)sulfinyl]nonyl}estra-1,3,5(10)-triene-3,17-diol), which is an estrogen receptor antagonist with no agonist effects, which works by down-regulating the estrogen receptor. It is administered as a once-monthly injection at 500 mg.

A further preferred non-tamoxifen SERM is provided by raloxifene ([6-hydroxy-2-(4-hydroxyphenyl)-benzothiophen-3-yl]-[4-[2-(1-piperidyl)ethoxy]phenyl]-methanone). It is an estrogen receptor antagonist in breast cells, including breast cancer cells. It can be orally administered at 60-240 mg/kg/day.

Yet a further preferred non-tamoxifen SERM is provided by lasofoxifene ((5R,6S)-6-phenyl-5-[4-(2-pyrrolidin-1-ylethoxy)phenyl]-5,6,7,8-tetrahydronaphthalen-2-ol). It is an estrogen receptor antagonist in breast cells, including breast cancer cells. It can be orally administered at 0.001 mg/kg-1.0 mg/kg/day.

A further preferred antiER directed therapy comprises the administration of an aromatase inhibitor. These non-steroidal inhibitors inhibit the synthesis of estrogen via reversible competition for the aromatase enzyme. Preferred aromatase inhibitors include anastrozole (2,2′-[5-(1H-1,2,4-triazol-1-ylmethyl)-1,3-phenylene]bis(2-methylpropanenitrile) and exemestane (6-Methylideneandrosta-1,4-diene-3,17-dione). Anastrozole can be orally administered at 1.0-10 mg/day. Exemestane can be orally administered at 25-50 mg/day

Yet a further preferred antiER directed therapy comprises the administration of gonadotropin-releasing hormone (GnRH), also known as Luteinizing-hormone-releasing hormone (LHRH) and luliberin. GnRH is a trophic peptide hormone responsible for the release of follicle-stimulating hormone (FSH) and luteinizing hormone (LH) from the anterior pituitary. GnRH is synthesized and released from neurons within the hypothalamus. The peptide belongs to gonadotropin-releasing hormone family. Administration of GnRH lowers the levels of oestrogen and progesterone, resulting in estrogen levels that resemble that of a menopausal or post-menopausal woman.

As is known to the skilled person, a GnRH-analogue, for example Leuprolide, is a synthetic peptide drug that is modeled after the human GnRH. A GnRH-analogue is designed to interact with the GnRH receptor and modify the release of pituitary gonadotropins FSH and LH for therapeutic purposes. The synthetic hormone is preferably injected (1 and 3 month depot injections are available) or prescribed as nasal spray. However, the nasal spray is rarely used, because a constant and regular drug level is difficult to maintain.

Yet a further preferred therapy comprises chemotherapy, which includes the use of a chemotherapeutic agent such as an alkylating agent such as nitrogen mustard, e.g. cyclophosphamide, mechlorethamine or mustine, uramustine or uracil mustard, melphalan, chlorambucil, ifosfamide; a nitrosourea such as carmustine, lomustine, streptozocin; an alkyl sulfonate such as busulfan, an ethylenime such as thiotepa and analogues thereof, a hydrazine/triazine such as dacarbazine, altretamine, mitozolomide, temozolomide, altretamine, procarbazine, dacarbazine and temozolomide, which are capable of causing DNA damage; an intercalating agent such as a platinum agent like cisplatin, carboplatin, nedaplatin, oxaliplatin and satraplatin; an antibiotic such as an anthracycline such as doxorubicin, daunorubicin, epirubicin and idarubicin; mitomycin-C, dactinomycin, bleomycin, adriamycin, mithramycin; an antimetabolite such as capecitabine and 5-fluorouracil, gemcitabine, a folate analogue such as methotrexate, hydroxyurea, mercaptopurine, thioguanine; a mitostatic agent such as eribulin, ixabepilone, irinotecan, vincristine, mitoxantrone, vinorelbine and a taxane such as paclitaxel and docetaxel; an inhibitor of the enzyme poly ADP ribose polymerase (PARP), a receptor tyrosine kinase inhibitor such as gefitinib, erlotinib, EKB-569, lapatinib, CI-1033, cetuximab, panitumumab, PKI-166, AEE788, sunitinib, sorafenib, dasatinib, nilotinib, pazopanib, vandetaniv, cediranib, afatinib, motesanib, CUDC-101, and imatinib mesylate; and kinase inhibitors such as a MEK inhibitor including CKI-27, RO-4987655, RO-5126766, PD-0325901, WX-554, AZD-8330, G-573, RG-7167, SF-2626, GDC-0623, RO-5068760, and AD-GL0001; a B-RAF inhibitor including CEP-32496, vemurafenib, GSK-2118436, ARQ-736, RG-7256, XL-281, DCC-2036, GDC-0879, AZ628, and an antibody fragment EphB4/Raf inhibitor; a serine/threonine kinase receptor inhibitor, including an Alk-1 inhibitor such as crizotinib, ASP-3026, LDK378, AF802, and CEP37440, and combinations thereof.

Said chemotherapy is preferably selected from a platinum agent like cisplatin, carboplatin, oxaliplatin and satraplatin; taxane including paclitaxel and docetaxel, a PARP inhibitor, doxorubicin, daunorubicin, epirubicin, cyclophosphamide, 5-fluorouracil, gemcitabine, eribulin, ixabepilone, methotrexate, mitomycin-C, mitoxantrone, vinorelbine, thiotepa, vincristine, capecitabine, a receptor tyrosine kinase inhibitor and/or irinotecan, and combinations thereof.

A preferred PARP inhibitor includes 3-aminobenzamide, 4-(3-(1-(cyclopropanecarbonyl)piperazine-4-carbonyl)-4-fluorobenzyl)phthalazin-1(2H)-one (AZD-2281), 8-fluoro-2-{4-[(methylamino)methyl]phenyl}-1,3,4,5-tetrahydro-6H-pyrrolo[4,3,2-ef][2]benzazepin-6-one phosphate (1:1) (AG014699), 2-[(2R)-2-Methylpyrrolidin-2-yl]-1H-benzimidazole-4-carboxamide dihydrochloride benzimidazole carboxamide (ABT-888), and (8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one (BMN-673).

More preferably, said chemotherapy comprises administration of a platinum agent and/or a PARP inhibitor. A most preferred platinum agent is cisplatin. A most preferred PARP inhibitor is ABT-888.

TABLE 1A

| Gene | Direction | Gene name |
|---|---|---|
| A1BG | Up | alpha-1-B glycoprotein |
| ABAT | Down | 4-aminobutyrate aminotransferase |
| AKT2 | Up | v-akt murine thymoma viral oncogene homolog 2 |
| ALDH3B1 | Up | aldehyde dehydrogenase 3 family, member B1 |
| AMFR | Up | autocrine motility factor receptor, E3 ubiquitin protein |
| | | ligase |
| ANKRD26 | Down | ankyrin repeat domain 26 |
| AP2S1 | Up | adaptor-related protein complex 2, sigma 1 subunit |
| ARHGAP35 | Down | Rho GTPase activating protein 35 |
| ASCL1 | Up | achaete-scute complex homolog 1 (Drosophila) |
| ASXL1 | Up | additional sex combs like 1 (Drosophila) |
| ATG2B | Down | autophagy related 2B |
| ATN1 | Down | atrophin 1 |
| ATP1B1 | Down | ATPase, Na+/K+ transporting, beta 1 polypeptide |
| BNIPL | Down | BCL2/adenovirus E1B 19 kD interacting protein like |
| BRF2 | Up | BRF2, RNA polymerase III transcription initiation |
| | | factor 50 kDa subunit |
| BUB1B | Up | BUB1 mitotic checkpoint serine/threonine kinase B |
| C12orf60 | Up | chromosome 12 open reading frame 60 |
| C17orf58 | Up | chromosome 17 open reading frame 58 |
| C19orf70 | Up | chromosome 19 open reading frame 70 |
| C1orf122 | Up | chromosome 1 open reading frame 122 |
| C22orf13 | Up | Chromosom22 open reading frame 3 |
| C2CD2L | Down | C2CD2-like |
| C8orf33 | Up | chromosome 8 open reading frame 33 |
| C9orf117 | Down | chromosome 9 open reading frame 117 |
| CACNG4 | Up | calcium channel, voltage-dependent, gamma subunit 4 |
| CACYBP | Up | calcyclin binding protein |
| CALCOCO1 | Down | calcium binding and coiled-coil domain 1 |
| CAP2 | Down | CAP, adenylate cyclase-associated protein, 2 (yeast) |
| CAPN2 | Down | calpain 2, (m/II) large subunit |
| CAPN8 | Down | calpain 8 |
| CAV2 | Down | caveolin 2 |
| CCDC117 | Up | coiled-coil domain containing 117 |
| CCDC47 | Up | coiled-coil domain containing 47 |
| CCDC51 | Up | coiled-coil domain containing 51 |
| CCDC57 | Up | coiled-coil domain containing 57 |
| CCDC88C | Up | coiled-coil domain containing 88C |
| CD7 | Up | cluster of differentiation 7 |
| CDC45 | Up | cell division cycle 45 |
| CDCA3 | Up | cell division cycle associated 3 |
| CELSR2 | Down | cadherin, EGF LAG seven-pass G-type receptor 2 |
| CENPA | Up | centromere protein A |
| CENPT | Up | centromere protein T |
| CERS1 | Up | ceramide synthase 1 |
| CHCHD4 | Up | coiled-coil-helix-coiled-coil-helix domain containing 4 |
| CHSY1 | Down | chondroitin sulfate synthase 1 |
| CHTOP | Down | chromatin target of PRMT1 |
| CIC | Down | capicua transcriptional repressor |
| CISH | Down | cytokine inducible SH2-containing protein |
| CLIC1 | Up | chloride intracellular channel 1 |
| COL18A1 | Down | collagen, type XVIII, alpha 1 |
| COPE | Up | coatomer protein complex, subunit epsilon |
| CORO1B | Up | coronin, actin binding protein, 1B |
| CRADD | Up | CASP2 and RIPK1 domain containing adaptor with |
| | | death domain |
| CREB3L4 | Down | cAMP responsive element binding protein 3-like 4 |
| CRTC2 | Down | CREB regulated transcription coactivator 2 |
| CSK | Up | c-src tyrosine kinase |
| CTNNBL1 | Up | catenin, beta like 1 |
| CTNND2 | Up | catenin (cadherin-associated protein), delta 2 |
| CYB5D1 | Down | cytochrome b5 domain containing 1 |
| DCAF10 | Down | DDB1 and CUL4 associated factor 10 |
| DDX49 | Up | DEAD (Asp-Glu-Ala-Asp) box polypeptide 49 |
| DEGS2 | Down | delta(4)-desaturase, sphingolipid 2 |
| DHRS3 | Up | dehydrogenase/reductase (SDR family) member 3 |
| DPAGT1 | Down | dolichyl-phosphate (UDP-N-acetylglucosamine) N- |
| | | acetylglucosaminephosphotransferase 1 (GlcNAc-1-P |
| | | transferase) |
| DVL3 | Up | dishevelled segment polarity protein 3 |
| E2F1 | Up | E2F transcription factor 1 |
| EFNA1 | Down | ephrin-A1 |
| EHF | Down | ets homologous factor |
| EIF3B | Up | eukaryotic translation initiation factor 3, subunit B |
| ELK1 | Up | ELK1, member of ETS oncogene family |
| ERCC1 | Down | excision repair cross-complementing rodent repair |
| | | deficiency, complementation group 1 (includes |
| | | overlapping antisense sequence) |
| ESR1 | Down | estrogen receptor 1 |
| ESRP2 | Up | epithelial splicing regulatory protein 2 |
| ETNK2 | Up | ethanolamine kinase 2 |
| FAM104A | Up | family with sequence similarity 104, member A |
| FAM114A1 | Down | family with sequence similarity 114, member A1 |
| FAM120A | Down | family with sequence similarity 120A |
| FAM126A | Down | family with sequence similarity 126, member A |
| FKBP4 | Up | FK506 binding protein 4, 59 kDa |
| FLT4 | Down | fms-related tyrosine kinase 4 |
| FOS | Down | FBJ murine osteosarcoma viral oncogene homolog |
| FUK | Up | fucokinase |
| GANC | Up | glucosidase, alpha; neutral C |
| GAPDH | Up | glyceraldehyde-3-phosphate dehydrogenase |
| GCET2 | Up | germinal center expressed transcript 2 |
| GGPS1 | Down | geranylgeranyl diphosphate synthase 1 |
| GNG7 | Down | guanine nucleotide binding protein (G protein), gamma 7 |
| H2AFJ | Up | H2A histone family, member J |
| H3F3B | Up | H3 histone, family 3B (H3.3B) |
| H3F3C, | Up | H3 histone, family 3C (H3.3C) |
| H3F3B | | H3 histone, family 3B (H3.3B) |
| HDAC11 | Up | histone deacetylase 11 |
| HIGD2A | Up | HIG1 hypoxia inducible domain family, member 2A |
| HIST1H2AG | Up | histone cluster 1, H2ag |
| HIST1H2BK | Up | histone cluster 1, H2bk |
| HIST1H3B | Up | histone cluster 1, H3b |
| HIST1H4I | Up | histone cluster 1, H4i |
| HMBOX1 | Down | homeobox containing 1 |
| HMG20B | Up | high mobility group 20B |
| HNRNPA2B1 | Down | heterogeneous nuclear ribonucleoprotein A2/B1 |
| HR | Up | hair growth associated |
| HSP90AB1 | Up | heat shock protein 90 kDa alpha (cytosolic), class B |
| | | member 1 |
| HSPB8 | Up | heat shock 22 kDa protein 8 |
| ICAM3 | Up | intercellular adhesion molecule 3 |
| IDH3A | Up | isocitrate dehydrogenase 3 (NAD+) alpha |
| IGFBP4 | Down | insulin-like growth factor binding protein 4 |
| ITPR1 | Down | inositol 1,4,5-trisphosphate receptor, type 1 |
| ITPRIPL2 | Down | inositol 1,4,5-trisphosphate receptor interacting protein- |
| | | like 2 |
| KDM4B | Down | lysine (K)-specific demethylase 4B |
| KIAA0430 | Down | KIAA0430 |
| KIAA1737 | Down | KIAA1737 |
| KRT8 | Up | keratin 8 |
| LAPTM4B | Up | lysosomal protein transmembrane 4 beta |
| LEF1 | Down | lymphoid enhancer-binding factor 1 |
| LETM1 | Up | leucine zipper-EF-hand containing transmembrane |
| | | protein 1 |
| LGALS2 | Up | lectin, galactoside-binding, soluble, 2 |
| LIN37 | Up | lin-37 homolog (C. elegans) |
| LYST | Down | lysosomal trafficking regulator |
| MAFG | Up | v-maf avian musculoaponeurotic fibrosarcoma oncogene |
| | | homolog G |
| MAN2C1 | Down | mannosidase, alpha, class 2C, member 1 |
| MANEAL | Up | mannosidase, endo-alpha-like |
| MAPK13 | Up | mitogen-activated protein kinase 13 |
| MAPT | Down | microtubule-associated protein tau |
| MDH1 | Up | malate dehydrogenase 1, NAD (soluble) |
| MDM2 | Up | MDM2 oncogene, E3 ubiquitin protein ligase |
| MFAP3L | Up | microfibrillar-associated protein 3-like |
| MMP25 | Up | matrix metallopeptidase 25 |
| MOCS2 | Up | molybdenum cofactor synthesis 2 |
| MRPS14 | Down | mitochondrial ribosomal protein S14 |
| MST1P9 | Down | macrophage stimulating 1 (hepatocyte growth factor- |
| | | like) pseudogene 9 |
| MYBL2 | Up | v-myb avian myeloblastosis viral oncogene homolog-like 2 |
| MYO5C | Down | myosin V-C |
| NDUFAF3 | Up | NADH dehydrogenase (ubiquinone) complex I, assembly |
| | | factor 3 |
| NDUFB9 | Up | NADH dehydrogenase (ubiquinone) 1 beta subcomplex, |
| | | 9, 22 kDa |
| NDUFS8 | Up | NADH dehydrogenase (ubiquinone) Fe—S protein 8, |
| | | 23 kDa (NADH-coenzyme Q reductase) |
| NKAIN1 | Up | Na+/K+ transporting ATPase interacting 1 |
| NPB | Up | neuropeptide B |
| NUF2 | Up | NUF2, NDC80 kinetochore complex component |
| OLFML2A | Down | olfactomedin-like 2A |
| PALLD | Down | palladin, cytoskeletal associated protein |
| PAN2 | Up | PAN2 poly(A) specific ribonuclease subunit homolog |
| | | (S. cerevisiae) |
| PARP6 | Down | poly (ADP-ribose) polymerase family, member 6 |
| PBXIP1 | Down | pre-B-cell leukemia homeobox interacting protein 1 |
| PCYT2 | Up | phosphate cytidylyltransferase 2, ethanolamine |
| PDCD6IP | Down | programmed cell death 6 interacting protein |
| PDCL3 | Up | phosducin-like 3 |
| PDF | Up | peptide deformylase (mitochondrial) |
| PDZK1 | Down | PDZ domain containing 1 |
| PFKFB3 | Down | 6-phosphofructo-2-kinase/fructose-2,6-biphosphatase 3 |
| PGR | Down | progesterone receptor |
| PHB | Up | prohibitin |
| PIN1 | Up | peptidylprolyl cis/trans isomerase, NIMA-interacting 1 |
| PIP | Down | prolactin-induced protein |
| PLA2G15 | Up | phospholipase A2, group XV |
| POGK | Down | pogo transposable element with KRAB domain |
| POLK | Down | polymerase (DNA directed) kappa |
| PPFIA1 | Up | protein tyrosine phosphatase, receptor type, f |
| | | polypeptide (PTPRF), interacting protein (liprin), alpha 1 |
| PPP1R12B | Down | protein phosphatase 1, regulatory subunit 12B |
| PRDX1 | Up | peroxiredoxin 1 |
| PSENEN | Up | presenilin enhancer gamma secretase subunit |
| PSMD5 | Down | proteasome (prosome, macropain) 26S subunit, non- |
| | | ATPase, 5 |
| PTPN6 | Up | protein tyrosine phosphatase, non-receptor type 6 |
| QSOX1 | Down | quiescin Q6 sulfhydryl oxidase 1 |
| RAB11FIP1 | Up | RAB11 family interacting protein 1 (class I) |
| RAB13 | Down | RAB13, member RAS oncogene family |
| RAB7L1 | Up | RAB7, member RAS oncogene family-like 1 |
| RALGPS2 | Down | Ral GEF with PH domain and SH3 binding motif 2 |
| RARA | Up | retinoic acid receptor, alpha |
| RCC1 | Up | regulator of chromosome condensation 1 |
| RGS19 | Up | regulator of G-protein signaling 19 |
| RNASEH2C | Up | ribonuclease H2, subunit C |
| RPL12 | Down | ribosomal protein L12 |
| RPL14 | Down | ribosomal protein L14 |
| RPL3 | Down | ribosomal protein L3 |
| RPLP0P6 | Up | ribosomal protein, large, P0 pseudogene 6 |
| RPS6KB2 | Up | ribosomal protein S6 kinase, 70 kDa, polypeptide 2 |
| RRNAD1 | Down | ribosomal RNA adenine dimethylase domain containing 1 |
| S100A6 | Up | S100 calcium binding protein A6 |
| SCGB2A2 | Down | secretoglobin, family 2A, member 2 |
| SCNN1A | Down | sodium channel, non-voltage-gated 1 alpha subunit |
| SDHB | Up | succinate dehydrogenase complex, subunit B, iron |
| | | sulfur (Ip) |
| SEC11C | Up | SEC11 homolog C (S. cerevisiae) |
| SELL | Up | selectin L |
| SEPT8 | Down | septin 8 |
| SH2B1 | Down | SH2B adaptor protein 1 |
| SIRT7 | Up | sirtuin 7 |
| SLC25A1 | Up | solute carrier family 25 (mitochondrial carrier; citrate |
| | | transporter), member 1 |
| SLC25A19 | Up | solute carrier family 25 (mitochondrial thiamine |
| | | pyrophosphate carrier), member 19 |
| SLC35E2B | Down | solute carrier family 35, member E2B |
| SLC38A1 | Up | solute carrier family 38, member 1 |
| SLC3A2 | Up | solute carrier family 3 (amino acid transporter heavy |
| | | chain), member 2 |
| SLC40A1 | Down | solute carrier family 40 (iron-regulated transporter), |
| | | member 1 |
| SLC4A2 | Up | solute carrier family 4 (anion exchanger), member 2 |
| SLC9A3R1 | Up | solute carrier family 9, subfamily A (NHE3, cation |
| | | proton antiporter 3), member 3 regulator 1 |
| SMARCC2 | Down | SWI/SNF related, matrix associated, actin dependent |
| | | regulator of chromatin, subfamily c, member 2 |
| SPAG9 | Up | sperm associated antigen 9 |
| SRRM2 | Down | serine/arginine repetitive matrix 2 |
| SSH3 | Down | slingshot protein phosphatase 3 |
| SSPN | Down | sarcospan |
| SSR3 | Up | signal sequence receptor, gamma (translocon-associated |
| | | protein gamma) |
| ST3GAL4 | Up | ST3 beta-galactoside alpha-2,3-sialyltransferase 4 |
| SUFU | Up | suppressor of fused homolog (Drosophila) |
| SYT5 | Up | synaptotagmin V |
| TARBP1 | Down | TAR (HIV-1) RNA binding protein 1 |
| TCEB2 | Up | transcription elongation factor B (SIII), polypeptide 2 |
| | | (18 kDa, elongin B) |
| TEX2 | Up | testis expressed 2 |
| TFPT | Up | TCF3 (E2A) fusion partner (in childhood Leukemia) |
| TGIF2 | Up | TGFB-induced factor homeobox 2 |
| THAP11 | Up | THAP domain containing 11 |
| THSD4 | Down | thrombospondin, type I, domain containing 4 |
| TIMP2 | Down | TIMP metallopeptidase inhibitor 2 |
| TMEM170A | Up | transmembrane protein 170A |
| TMEM63C | Down | transmembrane protein 63C |
| TOM1L1 | Up | target of myb1 (chicken)-like 1 |
| TOR1AIP1 | Down | torsin A interacting protein 1 |
| TRAPPC3 | Up | trafficking protein particle complex 3 |
| TRAPPC8 | Down | trafficking protein particle complex 8 |
| TRIM25 | Up | tripartite motif containing 25 |
| TRUB2 | Up | TruB pseudouridine (psi) synthase family member 2 |
| TSKU | Up | tsukushi, small leucine rich proteoglycan |
| TUBA1A | Up | tubulin, alpha 1a |
| TUBA1C | Up | tubulin, alpha 1c |
| TUBA1C, | Up | tubulin, alpha 1c |
| TUBA1A | | tubulin, alpha 1a |
| TXNRD1 | Up | thioredoxin reductase 1 |
| UFD1L | Up | ubiquitin fusion degradation 1 like (yeast) |
| USP5 | Up | ubiquitin specific peptidase 5 (isopeptidase T) |
| UXT | Up | ubiquitously-expressed, prefoldin-like chaperone |
| WBP11 | Up | WW domain binding protein 11 |
| WDR6 | Down | WD repeat domain 6 |
| WWC3 | Down | WWC family member 3 |
| WWP1 | Up | WW domain containing E3 ubiquitin protein ligase 1 |
| XPC | Down | xeroderma pigmentosum, complementation group C |
| ZFP106 | Down | zinc finger protein 106 |
| ZNF302 | Down | zinc finger protein 302 |
| ZNF608 | Down | zinc finger protein 608 |

TABLE 1B

| Probe set | Gene | Z-score | Affymetrix probe set sequences |
|---|---|---|---|
| 229819_at | A1BG | 0.382021523 | CCAACTACAGCTGCGTCTACGTGGA |
| | | | GCTGCGTCTACGTGGACCTAAAGCC |
| | | | TACGTGGACCTAAAGCCACCTTTCG |
| | | | GCTGCGCGAGGGCGAGACGAAGGCC |
| | | | CGAAGGCCGTGAAGACGGTCCGCAC |
| | | | CGAACCTCGAGCTGATCTTCGTGGG |
| | | | CACGCCGGCAACTACAGGTGCCGCT |
| | | | CACACCTTCGAATCGGAGCTCAGCG |
| | | | CTGTGGAGCTCCTGGTGGCAGAAAG |
| | | | GTGCTGTTGGTGTCCTCAGAAGTGC |
| | | | AAGTGCCGGGGATTCTGGACTGGCT |
| 206527_at | ABAT | −0.51888386 | GGATGACCCAGCAGACGTGATGACC |
| | | | AGGAGTTCAGGCCTAATGCTCCCTA |
| | | | CTACCGGATCTTCAACACGTGGCTG |
| | | | CCGTCCAAGAACCTGTTGCTGGCTG |
| | | | GCTGGCTGAGGTCATCAACATCATC |
| | | | TGAGAGGACGAGGCACCTTTTGCTC |
| | | | TCCTTCGATACTCCCGATGATTCCA |
| | | | TGACAAATCCATTCGTTTCCGTCCC |
| | | | CGTCCCACGCTGGTGTTCAGGGATC |
| | | | AAGAAGCCATTTCCACTACAGTGAG |
| | | | ACAGTGAGAAAGCCCGGATCCCAAC |
| 209459_s_at | ABAT | −0.68458777 | TAATGTATCTACATACCTACACCTA |
| | | | ATCTACATACCTACACCTATCTATA |
| | | | ACACCTATCTATATATAAGCTCATG |
| | | | GAAAACCATAGCTAAGTAGCATCGC |
| | | | GTAGCATCGCAGACTTAAGCGTACA |
| | | | AAGCGTACAAAGTGATCTTGTTCAC |
| | | | TCTTGTTCACAAGTAATCTGTTGAC |
| | | | ATCTGTTGACAGTGCCAATAAATGA |
| | | | CATGTCACAATGTAACGGATGACCA |
| | | | CGGATGACCATATGCACAATTCCAT |
| | | | CCTGTGTTAGTCAGTATTCTTAAAT |
| 209460_at | ABAT | −0.72504225 | AGAATTCTCAGCAGAGCTCAAGATT |
| | | | GATTGTAGAAACTCAGCAGAAGCTG |
| | | | GCTGGTAAAAACATGGGGAGCCCGG |
| | | | CTTCCGTGGCCGACAGTCTGGAAAT |
| | | | TGGCCGACAGTCTGGAAATGAATCC |
| | | | GAATCCATCATACATTAGTGCCATA |
| | | | GTGCCATAGAGTTTAGTAACCGTCC |
| | | | TAGTAACCGTCCAGCAAGTGTCATC |
| | | | AACCGTCCAGCAAGTGTCATCACTT |
| | | | AACAAGGTCCAGTAATAGCAAGTCT |
| | | | GTCCAGTAATAGCAAGTCTTAGTAC |
| 236664_at | AKT2 | 0.356840165 | AGGAAATTCACCCGAGGTCGCAGGG |
| | | | GGCCTTGAGTACTCATTTTGGTGCT |
| | | | ATTTTGGTGCTGATTACCTCTCTGC |
| | | | TAAATTGGTAGTTTCCTGCTCTTTT |
| | | | TTTCCTGCTCTTTTTGTGTAATCTT |
| | | | TAATGTGAAGCCTCTGGGGGCTGCC |
| | | | TGCCCTCGTGCACTGATGGTTGTGT |
| | | | GGCAGTGCGATTCCCTTTTAGCTGC |
| | | | TTAGCTGCTGCATGGGGGGAACTCA |
| | | | TTCCATGGGGTAGACCCCTCAACCG |
| | | | TTGACTTGGTTTCGTTTGGTGCTAC |
| 205640_at | ALDH3B1 | 0.505744977 | AAAACCTCCTGGGACTGTTGCAAGG |
| | | | GGGATTGAGGGATTGCTGAGCTGGA |
| | | | TTCTCAGTGGGGTGGCACGGAGCGG |
| | | | GGCAGGTGGGGCTGTGGTTATGCGA |
| | | | GGTTATGCGATAGGGTCTCCCTTCC |
| | | | TGTAACTCTTTATCCTCATGGTGCC |
| | | | TGCCCACTACGAGTCATACTCTTCC |
| | | | GCCAAAGCAGAATGCAGGGTTTCCT |
| | | | GCGGGGGTGCTTGAGAAACCTACAT |
| | | | TATCAACCTACAACTTTAGTCGGGA |
| | | | CAGGGGTGGACCTGAGTTTCGTCTC |
| 202204_s_at | AMFR | 0.3946814 | AATGCAGGTGTCCTGAGCACCACAC |
| | | | GTGGGGGAGGCGCACAGTGTGAGCC |
| | | | CCACGTCGTGGGGTAACATCTGTTA |
| | | | GAACTCTTGGTTCGATACCTGGAGC |
| | | | GGTGTGATGAAGTCACCCCTTTCTG |
| | | | CCTTTCTGTCCCACTACATCTGGGA |
| | | | TACATCTGGGACTGACTTTCCGAGC |
| | | | CAGTCCAAAGCCGGCTTGATTTCCG |
| | | | TTGATTTCCGTGAACTCTGGTGCTC |
| | | | TGCTCCTGCATCTCATGAGTGTGCC |
| | | | GCCTGTGGGTTTGGTCCTTGAACAA |
| 205706_s_at | ANKRD26 | −0.50628649 | GAGAGGCTAGCAGAGGTCAACACCA |
| | | | AGAGCAGATCTTTGTTCACCACTCT |
| | | | CAGTCATGGAGCCACCTTGTGTGGG |
| | | | GAAAACTTAGTGATCTCTACCTCAA |
| | | | TACCTCAAATCCACGGGCTTCAAAT |
| | | | GAACTACTTGAGCAAGATGCAGCAG |
| | | | ACTAGAGAACTCAAAGAAGCTGCTG |
| | | | GAATCTGGATCAATAGCTTCCCCTC |
| | | | TTCCCCTCTAGGGTCTACTGATGAG |
| | | | GGGTCTACTGATGAGTCAAATCTAA |
| | | | TTTATTACTGGGCTGTTTATGTGAC |
| 202120_x_at | AP2S1 | 0.489068174 | ACTTTAAGATCATTTACCGCCGCTA |
| | | | ACAAACTGGCTTACCTGGAGGGCAT |
| | | | GGAGGGCATTCACAACTTCGTGGAG |
| | | | TGGACCTGGTGTTCAACTTCTACAA |
| | | | GGTCGTGGACGAGATGTTCCTGGCT |
| | | | GAAATCCGAGAGACCAGCCAGACGA |
| | | | AAACAGCTGCTGATGCTACAGTCCC |
| | | | TCCTTCCCTCAACTGCCTAGGAGGA |
| | | | GAAGGGACCCAGCTGGGTCTGGGCC |
| | | | CAAGGGAGGAGACTTCACCCCACTT |
| | | | GCCGTTGTCGTGTGATTCCATAAGC |
| 208074_s_at | AP2S1 | 0.499416201 | ATCCAGAACCGGGCAGGCAAGACGC |
| | | | GCGCCTGGCCAAGTGGTACATGCAG |
| | | | GCCAAGTGGTACATGCAGTTTGATG |
| | | | GATCGAGGAGGTGCATGCCGTGGTC |
| | | | GACGCCAAACACACCAACTTTGTGG |
| | | | AAACGAATATTTCCACAATGTCTGT |
| | | | CAATGTCTGTGAACTGGACCTGGTG |
| | | | GACCTGGTGTTCAACTTCTACAAGG |
| | | | TCTACAAGGTTTACACGGTCGTGGA |
| | | | CTGATGCTACAGTCCCTGGAGTGAG |
| | | | GTCCCTGGAGTGAGGGCAGGCGAGC |
| 211047_x_at | AP2S1 | 0.453831888 | ACTTTAAGATCATTTACCGCCGCTA |
| | | | ATGACAACAACCTGGCTTACCTGGA |
| | | | GAGGCCATTCACAACTTCGTGGAGG |
| | | | TGGACCTGGTGTTCAACTTCTACAA |
| | | | GGTCGTGGACGAGATGTTCCTGGCT |
| | | | GAAATCCGAGAGACCAGCCAGACGA |
| | | | AAACAGCTGCTGATGCTACAGTCCC |
| | | | TGGAGTGAGGGCAGGCGAGCCCCAC |
| | | | ACAAGGGAGGAGACTGCACCCCACT |
| | | | GCCGTTGTCGTGTGATGCCATAAGC |
| | | | CTGTGCGTGGAGTCCCCAATAAACC |
| 202045_s_at | ARHGAP35 | −0.47676853 | TGCTGCGACCCAGATTCTTCTGCAG |
| | | | AGGATGTGTCTGTCTTTGTCACGGT |
| | | | GGGTGACATCATAGGAGCAGCTCGC |
| | | | CAGCTCGCTGGCCAGAAGGGGATGG |
| | | | CACACAAAACTTCACAGCAGGCCAG |
| | | | AGCAGGCCAGCTGCAGTGACTTGTC |
| | | | TAGGGTGCGGTGGCCAGGAGGGCCC |
| | | | TCGCTGCTTTCCCGAGGGCAGCGCA |
| | | | GCAGGGATCCGGGGAAGCTGCGGCA |
| | | | CGGCTTCGTGGCTCTGAGGTGTAAC |
| | | | CGGAGGACATCGTCTGTGTCCAGGT |
| 229394_s_at | ARHGAP35 | −0.75549803 | GTGTTAGTAGTCTGGCTGTGTGCCC |
| | | | CTGTGTGCCCAAAATTCTGTTTCGC |
| | | | GTGCCCAAAATTCTGTTTCGCAGCA |
| | | | GTTTCGCAGCAAAAGTGAAGACCTG |
| | | | TGGGTTTTTTGAGGCTCCAACCTGA |
| | | | GGCTCCAACCTGATTAGTGCATGGT |
| | | | CAATGAAGGCTGAGGCATCTCTGAC |
| | | | GGCATCTCTGACTGAGGTGTTTTTG |
| | | | GTACTTGTCTCAATGGGAATGGTGT |
| | | | AAAAGGCCTTATGTGATCTGTATCA |
| | | | GAAAATTTGGAATAGTGCTGCTGCC |
| 209985_s_at | ASCL1 | 0.407298116 | GCCACGGCTGGAGAGACCGAGACCC |
| | | | GAGACCCGGCGCAAGAGAGCGCAGC |
| | | | GAGAGCGCAGCCTTAGTAGGAGAGG |
| | | | GTAGGAGAGGAACGCGAGACGCGGC |
| | | | GAGACGCGGCAGAGCGCGTTCAGCA |
| | | | GCGCGTTCAGCACTGACTTTTGCTG |
| | | | AAACAAGAAGGCGCCAGCGGCAGCC |
| | | | GAAGCCAACCCGCGAAGGGAGGAGG |
| | | | TTTTTTTGCTCCCACTCTAAGAAGT |
| | | | TCCCACTCTAAGAAGTCTCCCGGGG |
| | | | GTCTCCCGGGGATTTTGTATATATT |
| 209987_s_at | ASCL1 | 0.658405716 | GGACGAGCATGACGCGGTGAGCGCC |
| | | | ACTACTCCAACGACTTGAACTCCAT |
| | | | TCGTCGGACGAGGGCTCTTACGACC |
| | | | TTCTCGACTTCACCAACTGGTTCTG |
| | | | GCCCTGGTGCGAATGGACTTTGGAA |
| | | | CAGGGTGATCGCACAACCTGCATCT |
| | | | ACCTGCATCTTTAGTGCTTTCTTGT |
| | | | TTCGCCCGAACTGATGCGCTGCAAA |
| | | | CAACTTCAGCGGCTTTGGCTACAGC |
| | | | AGCGCAACCGCGTCAAGTTGGTCAA |
| | | | CAAGTTGGTCAACCTGGGCTTTGCC |
| 209988_s_at | ASCL1 | 0.632471336 | GTATCTATCCTAACCAGTTCGGGGA |
| | | | CATGTAATGCTATTACCTCTGCATA |
| | | | GATGTGTAGTTCACCTTACAACTGC |
| | | | ACCTTACAACTGCAATTTTCCCTAT |
| | | | GCAATTTTCCCTATGTGGTTTTGTA |
| | | | TGTAAAGAACTCTCCTCATAGGTGA |
| | | | GAGATCAAGAGGCCACCAGTTGTAC |
| | | | CACCAGTTGTACTTCAGCACCAATG |
| | | | AGCACCAATGTGTCTTACTTTATAG |
| | | | ATGCAGCTACTGTCCAAACTCAAAG |
| | | | GCAGCCAGTTGGTTTTGATAGGTTG |
| 213768_s_at | ASCL1 | 0.522864871 | GAAGGGAGCAGCACACGCGTTATAG |
| | | | CGCGTTATAGTAACTCCCATCACCT |
| | | | CACCTCTAACACGCACAGCTGAAAG |
| | | | CGCCCTTTCTTAGAGTGCAGTTCTT |
| | | | CCCACCCCAATAAGCTGTAGACATT |
| | | | TGCTATTCTCAGCCCTTTGAAACTC |
| | | | AACCCCATCGCCAACTAAGCGAGGC |
| | | | GAAGCGCTCAGAACAGTATCTTTGC |
| | | | GTATCTTTGCACTCCAATCATTCAC |
| | | | GCAACTGGGACCTGAGTCAATGCGC |
| | | | TGCAAAAGCAGTGGGCTCCTGGCAG |
| 244519_at | ASXL1 | 0.402859825 | TTAGAAAACTACTCGGATGCTCCAA |
| | | | CTACTCGGATGCTCCAATGACACCA |
| | | | CAATGACACCAAAACAGATTCTGCA |
| | | | TCTCGCATGCCTCAATGCTATGCTA |
| | | | CGCATGCCTCAATGCTATGCTACAT |
| | | | GCCTCAATGCTATGCTACATTCCAA |
| | | | CAATGCTATGCTACATTCCAATTCA |
| | | | TGTTTTATAAACTGCCTGGCCGAAT |
| | | | ATAAACTGCCTGGCCGAATCAGCCT |
| | | | ATCAGCCTTTTCACGCTCAAGGTGT |
| | | | GCCTTTTCACGCTCAAGGTGTGAGC |
| 226684_at | ATG2B | −0.37898028 | TGTAAATGTCATCTCAGCTGGCTCA |
| | | | AGCTGGCTCAGTTATATCTCTAATG |
| | | | ATCTCTAATGTCCCGGGTAGCAGCA |
| | | | CAGCACCTCCCTCTAAAAATATGTT |
| | | | AATATGTTTACTTCGCTGTTTCACT |
| | | | AAATGGCAGCTTCCGATTTCTAGTT |
| | | | TGGTCACCCAGGGCTATTTGCTTTT |
| | | | AGGGGTGTCTAGTTCAGCTTTTATG |
| | | | GTTGATCCATCCTGACTTATTTTAG |
| | | | GACATTGAATTTATCTCACCACAAG |
| | | | GACTGTCTTTGCTAAGTTTCCTAAT |
| 40489_at | ATN1 | −0.45511501 | AAGCGACAAGCCACTGTAGAACCTG |
| | | | AAGCCACTGTAGAACCTGCGATCAA |
| | | | CACTGTAGAACCTGCGATCAAGAGA |
| | | | GTAGAACCTGCGATCAAGAGAGCAC |
| | | | GAACCTGCGATCAAGAGAGCACCAT |
| | | | AGCCAAGAGGGTGCTGCTCAGTTGC |
| | | | CCAAGAGGGTGCTGCTCAGTTGCAG |
| | | | GGTGCTGCTCAGTTGCAGGGCCTCC |
| | | | GCTGCTCAGTTGCAGGGCCTCCGCA |
| | | | CTGCTCAGTTGCAGGGCCTCCGCAG |
| | | | AGTTGCAGGGCCTCCGCAGCTGGAC |
| | | | CAGGGCCTCCGCAGCTGGACAGAGA |
| | | | ACAGAAAGCGCACAGAATCTTGGAC |
| | | | CTTGGACCAGGTCTCTCTTCCTTGT |
| | | | TGGACCAGGTCTCTCTTCCTTGTCC |
| | | | CTGCCCCGTTGGTGTGATTATTTCA |
| 201242_s_at | ATP1B1 | −0.638675 | AGAGCTGATCACAAGCACAAATCTT |
| | | | TGATCACAAGCACAAATCTTTCCCA |
| | | | CTTTCCCACTAGCCATTTAATAAGT |
| | | | AACCTACTAGTCTTGAACAAACTGT |
| | | | AACTGTCATACGTATGGGACCTACA |
| | | | GTATGGGACCTACACTTAATCTATA |
| | | | GGACCTACACTTAATCTATATGCTT |
| | | | ACACTTAATCTATATGCTTTACACT |
| | | | ATATGCTTTACACTAGCTTTCTGCA |
| | | | GCTTTACACTAGCTTTCTGCATTTA |
| | | | GCTTTCTGCATTTAATAGGTTAGAA |
| 201243_s_at | ATP1B1 | −0.70700285 | GGTGATGGGTTGTGTTATGCTTGTA |
| | | | GTTATGCTTGTATTGAATGCTGTCT |
| | | | GAATGCTGTCTTGACATCTCTTGCC |
| | | | CTTGTCCTCCGGTATGTTCTAAAGC |
| | | | TCCGGTATGTTCTAAAGCTGTGTCT |
| | | | AAGCTGTGTCTGAGATCTGGATCTG |
| | | | TCTGAGATCTGGATCTGCCCATCAC |
| | | | GAGGCATCACATGCTGGTGCTGTGT |
| | | | GGTGCTGTGTCTTTATGAATGTTTT |
| | | | GACTGGTGTTAAATGTTGTCTACAG |
| | | | GATCTTGTATTCAGTCAGGTTAAAA |
| 236534_at | BNIPL | −0.65529039 | ACTTTAGCTGTAGAACCTTGGGCAA |
| | | | AACTGGAGGGACTGTGATCCTTCCA |
| | | | GAAGAGGCTTACCTGACAGCCAGCC |
| | | | GAGTCAGCTCATTAAATCTTGAAGA |
| | | | TTTCCTTCTAAGTCATGTCTGCTGC |
| | | | TCTAAGTCATGTCTGCTGCCTGTGA |
| | | | TGCTGCCTGTGAGCCTGGGAAGGAG |
| | | | GAGCCTGGGAAGGAGTGCTTTCAAA |
| | | | GAGTGCTTTCAAAACCTGTATTTTT |
| | | | GCTCGGCCAGAGCTCTGGGTTTTAA |
| | | | CTGGGTTTTAATCCTACTTTAGCTG |
| 218954_s_at | BRF2 | 0.541780241 | GGAGACCCGAGAGAAGGAGCCACCG |
| | | | TCTTGCCACCCTGCATGTTGAAGTC |
| | | | TTGAAGTCCCCGAAGCGGATCTGCC |
| | | | TAGAACAGTATTTGCGTACCCCTCA |
| | | | TTTGCGTACCCCTCAGGAAGTTAGG |
| | | | TAGGGACTTTCAGAGAGCCCAGGCT |
| | | | GATATCCACTGGGAGCACTTCATCC |
| | | | TGTGCTGCTGCGGATGGCTGAGCAG |
| | | | CTGGCCTGGTTACGAGTTCTGAGAC |
| | | | GACTTGACAAACGGTCTGTGGTGAA |
| | | | GTGAAGCACATCGGTGACCTTCTCC |
| 218955_at | BRF2 | 0.673065335 | AGGAACCAAGAGGGGCTCTGCCATT |
| | | | CTCTGCCATTAGTTGGACCCTGGGT |
| | | | GACCCTGGGTCCTGGAGTAAAGTCA |
| | | | GAGATTCCCATCCCTTGGTGTGGGA |
| | | | AGAGCAAGTTGCCTATGTCCATGTT |
| | | | GTTCTGTGAGATGGCTTTCCTCATA |
| | | | GGCTCTTTGCTGCTGGTTTGAATTG |
| | | | GGTTTGAATTGGACACACTGCTGCG |
| | | | CTTCCCTCTGCTTGTGGAGTGGTTG |
| | | | GAACTGGGGAATTCTGGCCCTACGT |
| | | | TATGGTGTCATGAGATCCTCTACCT |
| 203755_at | BUB1B | 1.065024239 | TTCTTTGTGCGGATTCTGAATGCCA |
| | | | TGGGGTTTTTGACACTACATTCCAA |
| | | | GTTAACTAGTCCTGGGGCTTTGCTC |
| | | | GGGGCTTTGCTCTTTCAGTGAGCTA |
| | | | GAGCTAGGCAATCAAGTCTCACAGA |
| | | | GTCTCACAGATTGCTGCCTCAGAGC |
| | | | GGACACATTTAGATGCACTACCATT |
| | | | CACTACCATTGCTGTTCTACTTTTT |
| | | | GGTACAGGTATATTTTGACGTCACT |
| | | | GGCCTTGTCTAACTTTTGTGAAGAA |
| | | | GTTCTCTTATGATCACCATGTATTT |
| 229888_at | C120060 | 0.477972255 | TTCAAAAGTGCCCATACGCCAGTCA |
| | | | GCTAAACAGCAGTAACATCCTTGGG |
| | | | AACATCCTTGGGAGTCTGGAATCTT |
| | | | GAAATTCCCCATCATGAATCTTCAA |
| | | | AGAGCAATCAGATGTCACCACATCT |
| | | | ATCTGAGAGAACCAGAAGTCCTCCA |
| | | | AAAATCCCACAAAGTCAGCAGCAGA |
| | | | GGGACCAATCTTAGAGATCCTCCAA |
| | | | GAAAGCCAGTGACAAGTAGGGATGC |
| | | | GTTTCTAAGATCTTTTGGTGCCAAA |
| | | | GTCATCTGGCAAAACATTTACCTGT |
| 226901_at | C170058 | 0.409662665 | GTACATAACAGTAAGCGCACTAGTC |
| | | | ACAAGGGTCAAAGCCCAGGACAAGT |
| | | | AAGGGCCAGTGTGCAGATGGGTGGA |
| | | | GTACTAAAGGGCTTACTTCAGGCAA |
| | | | GGCAAAAGTGTTCCTGACGTACCAA |
| | | | CTCCCTGCTCCTAGTAATGTATGTT |
| | | | AATGTATGTTTTGTGCTGAACTGGC |
| | | | TGAACTGGCAGCTATCCCAATGTGA |
| | | | CAGACAATGATTTACACAGCTCAGA |
| | | | AGCTCAGATAATTGACCTGTCCAGT |
| | | | GTCCAGTTAACAGATCATTGCTTCA |
| 225823_at | C190070 | 0.42217782 | TCAAGGGAAGTGTGGCTGGGGGCGC |
| | | | CGCCGTCTACCTGGTGTACGACCAG |
| | | | CAGTTCAGCCAGTACGTGTGTCAGC |
| | | | ACGTGTGTCAGCAGACAGGCCTGCA |
| | | | CAAAGATTTACTTTCCCATCCGTGA |
| | | | GTGACTCCTGGAATGCAGGCATCAT |
| | | | TCATGACGGTGATGTCAGCTCTGTC |
| | | | GGGCTGGGAGTATGTGAAGGCGCGC |
| | | | GGCGCGCACCAAGTAGCGAGTCAGC |
| | | | GCCTGCCCCGGCCAGAACGGGCAGG |
| | | | GTGGTCGCTGATGAGGTTCCTCATC |
| 225480_at | C1orf122 | 0.598551981 | GAGGAGATGTTACGGCAGCTGGGCC |
| | | | AGGCGGCTTTCCAAAGGATGCTGGC |
| | | | GACTCTGAACAACTCCCTTCAGTAA |
| | | | CACTGGCAGTGGCTGGTACTTGGCT |
| | | | CTTGGCTCTCAGCCTGGAGTGGCAG |
| | | | AGCTCTGCTAGCAGCTGGGTTCACT |
| | | | AATGCAGCCAATGAATACCCAGTCT |
| | | | ATACCCAGTCTGATTACCCAGATTT |
| | | | AGCAGTGCTCGCCAGAGTGGTCTGG |
| | | | GTCTGGCCTGCTATGGGGGATCCAG |
| | | | GGATCCAGGTGGTGTTACATGTCCA |
| 223039_at | C22orf13 | 0.460898172 | GGGCTTTGTTCATTCTAGCCACGGG |
| | | | GTGCACATGCTGTTAGGGCTGTCAC |
| | | | GGGCTGTCACTAGGGAGTGGCCTTC |
| | | | GAGGGTGGTAACAGCACCTCAGTCC |
| | | | TTAGAAACACTCAGTCTCTGGTCCC |
| | | | TCTGGTCCCAGAGGATGGCTTCTCA |
| | | | TGGCTTCTCAGGGCATGCCACAAGT |
| | | | CATTCTCAAGACTCATCTGCCTAGG |
| | | | AGACACACTGTGTTGCATTCTTGCA |
| | | | ACAGCACATGACACCGACAGCTGCC |
| | | | CCAGCACAGCACCTGAAGCCATGTG |
| 204757_s_at | C2CD2L | −0.65986454 | TATATGTGTGGCTTAGGACCCTCCG |
| | | | GGACCCTCCGTGAACAGATGATAGA |
| | | | ATGATAGAGGGCATCTCTCCCAGGT |
| | | | CTTCTTTTCTGTCCCAGGAGGGTGG |
| | | | CCACTCAGACCAGCACCAGTGTCTG |
| | | | GAGAATGTTGGCAGCTCACAGAGAG |
| | | | TTACCGTTTTTTGTACTTGATGCCT |
| | | | TGTACTTGATGCCTTCTCTGTGAGC |
| | | | CTCTGTGAGCAGTGGCTCTGTGGGA |
| | | | TGATGGAGCCACGCAAGGCTGCACC |
| | | | ATTGCTGTGTGATGGCTTGGAATTT |
| 218187_s_at | C8orf33 | 0.611515101 | GATGCCTGTTGCAAAGTGGACCATG |
| | | | TGGACCATGGTCTAGCAGTAGCATC |
| | | | ATGGTCTAGCAGTAGCATCAGTGTC |
| | | | CTAGCAGTAGCATCAGTGTCAAGGA |
| | | | AACACCCACTACTTAGCAGACTGGG |
| | | | CCACTACTTAGCAGACTGGGAAAAG |
| | | | GAAAGTACTAAATGTCTGATATGCA |
| | | | GGACACATGACCCATGTGACCTTAC |
| | | | CACATGACCCATGTGACCTTACCTA |
| | | | CATGTGACCTTACCTATTATTGGAG |
| | | | ATTGGAGATGGTTCACATTCCTTAC |
| 222551_s_at | C8orf33 | 0.521449961 | GCTATTGGAGCAATCCGAACCCTGC |
| | | | TGCGCAGCAAAAGAACGCCCTTGCC |
| | | | TGGAAGCCGAATGGCGTGAGGCCCT |
| | | | CTGCTGCTTATTCAGCCCAGGTGCA |
| | | | GCAACCTGTAGATGGAGCCACCAGA |
| | | | AAGAGCCAAAGGGTCTGCAGGCCTC |
| | | | TGCAGGCCTCGCTCTATATGGAGAG |
| | | | GGGGTTTGTTTTGAGTGCAGAGCCT |
| | | | CCTTTCCAGGACTTCTGTTGTCAGA |
| | | | TCCCTGGCTGGTCCAAGGATTTGTA |
| | | | CAGATAGGCAAAAGACCCCGTTCGT |
| 231172_at | C9orf117 | −0.51594747 | AGCAGCCAATCGTGTTGCCAACTGT |
| | | | TGTTGCCAACTGTTTGGCGTCCACC |
| | | | GCCGCCATGCTTCTGAGGGGCGGAA |
| | | | TTCAGTAGCGCGGCGTCACAGTGTC |
| | | | GTCACAGTGTCCCTTCGGGACTTGT |
| | | | CCCTTCGGGACTTGTGTGGGACGCT |
| | | | GCTCCAAAACACATCGGCTCATGGC |
| | | | CTTCGGTTGGGAGGCCTTGTTATGC |
| | | | TATGGCCCTGACTTGCGGCGAAAAT |
| | | | GCGAAAATCTGGCAAGTCCTTTCCC |
| | | | CCTCTCCAGCTAATAAAAGTTTTCT |
| 221585_at | CACNG4 | 0.452208385 | CACTGCCATGACCAGGCCGAAGGCA |
| | | | GACCAGGCCGAAGGCAGGGAACGCC |
| | | | AAAGCAAGGCAGCCGTGCTGTTCTA |
| | | | CAAGGCAGCCGTGCTGTTCTAGTTC |
| | | | GCCCCAGAAGTTTCTATCATTCCAT |
| | | | GAAGTTTCTATCATTCCATGGAGAA |
| | | | GCTGTGTTCCAATGAATCCTACCTC |
| | | | TCTTGCCCAGTCCCAGGCAGAGTAA |
| | | | GCCCAGTCCCAGGCAGAGTAAGCAG |
| | | | GGCCCACCTAGGGACCAAGAAAGAG |
| | | | GAAGAAGGGGACGAGCCGGGAGCAA |
| 231737_at | CACNG4 | 0.56182315 | CTTTTTGTCACACAGGATGGCATGT |
| | | | GCATGTGATCCTCAAGACGACGAAC |
| | | | GCCGAGCTACAGGTACCGGCGACGG |
| | | | ACGTGTCGCCCATGGGCCTGAAGAT |
| | | | GCCTGAAGATCACAGGGGCCATCCC |
| | | | CCATGGGGGAGCTGTCCATGTACAC |
| | | | TCCATGTACACGCTGTCCAGGGAGC |
| | | | AGCTTCCTGCAGGTGCATGACTTTT |
| | | | GACTTTTTCCAGCAGGACCTGAAGG |
| | | | AAGGAAGGTTTCCACGTCAGCATGC |
| | | | TCAGCATGCTGAACCGACGGACGAC |
| 62987_r_at | CACNG4 | 0.398600737 | CCGGGCCTTCTCAGCCTTCTCCCCG |
| | | | GGGCCTTCTCAGCCTTCTCCCCGCG |
| | | | TCTCCCCGCGGCCAGCTGGGTCTCC |
| | | | GCGGCCAGCTGGGTCTCCGGGGACC |
| | | | GGCCAGCTGGGTCTCCGGGGACCCT |
| | | | GCCCTGGGCCGCCCATTCCTGGCCC |
| | | | TGGGCCGCCCATTCCTGGCCCTCCC |
| | | | CCCTCCCGCTGCATCTCAGACCTGA |
| | | | GCTGCATCTCAGACCTGACACCCAA |
| | | | TGCATCTCAGACCTGACACCCAACG |
| | | | GCATCTCAGACCTGACACCCAACGG |
| | | | TGGCCTGTGCCCACCTTCTCTCCCT |
| | | | CCTCCCTGGCCTCCAGAGGTGGCGT |
| | | | CCCCACCCCTGTGTGTTTCGCCAGT |
| | | | TACTGGTTTTGGGTTGGTTGTTCTG |
| | | | TGTGCTGGGAGACCGGACCCGGGGC |
| 201381_x_at | CACYBP | 0.669614252 | ATTAGTACCCTGGTCATTTTGTTCA |
| | | | GGGTTATATTGCATTCTCACGTGAA |
| | | | ATCTCTTGAAACCCATCTCTGTGGA |
| | | | AACCCATCTCTGTGGAAGGCAGTTC |
| | | | CAAGGTGGGATTACCTGACCCAGGT |
| | | | AAAGAGAAGCCCTCCTATGACACTG |
| | | | AGCCCTCCTATGACACTGAAACAGA |
| | | | GGAGACACGGAATTTTGAGACTTTA |
| | | | AAAGGCAATGAATTCTCCATTTCCT |
| | | | AAATATGCTTATTAAACACTCCTGC |
| | | | ACACTCCTGCAAAGATGGTTTTATT |
| 201382_at | CACYBP | 0.394076544 | TACTGAAACACATTATGCCTCTGTA |
| | | | ATGCCTCTGTAATTGGGGTTGACAC |
| | | | GGGGTTGACACATGAACAGAATAGC |
| | | | GAATAGCAGACACAATGCATATGAA |
| | | | TATAGATATATTCCAAGCCGCCTGA |
| | | | CAAGCCGCCTGACGATCTAATTGTA |
| | | | GACATTATATGTGACTTAAAACCTA |
| | | | ACTATTGATCAATTTTAACTACATA |
| | | | CCCACCATAACCCAAGGCAAACAAT |
| | | | AAACAATGTATTGACAGGATTCCAA |
| | | | CATGTAAAGATGCTCACCTTGTTCA |
| 210691_s_at | CACYBP | 0.67538078 | GAAGAGTTACTCCATGATTGTGAAC |
| | | | TGAACAATCTCTTGAAACCCATCTC |
| | | | ATCTCTGTGGAAGGCAGTTCAAAAA |
| | | | GACTGATACAGTTCTTATATTGTGT |
| | | | CAAGGTGGGATTACCTGACCCAGGT |
| | | | CTGACCCAGGTTGAAAAGGAGTGCA |
| | | | GAAACAGATCCTAGTGAGGGATTGA |
| | | | TGAAGCGAACCATTAATAAAGCCTG |
| | | | AAAGCCTGGGTGGAATCAAGAGAGA |
| | | | GTAAGGGAATATTGGTGAGCTGCAT |
| | | | AATTTGACAGATAGCTATTTACATA |
| 209002_s_at | CALCOCO1 | −0.66325252 | GCAGTGGCTGAATTTATCCCCTGAA |
| | | | GAGGCCTTCCCCTGTGGGAATAGAA |
| | | | TGGGAATAGAATCGTCCACTCCTAG |
| | | | AGCCCTGGTTGCTTCTGATACACAG |
| | | | TTCTGATACACAGCCACTGCACACA |
| | | | TACCCTCTCTTATTTGGAGTTTCCG |
| | | | TGGAGTTTCCGTTGGTTTACCTGAG |
| | | | TCTCTGGGGTCTGCACAGAGGCAGC |
| | | | CAGTTTCATTGGTTCCTCTTTCTGT |
| | | | GTGCCTTCTGTGAGGAATGGGGGGA |
| | | | GTCCCCCCACAGCAATAAAAGCTTC |
| 212551_at | CAP2 | −0.48832527 | AACTCGGCCTGGTGTTTGACAATGT |
| | | | GAAGTGATCAACTCCCAGGACATTC |
| | | | GGTTGCCACATATACCTCAGTGAAG |
| | | | AGATCGTGAGCGCCAAGTCATCTGA |
| | | | ATGAACATACTTATCCCTCAGGATG |
| | | | TTATGGCCTAACTTCCTGAGAGACC |
| | | | TGAATCCCCCTCTATCAAACAAACA |
| | | | GCCTCCAACGATTCTGTGCTATAGA |
| | | | AGATACAGCACTGTTTCTGGCACGC |
| | | | GCACGCCTCGTGGGCATTTTGAAAT |
| | | | TAACGTTTCCTCATGATTTGCCTTT |
| 212554_at | CAP2 | −0.50081085 | AATCAAGCTCAGTTATTATTTTCCA |
| | | | TTATGTCTTTAACGTTTTCTTATAG |
| | | | TTCTTATAGACTAATTTCCTCTTTT |
| | | | CTTGCTGCTCCTATTTTGTAGTCTT |
| | | | GATGCTTCTTCAGCGTAAGAGTAGC |
| | | | GAGTAGCTATGATATTCCTTTTTAT |
| | | | AAATCTGCAACTTCTTGGATCATAT |
| | | | GTATAATGCTTGCAGGCCCAGTACA |
| | | | ATATATTGTGCCTCTTACAGCCTTT |
| | | | GTGCCTCTTACAGCCTTTGGAATAC |
| | | | AATGCTCATGTACCAAGGTTTTGCT |
| 208683_at | CAPN2 | −0.93678657 | GACACGAGGCCCTTGGCAGGGAATA |
| | | | CAGTCCAAGATTACCATTTCCCATG |
| | | | TCACCTCTGTCGCTTGGGTTAAACA |
| | | | AATCGTTCTCCTTACAATCAAGTTC |
| | | | AATCAAGTTCTTGACCCTATTCGGC |
| | | | TTCGGCCTTATACATCTGGTCTTAC |
| | | | ATCCTGCGCTTGATCAACTGAACCA |
| | | | ATAAGCTGTTTGCCACCTCAAAACT |
| | | | TATGAACTTCACCACCACTAGTGTC |
| | | | ACCACTAGTGTCTGTCCATGGAGTT |
| | | | TGCCTTATCTTCTTCCAAATGTACT |
| 229030_at | CAPN8 | −0.79447996 | ACTGATTATAACCACTCGGGCACCA |
| | | | GATGCCCACGAGATGAGGACAGCCC |
| | | | ACAGCCAGGTGCAGCAGACCATTGC |
| | | | GCGGTATGCGTGCAGCAAGCTTGGC |
| | | | CATCAACTTTGACAGCTTCGTGGCT |
| | | | TCGTGGCTTGTATGATCCGCCTGGA |
| | | | CCTCTTCAAACTATTCAGCCTTCTG |
| | | | TGGTCTGACCCGGGGTTTCGGACAT |
| | | | GGTTTCGGACATCAGTGACACTCCC |
| | | | ACTGGTTGTTCATACCTTTCTTGCC |
| | | | CTTTCTTGCCCTGGGTCTATTTCAG |
| 203323_at | CAV2 | −0.57309235 | ATGAAGCTCATATCCTTTTGAAGGT |
| | | | GAGACATTTCAAAACTGCCCTAGGC |
| | | | CCTAGGCCATTGCAGCATCCTTAGA |
| | | | GATGGGACGCATAATCATTACCTTA |
| | | | ATTACCTTAAAGCATCACCACTCAT |
| | | | AAGCATCACCACTCATTTTGACCAT |
| | | | AAGGTCAATCAGCCTCATGACTTTA |
| | | | GCTATCCTTTCAAACAGCTATTGGC |
| | | | AAGTAACATGACTTCCTTATTTCTG |
| | | | AAATCCAGGCTTTATGTACAAACAT |
| | | | GATGAGCAGACTTCTCGGAATTCAT |
| 203324_s_at | CAV2 | −0.47190989 | AAAGCACACAACGATTATAGTAACT |
| | | | TCCTACAGGCCTATTTAACAAGATG |
| | | | AAATGTTGCTCTAATCAGATTGCTT |
| | | | ATGTAGCTCCCACAAGGTAAACTTC |
| | | | AAACTTCATTGGTAAGATTGCACTG |
| | | | GATTGCACTGTTCTGATTATGTAAG |
| | | | GTTGACACCACTTAGATTTAAAGGC |
| | | | AAGGCAGACAGTTTTGCTTTAGTAC |
| | | | TACCTTTACATATATAGTCACTGGC |
| | | | AGTCACTGGCATACTGAGAATATAC |
| | | | GAGAATATACAATGATCCTGGAAAT |
| 225644_at | CCDC117 | 0.4973068 | TTTGCCTTAAGAGTTCCCTAGGGAG |
| | | | TACCAGGGCTTTTCGTTTTGTGTAG |
| | | | GTAGCTTTTGCAGCATGGATCAAAC |
| | | | GGATCAAACATTGGCTTACTGTGCT |
| | | | ATTGGCTTACTGTGCTAATGTGTGA |
| | | | ATGTGTATTTTATCTGAGTTTGAGT |
| | | | GAGTAGGGTGCGTTGTGGATTTTGT |
| | | | GAAAGTCCAGTTCTCATAAATATTG |
| | | | GTTTATCAGCACGTTCATTTATTAT |
| | | | GGAATGTTCTGGAAGATGCTGTTAA |
| | | | TGAGAATCTGGTGTTACTGTATTTT |
| 217814_at | CCDC47 | 0.457459652 | GTATCTGCACGAGCACTTAGCTTGT |
| | | | CACTTAGCTTGTTCAGATCTCTGCA |
| | | | AGGTCATTGCTTGTACCAGGTAATT |
| | | | GGGTATTTTTTGTTGATGCTTTAGT |
| | | | GATGCTTTAGTGCAGGCCTGTTCTG |
| | | | GTGAAAACAGCATGTGCTGCTGCCT |
| | | | TTGTAACTGCATGGAAACTTTTCAC |
| | | | TTTTCACATGGGTTTTTCTCCAAGT |
| | | | TATAGTAGTGGCCTTGTTTTACAAA |
| | | | AAGTCCCATACATTTGGACCATGGC |
| | | | ATGAACTACCTATGGACATCTATTA |
| 222432_s_at | CCDC47 | 0.40877297 | CTGGAGGAGGCTGCATTGAGGCGTG |
| | | | GAAAGCCATGTAAAGCCATCCCAGA |
| | | | ATTTGAGTTCTGATGCCACCTGTAA |
| | | | TGCCACCTGTAAGCTCTGAATTCAC |
| | | | GAAAAACGCCAGTCCATTTCTCAAC |
| | | | CTCAACCTTAAATTTCAGACAGTCT |
| | | | TCATCTACTCTGTTTGGGGTTTGGG |
| | | | AGATACCTGGAAAGGGCTCTGTTTC |
| | | | TCATCAGTGCTTTTAGTACTTCAGT |
| | | | GTAGATAACCAGATTGTTGCTTTTT |
| | | | GACTGACTCTAAACCAAGATTCTGC |
| 218722_s_at | CCDC51 | 0.405090667 | GAACACCATCTATAGCACCCTGGTC |
| | | | GCACCCTGGTCACCTGTGTGACATT |
| | | | TGTGACATTTGTGGCCACACTGCCT |
| | | | GCTATTCAAAGCCAGCTAACCCCTG |
| | | | GAAGCGAGCCTTTGGGGGCATGTAC |
| | | | GGGCATGTACAACCTCAATCTGAAG |
| | | | GGAGCAGTATCTGTGTGGCTCACCA |
| | | | AGCAGGCATGCTTCGCTTTGTAGAC |
| | | | AGATGTAGATGTCCTTTCAGCTGCC |
| | | | AGTCATTCCAGGCAAGTCCATTCAT |
| | | | CAGCAGACGGGGCTATGCCCAGCTT |
| 227783_at | CCDC57 | 0.618837339 | TCCTCCAGCAGCCGACAGGAGGCCC |
| | | | AGGCCCGTCAAGATGCAGGCAGGCA |
| | | | AAGATGCAGGCAGGCATTGCCACCC |
| | | | GGCATTGCCACCCCAGGGATGAAGA |
| | | | TCCTGCAAAAGCTAAAGGCTGCCAG |
| | | | CCCCCAAGATCCGTAACTACAACAT |
| | | | ACAACATTATGGACTGACTTCCTCC |
| | | | AGCCGGCCCAGGAGGAAGGCCATGC |
| | | | CCAGGAGGAAGGCCATGCGTCTCTG |
| | | | GTGGGCACAGCGTGCAGGGTGGAGG |
| | | | TCTCGCCCAAGTGAGGCCTGTGTGC |
| 215343_at | CCDC88C | 0.401296785 | AAGGGATCAGAACTCTCGTGGGCCT |
| | | | CCTCCAGTGTGTCGCAAGTTTTTGC |
| | | | GAAAAACTCTCCGGCAGTAAAGCCT |
| | | | GTAAAGCCTAAAGTTCCACATCCAC |
| | | | TGATTTCTCTCCTAAGGGTATCCCG |
| | | | CCCGGAGTAACTTCTGCACATGGAT |
| | | | ACATGGATGCCTGGGACTTCACAGC |
| | | | GTCCAAACACATTAACTGCAGCATA |
| | | | TAGCATGTTCCCAATGATGACTTAC |
| | | | GATGACTTACAGCACTATGCCTTTT |
| | | | GCAACTACAATGACTGTACTCTCTA |
| 214049_x_at | CD7 | 0.494625916 | GAATTCGGCGGCATGTGTGGTGTAC |
| | | | GTGTACGAGGACATGTCGCACAGCC |
| | | | CAACCAGTACCAGTGACCCAGTGGG |
| | | | TCCCACGGCTGCAGCAGAGTTTGAA |
| | | | AGCAGAGTTTGAAGGGCCCAGCCGT |
| | | | AGCTCCAAGCAGACACACAGGCAGT |
| | | | CCCACGGTGCTTCTCAGTGGACAAT |
| | | | TCAGTGGACAATGATGCCTCCTCCG |
| | | | GAGGAAGCCTGACTGTCCTTTGGCT |
| | | | GAGGGCTTTTCTGTGGGATGGGCCT |
| | | | CCACCCAGCCGTACCAGAAATAAAG |
| 214551_s_at | CD7 | 0.359343344 | CCAGGCCATCACGGAGGTCAATGTC |
| | | | ATCACGGAGGTCAATGTCTACGGCT |
| | | | CGGAGGTCAATGTCTACGGCTCCGG |
| | | | GAGGAACAGTCCCAAGGATGGCACA |
| | | | CGTGTGTGCTGGCGAGGACACAGAT |
| | | | GTGCTGGCGAGGACACAGATAAAGA |
| | | | GGGATAAGAATTCGGCGGCATGTGT |
| | | | GAATTCGGCGGCATGTGTGGTGTAC |
| | | | GGCGGCATGTGTGGTGTACGAGGAC |
| | | | GTGGTGTACGAGGACATGTCGCACA |
| | | | TGTACGAGGACATGTCGCACAGCCG |
| 204126_s_at | CDC45 | 0.983080062 | GGCCTGGAACTCGCCAAGAAGCAGC |
| | | | TGCTCTCTCATGGAGGGCACTCCAG |
| | | | GGCACTCCAGATGTCATGCTGTTCT |
| | | | TGCTCAGCAAACACCTGCTCAAGTC |
| | | | GCTCAAGTCCTTTGTGTGTTCGACA |
| | | | GACAAAGAACCGGCGCTGCAAACTG |
| | | | GCATGGCACAGTGACCGTGGTGGGC |
| | | | CCCCAGAGACCGACAGCTCGGACAG |
| | | | GATGCTGCACAACCATTTTGACCTC |
| | | | AGTTTCTGGACGCACTTATTTCCCT |
| | | | TTTCCCTCCTGTCCTAGGAATTTGA |
| 221436_s_at | CDCA3 | 0.971627813 | GCACGGACACCTATGAAGACCAGCA |
| | | | CCCCAAGCCCACTGGTGAAACAGCT |
| | | | CCAGAGGCACCTTTATCTTCTGAAT |
| | | | CTGAATTGGACTTGCCTCTGGGTAC |
| | | | CCAGATCTTCAGGTTCTATGCGCAA |
| | | | GCAAGGTACTAGGGAGATCCCCCCT |
| | | | TCCTGCAGGATGACAACTCCCCTGG |
| | | | TACGACAGGGTAAGCGGCCTTCACC |
| | | | GGAGCCATTCTTGGAACTGGACGAC |
| | | | GAGCAAGGCCAGGACCATGACAAGG |
| | | | AAAATCAGCACTTTCCCTTGGTGGA |
| 223307_at | CDCA3 | 0.97567222 | AATGGCTTGTTTTCTTAGACTCCTC |
| | | | CCTCCTCAGCTACCAAACTGGGACT |
| | | | GCTACCAAACTGGGACTCACAGCTT |
| | | | GGACTCACAGCTTTATTGGGCTTTC |
| | | | TTATTGGGCTTTCTTTGTGTCTTGT |
| | | | TTCTTTGTGTCTTGTGTGTTTCTTT |
| | | | CCTGCATGGCCCCAGCAATGCAGTC |
| | | | ACCCAGGGCCTGGTGATATCTGTGT |
| | | | CCTGGTGATATCTGTGTCCTCTCAC |
| | | | CTTCTTTCCCAGGGATACTGAGGAA |
| | | | GGGATACTGAGGAATGGCTTGTTTT |
| 204029_at | CELSR2 | −0.38609522 | TTGGGATGGGTTCGTGTCCAGTCCC |
| | | | TCCAGTCCCGGGGGTCTGATATGGC |
| | | | CTGATATGGCCATCACAGGCTGGGT |
| | | | ACAGGCTGGGTGTTCCCAGCAGCCC |
| | | | GCCGACTGCTTTTCATCTGAGTCAC |
| | | | AGTCACCATTTACTCCAAGCATGTA |
| | | | AAGCATGTATTCCAGACTTGTCACT |
| | | | CACTGACTTTCCTTCTGGAGCAGGT |
| | | | GTTTCTCATTTGTGAGGCCAGCCTC |
| | | | TCCCCTCAGCAATTCCTGCAAAGGG |
| | | | GCTGGATGCTAACTTGATACTAACC |
| 36499_at | CELSR2 | −0.34862803 | CTCCCTGTGAAGAGAGAGTTAATAT |
| | | | TCCCAGCAGCCCTGGCTTGGGGGCT |
| | | | TGGCTTGGGGGCTTGACGCCCTTCC |
| | | | CTCTCCTCAGTTTTGCCGACTGCTT |
| | | | CCAAGCATGTATTCCAGACTTGTCA |
| | | | ATGTATTCCAGACTTGTCACTGACT |
| | | | CTTGTCACTGACTTTCCTTCTGGAG |
| | | | TTTCCTTCTGGAGCAGGTGGCTAGA |
| | | | GAAAGGCTCCTGTTTCTCATTTGTG |
| | | | TTCTCATTTGTGAGGCCAGCCTCTG |
| | | | CTCTGGCTTTTCTGCCGTGGATTCT |
| | | | TTAACTGGTTTTTACTACTGATGAC |
| | | | TAACTGGTTTTTACTACTGATGACT |
| | | | CCATCAGATTGTACAGTTTGGTTGT |
| | | | TACTACTGAATAAACTAGTTCTGTG |
| | | | ACTGAATAAACTAGTTCTGTGCGGG |
| 204962_s_at | CENPA | 1.01511874 | AGACCACTTTGAGCAGTTGCCTGGA |
| | | | GAAGGCTGGGCATTTCCATCATATA |
| | | | CATTTCCATCATATAGACCTCTGCC |
| | | | CCCTTCAGAGTAGCCTCACCATTAG |
| | | | CCTCACCATTAGTGGCAGCATCATG |
| | | | GAGTGGACTGTGCTTGTCAACGGAT |
| | | | GTCAACGGATGTGTAGCTTTTCAGA |
| | | | GCTTTGATGTTCTGGTTACTTCTAG |
| | | | TTACTTCTAGTAAATTCCTGTCAAA |
| | | | TCAACACCGTTCCAAAGGCCTGAAA |
| | | | GAGACTCCAAGGTTGACTTTAGTTT |
| 210821_x_at | CENPA | 0.920552048 | CCCTTCAGCCGCCTGGCAAGAGAAA |
| | | | GACTTCAATTGGCAAGCCCAGGCCC |
| | | | GGCCCTATTGGCCCTACAAGAGGCA |
| | | | GTTCATCTCTTTGAGGACGCCTATC |
| | | | ACATGCAGGCCGAGTTACTCTCTTC |
| | | | GATCCGGGGCCTTGAGGAGGGACTC |
| | | | CACCCAGTGTTTCTGTCAGTCTTTC |
| | | | TCTTTCCTGCTCAGCCAGGGGGGAT |
| | | | GACTCTCCAGAGCCATGACTAGATC |
| | | | TGGATTCTGCGATGCTGTCTGGACT |
| | | | TGTCTGGACTTTGCTGTCTCTGAAC |
| 226788_at | CENPT | 0.387999504 | CTCAGTACTGTCAACCAGTGCCCAG |
| | | | GGAGTTCAACAATGGCCTGCGGTCC |
| | | | AGGGCAGAGTCTAAGGCCCCAGCTT |
| | | | GATGGGGTCTGGGAGTCCAGCAGGC |
| | | | TTCACATTCCGTGCTTCTTGCGGAT |
| | | | TGACAGCCATGGCAAGCAGCCGATC |
| | | | CAGCTTGGCCTCAGTGAGACGCAGG |
| | | | CCCAGCTTCCACTTCAGGAGGAAAC |
| | | | GTCGGAGCCAGTATCAGGGAAGCCC |
| | | | CAACTGGGGGCCCATAGGGCACTCG |
| | | | CCAGGTCACCAGCAAGAGAGTCCAG |
| 229448_at | CERS1 | 0.51372688 | AAGGGAGAACCCATCAGATTCGCCT |
| | | | GGCCTCTGAGTTTGACAGGGGAGCC |
| | | | CTGGGAGCTCAGACTCAGTCCAGCC |
| | | | GTGTCTGGGCCAGGGATGAACGGAG |
| | | | GCAGAGTCGGGTGTGCAGTGTCTCA |
| | | | CCTGGAAGGTGCCGACCAGCCAGGC |
| | | | GCCCTCTGATTTGGCCGGTGGGGGC |
| | | | GGGCCATCGGTGACGTGGGAACGAT |
| | | | CTCAGAGCTCCGTGACGTTTTTTGG |
| | | | TGCAGACATTTAACACATCCGGGGC |
| | | | GTAGCCAGGCAGAGGAGCGTCAGAC |
| 229595_at | CHCHD4 | 0.662554312 | GTGAGCTGATTTATTCTGATTCATT |
| | | | GATGGGGCCATATCTACTAGCAGAG |
| | | | TGATTTCTGCAAACCCATCTTGACC |
| | | | GCAAACCCATCTTGACCTTGAGTAT |
| | | | AGGGGTACTGTACTTTATTCCTGAT |
| | | | GGTTTCCATGTAGGTGTTGAGCTCC |
| | | | TTTGGACCCTTCCATTCATAATCCC |
| | | | TTGCCCTGAATTTTGCCACTTTTAA |
| | | | GGCTGTTCCTTGTTATTCCGAAAGC |
| | | | ACTGGCTCTCAGTCTAGTCAGGTGC |
| | | | GGTGGGGACCTAATTATTACCAGAG |
| 203044_at | CHSY1 | −0.89770937 | TTTCATCCTGTCTGTGTTATGTGGG |
| | | | TTTGTTTTATCCTTTGTATCTGAAA |
| | | | TTCAGAGCTCTGCCATTTCTTGAGT |
| | | | GTATCGGGAGTGTGTTTAGTCTGTT |
| | | | TAAACCGATCTCCAAAGATTTCCTT |
| | | | AATTTTGGTGCTCATGTGTTTTGGG |
| | | | AAATTCTCAGATCAAATGTGCCTTA |
| | | | GACTTGCCATTTTAATACACGTCAT |
| | | | TACACGTCATTGGAGGGCTGCGTAT |
| | | | GTAAATAGCCTGATGCTCATTTGGA |
| | | | AACCATTTTGTCTCATTATTCCTGT |
| 202559_x_at | CHTOP | −0.39904932 | ACTTGCCACCAGCTTGTGCATTTAG |
| | | | CTAGGCCCCACTGCTCTAAGGGGCA |
| | | | GGGCATTTACTACAGCACCTATTAA |
| | | | GGACGAGGGAGAGGTGCCCTTGCTC |
| | | | TGCTCGCCCTGTATTGACCAAGGAG |
| | | | GGACACCTGGATGCTGAGTTGGATG |
| | | | GAGTTGGATGCCTACATGGCGCAGA |
| | | | ATGGCGCAGACAGATCCCGAAACCA |
| | | | AACCAATGATTGAAGCCTGCCCATC |
| | | | CCATCCTCCCATGAGAGACTCTTGT |
| | | | TAGGCTGTGGACTTACTTGCCACCA |
| 212784_at | CIC | −0.44453195 | TGGGGGCAGGAAGGTTATCTCCTCC |
| | | | TCCTCTCCAGTTTGGGGCGGAATGA |
| | | | TGAGGCCTGCTCCTCTTGTAAATAC |
| | | | CCAAGCCCCTGTACATAACCTGGAG |
| | | | TAACCTGGAGCGTGTGACCTTCAGA |
| | | | GACCTTCAGAGCTTTTCACTTTATG |
| | | | TGCAAAATGGCTCCTGTGAGGGCTG |
| | | | GGGAGGCGCCTGTGGAATAGGGGGA |
| | | | CAGAGGGGCTGGACTCAGGTTAGTT |
| | | | TGCACTTTGCCACAGGCACGGGGAG |
| | | | CTTACTGTGCCGAGAAGCCGCAATG |
| 221223_x_at | CISH | −0.56580607 | CTGAGCCCTGGTAGTCCAAAGACCC |
| | | | CTGGTTCTTCCCTGTGGAAAGCCCA |
| | | | AAAGCCCATCCTGAGACATCTTGCT |
| | | | GGCAATCCTGGATGTCCTGGTACTG |
| | | | CTCTGTGAATGTGTCCACTCTCTTC |
| | | | TTCTGCCCCCAGCCATATTTGGGGA |
| | | | AGAAAATGCAGCCGGAGCCTCAGTC |
| | | | CATAAAGCTGGTCTCACTGTGGCGC |
| | | | GCCACCACTGCAGTTCTGCTAGGTC |
| | | | GAACAGTTTGGTGGTCTTTTCTCTT |
| | | | TTTCTCTTCCACTGATTTTTCTGTA |
| 223377_x_at | CISH | −0.50821557 | TGAGCCCTGGTAGTCCAGAGACCCC |
| | | | CTGGTTCTTCCCTGTGGAAAGCCCA |
| | | | AAAGCCCATCCTGAGACATCTTGCT |
| | | | GGCAATCCTGGATGTCCTGGTACTG |
| | | | CACCCGTCTGTGAATGTGTCCACTC |
| | | | AGAAAATGCAGCCGGAGCCTCAGTC |
| | | | GCATAGAGCTGGTCTCACTGTGGCG |
| | | | GCCACCACTGCAGTTCTGCTAGGTC |
| | | | GAACAGTTTGGTGGTCTTTTCTCTT |
| | | | TTTCTCTTCCACTGATTTTTCTGTA |
| | | | GACATTATACCTTTATTACCTCTTT |
| 223961_s_at | CISH | −0.46650396 | CCTGGCCACCTGAACTGTATGGGCA |
| | | | GGAGGATGACATGCAGAGGAACTGA |
| | | | GATCGACAGTGACTAGTGACCCCTT |
| | | | GTGACTAGTGACCCCTTGTTGAGGG |
| | | | GGTAAGCCAGGCTAGGGGACTGCAC |
| | | | GGGACTGCACAATTATACACTATTT |
| | | | TTATTCTCCTTGGGGTTGGTGTCAG |
| | | | TGTGGAAAGCCCATCCTGAGACATC |
| | | | TCCTGAGACATCTTGCTGGAACCAA |
| | | | GGAACCAAGGCAATCCTGGATGTCC |
| | | | GTAAGAAAATGCAGCCGGAGCCTCA |
| 208659_at | CLIC1 | 0.557267522 | CAAAGGCCCTGGTGGTTTCCACATT |
| | | | ATTGCTACCCAATGGACACACTCCA |
| | | | GTGGGCAGGGAATCCTGGAGCACTT |
| | | | GGAGCACTTGTTCCGGGATGGTGTG |
| | | | GAAGATGAAGGTGTCTCTCAGAGGA |
| | | | TTTTTGGATGGCAACGAGCTCACCC |
| | | | GTACCGGGGATTCACCATCCCCGAG |
| | | | CCTTCCGGGGAGTGCATCGGTACTT |
| | | | TCGGTACTTGAGCAATGCCTACGCC |
| | | | GCCCGGGAAGAATTCGCTTCCACCT |
| | | | GCAAAGGCCCTCAAATAAGCCCCTC |
| 209081_s_at | COL18A1 | −0.41482881 | TGCCCATCGTCAACCTCAAGGACGA |
| | | | TTCTCCTTTGACGGCAAGGACGTCC |
| | | | GACGGCAAGGACGTCCTGAGGCACC |
| | | | CTGGCCCCAGAAGAGCGTGTGGCAT |
| | | | CAGGCTGACCGAGAGCTACTGTGAG |
| | | | AGAGCTACTGTGAGACGTGGCGGAC |
| | | | TGTGAGACGTGGCGGACGGAGGCTC |
| | | | CCTACATCGTGCTCTGCATTGAGAA |
| | | | TGAGAACAGCTTCATGACTGCCTCC |
| | | | GCTGCCATACTTTCCTGTATAGTTC |
| | | | ATACTTTCCTGTATAGTTCACGTTT |
| 209082_s_at | COL18A1 | −0.40498176 | TGGCTGGGACGTGGCTCAGCCAGCA |
| | | | TCAGCCAGCACTTGTCCAGCTGAGC |
| | | | GAGCGCCAGGATGGAACACGGCCAC |
| | | | GCACAGGACATGCGGTAGCCAGCAC |
| | | | GGTAGCCAGCACACAGGGCAGTGAG |
| | | | GCTCCAGATGCAGGGCAGTCATTGG |
| | | | GTCATTGGCTGTCTCCTAGGAAACC |
| | | | AGGTGCAACAAGGTCCTCTGTCAGT |
| | | | GAGTCATTCGTTCTGTGGAGGGACA |
| | | | CCTCAGGACTGCGACGAAACCGGTG |
| | | | GGGCTGGTTCTGTAATTGTGTGTGA |
| 201264_at | COPE | 0.575376523 | ACCTCGCCCGGAAGGAGCTGAAGAG |
| | | | GAGAATGCAGGACCTGGACGAGGAT |
| | | | GAAGCTGCAGGATGCCTACTACATC |
| | | | GATGCCTACTACATCTTCCAGGAGA |
| | | | GGAGATGGCTGACAAGTGCTCGCCC |
| | | | TGCTCAATGGGCAGGCGGCCTGCCA |
| | | | TGCTGCAGGAGGCGCTAGACAAGGA |
| | | | AGGATAGTGGCTACCCAGAGACGCT |
| | | | CCCAGAGACGCTGGTCAACCTCATC |
| | | | CCCATCCCTTCATCAAGGAGTACCA |
| | | | GCCCAGAGCTGTCAGGACCATGAAG |
| 221754_s_at | CORO1B | 0.395216845 | CAGCGGGACCTGAAGATCAGCCGGC |
| | | | CGCAACGTGTTGTCTGACAGCCGGC |
| | | | CCCTGAGGGCGCTGGTCAAGGAGCA |
| | | | TCAAGGAGCAGGGCGACCGCATCTG |
| | | | CAGCTGGGCCGCATGGAGAACGGGG |
| | | | TGGGCCGCATGGAGAACGGGGATGC |
| | | | GGAGAACGGGGATGCGTAGGGCCAC |
| | | | CCAGAGCCTCTGAGGCAGCGCAGGG |
| | | | CCTCCCCAGAGGAGGCGGGAGGGTG |
| | | | GAGGGTGGGCTCTATATTTTCATTC |
| | | | GTGGGCTCTATATTTTCATTCCAAA |
| 209833_at | CRADD | 0.525119204 | ATGACCGACCTGCCTGCAGGTGACA |
| | | | GGTGACAGATTGACTGGGATCCCCT |
| | | | TGAGTGGGAGCCCATGGTGCTGTCT |
| | | | GGGACTGTCCCAGACGGATATCTAC |
| | | | GGATATCTACCGCTGTAAGGCCAAC |
| | | | CAGCAACCGCTGGGGAGTGTGTCCC |
| | | | GTGTCCCTGAGTCATGTGGGCTTGA |
| | | | TGGGCTTGAATCCTGACTTTCACTC |
| | | | CAGGGTTTCCACTAGACATTACTTG |
| | | | CAGATTACTCAGCAGATCTCCCATG |
| | | | GATCTCCCATGTTGGCTCAACAATT |
| 226455_at | CREB3L4 | −0.41959118 | TTCAGTCCATTCCAGAGTCGACCAG |
| | | | GGGTCTGAGGATTACCAGCCTCACG |
| | | | GACTTCCAGAAATATCCTGACCCAC |
| | | | GAAGCCAAGACCCAGTGGGCGCATC |
| | | | TGTGAGCTGGAACAGACCTTCCTGG |
| | | | GGGATTCCTACTTAGGTGTCTGCCC |
| | | | CCCTCAGGGGTCCAAATCACTTCAG |
| | | | ACACCCCAAGAGATGTCCTTTAGTC |
| | | | CTTTAGTCTCTGCCTGAGGCCTAGT |
| | | | TGAGGCCTAGTCTGCATTTGTTTGC |
| | | | GAGGGTACCTCAAATACTTCTGTTA |
| 226307_at | CRTC2 | −0.39881009 | CTAAACATGCTGAGTGACCCCTGTG |
| | | | GCTGTGGAGGAGTCATTCCGCAGTG |
| | | | CAGTGACCGGCTCCAATGAGGGCAC |
| | | | CAATGAGGGCACCTCATCACCATCC |
| | | | TCCCCCTGGCAGGTAGAGACTCTAC |
| | | | CCAGATCCTCTTTCTAGCATGAATG |
| | | | GGCCCCTGAATTCTGCGCAAGGGAT |
| | | | GATGGGCCTGGGGGAACTCAAGGGA |
| | | | AGCACTTGTAACTTTGAACCGTCTG |
| | | | GAACCGTCTGTCTGGAGGTCAGAGC |
| | | | CTCTTCCCCTTGCAGTGGAGGAGAG |
| 202329_at | CSK | 0.478812142 | GCCACTCGCCTTCTTAGAGTTTTAT |
| | | | CACTCGCCTTCTTAGAGTTTTATTC |
| | | | TGAGATTTTTTTTCCGTGTGTTTAT |
| | | | GGAGAAAGAAAGTACCCAGCAAATG |
| | | | TGTTTGCGCTTGACCATGTTGCACT |
| | | | GCTTGACCATGTTGCACTGTTTGCA |
| | | | CATGTTGCACTGTTTGCATGCGCCC |
| | | | CCCGAGGCAGACGTCTGTCAGGGGC |
| | | | CAGACGTCTGTCAGGGGCTTGGATT |
| | | | CGTCTGTCAGGGGCTTGGATTTCGT |
| | | | TCAGGGGCTTGGATTTCGTGTGCCG |
| 221021_s_at | CTNNBL1 | 0.419323318 | GAACCTGAGAGGGCAGCAGCGGACC |
| | | | AGCGGACCCGGCTTCTGAATAAATT |
| | | | GGGTGCAATGCAGGTGGCGGACAAG |
| | | | GAAAAACACGACATGGTCCGGCGAG |
| | | | GGTCCGGCGAGGAGAGATCATCGAC |
| | | | CGAGGAGGAGTTCTACCTCCGGCGC |
| | | | TCCCCCAGATTCGCCAGAGGGTTCA |
| | | | AGAGGGTTCACCAGATCCTAAACAT |
| | | | GGAAGCTCCATCAAAATTGTCAGGC |
| | | | AGAGAACATCGGGGACGGCCGGAGC |
| | | | GAGCAAAAGCGCATCCTGGGCTTGC |
| 209617_s_at | CTNND2 | 0.700055961 | GCTCCGGGAACAGTGCATGTGCATG |
| | | | GTGCATGCATACCACAAGACATTTC |
| | | | TAGTTTGTTAAAGCCTGTTCCATAG |
| | | | ATGACAGTGGGCAGCACCTTTCTAG |
| | | | GCAGCACCTTTCTAGCGTGAGCTGT |
| | | | GTGCTTTATACTGAACGTGGTTGAT |
| | | | AGGAGAGACGAGGCATTCGGGCCGG |
| | | | GGCGTAAGGGTTATCGTTAAGCACA |
| | | | TACACACTGTGTGGGGGACGGCTTC |
| | | | GTGACTCTAGGCTTCAGGTTGCATT |
| | | | TGCATTGGGGTTCCTCTGTACAGCA |
| 209618_at | CTNND2 | 0.594537267 | GAAGCTATTTCATTTGCTGTTCATC |
| | | | TGTACTGTATCTATTCTTCTGACCA |
| | | | TATTCTTCTGACCATCTAGTGACTC |
| | | | GTGACTCAGGATATTAGGCCCAGTT |
| | | | TTTCCACACATTCACGCATACTTGG |
| | | | ATACTTGGATATCAACCCTCTCTAC |
| | | | ATCCCCCTCAGAACACGATAAACCA |
| | | | ACCATGGCCAATTCAGTTTCACTTT |
| | | | AAAATGTAAACTGCTCGCCTTATTC |
| | | | GAACATGGGCCGCGGGTAAACTAGC |
| | | | TAAACTAGCTTTGCTCTTTAGATGC |
| 226833_at | CYB5D1 | −0.52321368 | CATCACCTCTTGTCTAAACTGCTAC |
| | | | CTTCTGACTTCCATGCTGCAATGAG |
| | | | AACATCGAACTTTCCTTAGCTTCTT |
| | | | CTTCAGTGGCTTCCAACTGCTTTTG |
| | | | ATAAATCAGGCTCATCTCGCAACAC |
| | | | GTTCTCTATGGCCTAGACACACTGG |
| | | | AGTTCATTACATGTCTTGCCTCAGA |
| | | | CTTTCCACCTAGCTGATCCTAAATG |
| | | | TAAATGTTCCTTCCTCAGGGAGGTC |
| | | | GCTGGCTGCGCTGCTAGATTGTAAG |
| | | | TTCACATCTTGCTCACTGCTATATT |
| 219001_s_at | DCAF10 | −0.47812956 | GGGATTACCAAAGGCTGGAGCGATT |
| | | | TTAAGCTATGCACTAGCCTTGCCCT |
| | | | CCCTCTTAGGTTGCATTCTCTTTAG |
| | | | TTCTCTTTAGGCCACTGGTTCTCAA |
| | | | GGCCACTGGTTCTCAAACATTAGGG |
| | | | AACATTAGGGTGCACTGTAACCATT |
| | | | TGAAAAGTGCAGAAACCTGAGCCTC |
| | | | GTATTTTTCATAGCAACCTCAAGTA |
| | | | AGGGTCTCGGGATTGCAGGTTGAAA |
| | | | GAAAAACACAACCTTAATCAGCAGT |
| | | | GCAGTAAATTCTTCTCTACTCGGCC |
| 222804_x_at | DCAF10 | −0.55404981 | AAAGTTCTCTCCAACACATTGTCAG |
| | | | CATTGTCAGATTGCCTCAGGGTGCC |
| | | | GTGGACGGGTTTCTTTGTATCAGCC |
| | | | GGAACTCTTCTGGTGTTTGACTTAG |
| | | | GATCCTGGTTCTTATGGGTCCATGA |
| | | | GGTCATCCAGCATCATACAGGCATC |
| | | | ACAGGCATCTCCAAGTTAGACTCTA |
| | | | AGCATCATCTTTTGCAGCATTCCTC |
| | | | TGATCTGTGCCACTGAACTCCAGTT |
| | | | CAGTTCTTCTGGTCATTTTGCATGG |
| | | | TTTTGCATGGTAGCTCTTGTCACGT |
| 226511_at | DCAF10 | −0.53751669 | GAAACTCCCAGTTAAAGCCTAGGCT |
| | | | AGCCTAGGCTAGCAATTTTTTTTAG |
| | | | ACTAAGGATGCTGCTAGACTAAGGA |
| | | | CAGAGGGGTCTATCATGCTTTTAAG |
| | | | GAGTATTTTGGATGCCATTAAACTT |
| | | | GGCTTAAATTATCACGTATTGTTAC |
| | | | AATCATGTCCTAAGAATTTCTCCCA |
| | | | GAATTTCTCCCATTCAAATGATAAT |
| | | | TCAAAGTTACCATTACGCTGCTCTC |
| | | | GCTCTCTTGTAAATGAACTAGGGAT |
| | | | ATCTCACATTCACCTCCTATATGTA |
| 230679_at | DCAF10 | −0.40321124 | TTTCTCCCTCATTTAGATTTCATGG |
| | | | GGAAATTTGGAGTATTCCAGACAAT |
| | | | TTCCAGACAATATACTAGATACCCA |
| | | | TATACTAGATACCCAGAAACTTTTC |
| | | | AGAAACTTTTCTCAGTAGGTTCTGA |
| | | | TAGGTTCTGAGGTGTTTTAAGTTCT |
| | | | TTTAAGTTCTTATGCTAGACTGTAA |
| | | | TTATTTATTCTTGTATCCTCAGTGC |
| | | | CTGGTACAGGACTTGACACAGAGTA |
| | | | GGACTTGACACAGAGTAGTTGTTCA |
| | | | ATCTGGTCCAAAGTCTTTAAAATAG |
| 210811_s_at | DDX49 | 0.683776996 | GTGTGAGATCAAACTGGAGGCGGCC |
| | | | TCAAACTGGAGGCGGCCCACTTTGA |
| | | | GGCGGCCCACTTTGACGAAAAGAAG |
| | | | GGAGATCAACAAACGGAAGCAGCTG |
| | | | TGGAGGCCAAGCGCAAGGCTGAGCT |
| | | | AGCAGAAGAACCGGCGCTTCAAGGA |
| | | | GGAGGAGACGCTGAAGCGACAGAAG |
| | | | GACTCGTCCATGGAGCTGAGGGTCG |
| | | | GTCCATGGAGCTGAGGGTCGGAGGA |
| | | | GGGTGCCGCATACAGGAGGTGCTTA |
| | | | GCCGCATACAGGAGGTGCTTAATAA |
| 31807_at | DDX49 | 0.753262317 | CCACTTTGACGAAAAGAAGGAGATC |
| | | | GGAAGGACCCTGACCTGGAGGCCAA |
| | | | AAGGACCCTGACCTGGAGGCCAAGC |
| | | | AGAACCGGCGCTTCAAGGAGAAGGT |
| | | | ACGCTGAAGCGACAGAAGGCTGGCA |
| | | | TGCCCAGTCCTTGACTCGTCCATGG |
| | | | CCCAGTCCTTGACTCGTCCATGGAG |
| | | | CTGAGGGTCGGAGGAACCTTCCTTG |
| | | | AGTGCCCCACAGCAGAACCCGTGGG |
| | | | CAGCAGAACCCGTGGGCGCTCGTGT |
| | | | TTCCCTGAGCCCTGGCCAAGATTCA |
| | | | GCCCTGGCCAAGATTCAGGCTGCAG |
| | | | CAAGATTCAGGCTGCAGGGGAAGAA |
| | | | ACATGACCGGGAGGTTGTGACCCCA |
| | | | CATGACCGGGAGGTTGTGACCCCAA |
| | | | GGTGCCGCATACAGGAGGTGCTTAA |
| 236496_at | DEGS2 | −0.41597379 | CACTCCTGGGTGAAGGTGCTCTGGG |
| | | | GAAGGTGCTCTGGGATTTTGTGTTT |
| | | | TTTTGTGTTTGAGGACTCCCTGGGG |
| | | | GGCCCTATGCCAGGGTGAAGCGGGT |
| | | | GTGAAGCGGGTGTACAGGCTGGCAA |
| | | | GTGTACAGGCTGGCAAAAGATGGTC |
| | | | CAAAAGATGGTCTGTGAGCCCGGGC |
| | | | TGAGAAGCTACATTTCCTTCCTGTG |
| | | | GCCGCACACGCAGCGGGCAAGGAGA |
| | | | GCGGGCAAGGAGATACTGGGTGCGG |
| | | | GGAGATACTGGGTGCGGAAGATCGC |
| 202481_at | DHRS3 | 0.458263723 | GGTGAGCAGGACAGCTCCTGTCCCC |
| | | | TGTCCCCAGCGAAGAATCCGGCTGC |
| | | | TGATGGGTGTAACTGACCCCCACAG |
| | | | TGCTTCTCAAGTCTAACCAGCCTCA |
| | | | CAGCAGTGTGCATAGACCATTTCCA |
| | | | CCATGGACAATGCATGCCCTCGTTA |
| | | | GCATGCCCTCGTTATCTTGAAAAGC |
| | | | CTTCCACAGGCTGCACTCGAGGAGA |
| | | | GATCCACAAATTCTCAGGAACCTAC |
| | | | AGGAACCTACACCTGCATGAACACT |
| | | | TGAGGAGCCACGGAGTTTGGGGGCC |
| 209509_s_at | DPAGT1 | −0.45907357 | TCTCCATTCGATATCAGCTCGTTCG |
| | | | GCTCGTTCGACTCTTCTATGATGTC |
| | | | TACCTCACAGTCTCTAGGATTCCTG |
| | | | CTTTCTCTGTGATCATTGGCATCCT |
| | | | CAGCTTTTTTTGCAGTTATCCACAC |
| | | | CAGTTATCCACACTCACATTTCAGA |
| | | | GAGTCCTGACTCTCAAGGAACCACT |
| | | | CCAGGGCTAGGAACACAGGCTCCAC |
| | | | CAGGCTCCACGGTGACATGTCATTT |
| | | | ACTAAGCAGGGGGCCACATGCTCTC |
| | | | GGGCCACATGCTCTCAATGGAGACA |
| 201907_x_at | DVL3 | 0.341187495 | CGCCAGCAGTCAGCACAGCGAAGGC |
| | | | GCGAAGGCAGTCGGAGCAGTGGCTC |
| | | | CGGAGCAGTGGCTCCAACCGTAGCG |
| | | | AACCGTAGCGGTAGCGATCGGCGGA |
| | | | GCGGTAGCGATCGGCGGAAGGAGAA |
| | | | CCACACCACACGCAGCAGTCTGCGG |
| | | | CCACACGCAGCAGTCTGCGGGGGCC |
| | | | GTCCTTCCGCATGGCCATGGGAAAC |
| | | | ATGGGAAACCCCAGTGAGTTCTTTG |
| | | | GAGTTCTTTGTGGATGTGATGTGAT |
| | | | TGATGTGATGTGAGCAGGGCCCCTC |
| 2028_s_at | E2F1 | 0.949930222 | CAGGGCAGTGCCTGCTCCCAGAATC |
| | | | CTGCTCCCAGAATCTGGTGCTCTGA |
| | | | CCCAGAATCTGGTGCTCTGACCAGG |
| | | | AATCTGGTGCTCTGACCAGGCCAGG |
| | | | ACGGTGAGAGCACTTCTGTCTTAAA |
| | | | GAGAGCACTTCTGTCTTAAAGGTTT |
| | | | TATTTATCGAGGCCTCTTTGGTGAG |
| | | | ATCGAGGCCTCTTTGGTGAGCCTGG |
| | | | TCCCTCTACCCTTGAGCAAGGGCAG |
| | | | GGGTCCCTGAGCTGTTCTTCTGCCC |
| | | | CCTGAGCTGTTCTTCTGCCCCATAC |
| | | | TTCTGCCCCATACTGAAGGAACTGA |
| | | | CCCCATACTGAAGGAACTGAGGCCT |
| | | | AAGGAACTGAGGCCTGGGTGATTTA |
| | | | GAGACAGACTGACTGACAGCCATGG |
| | | | AGACTGACTGACAGCCATGGGTGGT |
| 204947_at | E2F1 | 0.927414606 | CTGGCTGGGCGTGTAGGACGGTGAG |
| | | | TAGGACGGTGAGAGCACTTCTGTCT |
| | | | ATTTATTTATCGAGGCCTCTTTGGT |
| | | | CTCCCTCTACCCTTGAGCAAGGGCA |
| | | | GGAACTGAGGCCTGGGTGATTTATT |
| | | | GACTGACTGACAGCCATGGGTGGTC |
| | | | GGTGGTCAGATGGTGGGGTGGGCCC |
| | | | GCTGCCCCCCAGGATGGATATGAGA |
| | | | TGGGGGACCTTCACTGATGTGGGCA |
| | | | ACCCTCCAATCTGCACTTTGATTTG |
| | | | TGATTTGCTTCCTAACAGCTCTGTT |
| 202023_at | EFNA1 | −0.79399154 | GCTGGAAGGGGCCACGTGGATGGGC |
| | | | AGAGGCAGCATGCTTGGGCTGACCC |
| | | | CTGTGCCAACCTGTTCTTAGAGTGT |
| | | | GAGTGTAGCTGTAAGGGCAGTGCCC |
| | | | GCAGTGCCCATGTGTACATTCTGCC |
| | | | ACATTCTGCCTAGAGTGTAGCCTAA |
| | | | GTGTAGCCTAAAGGGCAGGGCCCAC |
| | | | AGGGCCCACGTGTATAGTATCTGTA |
| | | | CCACCTTCACCTCGGAGGGACGGAG |
| | | | GAAGTGGAGACAGTCCTTTCCCACC |
| | | | GGCATGGTCCCTTAAGGCACAGTGG |
| 219850_s_at | EHF | −0.79952448 | GATTGAGAACCACCAGTTTAGCTAG |
| | | | GAACCACCAGTTTAGCTAGTCAATA |
| | | | GGATGGTGGTTTATTCTCAGAAGAA |
| | | | CCAGATGAGAGCCAATGTCAGATAA |
| | | | TTTGTCTTTTGGATTATCTGTTTAC |
| | | | TGGATTATCTGTTTACTGTCTCATC |
| | | | TACTGTCTCATCTGAACTGATCCCA |
| | | | GTCTCATCTGAACTGATCCCAGGTG |
| | | | GATCCCAGGTGAACGGTTTATTGCC |
| | | | GGTTTATTGCCTAGATTTGTACTCA |
| | | | GCCTAGATTTGTACTCAGAGGAATT |
| 222932_at | EHF | −0.6110743 | AAGGAGTTAAAAGCTTCTTCTCAAT |
| | | | TGAGCCATGCAATCTGGGAAGCACA |
| | | | GGAAGCACAGGAATAAGTAGACACT |
| | | | ATGAAGACATGTATCCATAAGAAGG |
| | | | ATAAGAAGGAGTGCTCTTCATCAAC |
| | | | TTCATCAACTAATAGAGCACCTACC |
| | | | TAGAGCACCTACCACAGTGTCATAC |
| | | | CACCTACCACAGTGTCATACCTGGT |
| | | | CACAGTGTCATACCTGGTAGAGGTG |
| | | | ATATATTCATGAGGCTGGAAGTAAG |
| | | | GAGGATGGGGCTTAGATAGTATCGA |
| 224189_x_at | EHF | −0.81521604 | ACATCACCAAATGTTCCCTGGGGGT |
| | | | ATTTGCCCTTGATTGAGAACCACCA |
| | | | GAACCACCAGTTTAGCTAGTCAATA |
| | | | CTTCAACCTCAACCTATCTTTATGT |
| | | | AGAGGAGCTTCTTTTCAGAACCCCA |
| | | | AGAACCCCAGATGAGAGCCAATGTC |
| | | | ATTTCCAGGGAAAATCCTCTTTGCA |
| | | | GATTATCTGTTTACTGTCTCATCTG |
| | | | GTCTCATCTGAACTGATCCCAGGTG |
| | | | GATCCCAGGTGAACGGTTTATTGCC |
| | | | GTTTATTGCCTAGATTTGTACTCAG |
| 225645_at | EHF | −0.81425955 | TGCCTGCTATGTGCACGGCATGGGC |
| | | | GCACGGCATGGGCCCATATGTGTGA |
| | | | GATCTCGGTAGTTACGTATTGGGCA |
| | | | AATTATCCTCAGTGTAGCTTCTTGG |
| | | | AAAACTCCTGTTGAGACTGTGTCTT |
| | | | GACTGTGTCTTATGAACCTCTGAAA |
| | | | GAACCTCTGAAACGTACAAGCCTTC |
| | | | AATCTTTCTGTAGTTATCTGCATAA |
| | | | CTGGCTCCTGGGTTGACAATTTGTG |
| | | | GAAACAACTCTATTGCTACTATTTA |
| | | | GTTTATTTGTTTGATGGGTCCCAGG |
| 232360_at | EHF | −0.91281478 | TGAAGTGGAACGGTGACTCTCTCTT |
| | | | CCTGCTAGGAGCCAGCTGGAAGAAT |
| | | | GAGATTCTGCAGATGACAGGATTCT |
| | | | GAAAGAGTGGTCTCACCTCCAAATT |
| | | | TGGTCTCACCTCCAAATTACCATGT |
| | | | GAAGCATAGGGTACCTGGTGTGCCT |
| | | | GTGTGCCTAATCCCTTATAAATGCC |
| | | | ATTTAATTCTTTCCTTATGGTGATA |
| | | | CGTAGAATACTTACTATCCTTGGAA |
| | | | TCCTTTGCAAACAGTCCAGTCACTT |
| | | | ACAGTCCAGTCACTTGCTTGTTAAA |
| 232361_s_at | EHF | −0.89914362 | CAAGTACCAGGTGTGGGAGTGGCTC |
| | | | CTCCTGGACACCAACCAGCTGGATG |
| | | | TGGACACCAACCAGCTGGATGCCAA |
| | | | ACACCAACCAGCTGGATGCCAATTG |
| | | | TGGATGCCAATTGTATCCCTTTCCA |
| | | | ATTGTATCCCTTTCCAAGAGTTCGA |
| | | | GTATCCCTTTCCAAGAGTTCGACAT |
| | | | TCCCTTTCCAAGAGTTCGACATCAA |
| | | | AGTTCGACATCAACGGCGAGCACCT |
| | | | CTCCTCTACAGCAACTTGCAGCATC |
| | | | CTCTACAGCAACTTGCAGCATCTGA |
| 203462_x_at | EIF3B | 0.456133148 | GGTGGACACTGACGAGCTGGACAGC |
| | | | GAGACCATTGAGTTCTTCGTCACTG |
| | | | CACTGAAGAAATCATTCCCCTCGGA |
| | | | AGGAGTGACCTGGAGCACTGTGCGC |
| | | | TGGATTCTGCCATTGCGACACATTT |
| | | | GCGACACATTTTTGTGCCTTTCAGC |
| | | | AGCCCCTGGTGTCTGCAGTGGGGGA |
| | | | GCTTCCACTTCTTTCTTGTTTGGAG |
| | | | GGCTCCGAAGACTTAGCGACGCACT |
| | | | CTGTACACAGCCGAGCAGCATTTCC |
| | | | TCCGTTGAAGGACTTGCATCCCCAT |
| 208688_x_at | EIF3B | 0.483912025 | TGAGCTACAGGACTCCCGAGTGTGA |
| | | | TGGATTCTGCCATTGCGACACATTT |
| | | | GCGACACATTTTTGTGCCTTTCAGC |
| | | | AGCCCCTGGTGTCTGCAGTGGGGGA |
| | | | CAGTGGGGGATTTAAGGCACCCGCT |
| | | | GCTTCCACTTCTTTCTTGTTTGGAG |
| | | | TTGGAGTTTTCTGTTGGAACCGCCG |
| | | | GGCTCCGAAGACTTAGCGACGCCAC |
| | | | CTGTACACAGCCGAGCAGCATTTCC |
| | | | TCCGTTGAAGGACTTGCATCCCCAT |
| | | | CACCGTGCAGGTTGTGGCCGGTTTT |
| 211501_s_at | EIF3B | 0.480989628 | GGAGAGAAGGCGCACCATGATGGAA |
| | | | GATGGAAGATTTCCGGAAGTACCGG |
| | | | GGAGCAGAAAAACGAGCGCCTGGAG |
| | | | AGCGCCTGGAGTTGCGAGGAGGGGT |
| | | | GTTGCGAGGAGGGGTGGACACTGAC |
| | | | GGTGGACACTGACGAGCTGGACAGC |
| | | | CGAGCTGGACAGCAACGTGGACGAC |
| | | | ACGTGGACGACTGGGAAGAGGAGAC |
| | | | GGAGACCATTGAGTTCTTCGTCACT |
| | | | ACCATTGAGTTCTTCGTCACTGAAG |
| | | | GTTTTCTCCGCAGGTTGAACATGGA |
| 203617_x_at | ELK1 | 0.503580012 | CTGCCTGTTTCCTCCCAATGGAGGG |
| | | | CCCCGCTGCCATTTTGATAGTATAA |
| | | | GGGGAGAGGGAGTCATCTCTTCCTA |
| | | | GTCATCTCTTCCTATATTTGGTGGG |
| | | | GATTTGGGGGGGAATCTTCTGCCTC |
| | | | AACATGAATTTTCAGTTCCCTCCCT |
| | | | CAAAGGACCCTTTCAATGTCCCTGG |
| | | | GACATAAAGCCTGTCCTGTCTCTAT |
| | | | CTGTCCTGTCTCTATTCTAGGCAAG |
| | | | GGTTCAAAAGACTCCTGGGCTCACC |
| | | | GATTTGGGGGACAGTGCTACACTCG |
| 203719_at | ERCC1 | −0.54778716 | GGCTGTTTGATGTCCTGCACGAGCC |
| | | | TGCACGAGCCCTTCTTGAAAGTACC |
| | | | GCCCTTCTTGAAAGTACCCTGATGA |
| | | | CCTTCTTGAAAGTACCCTGATGACC |
| | | | TTCTTGAAAGTACCCTGATGACCCC |
| | | | TCTTGAAAGTACCCTGATGACCCCA |
| | | | AAGATCTGGCCTTATGCCCAGGCCT |
| | | | CCCTCAGAAAGCCCGGAGGCTGTTT |
| | | | CTCAGAAAGCCCGGAGGCTGTTTGA |
| | | | GAAAGCCCGGAGGCTGTTTGATGTC |
| | | | AAGCCCGGAGGCTGTTTGATGTCCT |
| 203720_s_at | ERCC1 | −0.47904914 | TTTGGCGACGTAATTCCCGACTATG |
| | | | TCCCGACTATGTGCTGGGCCAGAGC |
| | | | TACATCCATGGGCGGCTGCAGAGCC |
| | | | CCTGGGGAAGAACTTCGCCTTGCGG |
| | | | GAAGCTAGAGCAGGACTTCGTCTCC |
| | | | CTTCGTCTCCCGGGTGACTGAATGT |
| | | | GACTGAATGTCTGACCACCGTGAAG |
| | | | ACAAAACGGACAGTCAGACCCTCCT |
| | | | CCTCCTGACCACATTTGGATCTCTG |
| | | | TTGGATCTCTGGAACAGCTCATCGC |
| | | | CAGCTCATCGCCGCATCAAGAGAAG |
| 228131_at | ERCC1 | −0.47360973 | TACAAGGTTCATGCTTATGGCCTGA |
| | | | GAAAATAACCACATCCCAGGCTGAC |
| | | | ACAGAACATGTTCCACCAAGCCTGC |
| | | | GCCTGCAGAATGTCCAAATGTCCTA |
| | | | CTAAGAATGCAGCCCCCATTACTTA |
| | | | GCAGCCCCCATTACTTAAATATAAC |
| | | | GGTTGCAGGATTAATGGTCGTGGAT |
| | | | TAGTGAGCTTATCTGCACACTCCAA |
| | | | ATCTGCACACTCCAAGTTTAACTAT |
| | | | CTGCTTTCTGAGGACACTCTACTCT |
| | | | CTGAGGACACTCTACTCTGTAAAGG |
| 205225_at | ESR1 | −0.40964831 | ATTGCTGCCTCTATTATGGCACTTC |
| | | | GGCACTTCAATTTTGCACTGTCTTT |
| | | | GTAAATGCTGCCATGTTCCAAACCC |
| | | | GTGTTTAGAGCTGTGCACCCTAGAA |
| | | | ATTATGCCAGTTTCTGTTCTCTCAC |
| | | | TTTTTGTGCACTACATACTCTTCAG |
| | | | GATTAATATGCCCTTTTGCCGATGC |
| | | | TACTGATGTGACTCGGTTTTGTCGC |
| | | | TTTTGTCGCAGCTTTGCTTTGTTTA |
| | | | CACACTTGTAAACCTCTTTTGCACT |
| | | | GATGCTCGAGCACCTGTAAACAATT |
| 219395_at | ESRP2 | 0.660258078 | TGCCAGGGGTGGTCCCACCTAAAGA |
| | | | GATGGACTGTGCTGCAGTATCACCA |
| | | | GTATCACCAGAAGACATTAGGGGGC |
| | | | TAGGGGGCAGTAGGCCCCCACACAA |
| | | | TAAAGGGGAGGACTTTCTGCCAACT |
| | | | GCCTTGGGAAAGCCAGTTGCCCTGA |
| | | | ACACCATGGAATGTCCTTTGCACGC |
| | | | GTCCTTTGCACGCATTAAATGGTAC |
| | | | GGTACAGAACTGAAGCCTCGGAAGC |
| | | | GAAGCCTCGGAAGCAATTTGGAACT |
| | | | TTTGCCCCAAAGTGAGGGGCTCCAC |
| 219268_at | ETNK2 | 0.538626729 | CTCCAAACCAGATCCAATCAAACCT |
| | | | AATCAAACCTCAGCCCGAGGAAACA |
| | | | ACCTCAGCCCGAGGAAACATGCTCC |
| | | | TTGTGCTGTGGCTTAGCCGGAGGGG |
| | | | GCTTAGCCGGAGGGGACGTGGCCAA |
| | | | GCCAAGGGTGAGGTGGCCAAAACCA |
| | | | CTCCAGCTCTACTTTATGTCCTGAA |
| | | | TCCTGAAGCTGACCCGAGGTCTTCC |
| | | | ACCCGAGGTCTTCCTATCTGGAATG |
| | | | GAGGTCTTCCTATCTGGAATGACTA |
| | | | GGAATGACTAGAGGGAGCCAAGAGG |
| 225319_s_at | FAM104A | 0.570273337 | TACCATCTCCCAACTTTTAAAGCCA |
| | | | TTGAGCTTTCAAACACACATGCACA |
| | | | GGAGGATTCCTGCAGGCTTAACAGT |
| | | | GCAGGCTTAACAGTTGGCATCGTAC |
| | | | GGTTGGCAGTTAACTCTTTCACCCT |
| | | | TAACTCTTTCACCCTACTAAATTCA |
| | | | TAAATTCAAGAGCTCATCTCCACCC |
| | | | CACCCTGTCCTGTATATTTTCTACA |
| | | | TACTTGGTAGTGTCAGCGGGCATCT |
| | | | GGCATCTTTTACACCTTCTAGTAGC |
| | | | AGTAAAACCTTGTACTTCTCTATTG |
| 213455_at | FAM114A1 | −0.61972007 | TAAAATCGCCTCACAGATCACACTC |
| | | | GATCACACTCGCTGGTGGCAAATAT |
| | | | ATGGGAGGCTGCACAGAAGACCCTG |
| | | | CAGGAGGGGCATTGTCAGTGGCTGC |
| | | | CCCATGGACATCCCTACAGGTACTG |
| | | | GGTACTGTCATGTGAAGCCTTGCCT |
| | | | TGAAGCCTTGCCTAGTAGTTCTCTC |
| | | | AAACCTCTTATTCACATTTGCTTTG |
| | | | TTTGCTTTGATTCCCCGATGGAGTA |
| | | | AGTAGACTGCCTTTGTTCCATACAG |
| | | | ATATCATCCTACTTCTTATTAGCAT |
| 226697_at | FAM114A1 | −0.45986055 | CACGATGGAGAGAACCGCGCACTAC |
| | | | CGCGCACTACGGGATGCTGTTTGAT |
| | | | ATATCAAGGCTTGTCACACCTGGAA |
| | | | GGAAGCCCTGGAAATTCTGTCCAAT |
| | | | CTCGCATGCTTACAGAGCTTCTCTT |
| | | | GGCCACACCTGACAAACTCAATAAG |
| | | | AAGAGGGCTCATGACTGGGTGGAAG |
| | | | GGAGGCCTTGATGCGTTGGAATTCA |
| | | | GTCCTTGCAGAAAGTGACCCGGGCT |
| | | | TGACCCGGGCTTTAAGCGGACCAAG |
| | | | GAACTGTTTCCTTGTCTCAGATGTT |
| 200767_s_at | FAM120A | −0.38030291 | TGCGGAGCCTTCTCAGGCAGTGACA |
| | | | TAGCAAGTCCCAGGGCGGAGTCCAA |
| | | | CCAACCTATACCTTCTCAGGGAGGC |
| | | | GTGGTTGGCCATTGGGCTGGGAGCA |
| | | | GGTGGTTTCTGTCGGAGGACCAGCT |
| | | | CAAGAGGAGTTATTTCCACCCCAGT |
| | | | AATTCAGGGCAGACCTCCTTATGCT |
| | | | GGGAATCGAAGTCCTCTGCTATGTC |
| | | | TCTGCTATGTCTTCAGACGGGTCCC |
| | | | ATGAACGGGAGCACGGGTGACGCCA |
| | | | CCCAGCCACTCTGAAAGTGCCTTGA |
| 200774_at | FAM120A | −0.52200803 | GACTAATACCATGCATCTGTGATCA |
| | | | GAGCTAAACTTCTGCATGGTTCATA |
| | | | AATATGCATGTTATCGTCCTTTCTT |
| | | | TCTTAACAGTATGTGCCCATTTGCA |
| | | | GTCATTGACTGATCTTGCTCTAACC |
| | | | GTGATTATTGACCTCTGTTGCATTT |
| | | | TTGCATTTATTCTAAAGCCCCCCAA |
| | | | AAATTATCTAGCCGTTTCGAATATC |
| | | | TTCGAATATCAACATTACCCTGGTG |
| | | | TACCCTGGTGTATTCACTGCTGTAT |
| | | | GCTGTATGCATTATTGTTCTTTGTT |
| 227239_at | FAM126A | −0.47305424 | TCTAGTCCTTTAATGAGCATGAATT |
| | | | TATACTTCTACATTTGTTGCTTAGT |
| | | | ATATTGTCTTCTATACTTTGTAACT |
| | | | ATTTCACGTATTGTTGCTTTCTCTT |
| | | | GTTGCTTTCTCTTATATGGAACTTA |
| | | | GGAACTTATTGTGTACCTCTTACCT |
| | | | GTATTCCTAGAGTTTACATTCCTAA |
| | | | ACGACGACTTTGGCTATTTTTGTGT |
| | | | GTTCCCTACCTTCTTAAGGCTATGG |
| | | | ATTTGTGTAAATGTTCTCCATATGT |
| | | | CAAGTGTTGCCTCTTGTTTTATTGA |
| 200894_s_at | FKBP4 | 0.480140894 | TCAACCTGGCCATGTGTCATCTGAA |
| | | | ATCTGAAACTACAGGCCTTCTCTGC |
| | | | CCTTCTCTGCTGCCATTGAAAGCTG |
| | | | AACAAGGCCCTAGAACTGGACAGCA |
| | | | GAATGACTTTGAACTGGCACGGGCT |
| | | | GGCACGGGCTGATTTCCAGAAGGTC |
| | | | TTTCCAGAAGGTCCTGCAGCTCTAC |
| | | | TGTGCCAGCAGCGGATCCGAAGGCA |
| | | | TCCGAAGGCAGCTTGCCCGGGAGAA |
| | | | GAAGAAGCTCTATGCCAATATGTTT |
| | | | ACACGGCAGGGAGCCAGTCTCAGGT |
| 200895_s_at | FKBP4 | 0.467716809 | TGGTTGGATGGTGGCTTTAGGGGAA |
| | | | GTAGGCTGGGGGATTGAGGTGGGGA |
| | | | TCATTTTAGCTGGTGTCAGCCCCTC |
| | | | CCTTCCTCCATTGCACATGAACATA |
| | | | TGTCCATCCATATATATTCATCAGA |
| | | | TGGAGAGGGAGACTCCTGGGCAGCC |
| | | | CATTTCCAAATGTGGCCTCCATGTG |
| | | | CACCCCCGACGGTGTGGCTGATGAT |
| | | | GATGTCTTCTGGTGTCATGGTGACC |
| | | | CTCTTCTCTGCACGTTGCTGAAGGT |
| | | | TGCACGTTGCTGAAGGTCCAGGCTT |
| 229902_at | FLT4 | −0.36328793 | CACTGCGCGTTACTCCAGGATATGC |
| | | | GCGCGTTACTCCAGGATATGCCGAG |
| | | | CTCCAGGATATGCCGAGTGCACGTA |
| | | | GCCGAGTGCACGTATAAGGTCATCT |
| | | | ACGTATAAGGTCATCTTCGTCGTCC |
| | | | TCTGCACGTCGTCCAACGTGGGACT |
| | | | ACGTCGTCCAACGTGGGACTGGCGT |
| | | | GTCCAACGTGGGACTGGCGTGTCGG |
| | | | TCTGCAGAGAACCAGCCTGGCTCCT |
| | | | GCCCAACCATCTCACCAGGAGAAAG |
| | | | CATCTCACCAGGAGAAAGAGCCACA |
| 209189_at | FOS | −0.9207879 | CTGCCCGAGCTGGTGCATTACAGAG |
| | | | GAGAAACACATCTTCCCTAGAGGGT |
| | | | GAGGGTTCCTGTAGACCTAGGGAGG |
| | | | AGGACCTTATCTGTGCGTGAAACAC |
| | | | GTGAAACACACCAGGCTGTGGGCCT |
| | | | GTGTGGACTCAAGTCCTTACCTCTT |
| | | | TCCTTACCTCTTCCGGAGATGTAGC |
| | | | TGTATTGTTCCCAGTGACACTTCAG |
| | | | TTAGTAGCATGTTGAGCCAGGCCTG |
| | | | TCTCCTTAGTCTTCTCATAGCATTA |
| | | | GTGTTCCTGGCAATAGTGTGTTCTG |
| 226072_at | FUK | 0.430111836 | GCCCTTTGAGGCATTCCCTATGGCT |
| | | | TACACTCAACCCTCATGTGAGCGTG |
| | | | TGCCATCCCAGGCCTTAACTAGCAA |
| | | | TACGGAGCGTGCCAAGTGACCTGGT |
| | | | GGAAGTGGGTTCTCAGGACTGGCAT |
| | | | GAAAACCTGGAGCTACAGTGTCCCC |
| | | | GACAGGGGCCTAGATGTAGCCTCTG |
| | | | GGAAGGTCCCAAGCTTAGTATCCCA |
| | | | TTAGTATCCCACGTGGCCTTTACAA |
| | | | ACAAATCCTATGGCTGGCCTTCTCA |
| | | | TTGGCATATGGCTGGGAGTCCCTTA |
| 235340_at | GANC | 0.375634848 | TTCTGTGTGCACTGCATACGCTGCA |
| | | | AGCCGTGGGAGTTATTCTCCCCTAG |
| | | | TCTCCCCTAGAGATCGACTTGGCAG |
| | | | GAAGGATTCTTTTCTCTTTCATGCT |
| | | | TGCTTCTCAGGCTCAATAGTTTCTA |
| | | | GAAATAAATACCCATGTACCCACCA |
| | | | ACCCACCACTGGACTTCAGAAGTAG |
| | | | GGCTGCGTGGGTCTGTTTTAACGTG |
| | | | CATGCAGCATTGGCGCTCTGGCTGC |
| | | | GCAGCAGCTGAGTTGCTCAAGGCCA |
| | | | GCTCAAGGCCAGTGTCCAAGTGGAC |
| 212581_x_at | GAPDH | 0.710511506 | CAAGGTCATCCCTGAGCTGAACGGG |
| | | | GTCCCCACTGCCAACGTGTCAGTGG |
| | | | GTGTCAGTGGTGGACCTGACCTGCC |
| | | | GACCTGCCGTCTAGAAAAACCTGCC |
| | | | ACACTGAGCACCAGGTGGTCTCCTC |
| | | | TCTCCTCTGACTTCAACAGCGACAC |
| | | | TTTGACGCTGGGGCTGGCATTGCCC |
| | | | CGACCACTTTGTCAAGCTCATTTCC |
| | | | GCAACAGGGTGGTGGACCTCATGGC |
| | | | TCCTCACAGTTGCCATGTAGACCCC |
| | | | CGCACCTTGTCATGTACCATCAATA |
| 213453_x_at | GAPDH | 0.746040983 | CAAGGTCATCCCTGAGCTGAACGGG |
| | | | GTGTCAGTGGTGGACCTGACCTGCC |
| | | | GACCTGCCGTCTAGAAAAACCTGCC |
| | | | TGGTGAAGCAGGCGTCGGAGGGCCC |
| | | | ACACTGAGCACCAGGTGGTCTCCTC |
| | | | TCTCCTCTGACTTCAACAGCGACAC |
| | | | TTTGACGCTGGGGCTGGCATTGCCC |
| | | | CGACCACTTTGTCAAGCTCATTTCC |
| | | | GCAACAGGGTGGTGGACCTCATGGC |
| | | | GCCTCCAAGGAGTAAGACCCCTGGA |
| | | | CCCTCCGGGAAACTGTGGCGTGATG |
| 217398_x_at | GAPDH | 0.716676445 | CGACCACTTTGTCAAGCTCATTTCC |
| | | | CAACGAATTTGGCCACACTCAGTCC |
| | | | TCCTCACAGTTGCCATGTAGACCCC |
| | | | CGCACCTTGTCATGTACCATCAATA |
| | | | GGACTCATGACCACAGTCCATGCCA |
| | | | CCCTCCGGGAAACTGTGGCGTGATG |
| | | | CAAGGTCATCCCTGAGCTGAACGGG |
| | | | CACTGCCAACGTGTCGGTGGTGGAC |
| | | | GACCTGCCGTCTAGAAAAACCTGCC |
| | | | ACACTGAGCACCAGGTGGTCTCCTC |
| | | | TCTCCTCTGACTTCAACAGCGACAC |
| AFFX- | GAPDH | 0.695543658 | TCATTTCCTGGTATGACAACGAATT |
| HUMGAPDH/M | | | ACAACGAATTTGGCTACAGCAACAG |
| 33197_3_at | | | GGGTGGTGGACCTCATGGCCCACAT |
| | | | TCATGGCCCACATGGCCTCCAAGGA |
| | | | ACATGGCCTCCAAGGAGTAAGACCC |
| | | | AGGAGTAAGACCCCTGGACCACCAG |
| | | | GCCCCAGCAAGAGCACAAGAGGAAG |
| | | | GAGAGAGACCCTCACTGCTGGGGAG |
| | | | CCTCACTGCTGGGGAGTCCCTGCCA |
| | | | CCTCCTCACAGTTGCCATGTAGACC |
| | | | AGTTGCCATGTAGACCCCTTGAAGA |
| | | | CATGTAGACCCCTTGAAGAGGGGAG |
| | | | TAGGGAGCCGCACCTTGTCATGTAC |
| | | | GCCGCACCTTGTCATGTACCATCAA |
| | | | TGTCATGTACCATCAATAAAGTACC |
| | | | CCTCTGACTTCAACAGCGACACCCA |
| | | | GGGCTGGCATTGCCCTCAACGACCA |
| | | | CCCTCAACGACCACTTTGTCAAGCT |
| | | | ACCACTTTGTCAAGCTCATTTCCTG |
| | | | TTGTCAAGCTCATTTCCTGGTATGA |
| | | | TCATTTCCTGGTATGACAACGAATT |
| | | | ACAACGAATTTGGCTACAGCAACAG |
| | | | GGGTGGTGGACCTCATGGCCCACAT |
| | | | TCATGGCCCACATGGCCTCCAAGGA |
| | | | ACATGGCCTCCAAGGAGTAAGACCC |
| | | | AGGAGTAAGACCCCTGGACCACCAG |
| | | | GCCCCAGCAAGAGCACAAGAGGAAG |
| | | | GAGAGAGACCCTCACTGCTGGGGAG |
| | | | CCTCACTGCTGGGGAGTCCCTGCCA |
| | | | CCTCCTCACAGTTGCCATGTAGACC |
| | | | AGTTGCCATGTAGACCCCTTGAAGA |
| | | | CATGTAGACCCCTTGAAGAGGGGAG |
| | | | TAGGGAGCCGCACCTTGTCATGTAC |
| | | | GCCGCACCTTGTCATGTACCATCAA |
| | | | TGTCATGTACCATCAATAAAGTACC |
| | | | CCTCTGACTTCAACAGCGACACCCA |
| | | | GGGCTGGCATTGCCCTCAACGACCA |
| | | | CCCTCAACGACCACTTTGTCAAGCT |
| | | | ACCACTTTGTCAAGCTCATTTCCTG |
| | | | TTGTCAAGCTCATTTCCTGGTATGA |
| AFFX- | GAPDH | 0.615788823 | GCGCCTGGTCACCAGGGCTGCTTTT |
| HUMGAPDH/M | | | GGTCACCAGGGCTGCTTTTAACTCT |
| 33197_5_at | | | TGCTTTTAACTCTGGTAAAGTGGAT |
| | | | GGATATTGTTGCCATCAATGACCCC |
| | | | CATCAATGACCCCTTCATTGACCTC |
| | | | CTTCATTGACCTCAACTACATGGTT |
| | | | CAACTACATGGTTTACATGTTCCAA |
| | | | GGTTTACATGTTCCAATATGATTCC |
| | | | CCAATATGATTCCACCCATGGCAAA |
| | | | TGATTCCACCCATGGCAAATTCCAT |
| | | | ATTCCATGGCACCGTCAAGGCTGAG |
| | | | TGGCACCGTCAAGGCTGAGAACGGG |
| | | | CATCAATGGAAATCCCATCACCATC |
| | | | TCCCATCACCATCTTCCAGGAGCGA |
| | | | CTTCCAGGAGCGAGATCCCTCCAAA |
| | | | GCGAGATCCCTCCAAAATCAAGTGG |
| | | | CGATGCTGGCGCTGAGTACGTCGTG |
| | | | CGTGGAGTCCACTGGCGTCTTCACC |
| | | | CTTCACCACCATGGAGAAGGCTGGG |
| | | | CGGATTTGGTCGTATTGGGCGCCTG |
| | | | GCGCCTGGTCACCAGGGCTGCTTTT |
| | | | GGTCACCAGGGCTGCTTTTAACTCT |
| | | | TGCTTTTAACTCTGGTAAAGTGGAT |
| | | | GGATATTGTTGCCATCAATGACCCC |
| | | | CATCAATGACCCCTTCATTGACCTC |
| | | | CTTCATTGACCTCAACTACATGGTT |
| | | | CAACTACATGGTTTACATGTTCCAA |
| | | | GGTTTACATGTTCCAATATGATTCC |
| | | | CCAATATGATTCCACCCATGGCAAA |
| | | | TGATTCCACCCATGGCAAATTCCAT |
| | | | ATTCCATGGCACCGTCAAGGCTGAG |
| | | | TGGCACCGTCAAGGCTGAGAACGGG |
| | | | CATCAATGGAAATCCCATCACCATC |
| | | | TCCCATCACCATCTTCCAGGAGCGA |
| | | | CTTCCAGGAGCGAGATCCCTCCAAA |
| | | | GCGAGATCCCTCCAAAATCAAGTGG |
| | | | CGATGCTGGCGCTGAGTACGTCGTG |
| | | | CGTGGAGTCCACTGGCGTCTTCACC |
| | | | CTTCACCACCATGGAGAAGGCTGGG |
| | | | CGGATTTGGTCGTATTGGGCGCCTG |
| AFFX- | GAPDH | 0.680189706 | AAGATCATCAGCAATGCCTCCTGCA |
| HUMGAPDH/M | | | ACCAACTGCTTAGCACCCCTGGCCA |
| 33197_M_at | | | TTAGCACCCCTGGCCAAGGTCATCC |
| | | | GACAACTTTGGTATCGTGGAAGGAC |
| | | | GTGGAAGGACTCATGACCACAGTCC |
| | | | ATCACTGCCACCCAGAAGACTGTGG |
| | | | GCCACCCAGAAGACTGTGGATGGCC |
| | | | CCCTCCGGGAAACTGTGGCGTGATG |
| | | | GGCCGCGGGGCTCTCCAGAACATCA |
| | | | GCCTCTACTGGCGCTGCCAAGGCTG |
| | | | GTGGGCAAGGTCATCCCTGAGCTGA |
| | | | GTCATCCCTGAGCTGAACGGGAAGC |
| | | | GAGCTGAACGGGAAGCTCACTGGCA |
| | | | AAGCTCACTGGCATGGCCTTCCGTG |
| | | | ACTGGCATGGCCTTCCGTGTCCCCA |
| | | | ACTGCCAACGTGTCAGTGGTGGACC |
| | | | AACGTGTCAGTGGTGGACCTGACCT |
| | | | GTGGACCTGACCTGCCGTCTAGAAA |
| | | | CTGACCTGCCGTCTAGAAAAACCTG |
| | | | GAAAAACCTGCCAAATATGATGACA |
| | | | AAGATCATCAGCAATGCCTCCTGCA |
| | | | ACCAACTGCTTAGCACCCCTGGCCA |
| | | | TTAGCACCCCTGGCCAAGGTCATCC |
| | | | GACAACTTTGGTATCGTGGAAGGAC |
| | | | GTGGAAGGACTCATGACCACAGTCC |
| | | | ATCACTGCCACCCAGAAGACTGTGG |
| | | | GCCACCCAGAAGACTGTGGATGGCC |
| | | | CCCTCCGGGAAACTGTGGCGTGATG |
| | | | GGCCGCGGGGCTCTCCAGAACATCA |
| | | | GCCTCTACTGGCGCTGCCAAGGCTG |
| | | | GTGGGCAAGGTCATCCCTGAGCTGA |
| | | | GTCATCCCTGAGCTGAACGGGAAGC |
| | | | GAGCTGAACGGGAAGCTCACTGGCA |
| | | | AAGCTCACTGGCATGGCCTTCCGTG |
| | | | ACTGGCATGGCCTTCCGTGTCCCCA |
| | | | ACTGCCAACGTGTCAGTGGTGGACC |
| | | | AACGTGTCAGTGGTGGACCTGACCT |
| | | | GTGGACCTGACCTGCCGTCTAGAAA |
| | | | CTGACCTGCCGTCTAGAAAAACCTG |
| | | | GAAAAACCTGCCAAATATGATGACA |
| 235310_at | GCET2 | 0.36020273 | CGATCCTTGGAGATCCCGTAATCCC |
| | | | TTTGGAGCCTGATTTCCTACTGACT |
| | | | TTTCCTACTGACTTCCAATTTAGTG |
| | | | TGCTCCCCCAGTATGCTAAATAGAA |
| | | | AATAGAAAGCCCTCTGCAATATATT |
| | | | GATTATTTACTTTCTCTTATCTTTT |
| | | | TTATCTTTTCCTTAGTGTTCCTCAA |
| | | | AAATTATATCTATCCTCTAAACCAG |
| | | | AGGGATCAGCAAACTATAACCCCCA |
| | | | ACTCATTTGTTTACCTACTATCTAT |
| | | | TGACATGGACCATAGGCCCTAAAGA |
| 202321_at | GGPS1 | −0.41410157 | GAGTAGGCATCTTTAATCGCCCTGA |
| | | | GCCTGAGAGGGCCTGACTGAAAAGT |
| | | | TCTGTAGTTTCTACACCCAAGCCAC |
| | | | CACCCAAGCCACTGAAGTCATCTGT |
| | | | AATATTTGATTTGTTGACATCCCAA |
| | | | ACATGTTTTGCTTGGTTCTATAGTA |
| | | | GTTACTTAGGATCTATTTACCATAT |
| | | | GTATGAGAAATCCTCACCCAAGCAT |
| | | | TCACCCAAGCATTCAACCTAAATCT |
| | | | TTGGGTGCTGTCTTTAGTAACTTTT |
| | | | TGAACTTTATGAACCCATACTTTTA |
| 202322_s_at | GGPS1 | −0.46937914 | GATGCACGTGGTGGGAACCCTGAGC |
| | | | GGAACCCTGAGCTAGTAGCCTTAGT |
| | | | AAGCCATTCTTGATTGGACCTCATA |
| | | | TTCATTTAGAAGCCCCTCTGTACAG |
| | | | AAAGCAGCCACAGTTATGTAGGTCT |
| | | | AGTGACAGGACATTGCCACCAACTC |
| | | | AACTCTATCCTACTACCATCAATGT |
| | | | GTTCTCATTTCCTACTATTCATGCT |
| | | | TTGGTCAAGGCCTGAAAGCACCCAG |
| | | | CCAGGTGCAGAATATCTTGCGCCAG |
| | | | GAATACACTCGTAATACCCTTAAAG |
| 206896_s_at | GNG7 | −0.37146828 | TCTCTGTCTCAGGCAGGGCATCATT |
| | | | GGGCATCATTCAGTAATTAGCTCAA |
| | | | CAAACAAAACATCTCAAGTCCCCAA |
| | | | TCCCACCGCCCGGATGGGGTAGAAT |
| | | | AGGGATGGAGGCTTTACGGCCACTT |
| | | | AAAACTCTCGATTGCCGTTTCAATT |
| | | | CCGTTTCAATTGTGGACCGGCGCCG |
| | | | GACTTCGCCCGGTGGCAATAGTTCC |
| | | | GTTCCGGGAGAATTGGCCATTGGTA |
| | | | GACTTCATAGGGTCACTGGAATGCT |
| | | | GGGGCGGGAGGTGACATCATGAAGT |
| 220936_s_at | H2AFJ | 0.477249577 | TTATTGGGCAGGTTCGAGATGTTCT |
| | | | GATGTTCTGCTATTTACTCTGTGGT |
| | | | AATGCCTCATTGTTAGAACTACTAC |
| | | | GAACTACTACTCACAGTTACCACTT |
| | | | CTCACAGTTACCACTTGGGGTCAGT |
| | | | AAAATGGGCATAATAGTTTACCTCA |
| | | | GTGAGGACACTAAGATTCCCATATA |
| | | | GCGGTAGTTGATGGGAGCTGTTGAA |
| | | | GGTAAACAGCATTCTAGCAATCCTT |
| | | | GCAATCCTTCGACTTTTGTGATAGC |
| | | | GGACATCCACAATTCAATGTATAAC |
| 224301_x_at | H2AFJ | 0.591989591 | GAACTACGCGGAGCGAGTGGGCGCC |
| | | | TGTACCTGGCGGCGGTGTTGGAGTA |
| | | | TTACGGCGGAGATCCTGGAGCTGGC |
| | | | AGAAGACCAGGATAATTCCCCGCCA |
| | | | CTCGCCATCCGCAACGACGAGGAGT |
| | | | GCTGGGCAAAGTGACCATCGCTCAG |
| | | | GTGCTGCTGCCCAAGAAGACGGAGA |
| | | | CCCCCAGCAAAGGCCCTTTTCATGG |
| | | | GTCGTCCCGCAATGCTTTTGAATGT |
| | | | GTGCTGGATGTCATGGAGGGCCGGT |
| | | | GACATCTAGCGGGGAGGTGGGCGGC |
| 225245_x_at | H2AFJ | 0.611706619 | GTGATCATGTCCGGTCGCGGGAAAC |
| | | | GAACTACGCGGAGCGAGTGGGCGCC |
| | | | TGTACCTGGCGGCGGTGTTGGAGTA |
| | | | TTACGGCGGAGATCCTGGAGCTGGC |
| | | | AGAAGACCAGGATAATTCCCCGCCA |
| | | | CTCGCCATCCGCAACGACGAGGAGT |
| | | | GCTGGGCAAAGTGACCATCGCTCAG |
| | | | GTGCTGCTGCCCAAGAAGACGGAGA |
| | | | GTCGTCCCGCAATGCTTTTGAATGT |
| | | | GTGCTGGATGTCATGGAGGGCCGGT |
| | | | GACATCTAGCGGGGAGGTGGGCGGC |
| 228213_at | H2AFJ | 0.448371938 | TCCCCGAGGACTGGTCTGTTTAGTT |
| | | | GAGGACTGGTCTGTTTAGTTGTGCC |
| | | | AAAAGGCTTAGTCAGGCCCCATAAT |
| | | | TATGCAAACTTCACAATGCCCCTTC |
| | | | CAATGCCCCTTCCAGTGGTTGAAAG |
| | | | GGTTGAAAGGTCGCATACCATGCTG |
| | | | GTGTAAGAACTTTAGCTCTCTGCAA |
| | | | TTAGCTCTCTGCAATGAGACTTAAA |
| | | | AAATTCAGATTCACTCTACTCCTTA |
| | | | CTCTACTCCTTATTAGTTATCTGAT |
| | | | GAAACTTAATCTCTTTAAACCTCAG |
| 211999_at | H3F3B | 0.441145226 | CTTCTGACTGCACTTGTTCTCATAG |
| | | | ATGCTATGCGCATTTATACCTTGCA |
| | | | TACCTTGCATAAGTCCTCATTCTAC |
| | | | CTCATTCTACCACATGTTAACCCTC |
| | | | GTTAACCCTCTAGCTGATAATGCAA |
| | | | AACGAGTTATTCACACCAGCATCAT |
| | | | CATTGTGTTGTGTGGTTGGTCTCAT |
| | | | ACTAGGTTGAGTTTTTCTCCTCTGC |
| | | | CAGTACCGAAGTTCTTTTTCTTGTG |
| | | | GGGAGGAGCACAAAACTCCAGCCCA |
| | | | CCCACTGAACCTCTGCCAATTAAGA |
| 209069_s_at | H3F3C, | 0.336867187 | GTTGGTGAGGGAGATCGCGCAGGAT |
| | H3F3B | | CCGACCTGAGGTTTCAGAGCGCAGC |
| | | | ATCGGTGCGCTGCAGGAGGCTAGCG |
| | | | GGGTCTGTTCGAAGATACCAACCTG |
| | | | TGTGCCATCCACGCTAAGAGAGTCA |
| | | | CGCTAAGAGAGTCACCATCATGCCC |
| | | | CCCAAAGACATCCAGTTGGCTCGCC |
| | | | TTGGCTCGCCGGATACGGGGAGAGA |
| | | | GAAGGCAGTTTTTATGGCGTTTTGT |
| | | | AGGGATGGGTGATACTTCTTGCTTC |
| | | | ATGTGTACAGGGTCCTTTTGCAATA |
| 227679_at | HDAC11 | 0.465171926 | AGCCCTACTCATGGGGACATTCAGG |
| | | | GGAAGTGGGCGGGGGAGCATCCACC |
| | | | AAGTGGGTCCATTGAGGTGGCCCTG |
| | | | ACCCCGAGGCTCTAACAATGCACTC |
| | | | AACAATGCACTCTGAGATCCCTACC |
| | | | CTGACTCGAGGCACCTAACATCCAT |
| | | | CAACACAGGCCAGCGACTTCTGGGG |
| | | | CATGGTTTGTCACTGTTGAGCTTCT |
| | | | GAGCTTCTGTTCCTAGAGAATCCTA |
| | | | TAGAGGCTTGATTGGCCCAGGCTGC |
| | | | GTAGCGCAAGGCCTGACATGGGTAG |
| 209328_x_at | HIGD2A | 0.426198785 | ACTATAGACTCGAAGGATCCACAAG |
| | | | ACCATCGAAGCCTCCAGTCATTGAG |
| | | | GAAGCCTCCAGTCATTGAGGGGCTG |
| | | | CTCCAGTCATTGAGGGGCTGAGCCC |
| | | | CTCGAAGGATCCACAAGTTTGTACA |
| | | | TGAGGGGCTGAGCCCCACTGTTTAC |
| | | | GAGCCCCACTGTTTACAGGAATCCA |
| | | | TTACAGGAATCCAGAGAGTTTCAAG |
| | | | GCAGGCTTGTAAAACGACGGCCAGT |
| | | | GACGGCCAGTAACTATAACGGTCCT |
| | | | GCCAGTAACTATAACGGTCCTAAGG |
| 207156_at | HIST1H2AG | 0.410568514 | CCGGCCCACTCTGAAGTAATCTTAA |
| | | | TAAGAAGACGTTAACTCATTTTTCT |
| | | | TTTTCTTGTGTATTGTAGACACTTT |
| | | | TGTAGACACTTTTGGCTGTCTGGTA |
| | | | GGCTGTCTGGTAACATGGAAAATCT |
| | | | ACATTACATGATTTGGTGAGCCTAA |
| | | | GTGAGCCTAATTGCTGTTACTAATT |
| | | | AATGTTTCACGATAACTCAGCAATT |
| | | | TCACGATAACTCAGCAATTGTAATG |
| | | | AACATCTAATGTCTTTTGGGTTACA |
| | | | AACAGGTACTGAGATTTGTGGCCTA |
| 208579_x_at | HIST1H2BK | 0.532020648 | AAGCGCAGCCGCAAGGAGAGCTACT |
| | | | GAGAGCTACTCCGTATACGTGTACA |
| | | | CAAGGTGCTGAAGCAGGTCCACCCC |
| | | | GCATCTCCTCTAAGGCCATGGGAAT |
| | | | TGGGAATCATGAACTCCTTCGTCAA |
| | | | TCGTCAACGACATCTTCGAACGCAT |
| | | | TCGAACGCATCGCAGGTGAGGCTTC |
| | | | GCATTACAACAAGCGCTCGACCATC |
| | | | TCGACCATCACCTCCAGGGAGATCC |
| | | | TCACCAAGTACACCAGCGCTAAGTA |
| | | | GAAGGACGGCAGGAAGCGCAAGCGC |
| 209806_at | HIST1H2BK | 0.510579653 | TAAACTTGCCAAGGAGGGACTTTCT |
| | | | ACAATTGCCTTCGGTTACCTCATTA |
| | | | GGTTACCTCATTATCTACTGCAGAA |
| | | | GACGAGAATGCAACCATACCTAGAT |
| | | | ACCTAGATGGACTTTTCCACAAGCT |
| | | | CAAGCTAAAGCTGGCCTCTTGATCT |
| | | | TCCATTCCTTCTCTCTAATAATCAT |
| | | | TACTGTTCCTCAAAGAATTGTCTAC |
| | | | TCTCCTCTTTTGCCTCTGAGAAAGA |
| | | | GGGTAATATTCTGTGGTCCTCAGCC |
| | | | TCCTCAGCCCTGTACCTTAATAAAT |
| 208576_s_at | HIST1H3B | 0.507441215 | ATGGCTCGTACTAAACAGACAGCTC |
| | | | GCTACCAAAAGTCGACCGAGTTGCT |
| | | | AGTCGACCGAGTTGCTGATTCGGAA |
| | | | GTTGCTGATTCGGAAGCTGCCGTTC |
| | | | GAAATCGCCCAAGACTTCAAGACCG |
| | | | GCCCAAGACTTCAAGACCGATCTTC |
| | | | CAGACAGCTCGGAAATCCACCGGCG |
| | | | GGAGGCTTGTGAGGCCTACTTGGTA |
| | | | TGAGGACACAAACCTTTGCGCCATC |
| | | | CGAGTGACTATTATGCCCAAAGACA |
| | | | AATCCACCGGCGGTAAAGCGCCACG |
| 214634_at | HIST1H41 | 0.553652376 | GAGTCTCTTAATAGGGCCATTGTCA |
| | | | ACAGGTGACCTTGGGCCGAGATTTT |
| | | | ACTTCTGGCGGCTGCCTGGAAATTG |
| | | | TGGAAATTGCCTGCAGCCGGTTTAC |
| | | | TAGAAAGCCAAGGGGTCTGCGGTCC |
| | | | GTCTGCGGTCCAAATAGGGGCGGGC |
| | | | GGGCTAGATAATTAACTTCCCTCTG |
| | | | TTCCCTCTGGACCTTCAAATACGTC |
| | | | GGGCTCCACTAAATGCTAGAACCTC |
| | | | GGAGGGGGACAGACCATGCTTTTAC |
| | | | AATGCGCTGGTGACACACCACTTAT |
| 219269_at | HMBOX1 | −0.53153352 | AGGCTAGAAAATCTTGCTGCTCCGT |
| | | | GCTCCGTCTTAGCATTCCAAGAGAG |
| | | | AGATAGCCCTCAGTTCTCAAATATT |
| | | | TTGTAACACTAGTCTGTACTCCCTT |
| | | | TTTTCCTTCCCCAAGACTGATAGGA |
| | | | GGATGCAAGCTGAGGTCGTGGCACA |
| | | | GAATCCCCACCTCAGCGTGAGGATA |
| | | | TAAGCCGTGCCTCATTATAGCCACA |
| | | | GATTATACTTCTTTGGGTGCTGTGC |
| | | | GAAGTTAACATGCCTGACACAGACA |
| | | | AGATAAAATACTGCCTTCTGCCTTT |
| 225504_at | HMBOX1 | −0.80276841 | TGCTGCCTTTCTTCAGATCAGGTTA |
| | | | CAGATCAGGTTACCACAATGCCTCC |
| | | | CCCACTTTGCCGGTGCTAAAACACA |
| | | | GAAAGACAAGCTCCGGGTGTCCAGG |
| | | | TGACGGGCCAACCATGTGGCAGGTC |
| | | | GCTCCACAGTGGTCCCACTAATGGG |
| | | | GGGAGAGTGATACTGCACCTTCACC |
| | | | ACCTTCACCCGTAGGACTCATATTT |
| | | | GTAGCAAAAAGCCCTTGTTTCTAGA |
| | | | AGTCCTGTATCATTGTATCTCCTAT |
| | | | ATCTCCTATTCTGGATTAGTGCCTT |
| 209113_s_at | HMG20B | 0.344160466 | CCCACCCCGTGGACGAGAGGCTGGG |
| | | | TGGACGAGAGGCTGGGGGTCCACCC |
| | | | TTCGATGTTCCCATCTTCACTGAAG |
| | | | TTCACTGAAGAGTTCTTGGACCAAA |
| | | | GGACCAAAACAAAGCGCGTGAGGCG |
| | | | TCGGCGCTTGCGGAAGATGAATGTG |
| | | | TGTGGCCTTCGAGGAGCAGAACGCG |
| | | | AGAACGCGGTACTGCAGAGGCACAC |
| | | | CCAGCACGAGAAGCTCATCGTCCGC |
| | | | GCTCATCGTCCGCATCAAGGAAATC |
| | | | GCCAGCGAGCACCTGTGAGGAGTGG |
| 225107_at | HNRNPA2B1 | −0.40053 | TAATTCTAGTTCAGTGTCTTACCCT |
| | | | GTTCAGTGTCTTACCCTGAAGAGAA |
| | | | GAGAAAGTTGTAGGTTGGCTGTTGA |
| | | | TGGCTGTTGAAATTCATTCCTTAGA |
| | | | GATATGATCAGTTTGATTGCCCGGC |
| | | | TTGATTGCCCGGCTTTATTGCCTTT |
| | | | GGAATGTGATACTCAGGGCTTACTC |
| | | | CAGGGCTTACTCTATACACCAATGA |
| | | | ACACCAATGAGTCTTCTTTGATCCT |
| | | | AAGACCACCACTGAAGTTGTTTAGG |
| | | | GATAAACTTCTTCAGATACTTTTTT |
| 225932_s_at | HNRNPA2B1 | −0.39306534 | ACCATGGACAAGTATATTCTGCTGC |
| | | | TGGACAAGTATATTCTGCTGCCACA |
| | | | GCCACAAAGACTGTAAAGTGCTTCA |
| | | | AGTGCTTCATTTCAACAGCTGAGGC |
| | | | TCATTTCAACAGCTGAGGCAAGCCA |
| | | | GAGGCAAGCCAAGTGATCATTAATA |
| | | | TAAAGCTTTTCTTGGTTCCTTCAGT |
| | | | TCCTTCAGTGGTGTTGGTAGTAAAA |
| | | | GTGGTCAACCACAGAGTCTTCAAGA |
| | | | GAAAGTAGTTCTTGTTGGTGCCTTC |
| | | | GTTCTTGTTGGTGCCTTCATTTAAA |
| 210086_at | HR | 0.353065176 | ACCACTCTGGGCACAAGCAGGGCAC |
| | | | CCCTTAAGCCAACAACCACAGTGCC |
| | | | CCAGGCCCGCACTGGGGGCAATTGA |
| | | | TCCGAGACCCAGGAGACAAACAGCC |
| | | | GGGGAAACTTGGGAATCATTCTGGC |
| | | | ATTCTGGCTTAAACAACACCTCCTC |
| | | | GGCTCACTGCAGGCATGCTGAACAA |
| | | | GGCATGCTGAACAAGGGGCCTCCAA |
| | | | GAGAGGGTGGCATCAGGAGCTGCTC |
| | | | GCATGGGCGATGTCACTCATGCCCT |
| | | | TCCCTCCTTCATGATTTCCATTAAA |
| 220163_s_at | HR | 0.376939592 | CACCGGGCACAGAAAGACTTCCTTT |
| | | | CCAGGTCAGCACTGTGTGGCACGTG |
| | | | CCCCAGGCAGCTGCTACCTGGATGC |
| | | | TGCTACCTGGATGCAGGGCTGCGGC |
| | | | GGTGCAGGGCCTGGTGAGCACAGTC |
| | | | TGGTGAGCACAGTCAGCGTCACTCA |
| | | | CCACCTGCTTTATGCCCAGATGGAC |
| | | | GAAGGTGGCCGTGGGGACATTACAG |
| | | | GATGCTAGGTGTCTGGGATCGGGGT |
| | | | GTGGGGACAGGTAGACCAGGTGCTC |
| | | | GCCCAGGCACAACTTCAGCAGGGGA |
| 200064_at | HSP90AB1 | 0.702960924 | AATAGACTTGTGTCTTCACCTTGCT |
| | | | GTCTTCACCTTGCTGCATTGTGACC |
| | | | GTGACCAGCACCTACGGCTGGACAG |
| | | | GAGCGGATCATGAAAGCCCAGGCAC |
| | | | AAAAGCACCTGGAGATCAACCCTGA |
| | | | TGGTGGTGCTGCTGTTTGAAACCGC |
| | | | CAACCGCATCTATCGCATGATCAAG |
| | | | GCAGAGGAACCCAATGCTGCAGTTC |
| | | | TCCCCCCTCTCGAGGGCGATGAGGA |
| | | | GGGCGATGAGGATGCGTCTCGCATG |
| | | | AACTTGTGCCCTTGTATAGTGTCCC |
| | | | AATAGACTTGTGTCTTCACCTTGCT |
| | | | GTCTTCACCTTGCTGCATTGTGACC |
| | | | GTGACCAGCACCTACGGCTGGACAG |
| | | | GAGCGGATCATGAAAGCCCAGGCAC |
| | | | AAAAGCACCTGGAGATCAACCCTGA |
| | | | TGGTGGTGCTGCTGTTTGAAACCGC |
| | | | CAACCGCATCTATCGCATGATCAAG |
| | | | GCAGAGGAACCCAATGCTGCAGTTC |
| | | | TCCCCCCTCTCGAGGGCGATGAGGA |
| | | | GGGCGATGAGGATGCGTCTCGCATG |
| | | | AACTTGTGCCCTTGTATAGTGTCCC |
| 214359_s_at | HSP90AB1 | 0.545555043 | CATACCTCCCAGTCTGGAGATGAGA |
| | | | ATCTCTGTCAGAGTATGTTTCTCGC |
| | | | ATGTTTCTCGCATGAAGGAGACACA |
| | | | GGAGACACAGAAGTCCATCTATTAC |
| | | | GAAGTCCATCTATTACATCACTGGT |
| | | | CCATCTATTACATCACTGGTGAGAG |
| | | | GGTGAGAGCAAAGAGCAGGTGGCCA |
| | | | AAGAGCAGGTGGCCAACTCAGCTTT |
| | | | CCCTGCTGGTGTCTAGTGTTTTTTT |
| | | | AATCTCAAGCTTGGAATCCACGAAG |
| | | | GGAATCCACGAAGACTCCACTAACC |
| 221667_s_at | HSPB8 | 0.486202313 | GGGACTTAACATTTCACGTTGTATC |
| | | | ACGTTGTATCTTACTTGCAGTGAAT |
| | | | TGCAAGGGTTACTTTTCTCTGGGGA |
| | | | CCATGCCGCATGGTTTGGTTAATGA |
| | | | GCTTCCACATGCCTGGCCTAAAATG |
| | | | ATACAGGTCTTATATCCCCATATGG |
| | | | TGGAATTTATCCATCAACCACATAA |
| | | | TCAAAGTTTCCACATTAGCACTCCC |
| | | | TAAGGACGCTGGGAGCCTGTCAGTT |
| | | | GTTTATGATCTGACCTAGGTCCCCC |
| | | | TATGGGCGGGACGTGTGTGTCATTA |
| 204949_at | ICAM3 | 0.480810421 | GTACCCCGAGCTGCGGTGTTTGAAG |
| | | | CTCCAGCCGGGAGGTGCCGGTGGGG |
| | | | TCCCGTTCTTCGTCAACGTAACACA |
| | | | TGGTACTTATCAGTGCCAAGCGTCC |
| | | | AGCGTCCAGCTCACGAGGCAAATAC |
| | | | GGGCGTGGTGACTATCGTACTGGCC |
| | | | AATGTACGTCTTCAGGGAGCACCAA |
| | | | ACCAACGGAGCGGCAGTTACCATGT |
| | | | TAGGGAGGAGAGCACCTATCTGCCC |
| | | | TCACGTCTATGCAGCCGACAGAAGC |
| | | | ATTCCGCACCAATAAAGCCTTCAAA |
| 202069_s_at | IDH3A | 0.731202774 | CAGTCACTCTAAATGGACACCACAT |
| | | | TGGACACCACATGAACCTCTGTTTA |
| | | | ACCTCTGTTTAGAATACCTACGTAT |
| | | | GTATGCATTGGTTTGCTTGTTTCTT |
| | | | TTGCTTGTTTCTTGACAGTACATTT |
| | | | TTAGATCTGGCCTTTTCTTAACAAA |
| | | | GATGCAGGTGGATGTCCCTAGGTCT |
| | | | CAAAGAACTTTTTCCAAGTGCTTGT |
| | | | GAGTGGACTGTATCATTTGCTATTC |
| | | | GCACAAAATGACACTCTTCTAAAAC |
| | | | TGGGCACAAGAGAATTTTCCTGGGA |
| 202070_s_at | IDH3A | 0.909136128 | ATCCCAAAGCACCAATTACTGCCCT |
| | | | CCTCTGCCTCAGCAGTACCAGTATA |
| | | | GACTGGAGGCAACTCAGCCTGAGTT |
| | | | GAGCTTGAGCTTGGGCTTAGGCTTG |
| | | | TAGGCTTGGGCTCAGCTTTTGACCC |
| | | | ATCTTCAGACACTCACTATTTTCAT |
| | | | TCCCCACAACCAAAGACAACTCATG |
| | | | TCCTTTGGCCCTTGTGTAACATTGC |
| | | | GGCTTTGCAAAATGTACCCAGGTCA |
| | | | TTTAGCAATGATATCCCTGTCTGGG |
| | | | CCTGTCTGGGTCACTTTTTAAGCTT |
| 201508_at | IGFBP4 | −0.68950982 | AGAGACATGTACCTTGACCATCGTC |
| | | | CTTCCTCTCAAGCTAGCCCAGAGGG |
| | | | TAATGGTCACGAGGTCCAGACCCAC |
| | | | CCCAAAGCTCAGACTTGCCAGGCTC |
| | | | GTCCTTCCTTTAGGTCTGGTTGTTG |
| | | | CCATCTGCTTGGTTGGCTGGCAGCT |
| | | | GGAGAAGACCCACGTGCTAGGGGAT |
| | | | GCTAGGGGATGAGGGGCTTCCTGGG |
| | | | ACCCCATTTGTGGTCACAGCCATGA |
| | | | TCACCGGGATGAACCTATCCTTCCA |
| | | | GGCATCTTCTGGCTTGACTGGATGG |
| 203710_at | ITPR1 | −0.39512527 | CTCCGTCTCCTAGTGATAATGCTCC |
| | | | TAATGCTCCAAGTCTATGAACTGTT |
| | | | GGGAACTTTCTATGCAATGTTCAGG |
| | | | GGATAAATCGATACTGCTGGCCAAT |
| | | | CGATACTGCTGGCCAATCAGTGTCA |
| | | | TCTCCTGGGTAAATTTTGATGTCGC |
| | | | TTCTTCATCTGAACCAACATGCTAC |
| | | | TGACCACAGACATGTTATTCTTCTG |
| | | | GAAAGAGCCACATTTTGGTTTTATT |
| | | | TGAAATCTTTTATATCTGTTGCCTA |
| | | | ATCTGTTGCCTAGTTTTGTACATGG |
| 227514_at | ITPRIPL2 | −0.56159871 | GAAATATTTCTAGCAGTGTCAGTGA |
| | | | GTAACATACTGTTCTTGTAGTTTTT |
| | | | CAGGTTAATTACCCAAAGCCTCATC |
| | | | GCCATGCTGAGCAATTGTTCTCTGT |
| | | | TGTTCTCTGTACATGGTAACCAAAA |
| | | | GCCAGGCATGATGGTTGCCCAAGAC |
| | | | ATGGTTGCCCAAGACAGTTAAATTA |
| | | | AATTCTGTATTTTATTAGGGCTCTG |
| | | | TTAGGGCTCTGTTATGTCCTTCATC |
| | | | CTCTGTTATGTCCTTCATCTGAAAT |
| | | | GGTGTATGCTTGGTACTGGAGATTC |
| 227792_at | ITPRIPL2 | −0.59300408 | ATTTTTTTATACCTACATAGCACAT |
| | | | TGAAGTATCTACTATTCTGGAATAT |
| | | | GGTGCCTTGATTCAGTTGCGTGACT |
| | | | GCGTGACTTAGAACATTCATCCTAT |
| | | | TGTTTTTGGTTGCAGTCTGGCGGCT |
| | | | GCAGGCATAGCGTCGGTTTTGTTCC |
| | | | CAGATATGGTTCAGCTGCTACAATT |
| | | | ACAGTCAAGACCTGCCATTCGTTTT |
| | | | CATTCGTTTTCTCTTGCAGGTTGGA |
| | | | TTGCACTTTGAATCATGTGGGTCAT |
| | | | ATGTGGGTCATTTGGGGACCTTGTT |
| 227954_at | ITPRIPL2 | −0.46044034 | GGAAAGGCAATCGAGAGTTGGTTAG |
| | | | GATCAATTCACTCATTTTGTGGTAA |
| | | | AGGGAAGCCAGTTATATTTATTATT |
| | | | GTTCTGTGTAGACGGATTCTGTAGA |
| | | | GGATGTGGCTTTTAGAGAAGTCCAG |
| | | | GAAGCAAGAACTAGCTGCAGGGAAA |
| | | | GCAGGGAAAGTTCCTTCTGTCGGTT |
| | | | TTTAGACACAGATCTCTCTGCCCAA |
| | | | CAGATCTCTCTGCCCAAATTAAAAA |
| | | | GACACAATTACTTGCTAGGTACTGG |
| | | | GGTACTGGGTTCCTGATTGTCTTTA |
| 212492_s_at | KDM4B | −0.68721271 | GAGGAGGAGCTGCCCAAGAGGGTCC |
| | | | TGCCACGGAGGACTCCGGGCGGAGC |
| | | | ACCCCACTCAACTACTCAGAATTTT |
| | | | TAAACCATGTAAGCTCTCTTCTTCT |
| | | | GCTCTCTTCTTCTCGAAAAGGTGCT |
| | | | AAAAGGTGCTACTGCAATGCCCTAC |
| | | | TACTGCAATGCCCTACTGAGCAACC |
| | | | GCCCTACTGAGCAACCTTTGAGATT |
| | | | CTTTGAGATTGTCACTTCTGTACAT |
| | | | TGTCACTTCTGTACATAAACCACCT |
| | | | AAACCACCTTTGTGAGGCTCTTTCT |
| 212495_at | KDM4B | −0.66054335 | TAAAAGAGTGTCCTAACAGTCCCCG |
| | | | TCCTAACAGTCCCCGGGCTAGAGAG |
| | | | AACAGTCCCCGGGCTAGAGAGGACT |
| | | | TCCCCGGGCTAGAGAGGACTAAGGA |
| | | | GAGAGTGTTACGCAGGAGCAAGCCT |
| | | | GTGTTACGCAGGAGCAAGCCTTTCA |
| | | | AAAACGTGGAGGTGTCCCTCTGCAC |
| | | | CGTGGCGCTGACACTGTATTCTTAT |
| | | | GGCGCTGACACTGTATTCTTATGTT |
| | | | CACTGTATTCTTATGTTGTTTGAAA |
| | | | TGTAAAGAAGCGGGCGGGTGCCCCT |
| 212496_s_at | KDM4B | −0.71421129 | CAGAAGGGCAGGCCGGAGCTGCACA |
| | | | TCTCTGTGTCTTACTCTGTGCAAAG |
| | | | GTGTCTTACTCTGTGCAAAGACGCG |
| | | | CTTACTCTGTGCAAAGACGCGGCAA |
| | | | GCAAAGACGCGGCAAAACCCAGTGC |
| | | | CAAAGACGCGGCAAAACCCAGTGCC |
| | | | CCCACCCGAGATGAAGGATACGCTG |
| | | | GGATACGCTGTATTTTTTGCCTAAT |
| | | | ACGCTGTATTTTTTGCCTAATGTCC |
| | | | GTCCCTGCCTCTAGGTTCATAATGA |
| | | | TCCCTGCCTCTAGGTTCATAATGAA |
| 215616_s_at | KDM4B | −0.59551407 | CCAGGCCCTTCTGGTTGGTAGTGAG |
| | | | GCCCTTCTGGTTGGTAGTGAGTGTG |
| | | | TAGTGAGTGTGGACAGCTTCCCAGC |
| | | | CAGCTCTTCGGGTACAACCCTGAGC |
| | | | GGTACAACCCTGAGCAGGTCGGGGG |
| | | | CTGAGCAGGTCGGGGGACACAGGGC |
| | | | CCTGCTTCCGGGCAGGGACGAGGCC |
| | | | CTGCTGTCACCTGAGGGGAATCTGC |
| | | | GGAATCTGCTTCTTAGGAGTGGGTT |
| | | | GGAGTGGGTTGAGCTGATAGAGAAA |
| | | | GAGAAAAAACGGCCTTCAGCCCAGG |
| 202386_s_at | KIAA0430 | −0.75937109 | TGGACTTCAGTTCTGCTAGCATGTA |
| | | | GCTAGCATGTAAAGAGTGGTGGACT |
| | | | GTATCATTACAAGTCACCTGGAACA |
| | | | GAACAGGTTCTTTGGGCAACAGACA |
| | | | CTGTCCCGTGAGTGTGTCTGAGTAC |
| | | | GTGTGTCTGAGTACCATTCACTGGA |
| | | | TTCACTGGAGTTGCTGCTTAGGTCT |
| | | | TGTGACTCTTAACAATTGCTGTCTG |
| | | | GCACATTGTGTTCATAATGTACTCC |
| | | | TAATGTACTCCACAATGGCCAGTCC |
| | | | GCCAGTCCAATTGCTATCTATTTTT |
| 225623_at | KIAA1737 | −0.69265964 | TGTTCTCTGTTGGAGCTGTAAGCAG |
| | | | GGAAGGAGAGATCCATTGAGTCCAG |
| | | | GAGTCCAGAAGCCAGATCAGCAAAT |
| | | | GACCAGAAAGATCTCCATCGGTTGC |
| | | | CATCGGTTGCCCAAGGCTGTAAGTA |
| | | | GTAGTGATGGTTTTAGCGATGAATA |
| | | | ATTGGCTATGAAGTACTGTGGCAGA |
| | | | GAGAAGCCATTTTTAGCTCAGAGCA |
| | | | GAACTTTTGGCAGATTTTGTTGGCA |
| | | | TTATTACACTCATTGGTTTTTATTG |
| | | | TTTCTACTATGGTTCCTTTAGCAGA |
| 209008_x_at | KRT8 | 0.370951507 | GAGCAGCGTGGAGAGCTGGCCATTA |
| | | | AGGATGCCAACGCCAAGTTGTCCGA |
| | | | GGGCCAAGCAGGACATGGCGCGGCA |
| | | | CGGCAGCTGCGTGAGTACCAGGAGC |
| | | | TCGCCACCTACAGGAAGCTGCTGGA |
| | | | GCGGCTATGCAGGTGGTCTGAGCTC |
| | | | TCCTCCAGGGCCGTGGTTGTGAAGA |
| | | | TGGGAAGCTGGTGTCTGAGTCCTCT |
| | | | GCCCAAGTGAACAGCTGCGGCAGCC |
| | | | TGAACCGGAACATCAGCCGGCTCCA |
| | | | GCCGGCTCCAGGCTGAGATTGAGGG |
| 208029_s_at | LAPTM4B | 0.594307186 | ATTTTCTCCATGGCCTGAATTAAGA |
| | | | AAGACCATTAGAAAGCACCAGGCCG |
| | | | CTGACTGTTCTTGTGGATCTTGTGT |
| | | | TGTGGATCTTGTGTCCAGGGACATG |
| | | | ACATGGGGTGACATGCCTCGTATGT |
| | | | GTGACATGCCTCGTATGTGTTAGAG |
| | | | GTGGAATGGATGTGTTTGGCGCTGC |
| | | | TTGGCGCTGCATGGGATCTGGTGCC |
| | | | TGCCCTAGATTGGTTCAAGGAGGTC |
| | | | GGTTCAAGGAGGTCATCCAACTGAC |
| | | | GAGGTCATCCAACTGACTTTATCAA |
| 208767_s_at | LAPTM4B | 0.592105853 | GTAGAATTCTTCCTGTACGATTGGG |
| | | | TTCACTAACCTTCCCTAGGCATTGA |
| | | | AACTTCCCCCAAATCTGATGGACCT |
| | | | GGACCTAGAAGTCTGCTTTTGTACC |
| | | | TCTGTTCCCTCTCTTTTGAAAATGT |
| | | | GGGTTACTTGATTAGCTGTGTTTGG |
| | | | TGGAACTGCTACCGATACATCAATG |
| | | | ACTCCTCTGATGTCCTGGTTTATGT |
| | | | GCAATGACACTACGGTGCTGCTACC |
| | | | TGCCACTGTGAATGGTGCTGCCAAG |
| | | | GTCTGCCTAAGCCTTCAAGTGGGCG |
| 214039_s_at | LAPTM4B | 0.63024599 | ATATTTGATATACTTCTGCCTAACA |
| | | | TATACTTCTGCCTAACAACATGGAA |
| | | | CATCCTACTGCTTTGAACTTCCAAG |
| | | | GAACTTCCAAGTATGTCTAGTCACC |
| | | | CCAAGTATGTCTAGTCACCTTTTAA |
| | | | GAAAAATGAGGATTGCCTTCCTTGT |
| | | | TCCTTGTATGCGCTTTTTACCTTGA |
| | | | TTTTTACCTTGACTACCTGAATTGC |
| | | | GACTACCTGAATTGCAAGGGATTTT |
| | | | GTTACAAAGTCAGCAACTCTCCTGT |
| | | | ACTCTCCTGTTGGTTCATTATTGAA |
| 221558_s_at | LEF1 | −0.37358809 | AGCTTGTCTGGTAAGTGGCTTCTCT |
| | | | TGTAACACATAGTGGCTTCTCCGCC |
| | | | CTTCTCCGCCCTTGTAAGGTGTTCA |
| | | | CAAACCCCACTCTGTTGGTAGCAAT |
| | | | GTAGCAATTGGCAGCCCTATTTCAG |
| | | | AAACCTTAACAGATGCGTTCAGCAG |
| | | | CGTTCAGCAGACTGGTTTGCAGTGA |
| | | | AGCCCAGCACTTGAATTGTTATTAC |
| | | | TGAGCATTGATGTACCCATTTTTTA |
| | | | ACTGTCATCCTAACGTTTGTCATTC |
| | | | AACGTTTGTCATTCCAGTTTGAGTT |
| 218939_at | LETM1 | 0.681360884 | GCTCCTTCAGCAAGCAGGCTAGTCA |
| | | | TGGGGCTTCAAGGGCAATACCCCCG |
| | | | GCAATACCCCCGTGCTTAGGGTTTG |
| | | | GGTTCCTGGCAAAAATGTACCTCCA |
| | | | TCCAGGGGCCTCCAAGCATAGGATT |
| | | | GAAGACAGGAACGGCACAGGCGTCC |
| | | | GAAAGCAGCTGCACTCAGACAATGC |
| | | | AATGCCTTCTCCATTACTTGAAGCT |
| | | | CTTGAAGCTTCTTTCTGTTCAGCCA |
| | | | ACCTTTGTGCAGGGACAGTTGGCTT |
| | | | GGCTTCCAGAGGTTTCAGCTTTCAG |
| 222006_at | LETM1 | 0.446584363 | GATTACGGGCAAGTTTTTATTAGAG |
| | | | ACTCTCAGTTCTAACGCAGGGATTC |
| | | | GATTCAGGAATTGGGCTTTCAGACT |
| | | | TCCTTCTGCAGTGTCACAGTCCAGA |
| | | | GCAGTGTCACAGTCCAGACTTTTTT |
| | | | CACAGTGGGTCCCAGGGCTAGCAGG |
| | | | TCCCAGGGCTAGCAGGAGCGTGCTG |
| | | | GGTGCTGGCAGGAGTGTGCTGGTGA |
| | | | GGCAGGAGTGTGCTGGTGAGCCGGC |
| | | | GCTCTGTCTTTGTAAATCCTTCAGG |
| | | | TGTCTTTGTAAATCCTTCAGGGGTC |
| 208450_at | LGALS2 | 0.461084643 | ATCGCCGATGGCACTGATGGCTTTG |
| | | | GACAAGCTGAACCTGCATTTCAACC |
| | | | TTCAGCGAATCCACCATTGTCTGCA |
| | | | ATTGTCTGCAACTCATTGGACGGCA |
| | | | GCAACTGGGGGCAAGAACAACGGGA |
| | | | GGAGCTGTCACCATGACGGGGGAAC |
| | | | GCCAGATGGGCACGAGCTGACTTTT |
| | | | GAGCTACCTGAGCGTAAGGGGCGGG |
| | | | TAAGGGGCGGGTTCAACATGTCCTC |
| | | | GACATGAAGCCGGGGTCAACCCTGA |
| | | | GAAGATCACAGGCAGCATCGCCGAT |
| 213526_s_at | LIN37 | 0.517736033 | GAGGAGGGCTCAGAGGTAACCAACA |
| | | | GGTAACCAACAGCAAGAGTCGTGAT |
| | | | GTCGTGATGTGTACAAGCTGCCGCC |
| | | | CACACTCATCTATCGCAACATGCAG |
| | | | ATCTATCGCAACATGCAGCGCTGGA |
| | | | CATGCAGCGCTGGAAACGCATCCGC |
| | | | AAACGCATCCGCCAGAGGTGGAAGG |
| | | | AGCTTCGTTACTCAGAAAGCATGAA |
| | | | GAAAGCATGAAGATCCTACGAGAGA |
| | | | GATCCTACGAGAGATGTACGAACGA |
| | | | ACGAGAGATGTACGAACGACAGTGA |
| 203518_at | LYST | −0.6194195 | TTCCAAAGTCTCTGCTGTCAAGATA |
| | | | GATTCGAGAGAAAGCACGTGGCCAT |
| | | | ACGTGGCCATGTATGCTTTAACCTT |
| | | | ACATGTAGTGATACCTAGGCTGCAT |
| | | | TAGGCTGCATTTAGATCACCGTGTG |
| | | | ATCACCGTGTGCTCAGGCCAGGTGT |
| | | | GAATCCTGAGGTCCATGGAGGTGCA |
| | | | GAGATTACTCCTATTCACGTTGAAG |
| | | | ATAGGGTTGCTACTCATCTTTTTTT |
| | | | GCTCTGTTACCTTTATATACGCTGC |
| | | | TATACGCTGCCTCTTCAATTTGGAA |
| 210943_s_at | LYST | −0.42037139 | GAGCTAACCCTTCTTTTGAGAATAT |
| | | | AAGTGATACTACTATGAGCCCTTCA |
| | | | TATGAGCCCTTCACAGTATCTAACC |
| | | | CAGTATCTAACCTTCCCTTTACTGC |
| | | | CTTTACTGCACGCTCCAAATTTAAG |
| | | | AGAGCACGAGTTTCACGGAGCAAGA |
| | | | GGCTGATAGAGAGAGTTTTCCCCAT |
| | | | GCTGCTTTCATCTTGGCACATAGCC |
| | | | CACCTGCCGTTGCTGGGGCAAAACT |
| | | | CTGCTGGCCACACCTATCAGAAGGT |
| | | | AGTGTTTCCCTGTGGTTTAATGTGG |
| 204970_s_at | MAFG | 0.587513144 | GCTGGGAAGGATTCACTCTCTTTAG |
| | | | ACTCTCTTTAGCCCCAGGGGAGCAG |
| | | | TGAAGAGATGGGCTCTGCTCTGAGA |
| | | | GTAGGGCGGGCTTGAAGGCCCTGAT |
| | | | GGCCCTGATGGGTGGACCACCAGCC |
| | | | CTGACCCGTTGCACTGAACAAGACC |
| | | | CTGGTTGTGCGCTTAACGTGAGGGT |
| | | | AACGTGAGGGTGGGTCCAGTGTGCC |
| | | | GGTCCCGTGTCACTGTTTACATGAC |
| | | | GTGTGGTTATATAGCCCTTTATTTA |
| | | | TTGTAAACTTACGGACACCTCTTTG |
| 224466_s_at | MAFG | 0.46668662 | GAGGTGGGTCCAAGCAGAGTTGATC |
| | | | AGCAGAGTTGATCAGTCCCTGCCTG |
| | | | TGCCCTGCCTAGGTCTAGCCAGGGG |
| | | | GAGAAGACCCCGGGATCTGCTGGGG |
| | | | TGGATGCGGGGTGAGGCCCGAGCGC |
| | | | CGCTCACACTTCGTGTAGGGCGGGC |
| | | | CTGCCCTGCGCAGTATTTATTGCTA |
| | | | ATTATTGTCCAGGAGGGGCAGCACT |
| | | | TCTGCTAGTCCCTGAAGCCTTTAAC |
| | | | TGAAGCCTTTAACCAAACGGGAGTG |
| | | | AACAGGACAAGGGCTGCCCGCGTGT |
| 203668_at | MAN2C1 | −0.4312488 | CCACGAGTTCACCTATGCACTGATG |
| | | | CGCACAAGGGCTCTTTCCAGGATGC |
| | | | GGATGCTGGCGTTATCCAAGCTGCC |
| | | | AAGCTGCCTACAGCCTAAACTTCCC |
| | | | TTCACCCGCGGTCGTATTGGAGACC |
| | | | CCTGAGGCTGTATGAGGCCCACGGC |
| | | | CGGCAGCCACGTGGACTGCTGGCTG |
| | | | TCTGCGATCTCTTGGAGCGACCAGA |
| | | | CTCTGGGGACTCCTAATTTCTGCTT |
| | | | GCTTCCCCAGCCTAAAGCAGGGATC |
| | | | AGCAGGGATCAGTCTTTTCTTGTGG |
| 226132_s_at | MANEAL | 0.579062432 | TGGAAGGGTGTCAGGGTCTGGGCTC |
| | | | ATCATCTGTCTTCTCTAAGTTAGGG |
| | | | AGGAAATCATCCTGAGCACTCACAG |
| | | | GAGCACTCACAGGTTCATTTAACAC |
| | | | TTTAACACTCACTCATCAAGCACCT |
| | | | GAAGAGTTCCTGTCCTGAAGCTTCC |
| | | | TCTGGTGTGGCCTTGTAGCTAGTGC |
| | | | GTGCCTGGGCACAGGTGTTTTTCTT |
| | | | GTTTTACCTAGTGCTGGGAGTTCAG |
| | | | TGGGAGTTCAGTTCTTTTTCCTCTA |
| | | | AGAGCCTAATTTTTCCCAGATGCAT |
| 210058_at | MAPK13 | 0.453157102 | GGGTCCTTCTCCTTATGTGGGAAAT |
| | | | GTCGGTTGGGAGAAACTAGCTCTGA |
| | | | CTAGCTCTGATCCTAACAGGCCACG |
| | | | ACAGGCCACGTTAAACTGCCCATCT |
| | | | AAACTGCCCATCTGGAGAATCGCCT |
| | | | CTGGAGAATCGCCTGCAGGTGGGGC |
| | | | GGATGCTCTAACGAATTACCACAAA |
| | | | TTCCCCAGCTTATTGCTGCATCACT |
| | | | GTTCTCTCCTCTTTTAACAACAGTC |
| | | | CCCACCCTAATCCTGTGTGATCTTA |
| | | | GTGTGATCTTATCTTGATCCTTATT |
| 210059_s_at | MAPK13 | 0.449684703 | CCGGGGGCCTATGGCAGTGATGCTG |
| | | | GGGGCCTATGGCAGTGATGCTGTGT |
| | | | CCTATGGCAGTGATGCTGTGTTGGT |
| | | | CAAACCTGGTGGATTGAAACAGCAG |
| | | | GAAACAGCAGAACTTGATTCCCTTA |
| | | | AGCAGAACTTGATTCCCTTACAGTT |
| | | | CAGGGCTGTGGTCCCTTTGAAGGCT |
| | | | CCTTGGCTCTTTTTAGCTTGTGGCG |
| | | | TGGCTCTTTTTAGCTTGTGGCGGCA |
| | | | TTAGCTTGTGGCGGCAGTGGGCAGT |
| | | | TGATCCTTATTAATTAAACCTGCAA |
| 203928_x_at | MAPT | −0.7271058 | GAGTCCAGTCGAAGATTGGGTCCCT |
| | | | TCCCTGGACAATATCACCCACGTCC |
| | | | AAGACAGACCACGGGGCGGAGATCG |
| | | | CGGAGATCGTGTACAAGTCGCCAGT |
| | | | AGTCGCCAGTGGTGTCTGGGGACAC |
| | | | TCCACCGGCAGCATCGACATGGTAG |
| | | | GCTAGCTGACGAGGTGTCTGCCTCC |
| | | | GCCTCCCTGGCCAAGCAGGGTTTGT |
| | | | TGTGATCAGGCCCCTGGGGCGGTCA |
| | | | GCTCCTCGCAGTTCGGTTAATTGGT |
| | | | ATCACTTAACCTGCTTTTGTCACTC |
| 203929_s_at | MAPT | −0.74252419 | CAGGCTGGGTGTCTTGGTTGTCAGT |
| | | | GGATGGAAGGGCAAGGCACCCAGGG |
| | | | ATGGAAGGGCAAGGCACCCAGGGCA |
| | | | CTGCTCAGCTCCACATGCATAGTAT |
| | | | CTCAGCTCCACATGCATAGTATCAG |
| | | | GCTCCACATGCATAGTATCAGCCCT |
| | | | TCCACACCCGACAAAGGGGAACACA |
| | | | AGTTGTAGTTGGATTTGTCTGTTTA |
| | | | GTTGGATTTGTCTGTTTATGCTTGG |
| | | | GATTTGTCTGTTTATGCTTGGATTC |
| | | | TGTCTGTTTATGCTTGGATTCACCA |
| 203930_s_at | MAPT | −0.5823235 | GCCTGGCAGGAGGGTTGGCACTTCG |
| | | | GACTGACCTTGATGTCTTGAGAGCG |
| | | | TTGATGTCTTGAGAGCGCTGGCCTC |
| | | | TCTGAAGGTTGGAACTGCTGCCATG |
| | | | ACTGCTGCCATGATTTTGGCCACTT |
| | | | CCACTTTGCAGACCTGGGACTTTAG |
| | | | GCAGACCTGGGACTTTAGGGCTAAC |
| | | | TAGGGCTAACCAGTTCTCTTTGTAA |
| | | | ACCAGTTCTCTTTGTAAGGACTTGT |
| | | | TAAGGACTTGTGCCTCTTGGGAGAC |
| | | | GCATCTCTGGAGTGTGTGGGGGTCT |
| 206401_s_at | MAPT | −0.75694157 | GCAGCATCGACATGGTAGACTCGCC |
| | | | GCTAGCTGACGAGGTGTCTGCCTCC |
| | | | AGGTGGCAGTGGTCCGTACTCCACC |
| | | | GTCCAAGATCGGCTCCACTGAGAAC |
| | | | ACTGAGAACCTGAAGCACCAGCCGG |
| | | | GACCTGAGCAAGGTGACCTCCAAGT |
| | | | GGCTCATTAGGCAACATCCATCATA |
| | | | GAGTCCAGTCGAAGATTGGGTCCCT |
| | | | TCCCTGGACAATATCACCCACGTCC |
| | | | CGGAGATCGTGTACAAGTCGCCAGT |
| | | | AGTCGCCAGTGGTGTCTGGGGACAC |
| 225379_at | MAPT | −0.68822477 | TATGGACATCTGGTTGCTTTGGCCT |
| | | | TCAGGGGTCCTAAGCCCACAATCAT |
| | | | TCATGCCTCCCTAAGACCTTGGCAT |
| | | | GCTCCAGACACACAGCCTGTGCTTT |
| | | | TTGGAGCTGAGATCACTCGCTTCAC |
| | | | TCCTCATCTTTGTTCTCCAAGTAAA |
| | | | GTAAAGCCACGAGGTCGGGGCGAGG |
| | | | GCAGAGGTGATCACCTGCGTGTCCC |
| | | | GCCTCACCTCCTAATAGACTTAGCC |
| | | | GAGCAGGACTATTTCTGGCACTTGC |
| | | | GCAAGTCCCATGATTTCTTCGGTAA |
| 200978_at | MDH1 | 0.502702999 | TTTCCTCTGCCTGACTAGACAATGA |
| | | | GAATTTGTCACGACTGTGCAGCAGC |
| | | | GCTGCTGTCATCAAGGCTCGAAAAC |
| | | | AAACTATCCAGTGCCATGTCTGCTG |
| | | | CTGCAAAAGCCATCTGTGACCACGT |
| | | | GTGACCACGTCAGGGACATCTGGTT |
| | | | GTTTGTGTCCATGGGTGTTATCTCT |
| | | | TGATGGCAACTCCTATGGTGTTCCT |
| | | | TCCTGATGATCTGCTCTACTCATTC |
| | | | CTACTCATTCCCTGTTGTAATCAAG |
| | | | AAGGTCTCCCTATTAATGATTTCTC |
| 217542_at | MDM2 | 0.35395489 | AGTTTTTAGTTGCGCTTTATGGGTG |
| | | | TGCGCTTTATGGGTGGATGCTGAAT |
| | | | GTGATCATATTGTCTACCATGTAGC |
| | | | GTCTACCATGTAGCCAGCTTTCAAT |
| | | | GTAGCCAGCTTTCAATTATATGTAA |
| | | | TAAGAGGGACTTTTTGACATTTACA |
| | | | GATATCTGAAAGCACCAGCACTTGG |
| | | | GCAAGCAGATGGGAGGCGTGTTCAG |
| | | | GAGGCGTGTTCAGTAACTTATTCAT |
| | | | GAAATGATTGCTGTACTCAAATATT |
| | | | GAAAACCATAGTTGATTGCCTACAC |
| 238733_at | MDM2 | 0.353767856 | ACTTCTGCTTAAGAGGCTTCTATGT |
| | | | GGTACCTGTAATTTAGCCATTTCCT |
| | | | AGATGTAAGCTTGAGCCCATCCTCT |
| | | | GACTGTTAGTTTTCCAGTTCCTACT |
| | | | CAGTTCCTACTGGAGGCAAATTCTT |
| | | | GGCAAATTCTTTGTTTACCACTGTT |
| | | | GTTTACCACTGTTCTCTGTATTTCA |
| | | | AAAAGCCTTCTCTATATATCAGTAT |
| | | | GGGATGGTACGAGGCTGTATTATTT |
| | | | GAAATGGTCCCATAGCTTAGCATGT |
| | | | CCAGAAGGCATACTTTCCATCCATC |
| 244616_x_at | MDM2 | 0.571340098 | AAAAACTGGCTTTAAAGCAGGAGCT |
| | | | GGCCCCTAAGCCAGACGGGGACTAG |
| | | | AAGCCAGACGGGGACTAGCTTTGGC |
| | | | TGAGACGGAGTCTTGCTCTGTGGCT |
| | | | GCTCTGTGGCTCAGGCTGGAGTACA |
| | | | CTCCTGGCTGTGTTCAAGTGGTTCT |
| | | | AGCTGGGGTTAGAGCACCCTGTCAC |
| | | | CGCCCCGCTAATTTTGTATTTCTAG |
| | | | GATGAAGTTTCACTATGTTGGCCAG |
| | | | TAGTGTGTAGGTCTGTAGGCTTTTG |
| | | | TGTAGGCTTTTGATGGTAACCACAA |
| 210492_at | MFAP3L | 0.374647241 | GATCTCATCTTGTCTTGTTTTTCTA |
| | | | GTCTTGTTTTTCTAAGGCAGGAGAG |
| | | | TTTTTTTCCCTCATTGACACAGAAG |
| | | | TTTCCCTCATTGACACAGAAGACAA |
| | | | GACAAACACAGAAGTCTTTTTAAAG |
| | | | AATACATCCAATACATTATAGAGAC |
| | | | CATGGAGATGGCTGGAATAAACAAT |
| | | | ATAAACAGGAGCTTTGGAGCCAGCA |
| | | | GAGCCAGCACCCGTGATGTTAGTTC |
| | | | CACCCGTGATGTTAGTTCTTCTCAT |
| | | | GATGTTAGTTCTTCTCATGCAAATG |
| 207289_at | MMP25 | 0.354567436 | CCCCTCAAACTTCTGTGCACAAAGT |
| | | | TTCTGTGCACAAAGTGCTCCCTTCC |
| | | | GCCCCATCGGTGTGTAAGGTGGCCT |
| | | | CGGTGTGTAAGGTGGCCTATTCCTC |
| | | | TGTGTAAGGTGGCCTATTCCTCTGT |
| | | | GCCATGCTGACTGAGTGACTGGAGA |
| | | | CATGCTGACTGAGTGACTGGAGACA |
| | | | GACTGGAGACAGGGATGATGGAGAG |
| | | | GACAGGGATGATGGAGAGTTCATGA |
| | | | GCAGCAACTCTATGGTAGGGGGAGA |
| | | | ATGGTAGGGGGAGAGGGACCTGCCG |
| 207890_s_at | MMP25 | 0.563908849 | TCCTGGGAGGCCTTAGCTCTAGAGT |
| | | | CCACTCCCCACAGTTTTAGGATCTA |
| | | | GAACTATTCTTCTAGACTATCCCAC |
| | | | AGACTATCCCACATCAGAATCACTG |
| | | | GAATCCTCACTCAGGGTGGGGTCAG |
| | | | AATCTGCATTTTAACTAGTCGCGGG |
| | | | TAGTCGCGGGGATTGTGGGGGGCAG |
| | | | TCCACCCCAGGACCAATATGTTCAG |
| | | | GATGGCCTGAACCCCATGGGTAGAG |
| | | | TAGAGTCACTTAGGGGCCACTTCCT |
| | | | TAAGTTGCTGTCCAGCCTCAGTGAC |
| 218212_s_at | MOCS2 | 0.341587102 | GTGGTAGACATGTCCTTCCATGACT |
| | | | TCCTTCCATGACTAATTTCTAATTG |
| | | | CCCTCCTCAGTGACTTTAACTAGCT |
| | | | TAACTAGCTCAGAAACGTACTCCCC |
| | | | GTTCTGGGAGAGCATTGTTATTAAG |
| | | | GACAGTCTTGATATTATACATTTTC |
| | | | GAGTGCTTTTGGGCATCCAACAGTT |
| | | | GGCATCCAACAGTTAATCACTTATG |
| | | | TTTAGAGCATGCAATCTTAACTTTG |
| | | | TTTTCTCTCCACATCAGGATAGTTT |
| | | | ACTGAAGCACAATCTCTTATACTAG |
| 203801_at | MRPS14 | −0.6674614 | GAAGGCCTGAACTAACATTGTGGTA |
| | | | GATGGTTCTCTGGGTTCCTGATAAA |
| | | | AGGGCAATTCCAAGAGGGCAACTCC |
| | | | TTCAGGTTCCAGTCATGCGGTGTTG |
| | | | CGGTGTTGGAGATGCCTGTGTCATC |
| | | | GATGAAGACTAGTACGCAGCTGGAT |
| | | | GCAGCTGGATAGCAGAGTCCGAAAC |
| | | | GAATGTTCTAGCTAATATCTCAACT |
| | | | ACTTAGAATCCATCTCACTACCAAT |
| | | | TACCAATGGGCAAACACTTGTGTTC |
| | | | GTTTGAACATTTTGTGTACTTCCAA |
| 205614_x_at | MST1P9 | −0.40471778 | GCATGGAGAGCCAAGCCTACAGCGG |
| | | | TACAGCGGGTCCCAGTAGCCAAGAT |
| | | | CCTGCCCCCTGAATGGTATGTGGTG |
| | | | GTGCCTCCAGGGACCAAGTGTGAGA |
| | | | GGCCTTTCTGAATGTTATCTCCAAC |
| | | | TGCACTGAGGGACTGTTGGCCCCTG |
| | | | TGTGAGGGTGACTACGGGGGCCCAC |
| | | | GCTTTACCCACAACTGCTGGGTCCT |
| | | | TAATCCCCAACCGAGTATGCGCAAG |
| | | | TCACGCGTGTCTCTGTGTTTGTGGA |
| | | | TTAGGCCCAGCCTTGATGCCATATG |
| 213380_x_at | MST1P9 | −0.41073975 | CATGGAGAGCCAGGCCTACAGCGGG |
| | | | TACAGCGGGTCCCAGTAGCCAAGAT |
| | | | TAGCCAAGATGCTGTGTGGGCCCTC |
| | | | AAATGTGGCCTTGCTGAACGTCATC |
| | | | GAACGTCATCTCCAACCAGGAGTGT |
| | | | TGCACTGAGGGACTGTTGGCCCCTG |
| | | | GCTTTACCCACAACTGCTGGGTCCT |
| | | | GAATTAGAATCCCCAACCGAGTATG |
| | | | TCACGCGTGTCTCTGTGTTTGTGGA |
| | | | GAGACTGGGTTAGGCCCAGCCTTGA |
| | | | GCCTTGACGCCATATGCTTTGGGGA |
| 216320_x_at | MST1P9 | −0.46303812 | GCATGGAGAGCCAAGCCTACAGCGG |
| | | | TACAGCGGGTCCCAGTAGCCAAGAT |
| | | | CCTGCCCCCTGAATGGTATGTGGTG |
| | | | GTGCCTCCAGGGACCAAGTGTGAGA |
| | | | ATGTGGCCTTGCTGAATGTCATCTC |
| | | | TGCACTGAGGGACTGTTGGCCCCTG |
| | | | TGTGAGGGTGACTACGGGGGCCCAC |
| | | | GCTTTACCCACAACTGCTGGGTCCT |
| | | | TAATCCCCAACCGAGTATGCGCAAG |
| | | | TCACGCGTGTCTCTGTGTTTGTGGA |
| | | | TTAGGCCCAGCCTTGATGCCATATG |
| 201710_at | MYBL2 | 1.012936028 | CCCCTATGTCCAGTGCCTGGAAGAC |
| | | | ATGCAGGAGAAAGCCCGGCAGCTCC |
| | | | GACCCTCATCTTGTCCTGAGGTGTT |
| | | | TGAGGTGTTGAGGGTGTCACGAGCC |
| | | | GGTTGTGGGGGCAGAGGGGGTCTGT |
| | | | GGGTCTGTGAATCTGAGAGTCATTC |
| | | | CATTCAGGTGACCTCCTGCAGGGAG |
| | | | CCAGACTCTCAGGTGGAGGCAACAG |
| | | | GAGGCAACAGGGCCATGTGCTGCCC |
| | | | CGGCTCCTGGTGCTAACAACAAAGT |
| | | | AGACCCTGCTTAGGATGGGGGATGT |
| 218966_at | MYO5C | −0.60491631 | TCTTACCTGCCAACATATTCACCAT |
| | | | GCAACCTAAATTACTTTCGCTCTCT |
| | | | ACTTTCGCTCTCTAATCAGCATTTC |
| | | | ATTGTGTCGGACCCTACTTTTGAGA |
| | | | TGGGAACTGGCTATTCCTTGTCCCG |
| | | | TTGATAAGCACTCCTAGTCTCTGGC |
| | | | TAGTCTCTGGCCTGTGGATCCAGTG |
| | | | TGGATCCAGTGCTATTCTGTCACCA |
| | | | AAGAATCCCAATTGCACCTTCTGTT |
| | | | GCACCTTCTGTTTCTGACAGTCACA |
| | | | GCATCACCCTGCTAATACATAATAA |
| 209177_at | NDUFAF3 | 0.475161982 | TCTCGCCGGCGGATGACGAGCTGTA |
| | | | CGAGCTGTATCAGCGGACGCGCATC |
| | | | GAGGCCGCTCAGGCAATGTACATCG |
| | | | AACAGCCGCGGCTTCATGATAAACG |
| | | | TCCCGCACTCGGTGGTGCAGTGGAA |
| | | | ACATCACCGAAGACAGCTTTTCCCT |
| | | | GTTGCTGGAGCCCCGGATAGAGATC |
| | | | TGGAGACCGGACCGAGAGGCTGCAG |
| | | | CAGGAGGGACTTCACTTACATCTTT |
| | | | CAAGCTGCTCAATGAACCGCCAGGA |
| | | | CCGCCAGGAACTGACCTGCTGACTG |
| 222992_s_at | NDUFB9 | 0.731413576 | GTTGCGGCTTTATAAGCGGGCGCTA |
| | | | TCGAGTCGTGGTGCGTCCAGAGAGA |
| | | | AATACCGATACTTTGCTTGTTTGAT |
| | | | TGGGGGCACCTCCTATGAGAGATAC |
| | | | GAGATACGATTGCTACAAGGTCCCA |
| | | | GGTGCTTAGATGACTGGCATCCTTC |
| | | | AATGTATCCTGATTACTTTGCCAAG |
| | | | TGGTCCTTTAACTGAAGCTTTGCCC |
| | | | GATTTGCCCCCACTGTGGTGGTATA |
| | | | GTGGTATATTGTGACCAGACCCCGG |
| | | | GAGAGAGACCTCATCTTTCATGCTT |
| 203189_s_at | NDUFS8 | 0.664297427 | GTATGTGAACATGCAGGATCCCGAG |
| | | | GAACATGCAGGATCCCGAGATGGAC |
| | | | ATGCAGGATCCCGAGATGGACATGA |
| | | | GGATCCCGAGATGGACATGAAGTCA |
| | | | GAGATGGACATGAAGTCAGTGACTG |
| | | | GAAGTCAGTGACTGACCGGGCAGCC |
| | | | CCATCAACTACCCGTTCGAGAAGGG |
| | | | GTACCCATCCGGGGAGGAGCGTTGC |
| | | | CCCATCCGGGGAGGAGCGTTGCATT |
| | | | CATCCGGGGAGGAGCGTTGCATTGC |
| | | | AGGAGCGTTGCATTGCCTGCAAGCT |
| 203190_at | NDUFS8 | 0.518954457 | GCAGCCACCTACAAGTATGTGAACA |
| | | | CATCACCATCGAGGCTGAGCCAAGA |
| | | | GAGCCAAGAGCTGATGGCAGCCGCC |
| | | | CCCGCTATGACATCGACATGACCAA |
| | | | TGACCAAGTGCATCTACTGCGGCTT |
| | | | CCAACTTTGAGTTCTCCACGGAGAC |
| | | | TCCACGGAGACCCATGAGGAGCTGC |
| | | | GTTGCTCAACAACGGGGACAAGTGG |
| | | | GGGACAAGTGGGAGGCCGAGATCGC |
| | | | CCAACATCCAGGCTGACTACTTGTA |
| | | | CTGACTACTTGTATCGGTGACGCCC |
| 219438_at | NKAIN1 | 0.519264977 | ACTGCCTGGTGCGTCCATAGAGAGA |
| | | | GAACTGGGGGGCACCCAGATGGTGC |
| | | | TGCAGATGGTTTGCACACCTGAGCC |
| | | | CATTCCCTACTCTCTAAGGCCAAAA |
| | | | CACCATCCCAAATGCAAGCAGCCAG |
| | | | GGTGGGTACAGCTTGAGAGGGGGGC |
| | | | GCTTGAGAGGGGGGCAGCTCCCTCA |
| | | | ACTCAACGGGTGTAGCCACTGGTGC |
| | | | GCCACTGGTGCTTTGAAGCCTTTTG |
| | | | GACCAGGTTCTCTTTTCACTGGGAC |
| | | | CTCTTTTCACTGGGACCTTGCAAGG |
| 224010_at | NPB | 0.358813063 | CACACCGGATCCCTGATGTCTAGGG |
| | | | TCTAGGGAAGAGTCTTCTAGGTCCC |
| | | | CCTCCTGCCCTTGATCAAGAGACCA |
| | | | TCAAGAGACCAGTTCACTACTCAGA |
| | | | AGTTCACTACTCAGATGCACGTCTC |
| | | | TCCTTGGTGCCTTGACCATTCATGT |
| | | | TGGTGCCTTGACCATTCATGTGACC |
| | | | ATTCATGTGACCTTTTTGGCATCAC |
| | | | AGATCAAGTGTCTGCAGATGGGCCC |
| | | | CTGCAGATGGGCCCAGGGCCTGTAG |
| | | | GCCCAGGGCCTGTAGGCAAGGTGCC |
| 226414_s_at | NPB | 0.810447359 | TAAGTGCTGGAACGGCGTGGCCACT |
| | | | GCTCTGGGTGGCCAACGATGAGAAC |
| | | | ATGAGAACTGTGGCATCTGCAGGAT |
| | | | CATGCATTGCATCCTCAAGTGGCTG |
| | | | CAAGTGGCTGCACGCACAGCAGGTG |
| | | | TCTCGCTGGAGGGGCATCCTGAGAC |
| | | | CGCCCCTGAGCTGCAACAAGGTGGA |
| | | | GGGCTGGAGCTGCGTTTGTTTTGCC |
| | | | GTTTTGCCATCACTATGTTGACACT |
| | | | CACTATGTTGACACTTTTATCCAAT |
| | | | AAACTCATTAAACTACTCAAATCTT |
| 223381_at | NUF2 | 0.549670355 | GAGGTGCTGTCTATGAACGAGTAAC |
| | | | ACTGCTTTGGAGAAATACCACGACG |
| | | | ACCACGACGGTATTGAAAAGGCAGC |
| | | | GGCAGCAGAGGACTCCTATGCTAAG |
| | | | AGATGTTCAAAATGTCAACCTGATT |
| | | | ATGTCTTTTTGTAAATGGCTTGCCA |
| | | | TGGCTTGCCATCTTTTAATTTTCTA |
| | | | AATAATGTTGGCTTCATCAGTTTTT |
| | | | TCATCAGTTTTTATACACTCTCATA |
| | | | AATAACTTGTGCAGCTATTCATGTC |
| | | | TACTCTGCCCCTTGTTGTAAATAGT |
| 213075_at | OLFML2A | −0.45525209 | GGGAGCCCTGGGTTGGAATCCAGCC |
| | | | CCACCTCTTTTATGCCACAGGTTTG |
| | | | TCTCCCGCTCAGGGTAGGGCTGTGA |
| | | | TGAACTCCCTCTTACAGCTAAGAAC |
| | | | ATTATTCCTCCCCATTACAGGTGAT |
| | | | GAGAGCTTAAGCAACCTGCTCAGGG |
| | | | GTCACGTCTCCAACAGGCAGTAGAG |
| | | | GTTTTTGTACCAGAGTCCCAGACTA |
| | | | CCAATTGTGCTGAGTCTCCTACTAG |
| | | | CTAGACTCGCTTCATTCTAGCTTTC |
| | | | TCTAGCTTTCTGCTTTTACCTTTAC |
| 200897_s_at | PALLD | −0.70844818 | CTCTCTTAGCTCAGTTACTCAATTC |
| | | | ATCTGTGTACCACCCCATATATTTC |
| | | | TTCACATGTACAGCTTTCTACTTCT |
| | | | GAGCACCGGGTGGCAGATGTTCTAT |
| | | | GATGTTCTATGCAGTGTGGTTCAAG |
| | | | GTGGTTCAAGTTTCTTTGACCGCAC |
| | | | TTGACCGCACTTATATGCATTGCTA |
| | | | AAGATACCATACACAGTCTCTCATG |
| | | | TCTCTCATGGACCTATCTCTATTGT |
| | | | ATGTGACCTTTTTTTGCTGATTTGC |
| | | | TTAACTAGCATTATTTTGCCACCTT |
| 200906_s_at | PALLD | −0.72710984 | GTTGCTGATGGGTACCCAGTGCGGC |
| | | | CAGTGCGGCTGGAATGTCGTGTATT |
| | | | GTCGTGTATTGGGAGTGCCACCACC |
| | | | GTGCCACCACCTCAGATATTTTGGA |
| | | | GACCGAGTGAGCATGCACCAGGACA |
| | | | TGCCTGCTCATTCAGGGAGCCACAA |
| | | | AGGCTGGACGTTTACACCCAGTGGC |
| | | | GCATCAGCAGTCACAGAGCACCAAG |
| | | | CAGCACTTTCGGACCAGGGACTAGA |
| | | | TCAAAGCAGCGTTCCAACCTGAGGC |
| | | | GCCATTGCCTTGACCAACATATTCC |
| 200907_s_at | PALLD | −0.72999637 | AAACACTGCCATTCACAAGTCAAGG |
| | | | GGAACCCAGGGCCAGCTGGAAGTGT |
| | | | GTGGAGCACACATGCTGTGGAGCAC |
| | | | GCTGTGGAGCACACATGCTGTGGAG |
| | | | GCAGTGTGTCTGAGGTTTGTGTAGT |
| | | | GAAATTGCCTGTAGCATCTAGTCTA |
| | | | AAATTATTAGTTCACTTCCCTGCTG |
| | | | TGCTGCCATGAAACTTTGCCTTAAG |
| | | | GAAGGTGCTGGATTCCAAGGTTTGT |
| | | | AAGGCATCTCGGTAAAGACTGCTTT |
| | | | GACTGAGTTGATTCTGACCAGACTT |
| 209796_s_at | PAN2 | 0.441449841 | TGGAGCGACCCCATTACGCTAAAGA |
| | | | GAGCGACCCCATTACGCTAAAGATG |
| | | | GACCCCATTACGCTAAAGATGAAAG |
| | | | GAGCCAGGATCTCCACTGTGGAGCA |
| | | | GAATGGGAAATTGCCCAGGTGGACC |
| | | | ACCCCAAGAAGACCATTCAGATGGG |
| | | | GACCATTCAGATGGGATCTTTCCGG |
| | | | GATGGGATCTTTCCGGATCAATCCA |
| | | | GGATCTTTCCGGATCAATCCAGATG |
| | | | TTTCCGGATCAATCCAGATGGCAGC |
| | | | TCCAGATGGCAGCCAGTCAGTGGTG |
| 241867_at | PARP6 | −0.46940482 | CTCAGTCTTCCTGGCTTATGTCTTA |
| | | | GGCTTATGTCTTAGTTCATTTTCAG |
| | | | TTTTCAGTCTGCTTTTGTGCTTGTT |
| | | | GTGCTTGTTTGATGTAGTCTCTGTA |
| | | | AGTCTCTGTACAAGGTATAGTCACC |
| | | | GTCACCATGTAGTTGCATGTTCACT |
| | | | AAGGGGATTGTGCTAGATTCTTAAT |
| | | | AAAATGTAGTGCCATCAGGAGGCTG |
| | | | AGATGAGGTCCAGATTCTAATCAGG |
| | | | GAAAGTGCCAGAATCAGAGGCCTAA |
| | | | GATTTAGAGTTCTCTCAGTCTTCCT |
| 207838_x_at | PBXIP1 | −0.51747417 | GGGACTAAGGACAGCCATGACCCCC |
| | | | GCAGGAGGGCTTGACTTTCTTTGGC |
| | | | TTCTTTGGCACAGAGCTAGCCCCAG |
| | | | TAGCCCCAGTGCGGCAACAGGAGCT |
| | | | AAGAACATACTTGGCACGGCTGCCC |
| | | | GTGAGGATGGCATCTTCCGTCATGA |
| | | | CGCCTCCGATTCCGGGATTTTGTGG |
| | | | GACTTTGAGGACTTCATCTTCAGCC |
| | | | ATCTTCAGCCACTTCTTTGGAGACA |
| | | | AGCACTCACAGAGCCCAAGAGCTGC |
| | | | CCCACAGGGAATGGCCTTGGCCTTG |
| 212259_s_at | PBXIP1 | −0.58424221 | CAGCGTTATCTAACTCCTGGAGGGT |
| | | | TCCTGGAGGGTGGACTCTGTCCTGG |
| | | | GTTTGGTGTCCTCAGATATCTTTCA |
| | | | AGTAGAGCAAAATCACCAGCCCTGC |
| | | | CCCTGCACTGATGTCACTTTATGTA |
| | | | GGGGTCTGGGGAAGGCAATCTGATT |
| | | | GAGCTTTCATCCTCTTGAGTGTATG |
| | | | GAGTGTATGTCCCCATAGTGGGCCC |
| | | | CCAGCACGAGGACTTACCCTGGGGT |
| | | | GTTAGGTTTGGAAGCAGCTGTCCCT |
| | | | GCAGCTGTCCCTAGGGGGTGAAGTC |
| 214177_s_at | PBXIP1 | −0.5947096 | GTGGTTTCTAAGCACAGGGGACACC |
| | | | ACACCCCCTGCCTGAATGGATGGGT |
| | | | ATGGATGGGTCCATCCCAGGCACTG |
| | | | AACCCTAGGCCCTTGAGAAGCTGAT |
| | | | GAAGCTGATACTTCTCCTTTTGCTC |
| | | | CCCACCCCTGGGAGATGTAGCAAAT |
| | | | GTGGGTTTTGGAGTCTGAGCCTCAG |
| | | | CTGAGCCTCAGGCTCAAATCCAGGC |
| | | | GGCAAGTTAATCTCTGGGAACTTTG |
| | | | TCTCTGGGAACTTTGGGTTTCTTAT |
| | | | GTTTCTTATCCTCAAAAAAGGCGAT |
| 209577_at | PCYT2 | 0.40739165 | GGGAGCGCGATGGTGACTTCTAACC |
| | | | GGTGACTTCTAACCTGGCAGAGGCC |
| | | | TTGGACATAGGACTCTGCAGGGCCG |
| | | | GCCTACAAGGTGCCTGGTTTGCAGC |
| | | | CCGCTCTTTCCAGCAAAGCTGCTCA |
| | | | GCTGCTCAGAGAGGGTGTCCAGCAC |
| | | | GTGTCCAGCACAGTGGAGAGGCCGG |
| | | | GAAGTGAGACGGGCAGACGGCACCT |
| | | | GGTCACCCCTTTAGTTCTCTGGGTG |
| | | | TCTCTGGGTGTAGACCACACCACCT |
| | | | AGCGCCTGGCTCCAGGAAAACACGC |
| 230044_at | PCYT2 | 0.468900214 | CGGCCCCGTAGCAGCATTGGAGGCC |
| | | | GTAGCAGCATTGGAGGCCAGAGCCC |
| | | | GCGGAGGGAGAACCTACCCATCTCC |
| | | | TCTGGGGAGGCATGCTCTGGGCCTC |
| | | | AGAAGGGATGGGGGCAAGAGGAAGG |
| | | | CCCTCACAGATTTGGCTCTCGAGTT |
| | | | CACAGATTTGGCTCTCGAGTTGGGG |
| | | | GATTTGGCTCTCGAGTTGGGGAGCG |
| | | | GTTGGGGAGCGAAGGGCTGGGGGAG |
| | | | TTCTCATACCCAGGCTTGGGGATTT |
| | | | ATACCCAGGCTTGGGGATTTCCAGG |
| 222394_at | PDCD6IP | −0.51106369 | GATCTAAGAGAACTCTCCCTGTGCC |
| | | | GAAAAACAGTCACATGTCACGACAA |
| | | | GTCACGACAAACCAATCAATCTTTA |
| | | | TGAGATATTCCTGTATCCATACCCC |
| | | | GGATTTCACAGAGCCTTGTGTCCCT |
| | | | CACAGAGCCTTGTGTCCCTAAAGTT |
| | | | CCTAAAGTTCTGTCCCAGTCAGCAG |
| | | | GTCCCAGTCAGCAGTCTTTATAGTC |
| | | | GCAGTCTTTATAGTCCAAACAGATT |
| | | | CTTGAAGAATCTTGCTACAGCCAAA |
| | | | AAACCCTGCTAGGTAGTGTTATAAT |
| 219043_s_at | PDCL3 | 0.6367364 | TCATCTTGCACCTTTACAAACAAGG |
| | | | TGCACCTTTACAAACAAGGAATTCC |
| | | | ATCAAAGCCATTTCAACAACCTGCA |
| | | | AAGCCATTTCAACAACCTGCATACC |
| | | | TTCAACAACCTGCATACCCAATTAT |
| | | | CGATATTTGTTTACCTGGAAGGAGA |
| | | | GATATCAAGGCTCAGTTTATTGGTC |
| | | | AAGGCTCAGTTTATTGGTCCTCTGG |
| | | | CTCAGTTTATTGGTCCTCTGGTGTT |
| | | | AAACTGTCTGAATCTGGAGCAATTA |
| | | | CTGTCTGAATCTGGAGCAATTATGA |
| 219575_s_at | PDF | 0.576226091 | AAGGTGGGGTAATTGCATTCGTCTG |
| | | | TCTGCAGTAGACACGAGTTCCTCGG |
| | | | TCCTCGGACCTGTATAATCTCCCAA |
| | | | GGGCTGGACCCCAATGGAGAACAGG |
| | | | TCCAGCACGAGATGGACCACCTGCA |
| | | | ATGGACAGCAGGACGTTCACAAACG |
| | | | GCTTTGCTACTGGGGCTGAGGATTC |
| | | | GAGGATTCCGGATACCAAGACGCAA |
| | | | AAACACTTTCACTTTGAGCTGGGCA |
| | | | GAGCTGGGCAAATCTTACTTGGCAT |
| | | | TCAACTTGGATGGCTCGCATATGAC |
| 205380_at | PDZK1 | −0.51406251 | GTCAAACCATGACTCGCACATGGCA |
| | | | AAAGAACGGGCCCACAGTACAGCCT |
| | | | ATTTGATAGCTGTTTCTGGGTATTT |
| | | | GTGACCTGTTTACTGTCTCTTTAGA |
| | | | TCACCATGTGTGACTGTCTTCTGTT |
| | | | TTATCATTTGTCTTACAGGCGGCTA |
| | | | TACAGGCGGCTATTGCAGACGGCTA |
| | | | GATTTTTTTCATGTGATCTTTTCCA |
| | | | TTCCAAGCTTCAACTTAACTTAACT |
| | | | GTATGATGATGTCTCTTACTTCTAC |
| | | | TTACTTCTACAGGTTCCTTGAGCAC |
| 202464_s_at | PFKFB3 | −0.75010603 | TATTCTGTCCTGAGACCACGGGCAA |
| | | | TGTCCTGAGACCACGGGCAAAGCTC |
| | | | TTATTATTTTGATAGCAGATGTGCT |
| | | | GAGCCTCCTATGTGTGACTTATGAC |
| | | | TCTCTGTGTTCTGTGTATTTGTCTG |
| | | | GTGTTCTGTGTATTTGTCTGAATTA |
| | | | TTGTCTGAATTAATGACCTGGGATA |
| | | | GACCTGGGATATAAAGCTATGCTAG |
| | | | GCTATGCTAGCTTTCAAACAGGAGA |
| | | | GTATATTTTGCAGTTGCCAGACCAA |
| | | | GCAGTTGCCAGACCAATAAAATACC |
| 208305_at | PGR | −0.66800313 | GATGGAGATCCTACAAACACGTCAG |
| | | | AACACGTCAGTGGGCAGATGCTGTA |
| | | | ATTCTATTCATTATGCCTTACCATG |
| | | | TGCCTTACCATGTGGCAGATCCCAC |
| | | | GGCAGATCCCACAGGAGTTTGTCAA |
| | | | AGGAGTTGTGTCGAGCTCACAGCGT |
| | | | GCTCACAGCGTTTCTATCAACTTAC |
| | | | ACAACTTCATCTGTACTGCTTGAAT |
| | | | CCAGTCCCGGGCACTGAGTGTTGAA |
| | | | ATGATGTCTGAAGTTATTGCTGCAC |
| | | | ATTGCTGCACAATTACCCAAGATAT |
| 228554_at | PGR | −0.74820678 | CAGGGAATCTTTCTCATGACTCACG |
| | | | ATGACTCACGCCCTATTTAGTTATT |
| | | | ATTAATGCTACTACCCTATTTTGAG |
| | | | TAGGTCCCTAAGTACATTGTCCAGA |
| | | | TTTAGCCCCATATACTTCTTGAATC |
| | | | ATCTAAAGTCATACACCTTGCTCCT |
| | | | CCTTGCTCCTCATTTCTGAGTGGGA |
| | | | AATTGTTCTGAAGGTTTTTGCCAAG |
| | | | GTGATGGGGTGACAATGCAAAGCTG |
| | | | AGTGGGCACCTAATATCATCATCAT |
| | | | CAGTCTACTCAGCTTGACAAGTGTT |
| 200658_s_at | PHB | 0.758468503 | GCAGGGGATGGCCTGATCGAGCTGC |
| | | | CAGGGGATGGCCTGATCGAGCTGCG |
| | | | CAGCCCCGATGATTCTTAACACAGC |
| | | | GCAGGTGAGCGACGACCTTACAGAG |
| | | | TGAGCGACGACCTTACAGAGCGAGC |
| | | | TCCTGGATGACGTGTCCTTGACACA |
| | | | TGGATGACGTGTCCTTGACACATCT |
| | | | GACCTTCGGGAAGGAGTTCACAGAA |
| | | | TCGGGAAGGAGTTCACAGAAGCGGT |
| | | | GAGTTCACAGAAGCGGTGGAAGCCA |
| | | | GAGCAACAGAAAAAGGCGGCCATCA |
| 200659_s_at | PHB | 0.590980749 | GTCACTGATGGAAGGTTTGCGGATG |
| | | | GGATGAGGGCATGTGCGGCTGAACT |
| | | | CCAGCGGTTCCTGTGCAGATGCTGC |
| | | | GATGCTGCTGAAGAGAGGTGCCGGG |
| | | | GTCTGTCTGTTACCATAAGTCTGAT |
| | | | GAATCTGCCCCTGTTGAGGTGGGTG |
| | | | AGAGGAGGCCTGGACCGAGATGTGA |
| | | | CCCTCTCAGATACCCAGTGGAATTC |
| | | | TGAAGGATTGCATCCTGCTGGGGCT |
| | | | TGCTGGGGCTGAACATGCCTGCCAA |
| | | | GAACATGCCTGCCAAAGACGTGTCC |
| 202927_at | PIN1 | 0.598911611 | AGCCATTTGAAGACGCCTCGTTTGC |
| | | | ATTTGAAGACGCCTCGTTTGCGCTG |
| | | | TATTGTTCCCACAATGGCTGGGAGG |
| | | | CCGCCAGATTCTCCCTTAAGGAATT |
| | | | GATTCTCCCTTAAGGAATTGACTTC |
| | | | AAGGAATTGACTTCAGCAGGGGTGG |
| | | | GGTGCTGGAGGCAGACTCGAGGGCC |
| | | | GGAGGCAGACTCGAGGGCCGAATTG |
| | | | CAGACTCGAGGGCCGAATTGTTTCT |
| | | | TCAGTCGCAAAGGTGAACACTCATG |
| | | | AAAGGTGAACACTCATGCGGCAGCC |
| 206509_at | PIP | −0.87917581 | GGGGGCCAACAAAGCTCAGGACAAC |
| | | | GACATTCCCAAGTCAGTACGTCCAA |
| | | | AAAACTTACCTCATTAGCAGCATCC |
| | | | CAGCATCCCTCTACAAGGTGCATTT |
| | | | ATAAGTATACTGCCTGCCTATGTGA |
| | | | GACGACAATCCAAAAACCTTCTACT |
| | | | ATTGCAGCCGTCGTTGATGTTATTC |
| | | | ATTCGGGAATTAGGCATCTGCCCTG |
| | | | GCCCTGATGATGCTGCTGTAATCCC |
| | | | TAATGGAAGCCCTGTCTGTTTGCCA |
| | | | GTTTGCCACACCCAGGTGATTTCCT |
| 204458_at | PLA2G15 | 0.399043552 | GCCTTCTGGGAACCTATGGAGAAAG |
| | | | AGGGAATCCAAGGAAGCAGCCAAGG |
| | | | GGGTCTCACTAGTACCAAGTGGGTC |
| | | | GCACCCAGCTTAGTGCTGGGACTAG |
| | | | GGGACTAGCCCAGAAACTTGAATGG |
| | | | GGCAGTAGGCTCTAAGTGGGTGACT |
| | | | TGGGTGACTGGCCACAGGCCGAGAA |
| | | | GAAAAGGGTACAGCCTCTAGGTGGG |
| | | | CTGTTGCATACATGCCTGGCATCTG |
| | | | CCCACATGGGGCTCTGAGCAGGCTG |
| | | | GAGCAGGCTGTATCTGGATTCTGGC |
| 239392_s_at | POGK | −0.43817793 | TATGCTGAACATTTAGGGCCAGTAT |
| | | | GGGCCAGTATGTGTAACTGACATGC |
| | | | GGACAGTTGTACTCACTTTTGCTGG |
| | | | GTCTCAGTCCTGGAGCTATCTACAG |
| | | | GGAGCTATCTACAGTATGTTACCAG |
| | | | ATCTACAGTATGTTACCAGCGAGTA |
| | | | GAATAATAGCTTCTACTTGCTTTTC |
| | | | TACTTGCTTTTCCCTACAGAGTTCA |
| | | | GCTTTTCCCTACAGAGTTCAGGAGT |
| | | | TAAAACGTCATCTTAGTCTCATTAT |
| | | | TCATCTTAGTCTCATTATGACCTTC |
| 223260_s_at | POLK | −0.3539018 | GAAGAATGTTCTAGTCTCCCAAGCA |
| | | | TAGTCTCCCAAGCAAGTCTTTTAAT |
| | | | CAGAATTCTTCTTCTACTGTTTCAT |
| | | | ATTTAGACAAGAATACCGCCAGCCT |
| | | | ACCGCCAGCCTTACTTATGTGAAGT |
| | | | AACAGGCCAAGCTCTAGTTTGTCCT |
| | | | TAGTTTGTCCTGTTTGTAACGTAGA |
| | | | AAAGACTTCAGATCTAACCCTGTTC |
| | | | CTAACCCTGTTCAATGTGCATGTGG |
| | | | AAGCTCCAGAAGTACTGGTAGCTCA |
| | | | AAACAATCCCAAACATACCCTTGAT |
| 223261_at | POLK | −0.47165573 | GAATAAGCACTTGAATCAGTTTTTA |
| | | | GTCAATTATGTTGGTACTTTCCACA |
| | | | GAAGGATAAATTGTACCATCATTTT |
| | | | ATCATTTTATTATAATCCTCAAGAG |
| | | | GTATCTTAGTTACATTTCTATCAGT |
| | | | TACATTTCTATCAGTACTTTTATTA |
| | | | GTAGTTAGCTTAAGTAGTTTCTCCA |
| | | | GTAGTTTCTCCAAGTACTTTTGTGC |
| | | | TTCTCCAAGTACTTTTGTGCTATCA |
| | | | TTTGTGCTATCAATGAGTTCTTCTC |
| | | | AATAATTAGTTAGGCCAGGCACAAT |
| 202066_at | PPFIA1 | 0.411816725 | TAAAGAACGAACCTAGTGGGACATT |
| | | | GGGACATTTTTAGACTTTGATGCTC |
| | | | GATGCTCTAGCCATTTTGGATTGTG |
| | | | GGATTGTGTAAGTTGCAGATGTGGC |
| | | | TGCAGATGTGGCTTTTACTTTTTAA |
| | | | AAAGTGTGTCAGACCATGGCGTGGT |
| | | | ATGGCGTGGTATTTATTGTGCAGCA |
| | | | CAGAGGCAGCCTGTCTTTTCAGTTG |
| | | | TGTTTTTCTATTAATCTTTTGTCAA |
| | | | ATCTTTTGTCAACTTCCTGATTATG |
| | | | GTATGTACAGTCTACTTTTGAACTA |
| 210235_s_at | PPFIA1 | 0.350893924 | TCGAGTGATTCGCTGGATCCTGTCA |
| | | | GGATCCTGTCAATTGGCCTTAAAGA |
| | | | GAGCACTTCTGGCCTTAGATGAAAC |
| | | | GGCCTTAGATGAAACCTTCGACTTC |
| | | | GCACTGGCACTGCTGTTACAGATCC |
| | | | TTACAGATCCCGACGCAGAACACAC |
| | | | AGAACACACAGGCTCGTGCTGTCTT |
| | | | CAACCTTTTGGTCATGGGGACTGAT |
| | | | GCTTTAGGAGAGCACCTTCATGGAG |
| | | | AAAGGACATTCGTGGCTTAGCTGCT |
| | | | TCCCTGCAAACTTCCGGGTGACTTC |
| 210236_at | PPFIA1 | 0.48250638 | TGCAGATGGACGGTATGTGATGGGT |
| | | | TGATGGGTCACACTAACCTGTCACT |
| | | | GTCACACTAACCTGTCACTTGTTGG |
| | | | CTAACCTGTCACTTGTTGGGAGCAT |
| | | | TGTCACTTGTTGGGAGCATGAGCAG |
| | | | GCAGCTTTCTGTCTGGAACATTAAT |
| | | | AATAATGATCTAAAACGGCCTATTT |
| | | | AACGGCCTATTTAATATGTTACAAG |
| | | | TTTAATATGTTACAAGGCACTTGAG |
| | | | GGCACTTGAGTATGGTTGCATGTCC |
| | | | TGAGTATGGTTGCATGTCCAAATAT |
| 201957_at | PPP1R12B | −0.70564636 | GTTGTGCCTACCACTGGCTGGCACA |
| | | | ACTGGCTGGCACACCAGGGCAATGA |
| | | | GGGCAATGATTTCCCTGCAGAAGGA |
| | | | GAAAGAATGTTTCACCCTTGCATCC |
| | | | AGCTACAGCCTGTGCTCAGTTGAGT |
| | | | GTTCACACTCAGACTTTGGCTTTAT |
| | | | AAGAACCACCCTGAGGTTTCCATGC |
| | | | ATGCCTCTCCCATTTTAGTGGTAGC |
| | | | GGTAGCATTTTGTGTCTTTACTCCA |
| | | | TAGTTCCACCAAGGTTCACACACCA |
| | | | TTTGAGTGGCCTTTCAACCCTAAGA |
| 208680_at | PRDX1 | 0.644273963 | TTCTCACTTCTGTCATCTAGCATGG |
| | | | GGGACCCATGAACATTCCTTTGGTA |
| | | | TTTGGTATCAGACCCGAAGCGCACC |
| | | | GAAGCGCACCATTGCTCAGGATTAT |
| | | | GAAGGCATCTCGTTCAGGGGCCTTT |
| | | | AAGGGTATTCTTCGGCAGATCACTG |
| | | | GTTGCCGCTCTGTGGATGAGACTTT |
| | | | CTTTGAGACTAGTTCAGGCCTTCCA |
| | | | AGGCCTTCCAGTTCACTGACAAACA |
| | | | GTGAGCGCTGGGCTGTTTTAGTGCC |
| | | | TTTAGTGCCAGGCTGCGGTGGGCAG |
| 218302_at | PSENEN | 0.429531662 | TGCATCTGTTACTTAGGGTCAAGGC |
| | | | TAGGGTCAAGGCTTGGGTCTTGCCC |
| | | | CCCCAGCGCAGCTATGAACCTGGAG |
| | | | GAACCTGGAGCGAGTGTCCAATGAG |
| | | | AACCTGTGCCGGAAGTACTACCTGG |
| | | | CCTTTTCTCTGGTTGGTCAACATCT |
| | | | TTGGTCAACATCTTCTGGTTCTTCC |
| | | | CAGCCTACACAGAACAGAGCCAAAT |
| | | | ATCAAAGGCTATGTCTGGCGCTCAG |
| | | | TCTGGGTGATAGTGCTCACCTCCTG |
| | | | GCCGGAGGAAGTGAGCTCTCCTGGG |
| 203447_at | PSMD5 | −0.40381229 | GATTGCTGAGGGTTTTGCTTTGGAT |
| | | | TGCTTTGGATTTTTCATACCTATAA |
| | | | GTTCTTCTCTCTAAACAGCAAAGCC |
| | | | AGCAAAGCCAAAGCACTCTGCACAC |
| | | | GAGCATATTTCTTTTAGGCCGTGGT |
| | | | GTGAAGTTGATAAACCACCCCTGCT |
| | | | TCTAGTCCCCAGATTGATCATCTCC |
| | | | GGCAACGTGACTCTGTTTTTTGTGT |
| | | | TGTGTGTGTTTCCATGCTGACTAGT |
| | | | GACTAGTCCCCTACTGTTAATATCA |
| | | | TTAGGCTATAACCAGGTCTTTCCTG |
| 206687_s_at | PTPN6 | 0.515021453 | GGGCCTGGACTGTGACATTGACATC |
| | | | GACGGAGGCGCAGTACAAGTTCATC |
| | | | CCATCGCCCAGTTCATTGAAACCAC |
| | | | GCAGTCGCAGAAGGGCCAGGAGTCG |
| | | | GCCAGGAGTCGGAGTACGGGAACAT |
| | | | CCTATCCCCCAGCCATGAAGAATGC |
| | | | GAAGCAGCGGTCAGCAGACAAGGAG |
| | | | GAGGAAGTGAGCGGTGCTGTCCTCA |
| | | | ACCCTGTGGAAGCATTTCGCGATGG |
| | | | TTCGCGATGGACAGACTCACAACCT |
| | | | CACAACCTGAACCTAGGAGTGCCCC |
| 201482_at | QSOX1 | −0.59145376 | TGGAATGGAACTCCTCACTAGCTGC |
| | | | CTGCTCCCTTCCGGACAATGAAGAA |
| | | | CTCCTGGGTGGGGTTTGGCTTCAGG |
| | | | TGGTCTCCCAGGTGAGGCAAGCCAT |
| | | | TAGGGTGAGTGGCTTGCTTGGTGGG |
| | | | GTGGGACCTGACGAGTTGGTGGCAT |
| | | | GGGAAGGATGTGGGTCTCTAGTGCC |
| | | | CTTGCCCTGGCTTAGCTGCAGGAGA |
| | | | GAGAAGATGGCTGCTTTCACTTCCC |
| | | | TTTGGTCTCCAAGATGAATGCTCAT |
| | | | GGAGGGTGCCAGGTAGAAGCTAGGG |
| 219681_s_at | RAB11F1P1 | 0.430010968 | CAGGCTAATAGCGTGGTTGGGGGTG |
| | | | TGTCCTTGTTACATTGAGGTTAAGA |
| | | | GTGCAATCTCTTTCCAGGATTTCGT |
| | | | GGATTTCGTTTGCTGTGGCATTGGT |
| | | | GGCATTGGTTATATCAGAGCACTTT |
| | | | GCTTTTAATTATCTACAGCTATTTT |
| | | | TTCTCTCCTACAGTACTGGGACCAC |
| | | | CTGGGACCACTGTAAACTTCTCAGA |
| | | | AAACTTCTCAGATGACTTGTATTTT |
| | | | TTGTTGTTACTCACTTAAGACTGGA |
| | | | TTTTTTCCCTGGCTATGATAGAATC |
| 225177_at | RAB11F1P1 | 0.575553639 | GAAGGAGTAAGTCTGCCCTTTGCCA |
| | | | TGTGGACCCCGATTGGTGAGGGCTC |
| | | | GTGAGGGCTCTGCATATGCCTGTAT |
| | | | GTGTGTGCACATGCCGGTATGAAGA |
| | | | CAGGCATGTGCTTCTCAGTTTTGCT |
| | | | GTCCATGATGCTCAGCCACATACTG |
| | | | AATGTTAAATGACGCACCATCCTCC |
| | | | GAACTACTAATTATCTCTCAAGGCT |
| | | | GTATCCACCAAACTTAACTCCGTAT |
| | | | TAACTCCGTATCTCCATATGGTGTC |
| | | | ACTGAAGGATCGCCCAACGTTTTTG |
| 231830_x_at | RAB11F1P1 | 0.356254051 | TACAGTTCTCCAGGTGTGGAATGAT |
| | | | GTCAATACGATTGCTTGGCCTTTTC |
| | | | CAGCAACACTCCTTGTAAGGGGCAG |
| | | | TAAGGGGCAGAGACAGGGTCCACCA |
| | | | TCCACCAACTCCCCAAGATGAAGAA |
| | | | AGATGAAGAAGCCCCTTCAGGCCAG |
| | | | TCAGGCCAGTCGTGGTGGCTCATGC |
| | | | CAGCACTTTGCAAGGCCGAGGAGGG |
| | | | GGAGGCTGCAGCGAGCCAAGATCGT |
| | | | AGGAGACCATAGGATTTGGACCCCA |
| | | | GACCCCAAAGGGATGTGAACTGATC |
| 202252_at | RAB13 | −0.61976806 | GAGAGATGCCTCAGGCTTCAGACCT |
| | | | CTTCAGACCTTACCTGGGTTTTCAG |
| | | | AGGGTCCTGCAAAAGGCTAGCTCGG |
| | | | GCTAGCTCGGCACTACACTAGGGAA |
| | | | ACTAGGGAATTTGCTCCTGTTCTGT |
| | | | TCACTTGTCATGGTCTTTCTTGGTA |
| | | | GGTATTAAAGGCCACCATTTGCACA |
| | | | CAGGAAACGGCAACAAGCCTCCCAG |
| | | | GCCTCCCAGTACTGACCTGAAAACT |
| | | | GAAGAACACCAACAAGTGCTCCCTG |
| | | | CACCCCGGAAGCTGAACCTGAGGGA |
| 243777_at | RAB7L1 | 0.650862748 | AAGGAGCTGACTGGGATTCAGTCAC |
| | | | TGACTTGGAGCCGCTCGGGGGAAGT |
| | | | GACTTGGAGCCGCTCGGGGGAAGTC |
| | | | TTTCTCGGCAGTCAGGCCAGGAGGG |
| | | | CTTCCTCACAATTTGGTTTGTGCTG |
| | | | GTTTGTGCTGCAAGGGGAGGGTCCC |
| | | | GTGCTGCAAGGGGAGGGTCCCCATC |
| | | | GCAAGGGGAGGGTCCCCATCATCTG |
| | | | CAAGGGGAGGGTCCCCATCATCTGG |
| | | | GCCCCAGTGGTGTAAGGAGCTGACT |
| | | | GGTGTAAGGAGCTGACTGGGATTCA |
| 220338_at | RALGPS2 | −0.49372178 | GAGAGCTAACGTTTGATAGTTCTAA |
| | | | GAATCCTTATAGAATTTGTCTTTTA |
| | | | AAATTACTCTTCTTTAATGCTAAGT |
| | | | AATGCTAAGTATTGACACATCGTTG |
| | | | TTGACACATCGTTGTTTGTTTTTCA |
| | | | TTTCATTGTTTTTGCGGATTGAGAG |
| | | | GCGGATTGAGAGACTTGGTCCATCT |
| | | | ACTTGGTCCATCTTGTCTCAGGAGA |
| | | | GAAACCTTTCTCCAATGTAGCAGAA |
| | | | TATCCTCTTCCCTGTATTATAGCAA |
| | | | TTAAAGATTTTTGAGGCCGGGCACA |
| 227224_at | RALGPS2 | −0.57369561 | TTACAGACTCTAGCTTTCCTTATTA |
| | | | TATTAGCTTAAACTGGGGCCCTCAA |
| | | | CCCTCAAAGAGCAGCCTGTTGATCT |
| | | | TAAACTGTATACCTTTACTACTGAA |
| | | | TGGGTTCCATCCATTAGCTTTTTAA |
| | | | GATCTGGTATTGATTTCCTTCCTGT |
| | | | GGCACATTCCTTTACAACCAGTGTT |
| | | | TAAACCACCACGTAATCATCTTCTG |
| | | | AACAAGGGGTGCCAGTGTTGCCTAA |
| | | | GAGTTTAACTGTGTCCAGGTGGAGT |
| | | | GTATGACTTCTTTAGTGACCTTTTA |
| 227533_at | RALGPS2 | −0.73589294 | GCTGTGCTTTAGATACCAGATAACA |
| | | | GTTTCCCCTGAAGATATGACCTACT |
| | | | ATGACCTACTAGAACTACTCACATA |
| | | | GAGTTTCTGTACCTTGATTATTGAC |
| | | | GGGGTGGGGAACTGGTTCACAACAT |
| | | | GTAAGGACAGGTACCCAGTGATGAT |
| | | | TTTATTCTTTATCCCAATTAACTTG |
| | | | AGCACTCGATTGCACTATGACCTCC |
| | | | ATGACCTCCTTGAGTGATGTGCAGC |
| | | | GTGAGTGTGCGATCTTCAGTGTGTC |
| | | | CAGTGTGTCTGCATAAGCTAACTTA |
| 232112_at | RALGPS2 | −0.62900747 | TCTGGCGGTGCTGTGCTTGGAATAG |
| | | | AGATCGTAGCTAATTTGCATTTCTT |
| | | | AAAACATACTCTTGTGGATTCCATC |
| | | | GTGGATTCCATCAGGAGCTGGTTTT |
| | | | GGAGCTGGTTTTGAACCGAGGTGAA |
| | | | AATGTTAGTCATGTGAGTTCTTGGG |
| | | | ACATTATTTCCTGCAGGAGGTACAA |
| | | | AGGAGGTACAAAGGCTGTGTGTTCA |
| | | | GGCTGTGTGTTCATTTGCCAGACGC |
| | | | TGTGTTCATTTGCCAGACGCTTTTT |
| | | | TTCATTTGCCAGACGCTTTTTTTTT |
| 242458_at | RALGPS2 | −0.63334516 | ACAATAATTAGATCTTTTTCCAAGT |
| | | | CCCTTCTCCCAGTCATAGGTGGTTT |
| | | | GGTGGTTTTTATCATCAAGACAGAC |
| | | | CAGTGTTTGATGTGCATAATGCCAG |
| | | | TTCTCTCTTTTTGTTCAATATGAGA |
| | | | GATTCAGGATCATATTTGTTTAAAA |
| | | | CTGAAAATTTACTGTCGGTCTCTGA |
| | | | GTCGGTCTCTGACATGAAACCGTAT |
| | | | GAAACCGTATTTTGTCAGTAGTTGA |
| | | | GTAGTTGACCAAGCAGTTTTATGAG |
| | | | GAGAACTCTTCTATGCAATGATGCA |
| 203750_s_at | RARA | 0.417245729 | GCCTGACCACTGGGTGTGGACGGTG |
| | | | ACCACTGGGTGTGGACGGTGTGGGG |
| | | | GGGCAGCCCTGAAAGGACAGGCTCC |
| | | | GCAGCCCTGAAAGGACAGGCTCCTG |
| | | | TGCACCCACCATGAGGCATGGAGCA |
| | | | CCATGAGGCATGGAGCAGGGCAGAG |
| | | | GGAGCAGGGCAGAGCAAGGGCCCCG |
| | | | CCCCCACTGTGAAGGGGCTGGCCAG |
| | | | CACACACACACTGGACAGTAGATGG |
| | | | GGACAGTAGATGGGCCGACACACAC |
| | | | AGATGGGCCGACACACACTTGGCCC |
| 206499_s_at | RCC1 | 0.693335069 | TGTCCCTAACAGTCCACAGGCAAAC |
| | | | TCATAAGAGCCATCTGTCACGGACC |
| | | | ACCCACGCCCAGAGGAACGTGCAGA |
| | | | AAGTGATTCTCCCAGAAGCACAAAG |
| | | | AAGCACAAAGCATACTCTTGCCCCT |
| | | | CCCTCAGGTGTTGCTTGTGTACATC |
| | | | TGCTTGTGTACATCGTACCCATCCA |
| | | | AGCCAACGGCCTGGAATCGCAAAGA |
| | | | AAAGAGACACCACTCTGGGCAGAGC |
| | | | GGAGGGACAGAGTGTTGGAGGGCCA |
| | | | GGAGGGCCAGAGACTAGTCCTGAGA |
| 215747_s_at | RCC1 | 0.561781335 | CCCAGAACCTAACATCCTTCAAGAA |
| | | | AGGAAAAGCATACAGCCTGGGCCGG |
| | | | CCTGGGCCGGGCTGAGTATGGGCGG |
| | | | CCTCTGTGGGGTATGCTGTGACCAA |
| | | | CCAAGGATGGTCGTGTTTTCGCCTG |
| | | | CAACTACCAGCTGGGCACAGGGCAG |
| | | | CGCCTGGAGCCCTGTGGAGATGATG |
| | | | GAACCGTGTGGTCTTATCTGTGTCC |
| | | | GGGGCCAGCATACAGTCTTATTAGT |
| | | | AGAGCTGATGAAGCCTCTGAGGGCC |
| | | | CAGCTGCAGATGGCAGCGGGCCTCT |
| 204336_s_at | RGS19 | 0.391879986 | CAGTGGGGAGTGCTGTGTCTCCTGG |
| | | | GCCAAGCAGGAACTCCAGGTGCAGG |
| | | | TGGGGGCTCTTGCGTGGTGAGAGTA |
| | | | TGCGTGGTGAGAGTAGGGGTCCCCC |
| | | | TTGGTGGGGAACAGAACCTCCGCAT |
| | | | ACCTCCGCATCGTGTAGTTTTGTGA |
| | | | TACTTGAGCTGTCTGTACCCCAGAA |
| | | | TGTACCCCAGAATCAAACACAGAAC |
| | | | CTCAGAATCCTGCACTCAAGGTGGC |
| | | | TAAACCTGGAAACATGTCCTTACTA |
| | | | ACATGTCCTTACTAGGTGTTTTATC |
| 227543_at | RNASEH2C | 0.501348241 | TTCCTCACCCTCATAATGGACCTTA |
| | | | GACTGAGTTTCTTCAAGCATCCACT |
| | | | TCCACTTGTGCTACCAGGCTGAGAA |
| | | | GACCCCATCTGGGCATCATTTAACC |
| | | | AGATTCCTGTCTCTAATCCAGACCT |
| | | | TAGCTGGGACCTTGGGAGTGTCACC |
| | | | AAGACTTGAGTGGCCTGACTGGGTG |
| | | | GGTGCTTCCTAAGTCGGGGAGACCA |
| | | | TCCAACTCGTGCTGATAGCTGGCCG |
| | | | TGCACAGCCCTGAGTGGCTTCACAT |
| | | | TTCACATCTCTTGGTCAGTGTCTTC |
| 200088_x_at | RPL12 | −0.50184421 | GAAGTTCGACCCCAACGAGATCAAA |
| | | | AGTCGTATACCTGAGGTGCACCGGA |
| | | | GCAACGGGTGACTGGAAGGGCCTGA |
| | | | GACCATTCAGAACAGACAGGCCCAG |
| | | | GGCCCAGATTGAGGTGGTGCCTTCT |
| | | | TCGACAGATGCGGCACCGATCCTTA |
| | | | ACCGATCCTTAGCCAGAGAACTCTC |
| | | | AGATCCTGGGGACTGCCCAGTCAGT |
| | | | GGCTGTAATGTTGATGGCCGCCATC |
| | | | CGCCATCCTCATGACATCATCGATG |
| | | | GTGGTGCTGTGGAATGCCCAGCCAG |
| | | | GAAGTTCGACCCCAACGAGATCAAA |
| | | | AGTCGTATACCTGAGGTGCACCGGA |
| | | | GCAACGGGTGACTGGAAGGGCCTGA |
| | | | GACCATTCAGAACAGACAGGCCCAG |
| | | | GGCCCAGATTGAGGTGGTGCCTTCT |
| | | | TCGACAGATGCGGCACCGATCCTTA |
| | | | ACCGATCCTTAGCCAGAGAACTCTC |
| | | | AGATCCTGGGGACTGCCCAGTCAGT |
| | | | GGCTGTAATGTTGATGGCCGCCATC |
| | | | CGCCATCCTCATGACATCATCGATG |
| | | | GTGGTGCTGTGGAATGCCCAGCCAG |
| 200809_x_at | RPL12 | −0.4817517 | GAGGTGAAGTCGGTGCCACTTCTGC |
| | | | GCAACGGGTGACTGGAAGGGCCTGA |
| | | | GGCCCAGATTGAGGTGGTGCCTTCT |
| | | | CCCTGATCATCAAAGCCCTCAAGGA |
| | | | TCGTCCCGAATCCGGGTTCATCCGA |
| | | | TCGACAGATGCGGCACCGATCCTTA |
| | | | ACCGATCCTTAGCCAGAGAACTCTC |
| | | | AGATCCTGGGGACTGCCCAGTCAGT |
| | | | TGTTGATGGCCGCCATCCTCATGAC |
| | | | GTGGTGCTGTGGAATGCCCAGCCAG |
| | | | GAAGTTCGACCCCAACGAGATCAAA |
| 214271_x_at | RPL12 | −0.38430476 | GCAACGGGTGACTGGAAGGGCCTGA |
| | | | GACCATTCAGAACAGACAGGCCCAG |
| | | | GGCCCAGATTGAGGTGGTGCCTTCT |
| | | | CCCTGATCATCAAAGCCCTCAAGGA |
| | | | AACATTGCTCGACAGATGCGGCACC |
| | | | AGATCCTGGGGACTGCCCAGTCAGT |
| | | | TGTTGATGGCCGCCATCCTCATGAC |
| | | | AGTGGTGCTGTGGAATGCCCAGCCG |
| | | | TGCCCAGCCGTAAGTGACATTTTCA |
| | | | GTTACTGGTGGGGTGGGATAATCCT |
| | | | TTTTCTTTCCCACAGAGTTAAGCAC |
| 200074_s_at | RPL14 | −0.39457538 | TCAGAACAGGGCTTTGGTCGATGGA |
| | | | GGCTTTGGTCGATGGACCTTGCACT |
| | | | TGCCTTTCAAGTGCATGCAGCTCAC |
| | | | ATGCAGCTCACTGATTTCATCCTCA |
| | | | AAATGGGCAGCCACACGATGGGCCA |
| | | | GGCAGCCACACGATGGGCCAAGAAG |
| | | | AAGATGACAGATTTTGATCGTTTTA |
| | | | GAAGCTTCAAAAGGCAGCTCTCCTG |
| | | | TCCCAAAAAAGCACCTGGTACTAAG |
| | | | GCACCTGGTACTAAGGGTACTGCTG |
| | | | TGCTGCTGCTAAAGTTCCAGCAAAA |
| | | | TCAGAACAGGGCTTTGGTCGATGGA |
| | | | GGCTTTGGTCGATGGACCTTGCACT |
| | | | TGCCTTTCAAGTGCATGCAGCTCAC |
| | | | ATGCAGCTCACTGATTTCATCCTCA |
| | | | AAATGGGCAGCCACACGATGGGCCA |
| | | | GGCAGCCACACGATGGGCCAAGAAG |
| | | | AAGATGACAGATTTTGATCGTTTTA |
| | | | GAAGCTTCAAAAGGCAGCTCTCCTG |
| | | | TCCCAAAAAAGCACCTGGTACTAAG |
| | | | GCACCTGGTACTAAGGGTACTGCTG |
| | | | TGCTGCTGCTAAAGTTCCAGCAAAA |
| 213588_x_at | RPL14 | −0.42386316 | GGCAGACATCAATACAAAATGGGCA |
| | | | AGCAAAAAAGATCACCGCCGCGAGT |
| | | | GCCGCGAGTAAAAAGGCTCCAGCCC |
| | | | TAAAAAGGCTCCAGCCCAGAAGGTT |
| | | | CAGGCCAGAAAGCAGCGCCTGCTCC |
| | | | CGCCTGCTCCAAAAGCTCAGAAGGG |
| | | | GAAGGGTCAAAAAGCTCCAGCCCAG |
| | | | AAAAAGCTCCAGCCCAGAAAGCACC |
| | | | AGAAAGCACCTGCTCCAAAGGCATC |
| | | | ACCTGCTCCAAAGGCATCTGGCAAG |
| | | | CATCTGGCAAGAAAGCATAAGTGGC |
| 211073_x_at | RPL3 | −0.49096872 | GGAACCAAGAAGCGGGTGCTCACCC |
| | | | AAGTCCTTGCTGGTGCAGACGAAGC |
| | | | TTAAGTTCATTGACACCACCTCCAA |
| | | | GCCTGCGCAAGGTGGCCTGTATTGG |
| | | | TGTATTGGGGCATGGCATCCTGCTC |
| | | | GCACGCGCTGGGCAGAAAGGCTACC |
| | | | TACCATCACCGCACTGAGATCAACA |
| | | | GATTGGCCAGGGCTACCTTATCAAG |
| | | | GATCAAGAACAATGCCTCCACTGAC |
| | | | CTCCACTGACTATGACCTATCTGAC |
| | | | TCAACCCTCTGGGTGGCTTTGTCCA |
| 211666_x_at | RPL3 | −0.37973203 | TGTGGGCATTGTGGGCTACGTGGAA |
| | | | CTCCGGACCTTCAAGACTGTCTTTG |
| | | | GAAGGCCTTTACCAAGTACTGCAAG |
| | | | AGAAGTACTGCCAAGTCATCCGTGT |
| | | | GCTTCCTCTGCGCCAGAAGAAGGCC |
| | | | AGAAGGCCCACCTGATGGAGATCCA |
| | | | GCACTGTGGCCGAGAAGCTGGACTG |
| | | | GAGGCTTGAGCAGCAGGTACCTGTG |
| | | | CAAAGGGGTCACCAGTCGTTGGCAC |
| | | | GCCTGCGCAAGGTGGCCTGTATTGG |
| | | | CCTGTATTGGGGCATGGCATCCTGT |
| 212039_x_at | RPL3 | −0.43456801 | GCACGCGCTGGGCAGAAAGGCTACC |
| | | | TACCATCACCGCACTGAGATCAACA |
| | | | GATTGGCCAGGGCTACCTTATCAAG |
| | | | GATCAAGAACAATGCCTCCACTGAC |
| | | | CTCCACTGACTATGACCTATCTGAC |
| | | | CCTCTGGGTGGCTTTGTCCACTATG |
| | | | GGAACCAAGAAGCGGGTGCTCACCC |
| | | | AAGTCCTTGCTGGTGCAGACGAAGC |
| | | | GAAGCGGCGGGCTCTGGAGAAGATT |
| | | | TTAAGTTCATTGACACCACCTCCAA |
| | | | CTCCAAGTTTGGCCATGGCCGCTTC |
| 215963_x_at | RPL3 | −0.3611629 | GGAACCAAGAAGCGGGTGCTCACCC |
| | | | TGGTGCAGATGAAACGGCAGGCTCT |
| | | | TTAAGTTCATTGACACCACCTCCAA |
| | | | ACCACCTCCAAGTTTGGCCATGGCT |
| | | | CAAAGGGGTCACCAGTCGTTGGCAC |
| | | | TTGGCACACCAAGAAGCTGCCCTGC |
| | | | TGTATTGGGGCATGGCATCCTGCTC |
| | | | CTGGGGAGAAAGGCTACCGTCACCG |
| | | | GATTGGCCAGGGCTACCTTATCAAG |
| | | | GATCAAGAACAATGCCTCCACTGAC |
| | | | AGAGCACCAATCCTCTGGGTGGCTT |
| 211720_x_at | RPLP0P6 | 0.389281786 | ACGCTGCTGAACATGCTCAACATCT |
| | | | CTGGTCATCCAGCAGGTGTTCGACA |
| | | | GGCAGCATCTACAACCCTGAAGTGC |
| | | | CAGAGGAAACTCTGCATTCTCGCTT |
| | | | CTTCCTGGAGGGTGTCCGCAATGTT |
| | | | ATGTTGCCAGTGTCTGTCTGCAGAT |
| | | | GATTGGCTACCCAACTGTTGCATCA |
| | | | AGTACCCCATTCTATCATCAACGGG |
| | | | TGTGGAGACGGATTACACCTTCCCA |
| | | | AAGGTCAAGGCCTTCTTGGCTGATC |
| | | | GCAGCCCCAGCTAAGGTTGAAGCCA |
| 203777_s_at | RPS6KB2 | 0.687015289 | GCTTCACACGGCAGACGCCGGTGGA |
| | | | AGACGCCGGTGGACAGTCCTGATGA |
| | | | TCAGCGAGAGTGCCAACCAGGCCTT |
| | | | ATCAAGGAGGGCTTCTCCTTCCAGC |
| | | | TCCAGGGCGCTAGGAAGCCGGGTGG |
| | | | TGGGGGTGAGGGTAGCCCTTGAGCC |
| | | | TGTCCCTGCGGCTGTGAGAGCAGCA |
| | | | GTTCCAGAGACCTGGGGGTGTGTCT |
| | | | GGTGGGGTGTGAGTGCGTATGAAAG |
| | | | TGCGTATGAAAGTGTGTGTCTGCTG |
| | | | CTGAATCATGGGCACGGAGGGCCGC |
| 218914_at | RRNAD1 | −0.60664512 | CACTGGAGACAGTCATCCGACGGGC |
| | | | CAGGGTCCACGAGCTCAAGATTGAA |
| | | | ATATGTGCAGCGGGGGCTACAGCGA |
| | | | GGCTACAGCGAGTGGGGCTAGATCC |
| | | | TGGCCCAGGAGAACCGTGTGGTGGC |
| | | | TGGAGACGCTTATTCTACTGGACCG |
| | | | GAACTCTCTCCCAGAAACCTGGTTC |
| | | | GAGACTGAAGACAGCTGATGCAGCC |
| | | | CATCTCAGACCCCATCATCTGAAAG |
| | | | CAGTGGCAGAGTACATCTCATCCAG |
| | | | TCTCATCCAGAGAAACAGCATCCTG |
| 228923_at | S100A6 | 0.344698431 | CTCTCCAAATGAGGACCAGTAACTG |
| | | | GTAACTGAGAAGTAGCTGAGGAGAA |
| | | | GCAATGCCAAAGTGACATGGGTCCT |
| | | | AAAGTGACATGGGTCCTTGGTGATG |
| | | | GGGTCCTTGGTGATGAGGGAGCACA |
| | | | GGGGAAGAATCCAGGGTTGTCATCA |
| | | | AAGAATCCAGGGTTGTCATCACCAC |
| | | | GTCATCACCACTGAGTATGGATTTC |
| | | | CACTGAGTATGGATTTCACATTCTA |
| | | | CCCTGGTCCACATGTAGACCCTGAG |
| | | | ACATGTAGACCCTGAGCTGTAGACC |
| 206378_at | SCGB2A2 | −0.69528746 | CTGGCTGCCCCTTATTGGAGAATGT |
| | | | ATTTCCAAGACAATCAATCCACAAG |
| | | | GTTCATAGACGACAATGCCACTACA |
| | | | ACCAAACGGATGAAACTCTGAGCAA |
| | | | ATGACAGCAGTCTTTGTGATTTATT |
| | | | TAACTTTCTGCAAGACCTTTGGCTC |
| | | | AGACCTTTGGCTCACAGAACTGCAG |
| | | | GAGAAACCAACTACGGATTGCTGCA |
| | | | TACGGATTGCTGCAAACCACACCTT |
| | | | CTTCTCTTTCTTATGTCTTTTTACT |
| | | | GCAGCAGCCTCACCATGAAGTTGCT |
| 203453_at | SCNN1A | −0.5731827 | GACTCCCGAGGGCTAGGGCTAGAGC |
| | | | TTCATACCTCTACATGTCTGCTTGA |
| | | | CTGCCAGAGAACTCCTATGCATCCC |
| | | | TTACTTTTGTGAACGCTTCTGCCAC |
| | | | GTCTTCCCCAAAATTGATCACTCCG |
| | | | CTCCCGTAGCACACTATAACATCTG |
| | | | GCTGGAGTGTTGCTGTTGCACCATA |
| | | | GTTGCACCATACTTTCTTGTACATT |
| | | | TAAGTGCCTTGCGGTCAGGGACTGA |
| | | | GAATCTTGCCCGTTTATGTATGCTC |
| | | | TATGTATGCTCCATGTCTAGCCCAT |
| 202675_at | SDHB | 0.607788535 | ACCCTCTTCCACACATGTATGTGAT |
| | | | AAAGGATCTTGTTCCCGATTTGAGC |
| | | | GTTCCCGATTTGAGCAACTTCTATG |
| | | | TTTGAGCAACTTCTATGCACAGTAC |
| | | | AGGATGAATCTCAGGAAGGCAAGCA |
| | | | GCAGTCCATAGAAGAGCGTGAGAAA |
| | | | AAGAGCGTGAGAAACTGGACGGGCT |
| | | | GGAACGGAGACAAATATCTGGGGCC |
| | | | ATATCTGGGGCCTGCAGTTCTTATG |
| | | | CTGCAGTTCTTATGCAGGCCTATCG |
| | | | GATGACTTCACAGAGGAGCGCCTGG |
| 223299_at | SEC11C | 0.520144155 | TGGAAAGGCTTGATCGTGCTCACAG |
| | | | CACAGGCAGTGAGAGCCCCATCGTG |
| | | | CCCCATCGTGGTGGTGCTGAGTGGC |
| | | | TGAGTGGCAGTATGGAGCCGGCCTT |
| | | | TCACAGAGGAGACCTCCTGTTCCTC |
| | | | TCCTGTTCCTCACAAATTTCCGGGA |
| | | | GGGAAGACCCAATCAGAGCTGGTGA |
| | | | GACGAGACATTCCAATAGTTCACAG |
| | | | GGAAGAGCAAGAGGGTTTTTACCAT |
| | | | TATGCTCTTTTGGCTGTAATGGGTG |
| | | | ATTTGAGATGTTCCATTTTCTGTAT |
| 204563_at | SELL | 0.470833687 | CCTCGCCGTCTGTGAATTGGACCAT |
| | | | GGACCATCCTATTTAACTGGCTTCA |
| | | | TTTTCAGTTGGCTGACTTCCACACC |
| | | | CCACACCTAGCATCTCATGAGTGCC |
| | | | TAGCCTGCGCTGTTTTTTAGTTTGG |
| | | | TTTATGAGACCCATTCCTATTTCTT |
| | | | GTCAATGTTTCTTTTATCACGATAT |
| | | | GACCTTTTATCCACTTACCTAGATT |
| | | | CACCACTTCTTTTATAACTAGTCCT |
| | | | TAGTCCTTTACTAATCCAACCCATG |
| | | | CTCTTCCTGGCTTCTTACTGAAAGG |
| 208999_at | SEPT8 | −0.41582035 | AGTTAGCCCCCATAGAATGTGACCC |
| | | | GAATGTGACCCTGTCTGCAGAGTCT |
| | | | TGTCTGCAGAGTCTCATTTACCCCT |
| | | | GTTGGCTTTATTAGGGCTGTCTTAC |
| | | | GTTGGCATTTACTATCATGTCTTTA |
| | | | ATCACCATATAATTCGTTGCCCAAA |
| | | | AAAGGCATAAACCAGACCTGTCCCA |
| | | | GGGGCTCATGGATACGAGGCCTGAG |
| | | | GAAGTGTGGCTTGCTAGTCTGTTAC |
| | | | GCTTTTCTAAAATTGCTTCACGTGT |
| | | | CTTTTCCATTCACTTTGTACTTATT |
| 209000_s_at | SEPT8 | −0.41229454 | GTGAGGACGGACTGGGAGCCGGTAC |
| | | | GGAGCCGGTACAGACTCCAGTGTTT |
| | | | CCCCTCTCTATGCAAACACGTAAAA |
| | | | TCAGAGCCAGTGGCTGGTCTTCCAT |
| | | | TACAGTGTCACTATTCCCTGACGGA |
| | | | TTCCCTGACGGAGCTGTTATGTGCC |
| | | | TTATGTGCCGCTCTAGCGAAGGCCC |
| | | | CTAGGCCTAATTGTTCAGCGTGGAG |
| | | | AGATGGCAACTCACGTGGTGCCCTA |
| | | | GCGTGGTCTGGTATACATGCTGCAA |
| | | | TATCCTCTCCCATTATTTTCATAAG |
| 226627_at | SEPT8 | −0.47811452 | GAAACTCACCATAATAGTGCCGTCT |
| | | | GGGAGTCTGGTGGAACTGTGTTGGA |
| | | | TTAAGATACCTTTTCACTCTTCCGT |
| | | | TCCGTATGTCATGAGCCTTGTGCGT |
| | | | GGGTAGACTCTGTAAACACCTCCTT |
| | | | CACCTCCTTACTCACTATAGTCAAG |
| | | | ATAGTCAAGAAGTCCAGCGGCGTCC |
| | | | AGCGGCGTCCCAATATAGAGGTCCC |
| | | | GCAGTCTGTCCAGAATAGCCAGCTC |
| | | | ATCCTCAGCAGCTCATTCGGGGAAT |
| | | | GGGGAATAGTCAGAGCCATAGTGCT |
| 40149_at | SH2B1 | −0.40484824 | GCTGCAGCAGTCACCACTAGGGGGT |
| | | | CCCAGGGCCATTAACAACCAGTACT |
| | | | AGTACTCCTTCGTGTGAGCCAACCC |
| | | | GCTTCCTGACCCTTGTTGGCCAAGG |
| | | | CTTCCTGACCCTTGTTGGCCAAGGG |
| | | | TCCTGACCCTTGTTGGCCAAGGGCA |
| | | | CCCTTGTTGGCCAAGGGCATCTTTG |
| | | | TTGGCCAAGGGCATCTTTGATGGTA |
| | | | GCCAAGGGCATCTTTGATGGTACAA |
| | | | ATCTTTGATGGTACAAGCAGAGGCT |
| | | | TCTTTGATGGTACAAGCAGAGGCTC |
| | | | TTTGATGGTACAAGCAGAGGCTCGG |
| | | | GAGAGGCTCCCGTCACACACTACAG |
| | | | GGGGATTTGGGCTCCATGAGCTCCT |
| | | | CTTGAGGGGCTCTTCTGGTCAGCCC |
| | | | GAGGGGCTCTTCTGGTCAGCCCCAC |
| 218797_s_at | SIRT7 | 0.50897414 | CCCATCCTAGGGGGCTGGTTTGGCA |
| | | | GGCAGGGGCTGCACAAAACGCACAA |
| | | | GACGTAATCACGTGCTCGATGAAGA |
| | | | GCAGATGGCCAGTGTCACGGTGAAG |
| | | | TTTTCACCGTGACATTTTTAGCCAT |
| | | | GCCATTTGTCCTTGAGGAAGCCCCT |
| | | | GATACGGCCTGGCCATCGAGGACAC |
| | | | CCATCCGGCCTCTGTGTCAAGAGGT |
| | | | CCTCACCGTATTTCTACTACTACTT |
| | | | GAACTTTATAGAATCCTCTCTGTAC |
| | | | TGGATGTGCGGCAGAGGGGTGGCTC |
| 210010_s_at | SLC25A1 | 0.750588351 | GTGTGGAAGACGGACTAAGCCTAGA |
| | | | CTAGAGAGGCCGCAAGGGGACCGCC |
| | | | TGCAGTAGTGCCAAAAGGCCCCTTC |
| | | | TCTGTAGCCTGGTCTGTGCATTGTG |
| | | | GTGCATTGTGGCTGTCAAATCCATG |
| | | | CAGCCATGGCTGGATGTGCATCTGG |
| | | | GCTGGATGTGCATCTGGCCTATGAC |
| | | | TGCCTGTGTTTCATGTTCTGTGTCA |
| | | | TCATGTTCTGTGTCACGTGACCCTG |
| | | | CCTGGATGTGGCCATAGTGTTTGTC |
| | | | GAAGCTGCTCAACAAAGTGTGGAAG |
| 223222_at | SLC25A19 | 0.974852744 | GTTCTTCTCGTATGAATTCTTCTGT |
| | | | TTCTGTAATGTCTTCCACTGCATGA |
| | | | TCAGTCTCCACTGAGAGGTGCCGTC |
| | | | AAGCGGGGTAGCAGCCTTGAACCCA |
| | | | GGGACACCACCAGAAGGTCCAGGGC |
| | | | TCCAGGGCTCTCCCCATGAGAGAAT |
| | | | GGACGTGGTCTATGGTGAGCCAACG |
| | | | AACAGAACACACTCCTGGTCTGGAT |
| | | | GATGGGGCTGCTGCTTGAGTGCAGA |
| | | | CAGAGGGCTGCGGTAGGCCCTTTGC |
| | | | CTTTGCAGGAGTCAGGTCCCTACAC |
| 217122_s_at | SLC35E2B | −0.67710358 | GTCTCTGAAGTATTTCCTCCAGTTT |
| | | | GGGCCCCTATGTTTGAGTTTGATGG |
| | | | GGATCCTCACTCAACGAAAACTCGG |
| | | | CTCGGTTGGAAACTGTTCCGCCTGG |
| | | | GACTTGCTCATTTAGACTGTTCACG |
| | | | GAGTCTGAATCTGCCAACGTGGTGT |
| | | | TCAGGGCAACTTTCCCCATACAGGA |
| | | | TACATCAACAGTCTACGTCACAGCC |
| | | | GTGCTTTCTAGCAAACGGTTCTGTT |
| | | | TAGCGAGTCACTGTTGATTCTGCTG |
| | | | ATACCGTGTAACTAATCCCGTGGAT |
| 242367_at | SLC38A1 | 0.376727271 | AAATCATCTCTGCGGGCGTGAAAGC |
| | | | GTGCCCGATGCTTTCGGATGTTGCT |
| | | | GAAAAGTCCAGGTCTCCTGTGCTTC |
| | | | TTGTTTTCCTGTGACTTTGGTGTGT |
| | | | GATCTGGTTCCATTTTTACGAGAGC |
| | | | TTACGAGAGCCAGGAACCACGCACG |
| | | | GAGGTAAGGTGATTATCCGTTCCCG |
| | | | GCGACCGTGTTCTGGGAGTGTTTGA |
| | | | TGGTGAAAATTTCCTGTGTCCGCAA |
| | | | GTCCGCAAGGCCCAGAGGAGATCGT |
| | | | AGGAGATCGTGTGATGTCCGGGGGG |
| 200924_s_at | SLC3A2 | 0.42179558 | ACAGCCTATGGAGGCTCCAGTCATG |
| | | | GGCTCCAGTCATGCTGTGGGATGAG |
| | | | CTGTGGGATGAGTCCAGCTTCCCTG |
| | | | TTCCCTGACATCCCAGGGGCTGTAA |
| | | | GGGGCTGTAAGTGCCAACATGACTG |
| | | | AGCGGAGTAAGGAGCGCTCCCTACT |
| | | | TCCCTACTGCATGGGGACTTCCACG |
| | | | ACTGGGACCAGAATGAGCGTTTTCT |
| | | | GAGCGTTTTCTGGTAGTGCTTAACT |
| | | | TAACTTTGGGGATGTGGGCCTCTCG |
| | | | AACTGGAGCCTCACGAAGGGCTGCT |
| 223044_at | SLC40A1 | −0.72816035 | GGCAAGAATCCCAATTTAACTCATG |
| | | | GTAAGCCTTCAGCCTGGCAAGTTAC |
| | | | ACATGTAGAAAGCCCACACTTGTGA |
| | | | GTTATTTCTACATTGTTCTACAGCA |
| | | | AAAGTATCCCTTTCAAATGCCTTTG |
| | | | GCAACATGTCTGTACCAAGATGGTA |
| | | | GTACTTTGCCTTAACCGTTTATATG |
| | | | CACTTTCATGGAGACTGCAATACGT |
| | | | TGCTATGAGCACTTTCTTTATCCTT |
| | | | ATCCTTGGAGTTTAATCCTTTGCTT |
| | | | TTGCTTCATCTTTCTACAGTATGAC |
| 202111_at | SLC4A2 | 0.341046478 | GGCCTCTCCATAGTTATCGGGGATC |
| | | | TAGTTATCGGGGATCTGCTCCGGCA |
| | | | TTTCCTGTACATGGGAGTCACCTCC |
| | | | TCACCTCCCTTAACGGGATCCAGTT |
| | | | AGTTCTATGAGCGGCTGCATCTGCT |
| | | | CACCCAGATGTCACTTACGTCAAGA |
| | | | CGTATCTTCACCGACCGAGAGATGA |
| | | | GAGGCAGAGCCGGTGTTTGATGAGC |
| | | | ACAATGAGATGCCCATGCCTGTGTA |
| | | | ACAGCCGAGGGACCGATGGACGAGG |
| | | | GGACGAGGGGACAGGCTGGTGGGAT |
| 201349_at | SLC9A3 | R10.569257778 | AGAGAACTATGTTCTTCCCTGACTT |
| | | | GGAAGGTGAATGTGTTCCCGTCCTC |
| | | | CCGTCCTCCCGCAGTCAGAAAGGAG |
| | | | TCATGGGACCAGGCGAGAGGGCACC |
| | | | GATAAATGGGTCCAGGGCTGATCAA |
| | | | CTGCCGCTCTCAGTGGACAGGGCAT |
| | | | CATCTGTTATCCTGAACCTTGGCAG |
| | | | ACCTTGGCAGACACGTCTTGTTTTC |
| | | | TGGCCTCAGCCTTAAACTTTTGTTC |
| | | | GCAGCACGGGGAGGGTTTGGCTACC |
| | | | AGCCAGGTACCACCATTGTAAGGAA |
| 201320_at | SMARCC2 | −0.41120275 | TAATTTCGGGGATTTCTGTGGTAGG |
| | | | CCATGGACTCCTGGAAGGCACAGAG |
| | | | AGCACTTAAGCACCTCCATATTATG |
| | | | AAGCACCTCCATATTATGACTTGGT |
| | | | TATGACTTGGTGGGTCACCCCTTAG |
| | | | CCCTCTCCCACCAAGACTATGAGAA |
| | | | GACTATGAGAACTTCAGCTGATAGC |
| | | | GGGCTCCCCAGATGAGGATGCAGGG |
| | | | CTTCTCCCCTGTGACGGGAAGGCAG |
| | | | CGGGAAGGCAGGTGTGACTCCAGGC |
| | | | CCTTTCTTCTGTTCAAAGTTTTCTG |
| 212470_at | SPAG9 | 0.401936161 | TTCCCTCTATCCTTTTATTTAATGC |
| | | | ATATTACAAAATCCGTTCTACCATA |
| | | | AATCCGTTCTACCATAACAATACAG |
| | | | GTGTTACTGCACCAGTGTTATAGGT |
| | | | AGAATGTTTACTTCCTGCAAACTGG |
| | | | AAGCAATCCAGATGTGGTTTACTCT |
| | | | GGTTTACTCTGCCACAGTCTAATGT |
| | | | GCCACAGTCTAATGTCATTCACTTC |
| | | | GTCATTCACTTCATTTGATGGGGTC |
| | | | TGATGGGGTCACTTGTTAGCTGTCA |
| | | | GATGTATCTAAATGTCCCGAGAGGG |
| 207435_s_at | SRRM2 | −0.58358047 | CCTCCAGGTCTCCATAAATTGTCTT |
| | | | TGGAGCCACAAGGAGTGTCCCTTCT |
| | | | CCCCAGCAGAGCCGTGGGAGGGTCC |
| | | | TCTGCTCTCCTTTGAACCTTGGCAG |
| | | | TCCTGTGAAATGTTAATCTCCGTGA |
| | | | TAATCTCCGTGAGTTCTTCCTGGTT |
| | | | GGGGTGATTGTGATGGTGGTTGGGA |
| | | | TGGAATTAGTTGGTCCCTACTGTCC |
| | | | TACTGTCCCCCATGAGGTTGTGAAC |
| | | | TCCCCCATGAGGTTGTGAACCCCTC |
| | | | CTGTACAGCAAGAGCAACTTTTTCT |
| 208610_s_at | SRRM2 | −0.52612752 | CACGGGGCCATGTACAACGGGATCG |
| | | | GTGAGCGGCCTGACTACAAGGGAGA |
| | | | TGGTGAAGCGGCCTAATCCTGACAT |
| | | | TCGAGCTGCGATGCCTCGAGCTGGA |
| | | | GACCTTTCGACTCATGTTGCTGGAG |
| | | | GTCACGGAGACTCACCAGTTGGCAG |
| | | | AAGAATGAAAGACTCCGTGCTGCCT |
| | | | TCCGTGCTGCCTTTGGCATCAGTGA |
| | | | GATTCTTACGTAGATGGCAGCTCTT |
| | | | TCAGCGTCGTGCCCGAGAAGCTAAA |
| | | | GAAGCTAAACAACCAGCTCCTGAGC |
| 219919_s_at | SSH3 | −0.38068238 | GAAGAGGATCCACAACTCCTTGGAG |
| | | | GCCTGTCCAAGGGCTCAAGACTTTC |
| | | | CAAGACTTTCTAACTGGGATGTGGT |
| | | | TACCTTTGGGGGCAACAGCACCCTA |
| | | | TTCCTGGAACCAGCCAGGCCAGGCA |
| | | | GCCCCAGCCGCGGGAGGCTGGAAGG |
| | | | AGGCTGGAAGGGCTGGCAGATCGCT |
| | | | TGACACCACGCCAGATCACAGGGCA |
| | | | GGCACCAGGCCAGAGATAGTCTTCT |
| | | | TGGCCTCTGGCTAGTCAGTTTTTCA |
| | | | AGCCTTACAGTATCTGGCTTTGTAC |
| 204963_at | SSPN | −0.40007125 | AATTCTGAACTGTATCCATATTTTA |
| | | | GAACTTTATCAGTATGCTTTGTTGA |
| | | | TAATTGAGTTCAATTCGCCTCTCCG |
| | | | CCTCTCCGCATTGCCTATTGATACA |
| | | | GCATTGCCTATTGATACACTTTACT |
| | | | AAAACATTTTCCTGCTTGTCTTAGA |
| | | | GCGTTAAGTCGGTAAGCTAGAGGAT |
| | | | ATGTCCTCTAGATAAAACACCCGAT |
| | | | GACACATTGGAGAGCTTAGAGGATA |
| | | | ATCACACACAAAAGTTACACCAACA |
| | | | ACCTGTAAAATACCTTGTGCCCTAT |
| 204964_s_at | SSPN | −0.52431915 | GCTCCTCCCTGCTAGTCAGGGACAC |
| | | | GGATCATTGTCTGCTTAGTGGCCTA |
| | | | TGGCTTGTTTATGCTTTGTGTCTCA |
| | | | TTCGCAGCTCACACAGTTTACCTGT |
| | | | TTACCTGTGAGACCACACTCGACTC |
| | | | CACTCGACTCTTGCCAGTGCAAACT |
| | | | TCAGCAGGACCTTTGTTTACCGGGA |
| | | | CGGGATGTGACGGACTGTACCAGCG |
| | | | CTTGTTGGCCTGCTTTGTGATGTGG |
| | | | GTACCAGGTCTTCTATGTGGGTGTC |
| | | | GTGGGTGTCAGGATATGCTCCCTCA |
| 226932_at | SSPN | −0.42535696 | CTCAAATGATTATTATCCCCTTCAA |
| | | | ACTGGTCTGTACTTTGGTGTTGTGG |
| | | | ATGTTTTCTATTCATGTCCAGGGCA |
| | | | ACTTCCCTTTTTGCATGCAGTATGT |
| | | | AAAAGCTGCCCTGCAAAACCAATCC |
| | | | CAAAACCAATCCTTTCCTATCATGA |
| | | | GAAGAGTACCTTCATATTTTCTAGA |
| | | | GTCTCTGAACCGTTGCTACATAGCC |
| | | | GTTGGCTATCAGTTCTTGCTATTCT |
| | | | GTTCTTGCTATTCTCAGAGCACTCT |
| | | | AGAGCACTCTATCATGTTTTTAGGT |
| 237817_at | SSR3 | 0.476986005 | CTATGGGAATCTGTGTCCTTGCCTC |
| | | | AGAGCCAAAGCAAACCTGTCATTTT |
| | | | ACCTGTCATTTTTGATAGCTTCTGA |
| | | | GAAATAATAAGCCCCTGTACCTGTT |
| | | | GTACCTGTTATTTTTGGGCTCTGGG |
| | | | GGCTCTGGGGGTTGGGTGGATGGCC |
| | | | AATAGGTTCATTCCAGTGTATCTTA |
| | | | TAATAGTGTTTCAAGCTGCTGTTAA |
| | | | TGCTCTGGGAGTCAGTCCATTAAAT |
| | | | GGGCAAGAATCAGTCTTCTCTTATT |
| | | | GATGTTATCCAGGAATGTGCAGCCA |
| 203759_at | ST3GAL4 | 0.450622686 | TGCCAGTATGACCCACTTGGACTCA |
| | | | CCCTGGCTGCTCTTATGGAGCCGAG |
| | | | GGCTGCTCTTATGGAGCCGAGATCC |
| | | | GCCGAGATCCAGTCAGGGTGGGGGC |
| | | | CCAGTCAGGGTGGGGGCGCTGGAGC |
| | | | TGCCAGCACCAAGAGATTATTTAAT |
| | | | AGGCCAGTAGAGAATTCTGCCCACT |
| | | | ACCAAGGCCTAGACACGGCACTGGC |
| | | | GGGAAGAGCACTGGTGTGGGGGTTC |
| | | | TGGTGTGGGGGTTCCACCGAGAAGG |
| | | | CACCGAGAAGGGGACCTCATCTAGA |
| 224203_at | SUFU | 0.476269896 | TCTAACAGGTGCTCAACCTACTCCA |
| | | | CCACCACACTCCCGAGTGTCTTGGA |
| | | | GTCTTGGAGGGACAGCATCCTTTTT |
| | | | TCACCTCGCTCGCAAGTATCAAGAA |
| | | | GGTGGCCATACCTGGTTTGTGAATG |
| | | | TTGTGAGTTTCACCCAGTCTGGGGA |
| | | | GTCTGGGGAGTCTGTGAAGCATATG |
| | | | ACAGACACACTTTTTGTCCCTGCAT |
| | | | GCATGTCTACAGAATTTCTCCTCCT |
| | | | CAAACAAAGGACACCAACCACACTC |
| | | | CACTCCCCAGACTAAGCCGAGATAG |
| 206161_s_at | SYT5 | 0.439999734 | CAAGGTGCATCGGCAGACGCTGAAC |
| | | | TGAACCCTCACTTTGGGGAGACCTT |
| | | | GTCATGGCGGTGTACGACTTCGACC |
| | | | TGACGCCATCGGGGAGGTGCGGGTC |
| | | | CCGTCATCGTCCTGGAGGCTAAAAA |
| | | | CAGATCCATACGTCAAGGTCCACCT |
| | | | AAGAAGAACACTCTGAACCCCTATT |
| | | | ACCCCTATTACAACGAAGCTTTCAG |
| | | | GAAGCTTTCAGCTTCGAGGTGCCCT |
| | | | CTCGGACCCCTATGTGCGGGTCTAC |
| | | | GGAGGCGGTACGAGACCAAGGTGCA |
| 206162_x_at | SYT5 | 0.370742661 | CCTGTGACCAAGTCCAGAAGGTGCA |
| | | | AAGGTGCAGGTGGAGCTGACCGTGC |
| | | | CTGACCGTGCTGGACTACGACAAGC |
| | | | CTGGACTACGACAAGCTGGGCAAGA |
| | | | GCTGGGCAAGAACGAGGCCATCGGG |
| | | | AAGAACGAGGCCATCGGGAGGGTGG |
| | | | GGCCATCGGGAGGGTGGCCGTGGGG |
| | | | CCCAACGATGCCAATCACGACAACT |
| | | | TGCCAATCACGACAACTTTCCAGCA |
| | | | GACCCCGGGAAGGGAAGGCAGCCTG |
| | | | AAGGGAAGGCAGCCTGGTTTCTCCT |
| 202813_at | TARBP1 | −0.55713006 | ACCTAACCCAATATTGCTTTCCTGA |
| | | | TTGCTTTCCTGAGAAATCTCTGCTC |
| | | | GGAATTCCAGCAAATCTGATCCAAC |
| | | | GTGTGGAAATTCCTCAACAGGGCAT |
| | | | CAACAGGGCATTATCCGCTCCCTGA |
| | | | CGCTCCCTGAATGTCCATGTGAGTG |
| | | | GTCCATGTGAGTGGAGCCCTGCTGA |
| | | | GAGCCCTGCTGATCTGGGAGTACAC |
| | | | ATCTGGGAGTACACCAGGCAGCAGC |
| | | | CTCGCACGGAGATACCAAGCCATGA |
| | | | TGATGTGCCTTCCTTAGTGAACTGC |
| 213877_x_at | TCEB2 | 0.69305787 | AGAGATTTGGGAGTCTGCCTGGTTG |
| | | | TTTTGGGGCTTGTGCTTGGCAGTTC |
| | | | ATCCTGAGACCCTGGCTGAGAACTT |
| | | | TGCTGCTTAAAGGCACCATGGGGAC |
| | | | CCTCAGACCCAAGCCATTGTTAGCA |
| | | | GAGACACAAAGACCAGAGCCAGCCT |
| | | | GCCAGCCTCAGGGACAAGAGATTCC |
| | | | AGAGATTCCAGTTTTAGGCCTTTCT |
| | | | GCTGGAGCCAGTGTCCTGGTTTGAC |
| | | | CACCCACGCTGGGGGCTGTAATCAC |
| | | | ATCACGGAGGGAAGTGGCTGCCCCC |
| 218099_at | TEX2 | 0.440263979 | TGACAGGATGGGTCCTCTCATACAG |
| | | | TTTTTCCATCTGGCGTTTCTGTGTC |
| | | | GTTTCTGTGTCCTCCAGGTTTATAT |
| | | | GGGAGAGTTCCATGGGCAGATTTCC |
| | | | GAAGGCCAAAACGGAGAACTGCTCT |
| | | | AGACCAAAAGTTTGCTCAGCATCAC |
| | | | TCAGCATCACACTACATCTCAAAAT |
| | | | TAGTTTACAAGGTTGGGGGCTCTCT |
| | | | GGGCTCTCTTTGCTTCGAGAAGTAA |
| | | | GCTGCATTCAACGTCAAAATTACCT |
| | | | GCACCTTGCCTGAACATGACTTTAA |
| 218996_at | TFPT | 0.66617142 | GGCGGCGCCAGCGGGAATTAAATCG |
| | | | AAAGTACCAGGCACTAGGTCGGCGC |
| | | | GCGCTGCCGGGAGATCGAGCAGGTG |
| | | | AACGAGCGGGTCCTGAACAGGCTCC |
| | | | GGTTCCTCATGAGAGTGCTGGACTC |
| | | | GCTGGACTCCTACGGGGATGACTAC |
| | | | CAGCCAGTTCACCATTGTGCTGGAG |
| | | | GCCGAGCAGGAAATGCGCTGACTCC |
| | | | TGGCCCCGGTGCAGATTAAGGTTGA |
| | | | CCTGGATTCCAGTTGGGTTTCTCGG |
| | | | TCGGGGTCCAGACAAACTGCTGCCC |
| 216262_s_at | TGIF2 | 0.532627427 | TCGCCCATCTGTTGCTGTGGGAGTG |
| | | | GTGGGAGTGTGAACGGATCGCTGAA |
| | | | GCTTTGCTCTCTCTAGGTGGGCAAG |
| | | | CCGTGTGCCCCAGGGGGATCAGGGA |
| | | | GAACATGGCTTCATCCAGGTTAACT |
| | | | ATCCAGGTTAACTGATGCTGCCATT |
| | | | GGATGCCTGTAGTAGGGAACTCTGG |
| | | | TGGGCTGAGGTGGGATTTTCCCTCC |
| | | | GTGAGGGAGCCATGCTGCTGAATTC |
| | | | CTGGTTGGCATTTCCCCATTATGTA |
| | | | GTGTTGGGTAGGGCAGACTCTGCTT |
| 218724_s_at | TGIF2 | 0.403591471 | GCCTCCGCTCAGTGATGAGACCAAG |
| | | | GAGATCGGAGACAAGCATGGTGCTG |
| | | | GCTGGGCTCAGGAAAGCTGCCAAAT |
| | | | TTCAGTCCTATGTTGGGTCCAAGCT |
| | | | CTGTGCTGTTTCTGTCAAGCCAGGT |
| | | | GGACATTCCAAGTTCATATGCGTGA |
| | | | GGATGTAACAGAACCGACTCCAGTT |
| | | | GCTGTGGTTTGCATTCACGGCAGTA |
| | | | CACGGCAGTAGTTAGCCCAGGTGTG |
| | | | GAGTGCACTGCATGATAGCGTTCTG |
| | | | GGACCAGCTAAGTCTCTGCAGTAGT |
| 212910_at | THAP11 | 0.435755307 | ACACCAGCAATTATGACTTTGTCTA |
| | | | GAGGCTTCAGAACCACTGAACTTGA |
| | | | TGAACTTGAAACTTACCCTCTAGGG |
| | | | GGGATGCAGGTGGGATGTCCAGGGA |
| | | | GGTTGGTCAGCAGTCAGACAACTCT |
| | | | TGGGGACTGGTAAATCTGTGCCTCT |
| | | | GCCTCCTAGGACTTATTTTCCCAGG |
| | | | ATTTTCCCAGGAGGCCATTTACAAG |
| | | | AAGGGGATCTGGATGACCTGCTGAT |
| | | | GATCCAGCTTGCCAGGGACTTAGGT |
| | | | CCTGTTTTGTTTGCTACTGGTTACA |
| 222835_at | THSD4 | −0.4286182 | AAAAGCCCAGATTTCGGTAGCCATC |
| | | | TTTCTGCTTTCTTAGTGCCCATTAT |
| | | | TTTTTTCTTGGCCTGTGTACGGGAT |
| | | | TGTGTACGGGATTGCCTCATTTCCT |
| | | | CCTCATTTCCTGCTCTGAATTTTAA |
| | | | AAAGCTGTCATATGGTTTCCTCACA |
| | | | TAGTTTGCCGTTTTACTTTCATCCA |
| | | | AAGGAAATTGTGCCTCTTGCAGCCT |
| | | | TTAGTACTATCGATTCTTTCCACCC |
| | | | GACTTGCGGTTCTCTCTGTAGAAAA |
| | | | GAGTCAGTTCAGTTCCGTAAAGGTA |
| 226506_at | THSD4 | −0.37638707 | TAATCAGTCCAGTTCCCTGAGGTTT |
| | | | TACTCTGCTTTTCGACTCATTCAGG |
| | | | GTAGCATTGTACCTGAACCTGATTG |
| | | | TGGGAGGGTGTCTGTTATCCCTTTC |
| | | | CTTTGTCCCCGTTGTTAGACTGGCA |
| | | | TAGACTGGCAGCGTCAGTTGCTCGG |
| | | | GCCGTGGGTGAGGCAGGTGGCTGGC |
| | | | TGGCATTTACTGCTCTGACACTTCC |
| | | | CTGTGGGGCCTGTGAACTGCACAGC |
| | | | CAGCCAGGAGCAAGGAACCCACTAA |
| | | | CATGTCCCCTCTACAGTGTTAAATT |
| 232944_at | THSD4 | −0.44465042 | GCAACTGCCACATAATTGCCAAAAC |
| | | | AAATGAGCCATCACGTCACAAAGAG |
| | | | GGCGGTGAAACTACTCTGTGTGATA |
| | | | TACACTGGCGGATGCATGTCACTGT |
| | | | GCACTTGTCCAGACCCACAGAATGT |
| | | | GACCCACAGAATGTGCACTCCGAAG |
| | | | ACTCCGAAGTGTGAACCCTCGTATG |
| | | | GAACCCTCGTATGGACTATGAACTC |
| | | | GACATGTCCGTGTAGGGTCATCAGT |
| | | | AGGGTCATCAGTTACACATGTACCA |
| | | | TGGTAGTAAGGGACCCTGTGCCTGT |
| 224560_at | TIMP2 | −0.40585268 | TTGTTTTTGACATCAGCTGTAATCA |
| | | | CATCAGCTGTAATCATTCCTGTGCT |
| | | | CCCTTGGTAGGTATTAGACTTGCAC |
| | | | AACGCGTGGCCTATGCAGGTGGATT |
| | | | GGCCTATGCAGGTGGATTCCTTCAG |
| | | | TGCAGGTGGATTCCTTCAGGTCTTT |
| | | | ACAGGTTAAGAAGAGCCGGGTGGCA |
| | | | GTTAAGAAGAGCCGGGTGGCAGCTG |
| | | | GTGGCAGCTGACAGAGGAAGCCGCT |
| | | | GAGGAAGCCGCTCAAATACCTTCAC |
| | | | GAAGCCGCTCAAATACCTTCACAAT |
| 231579_s_at | TIMP2 | −0.44946961 | GAGTAGGTTCGGTCTGAAAGGTGTG |
| | | | GGCCTTTATATTTGATCCACACACG |
| | | | GATCCACACACGTTGGTCTTTTAAC |
| | | | CACGTTGGTCTTTTAACCGTGCTGA |
| | | | ATTTTCATCCTGCAAGCAACTCAAA |
| | | | ATTTTCAAATCTTTGCTTGATAAGT |
| | | | TGGACTTGCTGCCGTAATTTAAAGC |
| | | | CTGCCGTAATTTAAAGCTCTGTTGA |
| | | | GGAGCACTGTGTTTATGCTGGAATA |
| | | | ATGAAGTCTGAGACCTTCCGGTGCT |
| | | | ACCTTCCGGTGCTGGGAACACACAA |
| 228505_s_at | TMEM170A | 0.464931043 | CAGCTATTGCTGGAGTTTACCGAGC |
| | | | GGAGTTTACCGAGCAGCAGGGAAGG |
| | | | ATGATACCATTTGAAGCCCTCACAC |
| | | | GCACTGGACAGACATTTTGCGTCTT |
| | | | TTTTTACGGATTTTAGCTACTCTAT |
| | | | GCTACTCTATAGCATACATCCTTAT |
| | | | GAGTGTAGTGTTTTCTTAGTTCTTC |
| | | | ATTGAAGACTTATGTGGACTCCTAT |
| | | | GGACTCCTATTGTTCTCAACCAAAA |
| | | | TAAGCAGTTTTCATGTGTACCTTTA |
| | | | TACCTTTACCCAAGCCAAGTCAACA |
| 227733_at | TMEM63C | −0.4519704 | TCCAGTGTAGCCTGGCTCTGAGAGA |
| | | | TGGAGAAGGTTCCATAGTCCACTCT |
| | | | TAGTCCACTCTTAGGGGAACCAGCA |
| | | | CATGGTCACTACAGGATGGTGGAGC |
| | | | ATGGTGGAGCAGGGGGCATCTTTTA |
| | | | AGGAACCGGTATTGCCTAGAGCCTC |
| | | | ACTGCCCCTGGAAGCAAAGTGCCTA |
| | | | AAAGTGCCTATCAGCAGCGTTGCGT |
| | | | GAATGTGCCAGAATGCTGAACCTTC |
| | | | GCTGAACCTTCTTGTTAATGCTATG |
| | | | TAATGCTATGACCGTGCCTTGAATA |
| 240261_at | TOM1L1 | 0.452804108 | TGAGAGTCTCACTTTATAAAATGGG |
| | | | AAATGGGAACAATGATTGCCTTAAA |
| | | | ATTGCCTTAAAGGGTGGTTGTCAAG |
| | | | GTAGCAAAGCTTACAATGCATTTTA |
| | | | GAGTTACCTATCTTACCTGTTATCT |
| | | | GTTACCTATCTTACCTGTTATCTTC |
| | | | TATCTTACCTGTTATCTTCTGCTGT |
| | | | GTCCTTATTCCCAGCAAGGGTTTGG |
| | | | TATTCCCAGCAAGGGTTTGGCAAAT |
| | | | AAAACACAGGCTTTAAAGTCAGAGA |
| | | | CCTCTCAGCCACCTAACTTTTGAGA |
| 212408_at | TOR1AIP1 | −0.8187163 | GGAGTGCCTCAAGCCAAGATAGTGA |
| | | | ATAATTGAGCTTTCTCATCTGTCAA |
| | | | TCTGTCAAATGCTATGGTTTTCTTA |
| | | | CTAGATCTATCCACCTTGTTTTTTT |
| | | | AGAGTCAGTCATTGGCTTTGTCATT |
| | | | GGCTTTGTCATTTACCCTTTGAGAG |
| | | | ACCCTTTGAGAGTTCCACAAGTGGT |
| | | | TAGAGTGGTTTAACGTCTTTCCTCT |
| | | | GTCTTTCCTCTAGTACTACCAGTAT |
| | | | AATGTATACCCCTTACTGTAATTTG |
| | | | GTTCCTCTTAGAAGTCAGATCATCT |
| 212409_s_at | TOR1AIP1 | −0.60653651 | CAGGCTCTACTTTGATCTTCTACAA |
| | | | AGATGTAGCCTTAGTCCTGACTGTC |
| | | | CAAGTTCACCAATTCTAACACACCC |
| | | | ACACCCAACTCCTACAATCATATGG |
| | | | AATGGCCTCTGGAGCCGTATTTCTC |
| | | | TTCTCACTTAGTTCTGCCTGTGCAA |
| | | | GCCTGTGCAACCTGAAAATGCCCTG |
| | | | GAAAAATCATGTCCCAAGTTCTGAG |
| | | | AGTTCTGAGAATTGTTCACACTTTC |
| | | | TTCACACTTTCTAACCAGAGACAGA |
| | | | GAGACAGAATTCAGAGCTCTTTTTG |
| 216100_s_at | TOR1AIP1 | −0.61552544 | TTCAGTTTCCATTGAGAGCTCTGTT |
| | | | AAGGTATCTTAGGAGTGCAGATTAT |
| | | | TTTGATTCTGGGCTGAGTTATTACA |
| | | | GTTATTACAGTTATGGTATGACCAG |
| | | | TCCTTTCTTATGAACCTTCCTGATT |
| | | | GATTTTTTAACTTAGATTTCTCACT |
| | | | GATTTCTCACTAAGTTTCCTGAGTT |
| | | | AAGTTTCCTGAGTTATTAGTAAGAT |
| | | | CATTTAGGTGTGAGTTCCTTAGCTT |
| | | | CCTTAGCTTCTGCCTATAGGAACAT |
| | | | ATGAAAGGTCATCTAGGTGTGTGTT |
| 203511_s_at | TRAPPC3 | 0.419413644 | TGGGCTTTAACATTGGAGTCCGGCT |
| | | | GAAGATTTCTTGGCTCGGTCAAATG |
| | | | GGCATCACTCCAAGCATTACTAATT |
| | | | GCCCAGCTGGTGATGAATTCTCCCT |
| | | | GGAACTTCCTGATAACCACTCATCC |
| | | | TCCAATCTCTTGTGTGGGGTGTTGC |
| | | | GGTGTTGCGGGGAGCTTTGGAGATG |
| | | | TTGAGGACAATCTTCCAGCTGGAGA |
| | | | GAGGAATAACCATCCCTACAACTCG |
| | | | TGTTGGAATCAGCAGGCCTCTGTGC |
| | | | TCTTATAACCTGTTTCCATTCTCCA |
| 207305_s_at | TRAPPC8 | −0.475685 | AGTCAGCAGAATTCCATGCCTGCCC |
| | | | GCCTGCCCTGATCATCATCAGTAAT |
| | | | GATATCTGATCCCTGCAAAATACTT |
| | | | GATATCAGCATATTTGTGCACCTTA |
| | | | TGCACCTTATTAAGCCCCATCTTAA |
| | | | CAAAGTCTAAGTCTGCTGTTACAAC |
| | | | GAAAGGCCTTGTTGGCAGTACTCCT |
| | | | GCAGTACTCCTGTTAAGCCATTAGT |
| | | | AAGCCATTAGTCTCTAAATTCCAGC |
| | | | TGCTTCACACAGTTCCTTAAAATCA |
| | | | GAACTTTGGTCATAGAGTCTTCATA |
| 206911_at | TRIM25 | 0.464816775 | ACCAAGATCTCTGCCTGGCACAATA |
| | | | TCTCAACTGTGACCACGGCTTTGTC |
| | | | TTGCCGACAAGGTCCACCTGATGTA |
| | | | CTCCCCCAAGTAGGCAGGCTGTAGG |
| | | | GCTGTAGGCACTTGGGCTGACTGCC |
| | | | ACAGCAGGCAGAACTCTCCTTGGAT |
| | | | TTGTGGGCGAGGAGGCGTTTCCACC |
| | | | TATCAGGGCAGGGTGACCTACTCCC |
| | | | CCTACTCCCCATTGTTCTGGAAATC |
| | | | TGGAAATCTCCAGGCTGCTGGGCAG |
| | | | TGAAGTCATGAGTGCCCGATTCCTC |
| 223109_at | TRUB2 | 0.343758497 | GTGCGGGGCAGTGAATGCCCAGGCA |
| | | | GAAGAACTGCTATGAGCTGGACCTG |
| | | | GCTGGACCTGATAGCTGTGCAGAAA |
| | | | GTGAGAGCAGGGGCACCTTTTCTAC |
| | | | GGCACCTTTTCTACGTGTGACACAA |
| | | | AATGGACTTGACCCAACTGAGAAGG |
| | | | AAGTATTGGCAGACCAGGCGTGGTG |
| | | | TAGTGCGCTAGACGGCGCCTGTGAA |
| | | | CAGTGGGACCTAACCAACATCCAGG |
| | | | ACATCCAGGATGCTATCCGGGCTGC |
| | | | GGATGGTCCTGGGACTCCCAGGGCC |
| 218245_at | TSKU | 0.328423261 | CATCCAGACTGGAAACCTACCCATT |
| | | | TGAGCATCCTCTAGATGCTGCCCCA |
| | | | ATGCTGCCCCAAGGAGTTGCTGCAG |
| | | | TGCAGTTCTGGAGCCTCATCTGGCT |
| | | | ATCTGGCTGGGATCTCCAAGGGGCC |
| | | | TTACCCTCCCAGGAATGCCGTGAAA |
| | | | TAACGGAGTGTCACTTTCAACCGGC |
| | | | GTAATATTGTCCTGGGCCTGTGTTG |
| | | | GGGAAGCTGGGCATCAGTGGCCACA |
| | | | AGTGGCCACATGGGCATCAGGGGCT |
| | | | TCATCTATCTAACCGGTCCTTGATT |
| 201090_x_at | TUBA1A | 0.90557475 | AATACATGGCTTGCTGCCTGTTGTA |
| | | | ATGTCAATGCTGCCATTGCCACCAT |
| | | | AACCAAGCGCACGATCCAGTTTGTG |
| | | | TGCCCCACTGGCTTCAAGGTTGGCA |
| | | | AAGGTTGGCATCAACTACCAGCCTC |
| | | | ACACCACAGCCATTGCTGAGGCCTG |
| | | | GCCTGGACCACAAGTTTGACCTGAT |
| | | | GACCTGATGTATGCCAAGCGTGCCT |
| | | | GGCCCGTGAAGATATGGCTGCCCTT |
| | | | CTAATTATCCATTCCTTTTGGCCCT |
| | | | GTCATGCTCCCAGAATTTCAGCTTC |
| 211058_x_at | TUBA1A | 0.913630736 | ATGTCAATGCTGCCATTGCCACCAT |
| | | | TGCCCCACTGGCTTCAAGGTTGGCA |
| | | | AAGGTTGGCATCAACTACCAGCCTC |
| | | | CTCCCACTGTGGTGCCTGGTGGAGA |
| | | | ACACCACAGCCATTGCTGAGGCCTG |
| | | | GCCTGGACCACAAGTTTGACCTGAT |
| | | | GACCTGATGTATGCCAAGCGTGCCT |
| | | | GGCCCGTGAAGATATGGCTGCCCTT |
| | | | CTAATTATCCATTCCTTTTGGCCCT |
| | | | GTCATGCTCCCAGAATTTCAGCTTC |
| | | | TGTCTTTTCCATGTGTACCTGTAAT |
| 213646_x_at | TUBA1A | 0.827697475 | AAATGTGACCCTCGCCATGGTAAAT |
| | | | AATACATGGCTTGCTGCCTGTTGTA |
| | | | ATGTCAATGCTGCCATTGCCACCAT |
| | | | TGCCCCACTGGCTTCAAGGTTGGCA |
| | | | AAGGTTGGCATCAACTACCAGCCTC |
| | | | ACACCACAGCCATTGCTGAGGCCTG |
| | | | GCCTGGACCACAAGTTTGACCTGAT |
| | | | GACCTGATGTATGCCAAGCGTGCCT |
| | | | GGCCCGTGAAGATATGGCTGCCCTT |
| | | | CTAATTATCCATTCCTTTTGGCCCT |
| | | | GTCATGCTCCCAGAATTTCAGCTTC |
| 209251_x_at | TUBA1C | 0.858467073 | AAATGTGACCCTCGCCATGGTAAAT |
| | | | ACATGGCTTGCTGCCTGTTATACCG |
| | | | TATACCGTGGTGACGTGGTTCCCAA |
| | | | ATGTCAATGCTGCCATTGCCACCAT |
| | | | TGCCCCACTGGCTTCAAGGTTGGCA |
| | | | TTGGCATTAATTACCAGCCTCCCAC |
| | | | GCCTGGACCACAAGTTTGACCTGAT |
| | | | GACCTGATGTATGCCAAGCGTGCCT |
| | | | CGTGAGGACATGGCTGCCCTTGAGA |
| | | | GTGTGCTGTACTTTTACACTCCTTT |
| | | | TACACTCCTTTGTCTTGGAACTGTC |
| 211750_x_at | TUBA1C | 0.783244822 | AAATGTGACCCTCGCCATGGTAAAT |
| | | | ACATGGCTTGCTGCCTGTTATACCG |
| | | | TATACCGTGGTGACGTGGTTCCCAA |
| | | | ATGTCAATGCTGCCATTGCCACCAT |
| | | | TGCCCCACTGGCTTCAAGGTTGGCA |
| | | | TTGGCATTAATTACCAGCCTCCCAC |
| | | | GCAATACCACAGCTGTTGCCGAGGC |
| | | | GCCTGGACCACAAGTTTGACCTGAT |
| | | | GACCTGATGTATGCCAAGCGTGCCT |
| | | | CGTGAGGACATGGCTGCCCTTGAGA |
| | | | TACACTCCTTTGTCTTGGAACTGTC |
| 212639_x_at | TUBA1C, | 0.804293444 | AAATGTGACCCTCGCCATGGTAAAT |
| | TUBA1A | | CCGTGGTGACGTGGTTCCCAAAGAT |
| | | | ATGTCAATGCTGCCATTGCCACCAT |
| | | | AGTTTGTGGATTGGTGCCCCACTGG |
| | | | CTCCCACTGTGGTGCCTGGTGGAGA |
| | | | GAGAGCTGTGTGCATGCTGAGCAAC |
| | | | GCCTTTGTTCACTGGTACGTGGGTG |
| | | | GGCCCGTGAAGATATGGCTGCCCTT |
| | | | CTAATTATCCATTCCTTTTGGCCCT |
| | | | GATCACCAATGCTTGCTTTGAGCCA |
| | | | GCTTTGAGCCAGCCAACCAGATGGT |
| 201266_at | TXNRD1 | 0.770640577 | ACACGTGCTTGTGGACATCAGCCTC |
| | | | CCTGCCAGCAGTTCTTGAAGCTTCT |
| | | | ACCTGTATTTCTCAGTTGCAGCACT |
| | | | CCCATGCATCTGCCTGGCATTTAGG |
| | | | TGGCATTTAGGCAGCAGAGCCCCTG |
| | | | TCCTCATCTCATTTGGCTGTGTAAA |
| | | | GCAATTGAGGCAGTTGACCATATTC |
| | | | TCCAAGTCCACCAGTCTCTGAAATT |
| | | | GGAGTGGAATGTTCTATCCCCACAA |
| | | | TAGACTTGTCTTGTTCAGATTCTGT |
| | | | TCAGATTCTGTATTTACCCATTTTA |
| 209103_s_at | UFD1L | 0.84218285 | AAGTGAGGACTGTTGGCTGATTGGA |
| | | | AAACGCACTTAGGAACTTTGCCTGT |
| | | | GTCTGACCACCGGGGATGTGATTGC |
| | | | ACGAACTGCGTGTGATGGAGACCAA |
| | | | GAGACCAAACCCGACAAGGCAGTGT |
| | | | GTGTGACATGAACGTGGACTTTGAT |
| | | | TGCTCCCCTGGGCTACAAAGAACCC |
| | | | GACAAGTCCAGCATGAGGAGTCGAC |
| | | | CGCTTTCTCTGGATCTGGCAATAGA |
| | | | CCCTCCCCAATCAAGCCTGGAGATA |
| | | | TCACGTCCCCTTGTCAAAAAGGTTG |
| 206031_s_at | USP5 | 0.420391882 | CGCCCAAGGACCTGGGCTACATCTA |
| | | | GCTACATCTACTTCTACCAGAGAGT |
| | | | AGAGTGGCCAGCTAAGAGCCTGCCT |
| | | | TGCCTCACCCCTTACCAATGAGGGC |
| | | | CAATGAGGGCAGGGGAAGACCACCT |
| | | | AGACCACCTGGCATGAGGGAGAGGG |
| | | | CTGAGGGATGGACTTCAGCCCCTCT |
| | | | GGAGGCCGTGGGAGAATGGCTGGGC |
| | | | GGGGCAGCGATAGACTCTGGGGATG |
| | | | GTAAGGAGACTTTGTTGCTTCCCCT |
| | | | TGCGCGTGGGTGTAGCTTTGTGCAT |
| 218495_at | UXT | 0.496966797 | GAGAAAGTGCTGCGCTACGAGACCT |
| | | | GAGACCTTCATCAGTGACGTGCTGC |
| | | | GCAGCTGGCCAAATACCTTCAACTG |
| | | | GGATTTGGGCTGTAACTTCTTCGTT |
| | | | TCTTCGTTGACACAGTGGTCCCAGA |
| | | | CCAACCGTTCTTATTGCTGGCGGCC |
| | | | ATACTTCACGCATCTATGTGGCCCT |
| | | | GACACTGGCAGAAGCTCTCAAGTTC |
| | | | AAAGCCCATATCCACATGTTGCTAG |
| | | | ACAAGGCCTGCAGAATTTCCCAGAG |
| | | | TTCCCAGAGAAGCCTCACCATTGAC |
| 217821_s_at | WBP11 | 0.39110667 | ACCCAACTTGATTCAGCGACCCAAG |
| | | | GCGACCCAAGGCGGATGATACAAGT |
| | | | GATACAAGTGCAGCCACCATTGAGA |
| | | | GAAAGCCACAGCAACCATCAGTGCC |
| | | | TGCCAAGCCACAGATCACTAATCCC |
| | | | CAGAGATTACTCGATTTGTGCCCAC |
| | | | GAATAAAGGGGCTACTGCTGCTCCC |
| | | | AAAGCAGCACCCAAATCTGGTCCTT |
| | | | TGTTCCTGTCTCAGTACAAACTAAG |
| | | | GCTACTGTGACAGCTTTTGATGCCA |
| | | | GGCTTCTGTTCACAACAGTGGCCCA |
| 217822_at | WBP11 | 0.456975255 | TAGCCTTGTTCAGAATTTACTGCAC |
| | | | AAAAGGGTATTTCATCCAGAATAGA |
| | | | GATCAGTTATTGAAGCAGTGCTGCT |
| | | | AAGCAGTGCTGCTAACATCCATTCC |
| | | | CCTCCTCCAGTTCTTTGGAAATTTG |
| | | | GATCGGGGGATCTTAGTTGCTTATT |
| | | | TGCTTATTTGTTTTGACTCTTGTGT |
| | | | TGACTCTTGTGTGCTGTGGGCACTG |
| | | | GTGGGCACTGGAGTAGAGATTTCTG |
| | | | GGATCACAATGTCATTTCCTAATAC |
| | | | TATTTCCCACTGACCTAAACTTTCA |
| 217734_s_at | WDR6 | −0.50751083 | GCTCAGCATGCCTTGAGGGGAGGAG |
| | | | CGTGGGTTCCTGATGTCGGTGCAGG |
| | | | GATGACTTTGTGAACATTCCCAGGT |
| | | | CATTCCCAGGTATTGGAGCCTCTGT |
| | | | TGGAGCCTCTGTGGCCTTAAATGTG |
| | | | TGGAGGGAGACCCAGCATAGCCAGG |
| | | | TAGCCAGGCCAGTATGGAGCACCTC |
| | | | CTCACGCACAGCTCTCAGAAGCTGC |
| | | | GCTGCAGGCGGACGAACATCTGACC |
| | | | AAAGAGGTGTGGTCGAGGCTCCTGA |
| | | | ACAGAGACTGAGTCACTGGCCCATC |
| 219520_s_at | WWC3 | −0.45058724 | TATGTTTCAATCTGTCCATCTACCA |
| | | | ATCTACCAGGCCTCGCGATAAAAAC |
| | | | GTCTCAAAACCATCAGGATCCTGCC |
| | | | TCCTGCCACCAGGGTTCTTTTGAAA |
| | | | GGCTTTCACTTCATCTAATCACTGA |
| | | | AAGGAAGGCCAGAGAGCCGCGCAGT |
| | | | GACACCAAGCGCCCTATGTTGCTTG |
| | | | TGACGTGGTCTTGGAGCTTCTGACT |
| | | | TCTGACTAGTTCAGACTGCCACGCC |
| | | | GAAAATACCCCACATGCCAGAAAAG |
| | | | GTGAAGTCCTAGGTGTTTCCATCTA |
| 225273_at | WWC3 | −0.3762162 | GACGAACCCTTCGCTATAAGCAGTC |
| | | | ATAAGCAGTCATGCAGGTCTTCCCT |
| | | | TGGAGCTGGATCTCCAGGCGTCGAG |
| | | | GCCGAGCGGCAGACAAGACAGACCA |
| | | | GACTACCGTCATGAGCAGGCGGCTG |
| | | | GCCTCCAAGGAGATCTACCAGCTGC |
| | | | CAAAGAGCCCATCCAAGTGCAGACC |
| | | | GATAGCATTCTTCACAAGGCCAAGG |
| | | | GGCCAAGGATCAACATACCTCCTCT |
| | | | TCCCAGCCGACGACGTCTGATGGAG |
| | | | GAAGTATTTATCCACCTGTTTTATT |
| 212637_s_at | WWP1 | 0.331826249 | TGGATAGAACCATAACTTACACATG |
| | | | AAGTCATATACTAGATCCAATACTA |
| | | | GGAAGGATTCATTGAGCAGCATAGA |
| | | | GTTTGTTTACATGTTACTTTGAGAT |
| | | | CTTTGAGATGCTAGGTATTTGTGGA |
| | | | AAGAATCAGGCTCTTTTGTACTTTG |
| | | | GTTTTTAAATCTGTGATGCTTTTCA |
| | | | AATTGATGCAATTTCATACTTAGGA |
| | | | ATGTAAACTCTGCCACTTTTTTGTG |
| | | | GGTTTTTATGAAGCCAGATGGATTG |
| | | | AATATAAGGCTAATGATTTTCTGTT |
| 209375_at | XPC | −0.38045811 | AACTGAGGCAGCATGCACGGAGGCG |
| | | | AGGGGAGACGAGGCCAAGCTGAGGA |
| | | | AGCCCTTGTCAGATTCACCCAGGGT |
| | | | TTGCTAGGAGATACTCTTCTGCCTC |
| | | | GGAAGCCACCGGGAGATTTCTGGAT |
| | | | TGAATGCGCTGATCGTTTCTTCCAG |
| | | | CCAGTTAGAGTCTTCATCTGTCCGA |
| | | | TCATCTGTCCGACAAGTTCACTCGC |
| | | | TCAGGCTTACTAATGCTGCCCTCAC |
| | | | CCCTCACTGCCTCTTTGCAGTAGGG |
| | | | GGTCATCTGCTGGGATCTAGTTTTC |
| 217781_s_at | ZFP106 | −0.41254534 | GTTGTGGTGGAGGTGATTTGGGATA |
| | | | GGGATAGACTAGGTTTCCTTATGCA |
| | | | TGAGCTCCTCATAGAAACCAGACCT |
| | | | TTAGACAGTAACCTCTAACCTCACC |
| | | | CAAGCCCAAGTATATGGCCCTGCTG |
| | | | GGTTACCTGGTGACTACATTTCCCA |
| | | | ACATTTCCCAGATTCACTCTAAATT |
| | | | TATATGCCCTAGAGCTGCTCCAGCA |
| | | | GAAATCAGATGACACCTGACTGCAA |
| | | | GCAAATAGCCTTCTTACATTTTGGT |
| | | | CAAATCATCAGGTTCCTCGGGTTTA |
| 218490_s_at | ZNF302 | −0.59157756 | GAAAAATCTGTGTACATGTAGCAAA |
| | | | TAGGAATCTCCTGCAAACTCCTACA |
| | | | TTCCAAGTGCATCCCTTATTCTATA |
| | | | AGAGATGCAGCAAAGTGTTCACTAA |
| | | | GTTCACTAAGAGTGTTTATCTTGCC |
| | | | GAATGGTAGAGCAACCTGAAGGATT |
| | | | AAATCTTTGCAGTTATGCTATTTGT |
| | | | GCAGTAGCTTGCAGTTTCAGTTGAG |
| | | | GTTTCAGTTGAGTTCTACTTAGAAA |
| | | | GAAATTCTTTTTAGCTAGTGGGCAT |
| | | | GATATTTAGTCACCCAGAGGAGCCA |
| 229817_at | ZNF608 | −1.0089439 | GTATCAGTGTGCCTGAACCTTGCAT |
| | | | TGAACCTTGCATATCCTTCACATAT |
| | | | TTCCCATAAGCCCCTCAGAAAGGCT |
| | | | TTAGATGTCTATTTGGTGGCTCCTG |
| | | | GTGGCTCCTGTTAAAGACGCACCAG |
| | | | GACGCACCAGTGTAAAATGTTCCTG |
| | | | TCCTGTAGTCACTGTTTGTACTTGT |
| | | | GCATGGGGTTGCCAGTACCACAAAA |
| | | | GAGACATCTGTGATTGTTCTATTAC |
| | | | AGAGAGACTTTAACGCCATTGCCTG |
| | | | GCCATTGCCTGGTTACTTGTTTTAT |
| 232303_at | ZNF608 | −0.71185659 | ATGGCAGTAGCAAGCTTTTCTGTTG |
| | | | AATCTAGTATACCTTGCTTACCCAG |
| | | | TACCTTGCTTACCCAGGAGGATGGT |
| | | | GGAGGATGGTTGTTAGGTGGAAATT |
| | | | TAAATTCATAGGAACCAACTTTTGG |
| | | | TTTGGTAAGTAAGTAGTTCAGAGGC |
| | | | GTTCAGAGGCTACAAACTGTTGACT |
| | | | ATGTTTTCTTGCAGGATACCTTTTA |
| | | | TTACATGCAAGTTCAGATCACCTCT |
| | | | AAATCTGGGCCGGGAGTGAGCCACT |
| | | | GTAGCCTAGTGGTAGTGGGCACCTG |

TABLE 2
Patient and Tumor Characteristics of Patients with
Estrogen Receptor α positive breast cancers.

| Characteristic | Loi | Buffa | Wang |
|---|---|---|---|
| Sample size | 250 | 134 | 209 |
| Age, years | | | |
| Median (SD) | 63 (10) | 57 (10) | 54 (12) |
| Histologic | | | |
| Grade | | | |
| 1 | 47 | 30 | |
| 2 | 128 | 64 | |
| 3 | 40 | 27 | |
| Unknown | 35 | 13 | 209 |
| Tumor stage | | | |
| T1 | 108 | 55 | |
| T2 | 136 | 70 | |
| T3 | 6 | 9 | |
| Unknown | | | 209 |
| Lymph node | | | |
| status | | | |
| Negative | 110 | 78 | 209 |
| Positive | 132 | 56 | |
| Unknown | 8 | | |
| Adjuvant | Yes | Yes | No |
| tamoxifen | | | |
| Median follow- | 7 | 10 | 7 |
| up (years) | | | |

### EXAMPLES

### Example 1

### A shRNA Screen Identifies USP9X as a Tamoxifen Resistance Gene

### Materials and Methods

### Cell Lines and Culture Conditions

The human breast cancer cell lines ZR-75-1 (ATCC CRL-1500), MDA-MB-231 (ATCC HTB-26) and T47D (ATCC HTB133) were cultured in DMEM supplemented with 10% FCS, 2 mM glutamine, 100 μg/ml penicillin, 100 μg/ml streptomycin, and 1 nM estradiol at 37° C. in 5% CO2. In proliferation assays, estradiol was replaced by DMSO (vehicle), 1 μM 4OHtamoxifen (hereinafter: tamoxifen) or 10-7 M fulvestrant. Phoenix cells (ATCC CRL-3214) were cultured at 37° C. in 5% CO2 in DMEM with 10% FCS, 2 mM glutamine, 100 μg/ml penicillin, and 100 μg/ml streptomycin.

### Transfection and Retroviral Infection

Phoenix cells were transfected using calcium phosphate method. Viral supernatant was cleared through a 0.45 μm filter. Target cells were infected with the viral supernatant in the presence of polybrene (8 μg/ml) and the infection was repeated once. For transient transfection of ZR-75-1 cells Lipofectamine 2000 (Invitrogen) was used, according to the manufacturers protocol.

NKI shRNA Library

The construction of the library was described previously (Berns et al., 2004. Nature 428: 431-7). Briefly, the NKI shRNA library was designed to target 7914 human genes, using three shRNA vectors for every targeted gene. The shRNAs are cloned into a retroviral vector (pRetroSUPER (pRS)) to enable infection of target cells.

### Colony Formation Assay

Cells were infected with retroviral supernatant and selected with puromycin (2.0 μg/ml). When the selection was completed 5×104 cells were seeded in 10 cm dishes and cultured in DMEM with 1 μM 4OH-tamoxifen for 4-6 weeks. When colonies appeared, cells were fixed in MeOH/HAc (3:1) and subsequently stained with 50% MeOH/10% HAc/0.1% Coomassie.

shRNA Screen and Recovery of shRNA Inserts

ZR-75-1 cells stably expressing the murine ecotropic receptor were infected with retroviral supernatants containing a selection of the NKI pRS-shRNA library (12,540 shRNA vectors targeting 4180 genes divided in 44 pools—each pool contains 285 distinct short hairpin RNA's against 95 genes) or pRS as control (Berns et al., 2004. Nature 428: 431-7). After puromycin selection (2 μg/ml) 2×105 cells of each pool and control were plated in 15 cm dishes and cultured in DMEM with 1 μM 4OHtamoxifen for 4-6 weeks. Individual colonies that grew out in the presence of tamoxifen were isolated and expanded. Genomic DNA was isolated using DNAzol (Life Technologies). PCR amplification of the shRNA inserts was performed with Expand Long Template PCR system (Roche) and the use of pRS-fw primer: 5′-CCCTTGAACCTCCTCGTTCGACC-3′ and pRS-rev primer: 5′-GAGACGTGCTACTTCCATTTGTC-3′. Products were digested with EcoRI/XhoI and recloned into pRS. Hairpins were sequenced with Big Dye Terminator (Perkin Elmer) using pRS-seq primer: 5′-GCTGACGTCATCAACCCGCT-3′.

### Constructs

For retroviral transduction of human breast cancer cells, ZR-75-1 cells and T47D cells were transfected with pBabeHygro-Ecotropic Receptor and selected with hygromycin (100 μg/ml) and subsequently infected with the supernatant of the Phoenix ecotrophic virus packaging cell line.

The short hairpin sequence targeting USP9X recovered from the NKI shRNA library was:

GAACAGGAGAAACGGGTAT

For the generation of additional shRNA vectors targeting USP9X the following 19-mer sequences cloned in pRetroSuper were used:

| | | |
|---|---|---|
| USP9X II | 1800-1818 | GGAAATGCTTAGCTGAGAA |
| USP9X III | 2725-2743 | CCATGGTAATCATTACAGT |
| USP9X IV | 3601-3619 | CGAACAGGTTTGCTGTGAA |
| USP9X V | 4483-4501 | CTACATGATTCCTTCCATT |
| USP9X VI | 5020-5038 | GGAACAGTATGTCAAAGGA |

### Results

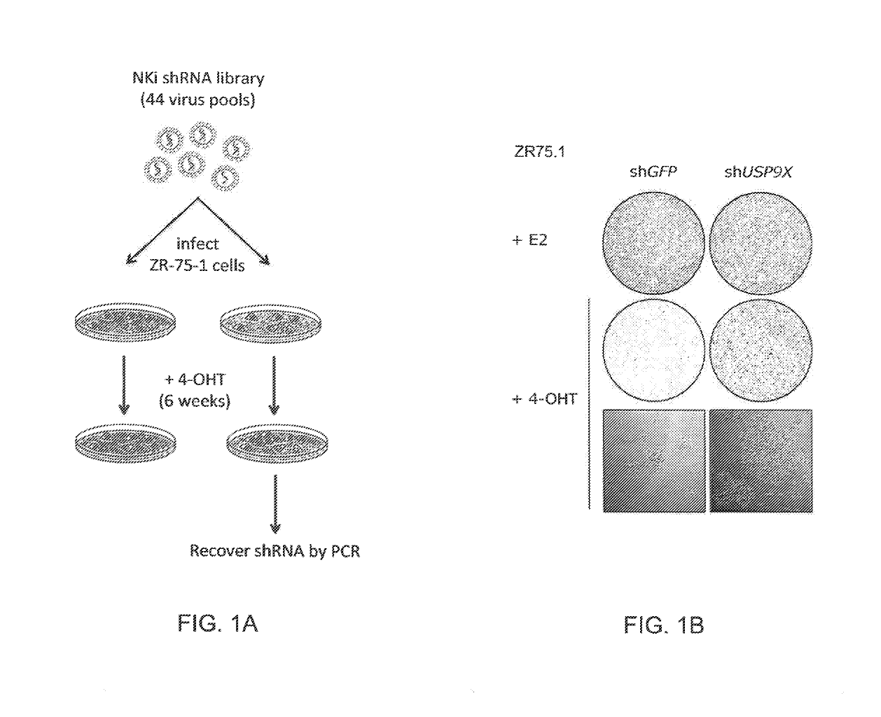

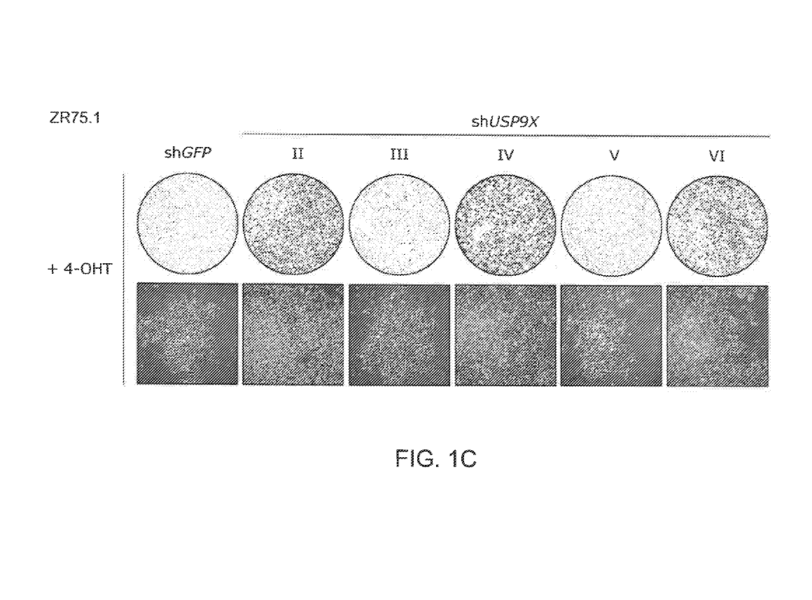

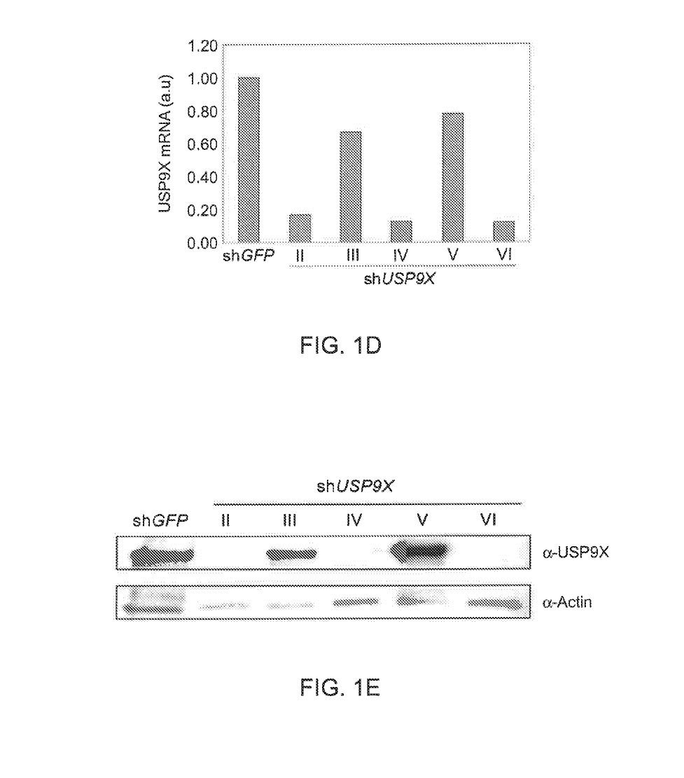

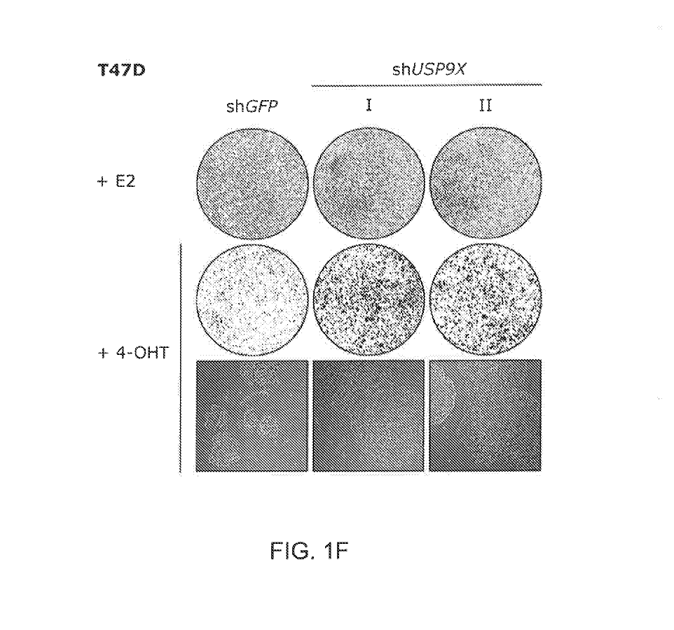

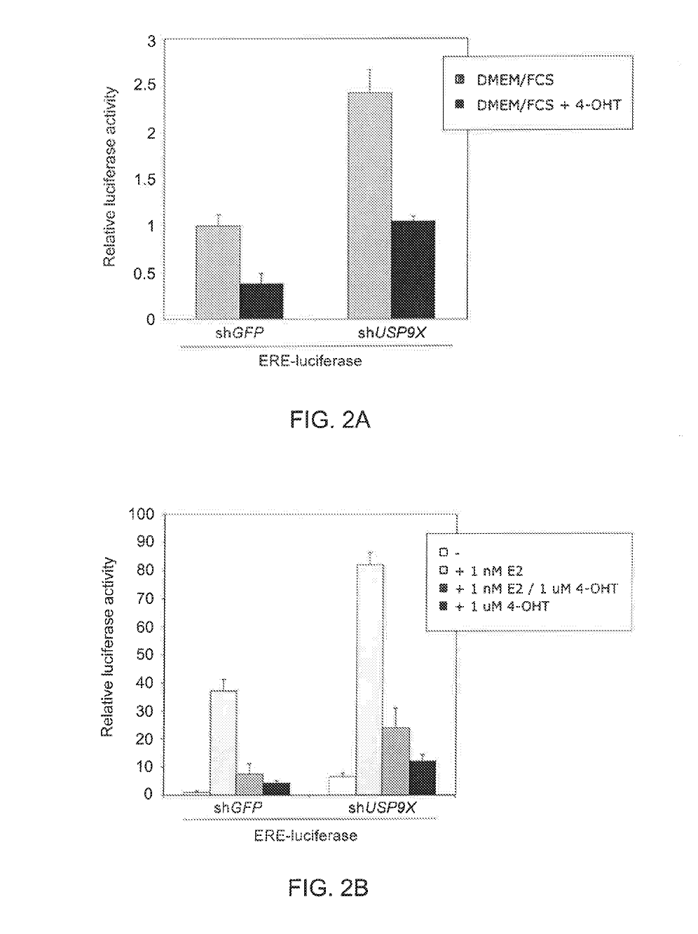

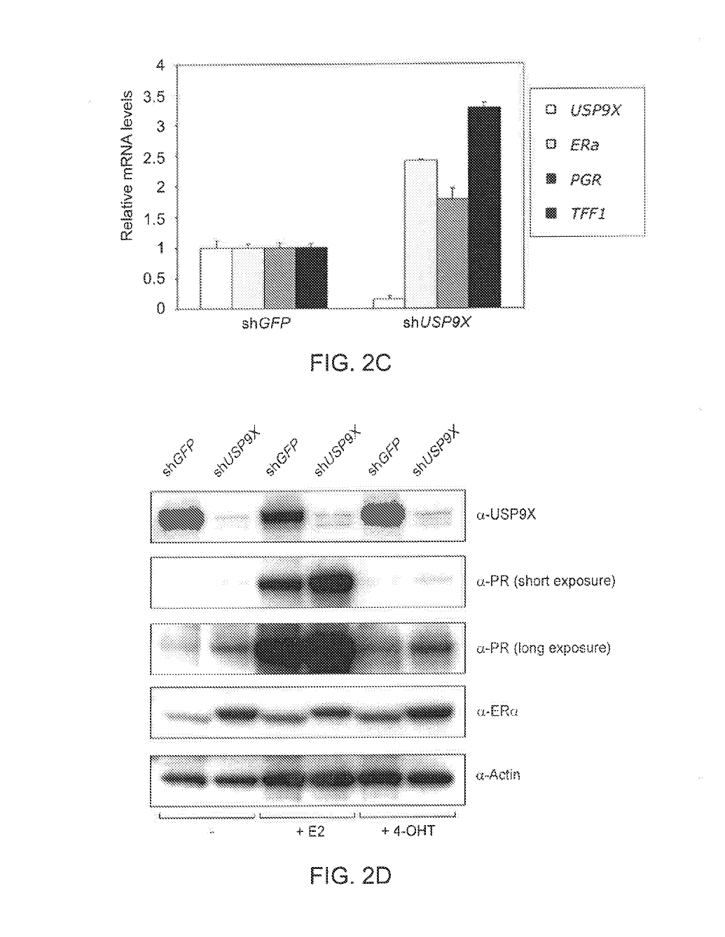

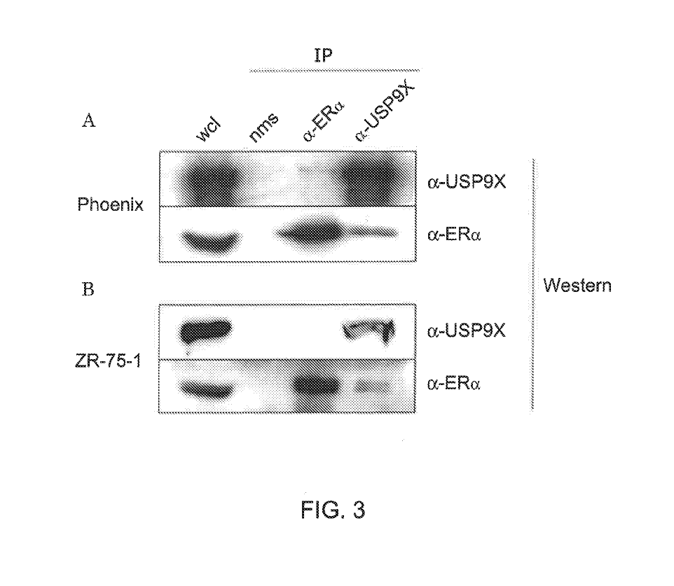

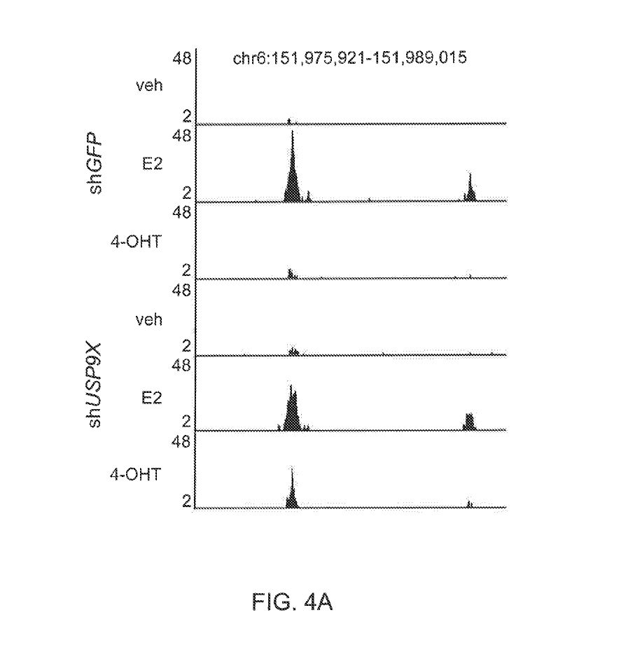

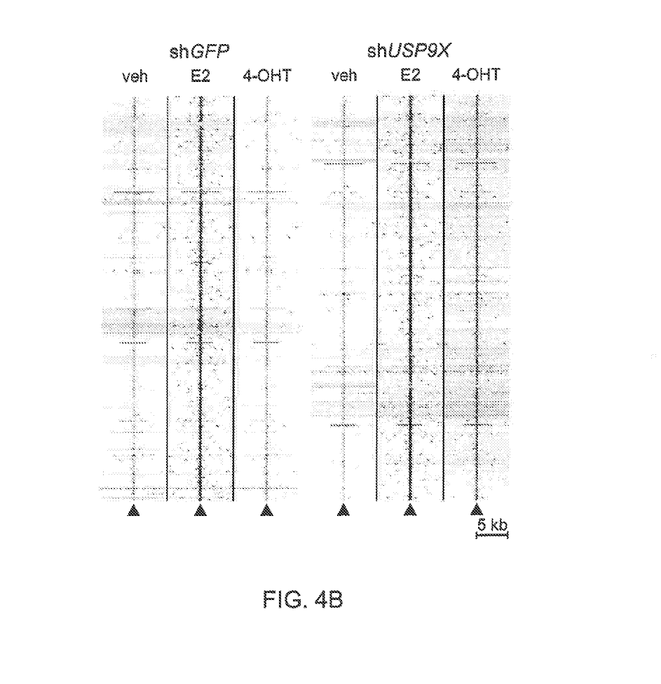

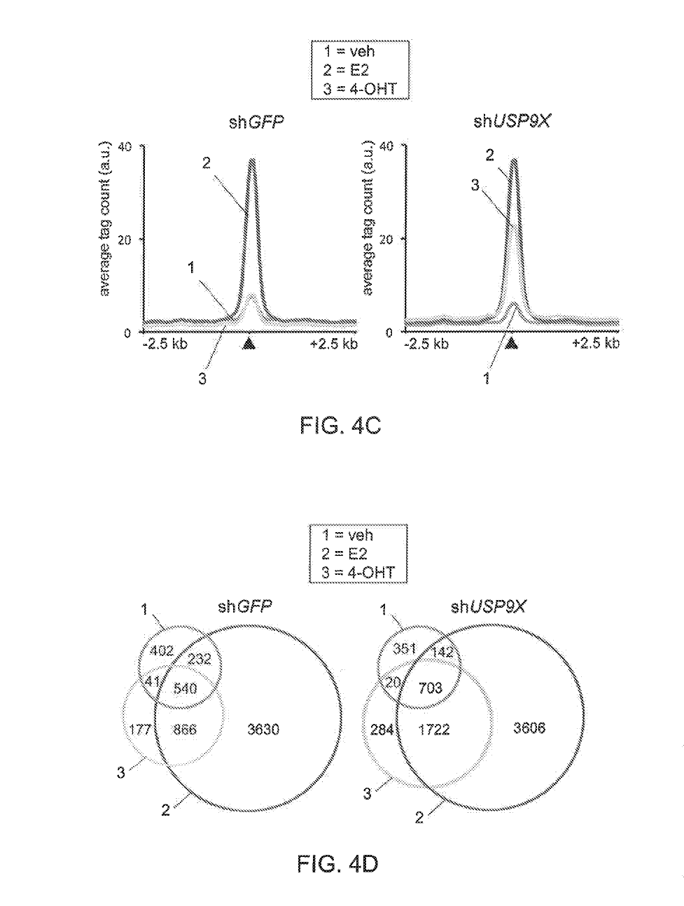

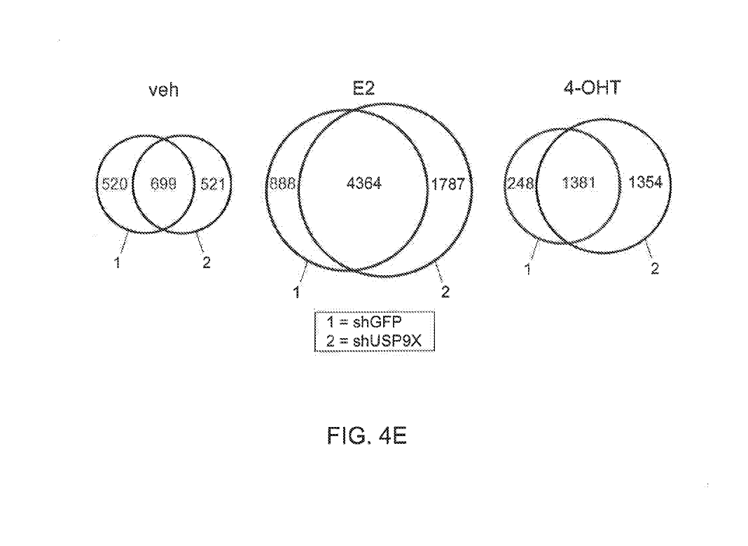

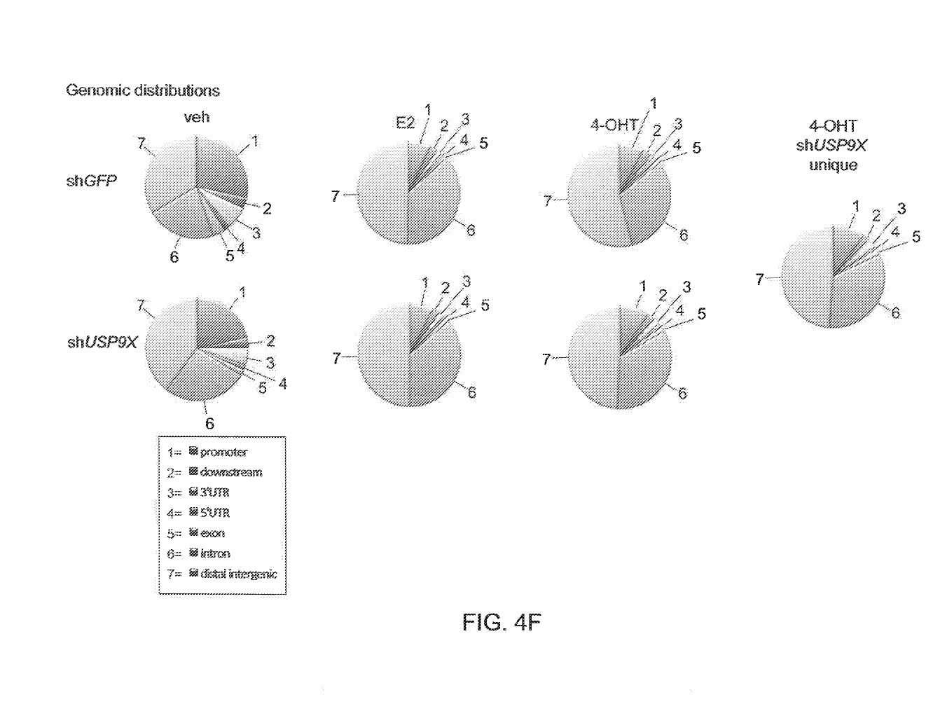

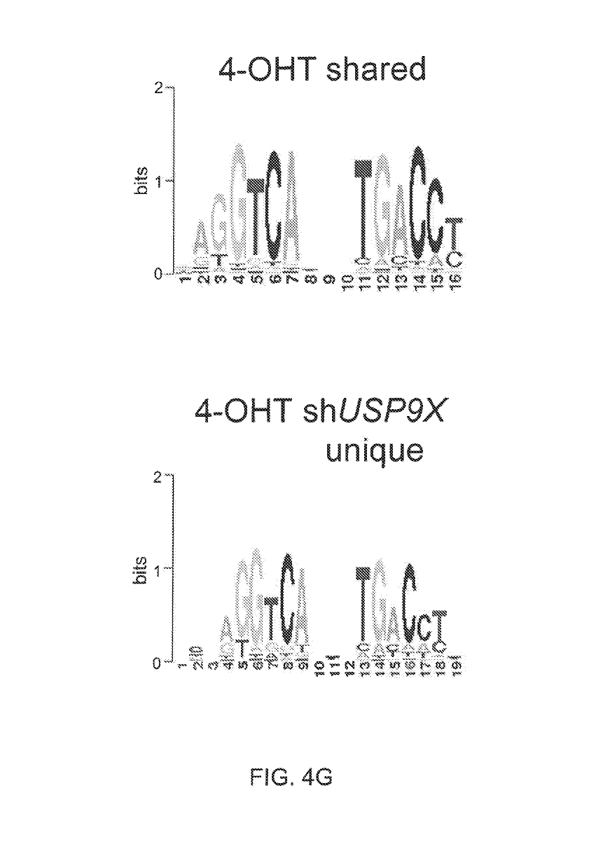

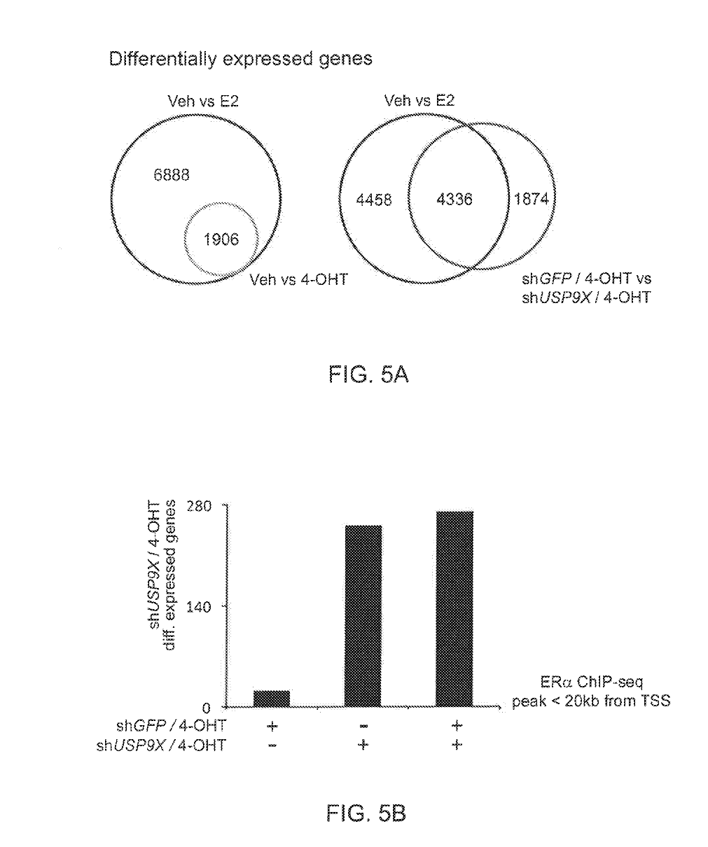

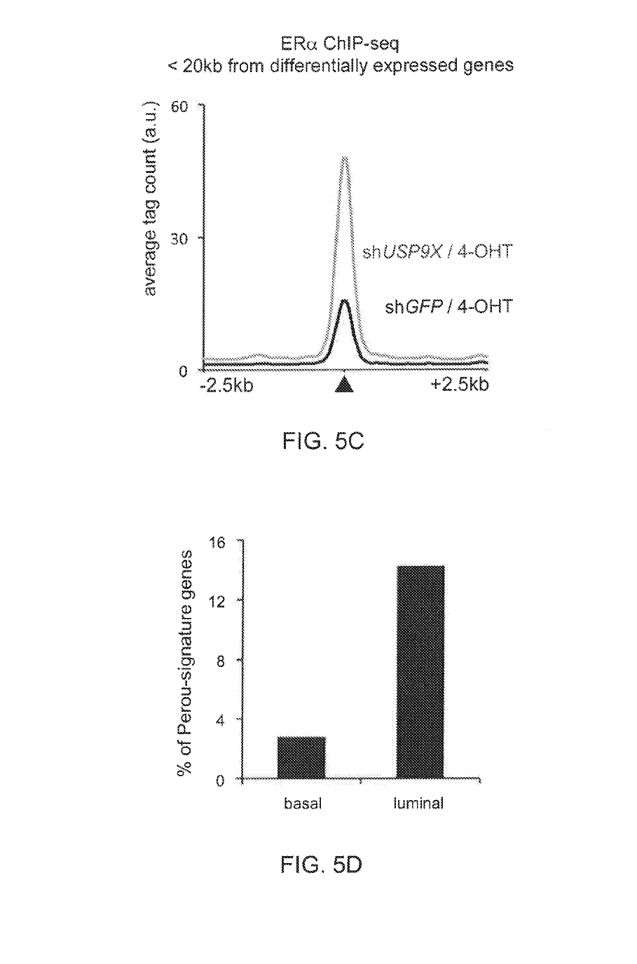

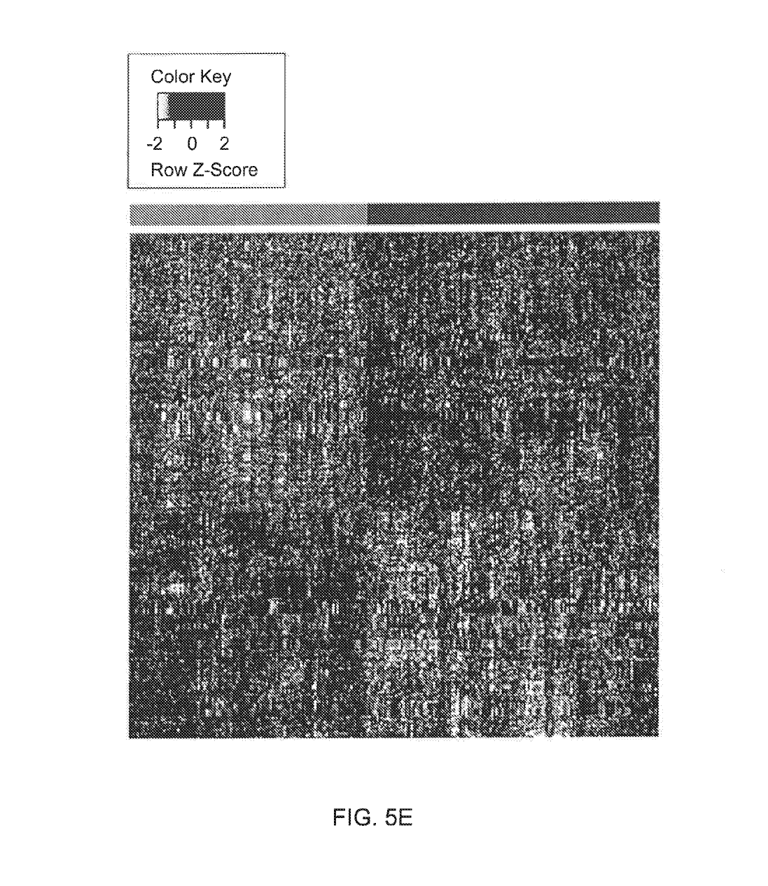

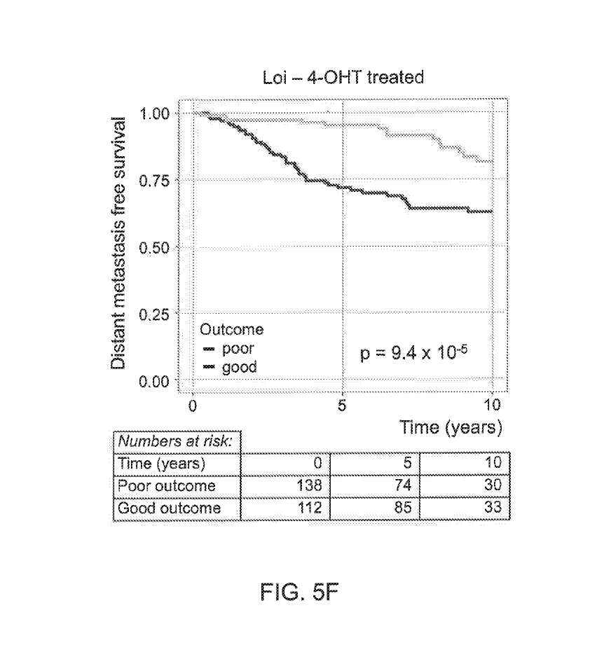

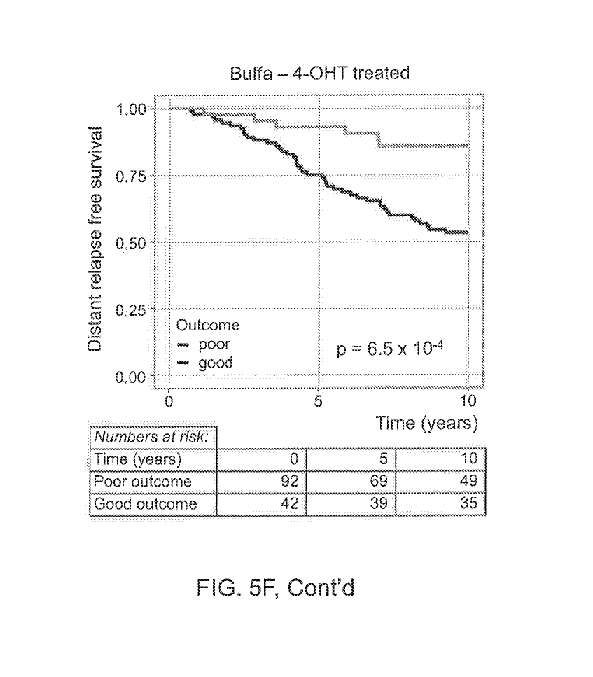

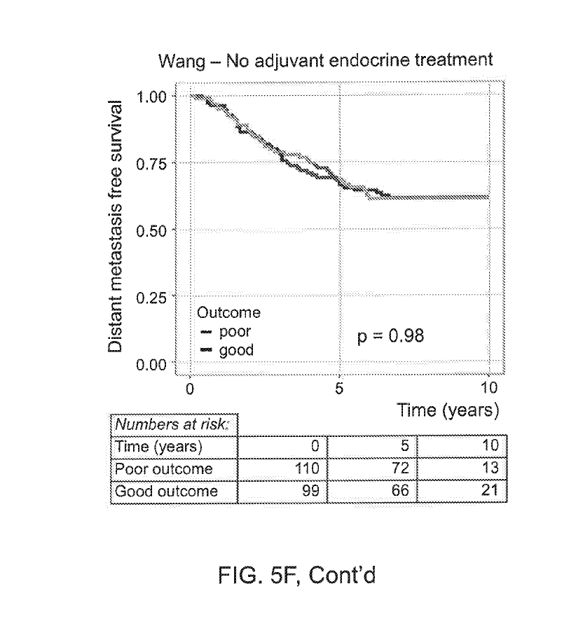

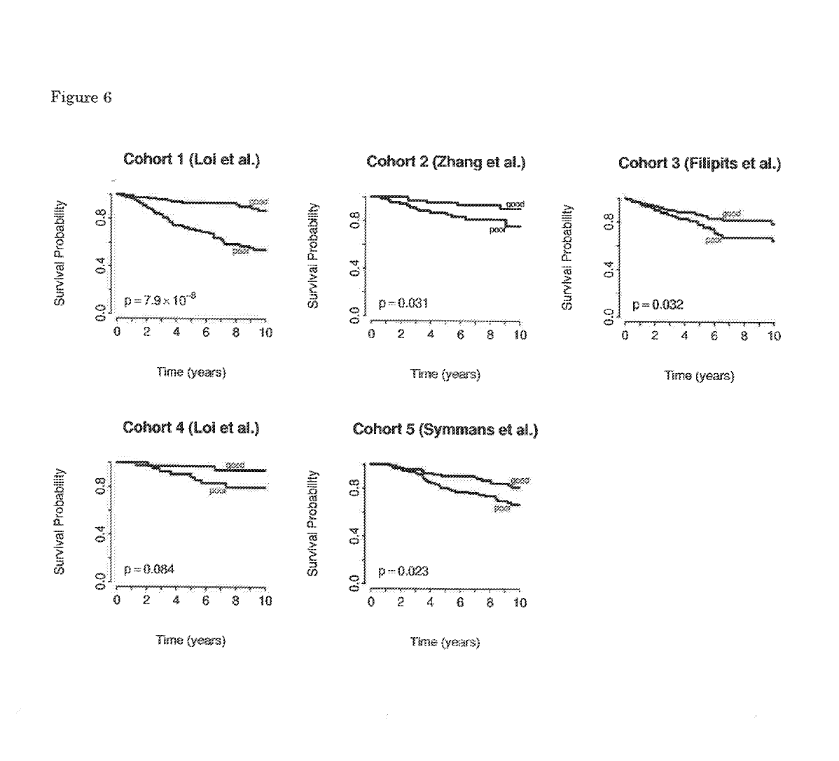

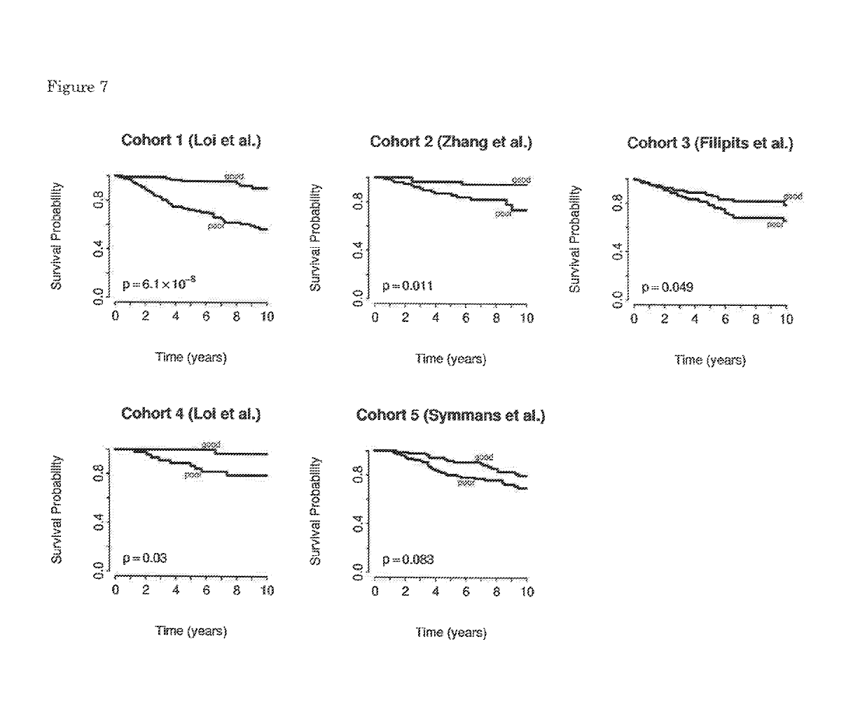

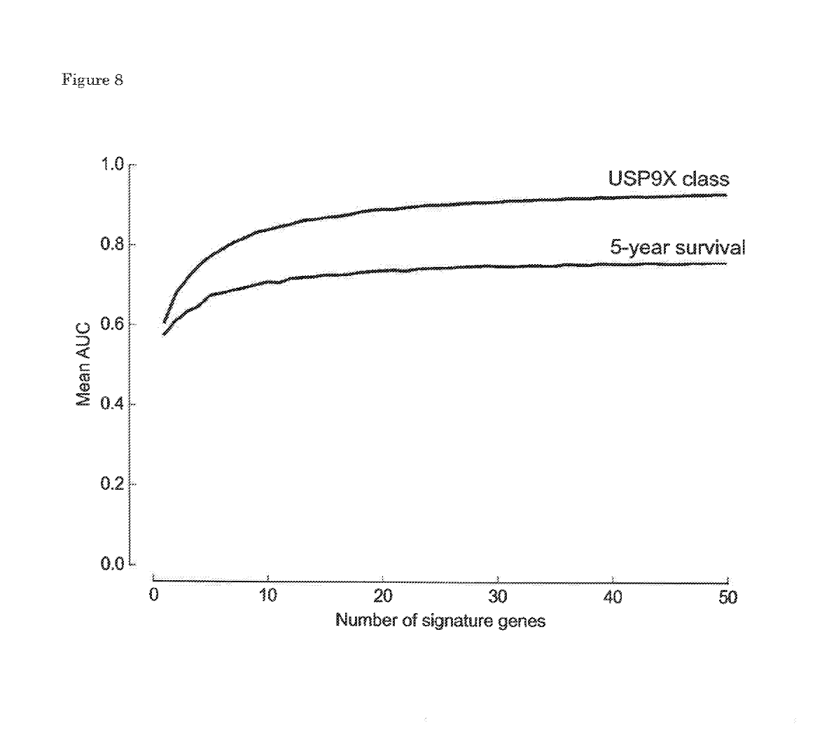

To identify genes causally involved in tamoxifen resistance, a loss-of-function genetic screen was performed in ZR-75-1 luminal breast cancer cells. We first stably expressed the murine ecotropic receptor (Scholz and Beato, 1996. Nucleic Acids Res 24: 979-980) in these cells and subsequently infected them with retroviral supernatants containing a selection of the NM pRS-shRNA library (12,540 shRNA vectors targeting 4180 genes) or pRS as control (Berns et al., 2004. Nature 428: 431-7) (FIG. 1A). Library-infected cells and control cells were plated at low density and cultured in DMEM with 1 μM 4OH-tamoxifen for 4-6 weeks. Individual colonies that grew out in the presence of tamoxifen were isolated and shRNA inserts of the vectors were recovered by PCR. These shRNA inserts were subsequently re-cloned and identified through DNA sequence analysis. This approach resulted the identification of USP9X, as a candidate tamoxifen resistance gene. A colony formation assay in ZR-75-1 cells (FIG. 1B) was performed with the shRNA identified in the screen to confirm the rescue from tamoxifen induced proliferation arrest.

To investigate whether the escape from tamoxifen induced proliferation arrest was the result of an “on target effect of the shRNA”, 5 additional shRNAs targeting different regions of the USP9X gene were designed and tested for their ability to confer tamoxifen resistance. FIG. 1C shows that three of these shRNAs had an identical phenotype to the original shRNA vector as cells grew out in the presence of tamoxifen treatment. Importantly, only the vectors that suppressed USP9X mRNA (FIG. 1D) and protein levels (FIG. 1E) induced tamoxifen resistance. To ask whether the rescue from tamoxifen induced proliferation arrest is independent of cellular context, we also tested two USP9X shRNA vectors for their ability to confer tamoxifen resistance in a second luminal breast cancer cell line: T47D. FIG. 1F shows that knockdown of USP9X in T47D cells enabled cell proliferation in the presence of tamoxifen as well, suggesting that USP9X suppression leads to tamoxifen resistance independent of the cellular context.

Importantly, knockdown of USP9X did not rescue cells from a proliferation arrest induced by the estrogen receptor downregulator fulvestrant, illustrating that shUSP9X-effects on cell proliferation are ERα-dependent (data not shown). In line with these data, knockdown of USP9X in the ERα negative cell line MDA-MB-231 did not induce cell proliferation, even resulting in a growth disadvantage in these cells (not shown).

### Example 2

### Knockdown of USP9X Increases ERα Activity

### Material and Methods

### Luciferase Assay

Monoclonal cell lines stably expressing pRS-USP9X or pRS-GFP as control were plated in triplicate in 6 wells plates in regular DMEM. The next morning cells were washed with PBS and fresh DMEM+10% FCS without Pen/Strep was added followed by Lipofectamine (Invitrogen) transfection according to the manufactures protocol with 1.75 μg ERE-TATA luciferase reporter plasmid vector and 0.5 μg pRL-CMV Renilla luciferase (Promega) per well. Eight hours after transfection cells were washed with PBS and supplied with fresh fenol red free DMEM with 10% charcoal stripped serum or DMEM with 10% FCS. 24 hours after transfection medium was refreshed with ligands as indicated and 48 hours after transfection cells were lysed with passive lysis buffer and the luciferase reaction was performed conform the manufactures protocol (Dual Luciferase Reporter Assay System, Promega). The Renilla luciferase activity was used to correct for differences in transfection efficiency. The relative reporter activity in the absence of ligand was used as a reference and set at 1.

### QRT-PCR (Quantitative Real Time PCR)

Total RNA was isolated using TRIzol (Invitrogen) or using the Quick RNA MiniPrep kit (R1055 Zymo Research). From the total RNA, cDNA was generated using Superscript II (Invitrogen) with random hexamer primers (Invitrogen). cDNA was diluted and the QRT reaction was performed using SYBR green PCR master mix (Applied Biosystems). All QRT reactions were run in parallel for GAPDH to control for amount cDNA input. The QRT reaction was followed by a melting curve to confirm the formation of a single PCR product. The QRT reactions were run at an AB7500 Fast Real Time PCR system (Applied Biosystems). The following PCR primer sequences were used:

| | |
|---|---|
| GAPD-81FW | AAG GTG AAG GTC GGA GTC AA |
| GAPD-188RV | AAT GAA GGG GTC ATT GAT GG |
| ESR1-120FW | ATG ATC AAC TGG GCG AAG AG |
| ESR1-212RV | CAG GAT CTC TAG CCA GGC AC |
| PGR-101FW | GTC CTT ACC TGT GGG AGC TG |
| PGR-191REV | CGA TGC AGT CAT TTC TTC CA |
| TFF1-51FW | GGA GAA CAA GGT GAT CTG CG |
| TTF1-160REV | AAT TCT GTC TTT CAC GGG GG |

### Results

Next we examined whether the rescue from tamoxifen-induced proliferation arrest was the result of increased ERα signaling. Therefore, ZR-75-1 cell lines stably expressing pRSUSP9X or control pRS-GFP were created. First, we tested whether knockdown of USP9X increased ERα activity, as judged by the activity of a reporter construct having Estrogen Responsive Elements linked to luciferase (ERE-luciferase), under the conditions used in the shRNA screen. FIG. 2A shows that USP9X knockdown (USP9XKD) cells have increased ERα transcriptional activity, both when cultured in normal culture media and when cultured in the presence of 4OH-tamoxifen. To rule out a residual effect of estradiol seen when cultured in regular DMEM with 4OH-tamoxifen (as fetal calf serum contains small amounts of estradiol, and the phenol red dye in the culture media has been shown to have weak estrogenic activity), we performed luciferase assays after 24 hours of serum starvation of cells in phenol red-free DMEM supplemented with 10% charcoal stripped (and hence steroid-free) serum, followed by 24 hours of treatment with either estradiol, estradiol+4OH-tamoxifen or 4OHtamoxifen alone. FIG. 2B shows that under all these conditions ERα signaling is about 2.5 times higher in the USP9XKD cell line as compared to the control cell line. Knockdown of USP9X also resulted in increased mRNA levels (FIG. 2C) and protein levels (FIG. 2D) of the ERα target genes Progesterone Receptor (PR), Trefoil factor 1 (TFF1/PS2) and of ERα itself (Eeckhoute et al., 2007. Cancer Res 67: 6477-83).

### Example 3

### Physical Interactions Between USP9X and ERα

### Materials and Methods

### Immunoprecipitation and Immunoblotting

For immunoprecipitation cells were lysed in ELB containing 250 mM NaCl, 0.1% NP-40, 50 mM Hepes pH 7.3, and Complete protease inhibitor cocktail from Roche. Supernatants of the lysates were incubated with either anti-USP9X (clone 1C4; Abnova/Sigma Aldrich), or anti-ERα (D-12; Santa Cruz) coupled to protein A/G sepharose beads. Normal mouse serum coupled to protein A/G sepharose beads was used as control. For Western blotting antibodies were used detecting USP9X (clone 1C4; Abnova/Sigma Aldrich), ERα (clone 1D5; Dako), Progesterone Receptor (clone 1A6; Novocastra), and beta-actin (clone AC-74; Sigma Aldrich A 5316).

### Results

Given the functional interaction between USP9X and ERα, we next tested whether ERα and USP9X physically interact. We expressed human ERα in Phoenix cells. Cells were lysed in mild detergent and the lysate was immunoprecipitated with anti-USP9X antibody or anti-ERα antibody and Western blotting was performed. As shown in FIG. 3A, exogenously expressed human ERα forms a complex with endogenous USP9X. Importantly, FIG. 3B shows that in the ERα-positive ZR-75-1 cells endogenous ERα also co-immunoprecipitates with endogenous USP9X, demonstrating the existence of a physical complex of these proteins under physiological conditions, which was recently also shown by mass spectrometry by Stanisic et al. (Stanisic et al., 2009. J Biol Chem 284: 16135-45).

### Example 4

### USP9X Loss Selectively Enhances ERα/Chromatin Interactions Upon 4OH-Tamoxifen Treatment

### Materials and Methods

### Chromatin Immunoprecipitations

Chromatin Immunoprecipitations (ChIP) were performed as described before (Schmidt et al., 2009. Methods 48: 240-8). For each ChIP, 10 μg of antibody was used, and 100 μl of Protein A magnetic beads (Invitrogen). The antibody used was raised against ERα (SC-543; Santa Cruz).

### Next Gen Sequencing and Enrichment Analysis

ChIP DNA was amplified as described (Schmidt et al., 2009. Methods 48: 240-8). Sequences were generated by the Illumina Hiseq 2000 genome analyser (using 50 bp reads), and aligned to the Human Reference Genome (assembly hg19, February 2009). Enriched regions of the genome were identified by comparing the ChIP samples to mixed input using the MACS peak caller (Zhang et al., 2008. Genome Biol 9: R137) version 1.3.7.1.

### Motif Analysis, Heatmaps and Genomic Distributions of Binding Events

ChIP-seq data snapshots were generated using the Integrative Genome Viewer IGV 2.1 (www.broadinstitute.org/igv/). Motif analyses were performed through the Cistrome (cistrome.org), applying the SeqPos motif tool (He et al., 2010. Nat Genet 42: 343-7). The genomic distributions of binding sites were analysed using the cis-regulatory element annotation system (CEAS) (Ji et al., 2006. Nucleic Acids Res 34: W551-4). The genes closest to the binding site on both strands were analysed. If the binding region is within a gene, CEAS software indicates whether it is in a 5′UTR, a 3′UTR, a coding exon, or an intron. Promoter is defined as 3 kb upstream from RefSeq 5′ start. If a binding site is >3 kb away from the RefSeq transcription start site, it is considered distal intergenic.

### Statistical Analysis

Normalised mRNA expression data for three patient series were downloaded from GEO: GSE6532 (Loi et al., 2007. J Clin Oncol 25: 1239-46), GSE22219 (Buffa et al., 2011. Cancer Res 71: 5635-45), and GSE2034 (Wang et al., 2005. Lancet 365: 671-9). From these, two sets of ERα-positive, tamoxifen-treated patients (Loi, n=250; Buffa, n=134), and one set of ERα-positive untreated patients (Wang, n=209) were extracted, for which followup was available. Probes in the Buffa and Wang data were median-centered before further processing. The Loi data had already been median-centered. The 526 genes of the USP9X knockdown tamoxifen signature were mapped to the corresponding microarray platforms by selecting all probes for matching genes, and ignoring genes not present on the array. For the Loi data, this selected 949 probe sets represent 488 different genes. For the Buffa data, 363 probes were selected representing 295 genes and for the Wang data, 792 probe sets representing 391 genes were available. 254 of the signature genes were present on all three array platforms. Patients were stratified into two groups by applying a hierarchical complete linkage clustering using Pearson correlation distance, and dividing by the first split of the clustering. Significant differences in distant metastasis free survival time between these two groups were tested for using the log-rank test. Survival times longer than ten years were right-censored. The array platform used for the untreated Wang data provides a subset of the probes available for the treated Loi data (792 out of 949).

To verify that this difference does not affect the comparison between treated and untreated, the Loi samples were additionally clustered based on this subset only. This clustering was found still to stratify patients according to prognosis (log-rank p=1.3×10-5). The directionality of USP9X knockdown tamoxifen classification genes in the good and poor outcome patient groups is shown in Table 1.

### Results

### Knockdown of USP9X Give Rise to Both Tamoxifen Resistance and ERα-Responsive Gene Activation.

The effects of USP9X knockdown on ERα/chromatin interactions were tested for hormone-depleted (vehicle), estradiol and tamoxifen-conditions, using chromatin immunoprecipitation, followed by high-throughput sequencing (ChIP-seq). ZR-75-1 cell lines stably expressing pRS-USP9X or pRS-GFP (control) were plated in hormone depleted medium for 72 hours. Typically, ERα ChIP-seq experiments are performed after a treatment for 45 minutes with ligand (Carroll et al., 2005. Cell 122: 33-43; Hurtado et al., 2011. Nat Genet 43: 27-33). Since USP9X suppression causes long-term resistance to tamoxifen, we were interested in ERα biology after prolonged ligand treatment and the effects of USP9X knockdown thereon. Therefore, the cells were treated with vehicle, estradiol or 4OH-tamoxifen for 48 hours before the ChIP assay.

In control cells, estradiol treatment greatly enhanced ERα/chromatin interactions, while this was far less pronounced when treating the cells with 4OH-tamoxifen. USP9X knockdown had no effect on ERα/chromatin interactions in vehicle and estradiol treated cells, but significantly increased chromatin binding intensity upon 4OH-tamoxifen treatment as exemplified in FIG. 4A. The stabilization of ERα/chromatin interactions in the presence of 4OH-tamoxifen could be generalized throughout the genome, as depicted in a heat map visualization (FIG. 4B) and expressed in a quantified format in a 2D graph (FIG. 4C). This increased intensity of ERα/chromatin interactions in 4OH-tamoxifen-treated cells also translated into a significant increase in the number of chromatin binding events, representing a subset of the estradiol-induced binding patterns under the same conditions (FIG. 4D). Comparing control with USP9XKD under the same ligand conditions showed a relative selectivity for gained sites, both for estradiol and 4OH-tamoxifen conditions, while this was not the case for vehicle-treated cells (FIG. 4E).

ERα rarely binds promoters (5%), and the vast majority of ERα binding events are found at distal enhancers (Carroll et al., 2005. Cell 122: 33-43.38). We could confirm these data for estradiol and 4OH-tamoxifen conditions, both in control and USP9XKD cells (FIG. 4F). Vehicle-treated cells showed enrichment of ERα binding to promoters as was found before (Zwart et al., 2011. EMBO J 30: 4764-76), which was not influenced by knockdown of USP9X. The gained ERα binding events for USP9XKD cells under tamoxifen conditions showed identical distributions as found for estradiol and tamoxifen-treated control cells. De novo DNA motif enrichment analyses provided ESR motifs, and ERα binding sites that were selectively induced by USP9X knockdown in the presence of 4OH-tamoxifen, and were practically identical to those shared between control cells and USP9XKD cells (FIG. 4G).

Collectively, these data show that USP9X knockdown induces ERα binding events, selectively in the presence of 4OH-tamoxifen, that represent a subset of estradiol-induced sites and do not deviate in normal ERα behaviour with respect to genomic distributions and DNA motif enrichment.

### Example 5

### RNA Expression Analysis

Transcriptome sequencing analysis of the cell line ZR-75-1 with stable USP9X knockdown or a control vector were performed using RNA-Seq. The reads (14-30 million 50 bp single-end) were mapped to the human reference genome (hg19) using TopHat (Trapnell et al., 2009. Bioinformatics 25: 1105-1127), which allows to span exon-exon splice junctions. TopHat was supplied with a known set of gene models (Ensembl version 64). The open-source tool HTSeq-Count was used to obtain gene expressions. This tool generates a list of the total number of uniquely mapped sequencing reads for each gene that is present in the provided Gene Transfer Format (GTF) file. In order to identify differentially expressed genes, the random sampling model in the R package DEGseq (Wang et al., 2010. Bioinformatics 26: 136-8.28) was used. We have taken a p-value of 0.05 as a cut-off to determine whether a gene is significantly differentially expressed. The input of this method is the absolute number of reads for a gene, which is the output of HTSeq-count. Genes with no expression across both samples in the comparison were discarded from the dataset. The expression levels of the remaining genes were added with 1 in order to avoid negative values after log 2 transformation during the normalization step within this method.

### Results

### USP9X and Global Gene Expression Analyses

Our ChIP-seq analyses indicate that USP9X knockdown selectively increases ERα/chromatin interactions in the presence of tamoxifen that are normally found for estradiol conditions. We therefore asked whether USP9X knockdown in tamoxifen-treated cells would also give rise to a typical estradiol-responsive gene set. To address this, we performed RNAseq on ZR-75-1 cells stably expressing pRS-USP9X or pRS-GFP (control) that—after hormone depletion for 72 hours—were treated for 48 hours with vehicle, estradiol or 4OHtamoxifen. Comparing gene expression in both cell lines, we found that estradiol-treatment led to an altered expression of 8794 genes as compared to vehicle, while after 4OHtamoxifen treatment 1906 genes were differentially expressed. All altered transcripts under 4OH-tamoxifen conditions represented a subset of the estradiol-responsive genes (FIG. 5A, left panel). 4OH-tamoxifen treatment in USP9XKD cells as compared to 4OH-tamoxifen treated control cells resulted in an altered expression of 6210 transcripts, 4336 of which were shared with estradiol-induction in control cells (FIG. 5A, right panel). The differentially expressed genes in 4OH-tamoxifen-treated USP9XKD cells specifically showed an increase in number (FIG. 5B) and intensity (FIG. 5C) of proximal ERα binding events.

The majority of genes that are differentially expressed upon tamoxifen treatment in the USP9XKD cells were shared with estradiol induction. ERα-positive breast tumors are hallmarked by a selective and specific enrichment of so-called ‘luminal-signature genes’ (Perou et al., 2000. Nature 406: 747-52). Therefore, the USP9X knockdown tamoxifen gene set, which was shared with the estradiol responsive gene list and which also showed enhanced proximal ERα binding events, (526 out of 4336 genes, see FIG. 5B right two columns), was tested for enrichment of ‘luminal’ over ‘basal’ genes, using the genes as defined by Perou et al (Perou et al., 2000. Nature 406: 747-52). A clear enrichment of luminal genes was found relative to basal signature genes, consistent with the notion that USP9X knockdown enhances ERα signaling (FIG. 5D).

### Example 6

### A USP9X Knockdown Tamoxifen Gene Expression Signature Identifies Breast Cancer Patients with a Poor Outcome after Adjuvant Tamoxifen Treatment

The RNA-seq analyses revealed that the majority of genes that were differentially expressed upon tamoxifen treatment in the USP9XKD cells were a subgroup of estradiol induced genes (4336 out of 8794). Furthermore, integrating these results with the ChIP-seq data showed that a subgroup of these genes (526 out of 4336) is enriched for proximal ERα binding events. This particular subgroup of genes is expected to represent a direct ERα target gene signature in contrast to the (potentially indirectly regulated) genes that were not enriched for ERα binding. Since these directly ERα regulated genes would also be the genes that are directly affected under tamoxifen resistant conditions, differential expression of these particular genes in breast tumors could hallmark tamoxifen unresponsiveness.

To test this hypothesis, we investigated whether these genes were differentially expressed in a publically available data set of 250 patients with primary ERα positive breast cancer with known outcome (Loi et al., 2007. J Clin Oncol 25: 1239-46). All these patients received adjuvant tamoxifen. For relevant clinicopathological parameters, see Table 2. As visualised in a heatmap (FIG. 5E), unsupervised clustering on the basis of our gene signature resulted in the identification of two distinct subgroups of patients. These subgroups of patients were subsequently analysed for differential distant metastasis-free survival after adjuvant tamoxifen treatment. FIG. 5F left panel shows that this gene set identifies a subgroup of breast cancer patients with a poor outcome after tamoxifen treatment (p=9.4×10-5). This data could be validated using a second cohort of ERα positive breast cancer patients (n=134) who received adjuvant tamoxifen treatment (Buffa et al., 2011. Cancer Res 71: 5635-45). FIG. 5F, middle panel, shows that our classifier successfully identified tamoxifen-treated breast cancer patients with a poor outcome (p=6.5×10-4). We then tested our signature on a cohort of primary ERα positive breast cancer patients (n=209) (Wang et al., 2005, Lancet 365: 671-9) who did not receive any adjuvant endocrine treatment. Importantly, in these patients, the USP9X knockdown tamoxifen gene expression signature did not correlate with outcome, indicating that the gene signature is not a prognostic signature (FIG. 5F right panel).

### Example 7

### Material and Methods

### Gene Expression Data

Gene expression data from five publically available studies were used for developing or validating the USP9X signature. All cohorts consist of ERα-positive, tamoxifen-treated breast cancer patients. Cohort 1 (GSE6532; Loi et al., 2007. J Clin Oncol 25: 1239-46) was used in our unsupervised clustering analysis to identify the two USP9X clusters. Furthermore, it was also used in the supervised gene selection procedures described below, and will henceforth be referred to as the training data. Data from four other studies were exclusively used for validating the trained classifier: cohort 2 (GSE12093; Zhang et al., 2009. Breast Cancer Res Treat 116: 303-9), cohort 3 (GSE26971; Filipits et al., 2011. Clin Cancer Res 17:6012-20), cohort 4 (GSE9195; Loi et al., 2008. BMC Genomics 9:239), and cohort 5 (GSE17705; Symmans et al., 2010. J Clin Oncol 28:4111-9). The complete data set for cohort 5 includes 102 samples that overlap with cohort 1. For the validation, we removed the overlapping samples from cohort 5.

### Training the USP9X Classifier

The training data were used for supervised training of a classifier that assigns new tumor samples to one of the two USP9X clusters. The two clusters identified by the unsupervised clustering of the training data were used as the gold standard. For training the classifier, we used the nearest shrunken centroid (NSC) method (Tibshirani et al., 2002. Proc Natl Acad Sci USA 99:6567-72). In short, class centroids are estimated based on the within-class means of the signature genes. Then, a shrinkage parameter is tuned to shrink the within-class means towards the overall means per gene. Genes for which the within-class mean is fully shrunk to the overall mean do not discriminate between the two classes, and are therefore not used for classification. Because of this, tuning the shrinkage parameter yields an optimised subset of genes to use for classification. We tuned the NSC shrinkage parameter to maximise the cross-validated area under the ROC curve (AUC), using a 10-fold cross validation (CV) procedure. We tested this gene selection procedure on the training set in a nested cross validation set-up. Within each outer-CV iteration, the shrinkage parameter was tuned on 90% of the training samples using an internal CV as described above. Subsequently, the selected shrinkage parameter was used to classify the remaining 10% of the training samples. The cross-validated AUC of the outer-CV was 0.95, which confirms the validity of the gene selection procedure. Subsequently, we trained the final classifier by estimating class centroids and tuning the shrinkage parameter on the entire training set. The best cross-validated AUC performance was obtained by selecting 155.

### Identification of a Minimal Gene Signature

We looked for even smaller sets of signature genes in a more stringent gene selection procedure. For this, we performed a similar CV procedure as above, but used L1-regularised logistic regression instead of NSC. This choice was made because L1 regularisation generally leads to sparser gene selections. We repeated the CV gene selection procedure 100,000 times, with randomly sampled fold assignments, and kept those genes that were selected in at least 99% of the iterations. Using this procedure, we selected 9 genes. Next, we tested for each subset of these 9 genes, whether clustering on the subset yields two clusters that show a significant difference in survival on the training set. A selection of 5 genes: MYBL2, IDH3A, CHSY1, BUB1B, CAPN2 gave rise to the largest survival differences among all subsets. However, most smaller subsets of these 5 do still separate good from poor survival to a large extent.

### Results

### Validation of the USP9X Classifier in Independent Patient Cohorts

The NSC classifier was trained on the training data, selecting 155 genes in the process. We subsequently used it to classify tumors from cohorts 2, 3, 4, and 5. None of these cohorts was used in training the classifier or selecting the genes. Survival curves for the classifications are shown in FIG. 6. The curves for cohort 1 are based on cross-validated predictions, i.e. the classifier used for classifying a tumor was not trained on data including that same tumor. On all cohorts but cohort 5, the two identified groups show a significant difference in survival. The results for cohort 5 show a strong trend towards significance but are hampered by the small number of events in this cohort.

### Validation of the Minimal Gene Signature in Independent Patient Cohorts

We also validated the minimal, 5 gene signature on the validation cohorts. A nearest centroid classifier for this signature was trained on the training data and subsequently used to classify the tumors in cohorts 2-5. The resulting survival curves are shown in FIG. 7. The performance of the minimal gene signature is mostly comparable to that of the 155-gene NSC classifier, although it is slightly better for some of the validation cohorts, and slightly worse for others.

### Example 8

### Materials and Methods

### Establishing the Minimum Required Number of Genes

To establish the minimum signature size that still allows successful stratification of patients, we randomly sampled smaller subsets of genes, and evaluated their classification performance. For each gene set size between 2 and 50, we drew 200 random subsets from the 155 genes selected for the USP9X classifier. Nearest centroid classifiers based on the random subsets were evaluated in a 10-fold cross validation set-up. Next, the mean of the cross-validated areas under the ROC curve (AUC) were estimated per subset size.

### Results

Mean AUCs per subset size are shown in FIG. 8 for two different evaluation criteria. One criterion is how well the random subsets are able to recover the USP9X classes defined by clustering on the larger signature. For this criterion, a mean AUC of 0.77 is achieved with random subsets of 5 genes. As the subset sizes grow towards 50, the mean AUC converges towards 0.95. The second criterion is how well the predicted classes separate poor survival from good survival. The figure shows the area under the time-dependent ROC curve evaluated at 5 years. With random subsets of 5 genes, an average AUC of 0.67 is achieved.

1-13. (canceled)

14. A method of typing a sample from a breast cancer patient that has been treated with tamoxifen, the method comprising:
determining a level of expression for USP9X and/or for at least two genes that are selected from Table 1 in a relevant sample from the breast cancer patient that has been treated with tamoxifen, whereby the sample comprises expression products from a cancer cell of the patient;
comparing said determined level of expression of USP9X or of the at least two genes to the level of expression of USP9X or the at least two genes in a reference;
typing said sample as being responsive to treatment with tamoxifen or not, based on the comparison of the determined levels of expression.

15. A method of typing a sample from a breast cancer patient according to claim 14, the method comprising:
determining a level of expression for at least two genes that are selected from Table 1 in a relevant sample from the breast cancer patient, whereby the sample comprises expression products from a cancer cell of the patient;
comparing said determined level of expression of the at least two genes to the level of expression of the at least two genes in a reference;
typing said sample as being responsive to treatment with tamoxifen or not, based on the comparison of the determined levels of expression.

16. The method according to claim 14, wherein the reference is a measure of the average level of said at least two genes in at least 10 independent individuals.

17. The method according to claim 14, whereby the sample is typed by determining a level of RNA expression for at least five genes that are selected from Table 1 and comparing said determined RNA level of expression to the level of RNA expression of the at least five genes in a reference.

18. The method according to claim 14, whereby a level of expression of at least ten genes from Table 1 is determined.

19. The method according to claim 14, whereby a level of expression of all genes from Table 1 is determined.

20. A method of assigning anti-estrogen receptor-directed therapy comprising tamoxifen to a breast cancer patient, comprising
typing a sample from the breast cancer patient with a method according to claim 14; and
assigning anti-estrogen receptor-directed therapy comprising tamoxifen to a patient of which the sample is typed as being responsive to treatment with tamoxifen.

21. A method of assigning further antiER directed therapy or chemotherapy to a breast cancer patient, comprising
typing a sample from the breast cancer patient with a method according to claim 14; and
assigning chemotherapy to a patient of which the sample is typed as being non-responsive to treatment with tamoxifen.

22. The method according to claim 21, whereby the further antiER directed therapy comprises the administration of a selective estrogen receptor modulator not being tamoxifen, an aromatase inhibitor, and/or GnRH or a GnRH-analogue.

23. The method according to claim 21, whereby the chemotherapy comprises anastrozole.

24. The method of claim 21, whereby the chemotherapy comprises administration of a platinum agent and/or a PARP inhibitor.

25. The method of claim 21, whereby the chemotherapy comprises cisplatin.

26. The method of claim 21, whereby the chemotherapy comprises ABT-888
